SEQUENCE LISTING (107 sequences)

SEQ ID NO: 1 - DNA, 3596 bp, synthetic construct
  source 1..3596: mol_type = other DNA
ggcactcatacatacactctggcaatgaggacactgagctcgcttctgaaatttgacaagataaccactaaaatctctttgaattctatgttgttgtgatcccatggctacagaggatcaggagttgacatagatactctttggatttcataccatgtggaggctttcttacttccacgtgaccttgactgagttttgaatagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 2 - protein, 805 aa, synthetic construct [sequence omitted]

SEQ ID NO: 3 - DNA, 1680 bp, synthetic construct
  source 1..1680: mol_type = other DNA
ctggccttaccagtgaccgccttgctcctgccgctggccttgctgctccacgccgccaggccgggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatcggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 340 aa, synthetic construct
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRI

SEQ ID NO: 5 - DNA, 2421 bp, synthetic construct
  source 1..2421: mol_type = other DNA
atggccttaccagtgaccgccttgctcctgccgctggccttgctgctccacgccgccaggccgggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggaggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - protein, 587 aa, synthetic construct
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 7 - DNA, 2655 bp, synthetic construct
  source 1..2655: mol_type = other DNA
atggccttaccagtgaccgccttgctcctgccgctggccttgctgctccacgccgccaggccgggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 722 aa, synthetic construct
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 9 - DNA, 246 bp, synthetic construct
  source 1..246: mol_type = other DNA
gacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 10 - protein, 82 aa, synthetic construct
DTRKMDPSKPSSNVAGVVIIVILLILTGAGLAAYFFYKKRRVHLPQEGAFENTLYFNSQSSPGTSDMKDLVGNIEQNEHSVI

SEQ ID NO: 11 - DNA, 288 bp, synthetic construct
  source 1..288: mol_type = other DNA
attgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 12 - protein, 96 aa, synthetic construct
IDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 13 - DNA, 1482 bp, synthetic construct
  source 1..1482: mol_type = other DNA
tttggatgtttttgtggtttcatcctcatcggtttgatattgtacataagtctggcgataaggaagagagttcaagagacaaagttcggaaatgcctttacagaggaagacagtgagctcgttgtaaactacatcgcaaaaaaaagcttctgtagaagagcaatagagctcacgttgcactcactcggtgtgtccgaagaactccagaataaactggaagacgtcgttatcgatcggaacctcctcatacttggaaaaatactgggagaaggagagttcggcagtgtcatggaaggtaacttgaaacaagaggatggtacctcactcaaggtagctgtcaagacgatgaaacttgataacagttcacaaagggagatcgaagaatttctgtctgaggccgcctgtatgaaagacttctcacatcctaatgtcatcagacttcttggcgtttgtatcgagatgtctagccaaggaatcccaaaacctatggtcatattgcctttcatgaaatatggcgatctgcatacatatttgctctactctagacttgagacagggcccaaacatattcctctccagacattgctcaagtttatggtcgatattgccctgggtatggagtacttgagcaaccgaaattttctgcatcgggatcttgccgcacgcaactgcatgctgcgcgatgacatgaccgtctgcgtggctgattttgggctgtcaaaaaaaatatattctggagactactaccgacaagggcggattgcaaagatgcccgtcaaatggattgcgattgaaagtttggcggacagggtatatacttccaaatcagatgtttgggcttttggagtcactatgtgggaaatagctacacgcggtatgaccccgtaccccggagtacaaaatcatgaaatgtatgactatctccttcatggacacaggctgaagcagcccgaggactgcctggacgaactgtatgaaataatgtattcttgttggcgaaccgatcccttggaccggcctactttcagtgtccttagattgcaacttgagaaattgctcgagtctttgccggatgtgcgaaaccaggcagacgtgatctatgtcaatacccaacttttggaaagttctgagggcctcgcacagggttctacccttgccccgttggatcttaacatagacccagacagcataattgcttcttgtacacctcgcgctgccatatcagttgtaacagcggaggtccatgatagtaaacctcacgagggtcgctatatcctgaacggcgggtcagaagaatgggaagacctgacatcagcgccgagcgccgccgttactgctgagaaaaactctgtcctgcccggagagcgcttggttcggaacggggtaagttggagccatagctcaatgctccccctgggttcaagtctcccggacgagcttctttttgcggacgactcatctgaggggtccgaagttctgatg

SEQ ID NO: 14 - protein, 494 aa, synthetic construct
FGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 15 - DNA, 195 bp, synthetic construct
  source 1..195: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttggg

SEQ ID NO: 16 - protein, 65 aa, synthetic construct
MEYHPDLENLDEDGYTQLHFDSQSNTRIAVVSEKGSCAASPPWRLIAVILGILCLVILVIAVVLG

SEQ ID NO: 17 - DNA, 741 bp, synthetic construct
  source 1..741: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggaccatggcgatctggcgctccaactctggaagtaacacccttgaaaatggttacttcctcagtaggaacaaagagaaccattcccagccgacacagtcaagccttgaagattcagtcacccctacaaaggccgtaaaaacgacaggtgtcctgtcctctccgtgtccgcctaactggatcatctacgagaaaagttgttatctgtttagcatgagccttaacagttgggatggctcaaaaaggcagtgctggcaactggggagcaaccttttgaagatagacagttccaacgaactgggcttcatagtcaaacaggtgtcctctcaacctgataactcattctggatcgggctcagtcgaccccaaactgaggttccatggctttgggaagacggcagcactttctcttcaaatttgtttcaaataagaaccaccgctacgcaggagaatccgagtccgaactgtgtttggattcacgtctcagtcatttacgaccaactttgtagtgtccctagctattccatctgcgagaaaaagttcagtatg

SEQ ID NO: 18 - protein, 247 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 19 - DNA, 126 bp, synthetic construct
  source 1..126: mol_type = other DNA
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 20 - protein, 42 aa, synthetic construct
RLKIQVRKAAITSYEKSDGVYTGLSTRNQETYETLKHEKPPQ

SEQ ID NO: 21 - DNA, 213 bp, synthetic construct
  source 1..213: mol_type = other DNA
atggaacaatgggatcattttcacaaccaacaggaggacactgacagttgttccgagtccgtaaaatttgatgctcggtccatgacggctctcctcccccccaatccgaaaaattccccctctcttcaagaaaagctgaagagtttcaagttcagtttggctgtagtcgtcatttatctgatactcttgaccgctggggctgggctgctggtg

SEQ ID NO: 22 - protein, 76 aa, synthetic construct
MEQWDHFHNQQEDTDSCSESVKFDARSMTALLPPNPKNSPSLQEKLKSFKAALIALYLLVFAVLIPLIGIVAAQLL

SEQ ID NO: 23 - DNA, 192 bp, synthetic construct
  source 1..192: mol_type = other DNA
atgaggaataagaaaatactcaaggaagacgaacttctgagtgaaactcaacaggctgcattccatcaaatcgcaatggagccttttgaaattaacgtcccgaagcccaaaagacggaacggtgtcaacttctccttggcagttgttgtaatttatcttatcctgcttactgccggagcggggctgctggtt

SEQ ID NO: 24 - protein, 64 aa, synthetic construct
MRNKKILKEDELLSETQQAAFHQIAMEPFEINVPKPKRRNGVNFSLAVVVIYLILLTAGAGLLV

SEQ ID NO: 25 - DNA, 243 bp, synthetic construct
  source 1..243: mol_type = other DNA
atggaaaataaagcaatgtacctccacaccgtatcagactgtgacacgtcctctatctgtgaggattcttttgacggtcgaagtcttagtaagttgaatctgtgcgaggatgggccctgtcacaagaggagagcctcaatctgctgcacacaactggggtcacttagtgctctgaagcatgctgttttggggttgtatcttctggtgttcctcatcctggtcggtatatttatattggcggta

SEQ ID NO: 26 - protein, 81 aa, synthetic construct
MENKAMYLHTVSDCDTSSICEDSFDGRSLSKLNLCEDGPCHKRRASICCTQLGSLSALKHAVLGLYLLVFLILVGIFILAV

SEQ ID NO: 27 - DNA, 174 bp, synthetic construct
  source 1..174: mol_type = other DNA
atgaaggatgatttcgcagaagaagaagaagtacaatccttcgggtacaagcgcttcgggattcaagagggtacacaatgcacaaaatgcaagaacaattgggcccttaagttctcaataatactcctttacatactttgtgcgcttcttacgataactgtagcgatcctggga

SEQ ID NO: 28 - protein, 58 aa, synthetic construct
MKDDFAEEEEVQSFGYKRFGIQEGTQCTKCKNNWALKFSIILLYILCALLTITVAILG

SEQ ID NO: 29 - DNA, 183 bp, synthetic construct
  source 1..183: mol_type = other DNA
gggtgcgatcggaactgcggacttatcgctggcgcggttattggggcagtgctcgctgtgtttggtggcattctcatgccagtccttctcggactgattgaaatgatccttttgtctgttggagttgtaatgttcgtagcctttatgatctcatactgcgcgtgcagatcaaagactatcaag

SEQ ID NO: 30 - protein, 61 aa, synthetic construct
GCDRNCGLIAGAVIGAVLAVFGGILMPVLLGLIEMILLSVGVVMFVAFMISYCACRSKTIK

SEQ ID NO: 31 - DNA, 423 bp, synthetic construct
  source 1..423: mol_type = other DNA
atgggatgctctgcaaaggcaagatgggcagcaggggcgttgggcgtcgcgggcctgctgtgtgctgttcttggggctgttatgatagttatggtcgcgcagtatgtgctcttggcactggggtgtgtcctcctgcttgttccagttatatgccagatacgaagtcaagttggggcgggtcaaagggcagctagagcagattcccatagcctcgcgtgttggggtaaaggtgcatccgaccgaaccctttggcctactgctgcgtggtcacctccacctgctgcggtgcttcgcctgtgtcgctcaggtagcggtcactgttggggccttcgaagtacattggcgtctttcgcgtgccgcgtggcaaccaccttgccggtcctggaagggctgggcccttctctcggaggaggtactggtagc

SEQ ID NO: 32 - protein, 141 aa, synthetic construct
MGCSAKARWAAGALGVAGLLCAVLGAVMIVMVAQYVLLALGCVLLLVPVICQIRSQVGAGQRAARADSHSLACWGKGASDRTLWPTAAWSPPPAAVLRLCRSGSGHCWGLRSTLASFACRVATTLPVLEGLGPSLGGGTGS

SEQ ID NO: 33 - DNA, 105 bp, synthetic construct
  source 1..105: mol_type = other DNA
attttgctgccgctgataatcggacttattctcttggggctcctggctctcgttttgatcgcgttctgcatcataaggcggcgccccagcgcataccaggctctg

SEQ ID NO: 34 - protein, 35 aa, synthetic construct
ILLPLIIGLILLGLLALVLIAFCIIRRRPSAYQAL

SEQ ID NO: 35 - DNA, 171 bp, synthetic construct
  source 1..171: mol_type = other DNA
atgacctttgacgacctgaagatacagacggtgaaagatcaacctgatgagaaatcaaatgggaaaaaagcgaaaggtttgcagtttctttattccccctggtggtgccttgcagcggcaacccttggtgttctttgtctggggcttgttgtgacgatcatggtgctgggc

SEQ ID NO: 36 - protein, 57 aa, synthetic construct
MTFDDLKIQTVKDQPDEKSNGKKAKGLQFLYSPWWCLAAATLGVLCLGLVVTIMVLG

SEQ ID NO: 37 - DNA, 1227 bp, synthetic construct
  source 1..1227: mol_type = other DNA
gcactgatagctggctcccttgtgcccttgctcctccttttccttggcttggcttgttgcgcgtgttgctgctgggccccgcggtcagatcttaaggataggcctgcaagggatggcgcaacagtgagtaggatgaaactgcaggtgtggggaaccttgacatccctcggctcaactctgccttgcaggtccttgagcagccataaattgccttgggttacggttagtcatcatgacccggaggtcccttttaaccattctttcatagaaccaccgagcgccggatgggcgactgatgactctttcagttctgatccagaaagtggtgaggcggacgaggtacccgcgtattgtgtacccccacaagaggggatggttcctgtcgctcaggccggtagcagcgaggcgtctttggccgcaggcgcttttcctccgcctgaggacgcctccactccttttgcaattccgagaacctcaagcctcgcccgggctaagagaccttccgtctccttcgccgagggaacgaagttcgcaccacaaagcagaaggagctcaggtgagctctcatctccactcagaaaacctaaaagactgtcccgaggtgcccaaagtggtccagaaggacgcgaggcagaagaaagtaccggcccggaggaagctgaagcgccagaatcattcccggcagcggcctccccgggagattcagccaccggacacagacgaccaccgttgggtggccgcaccgtagccgaacatgtcgaagccatcgaaggctccgtacaagagtcttctggtccagtgactacaatatatatgctcgcagggaagccgcgaggttcagagggtccggttagatctgtttttcgccactttggcagcttccagaaaggacaggcagaggcgaaggtaaaaagggctattccgaagccgccccgccaggcactcaatcggaaaaagggaagcccaggtcttgctagtgggagtgtgggtcaatctcccaatagtgctccgaaagcaggtctgccaggcgcgaccggtcccatggcggtcagaccagaagaggctgtgagaggcttgggagcgggaactgagagttcacggcgcgcgcaggagccagtgtctggctgcggaagtccagagcaagacccccaaaaacaggcagaagaggaaagacaagaggagcccgaatatgaaaatgttgtccctatatcacggcctcctgagccc

SEQ ID NO: 38 - protein, 409 aa, synthetic construct
ALIAGSLVPLLLLFLGLACCACCCWAPRSDLKDRPARDGATVSRMKLQVWGTLTSLGSTLPCRSLSSHKLPWVTVSHHDPEVPFNHSFIEPPSAGWATDDSFSSDPESGEADEVPAYCVPPQEGMVPVAQAGSSEASLAAGAFPPPEDASTPFAIPRTSSLARAKRPSVSFAEGTKFAPQSRRSSGELSSPLRKPKRLSRGAQSGPEGREAEESTGPEEAEAPESFPAAASPGDSATGHRRPPLGGRTVAEHVEAIEGSVQESSGPVTTIYMLAGKPRGSEGPVRSVFRHFGSFQKGQAEAKVKRAIPKPPRQALNRKKGSPGLASGSVGQSPNSAPKAGLPGATGPMAVRPEEAVRGLGAGTESSRRAQEPVSGCGSPEQDPQKQAEEERQEEPEYENVVPISRPPEP

SEQ ID NO: 39 - DNA, 849 bp, synthetic construct
  source 1..849: mol_type = other DNA
gctatcgcggggatcattatattggtcttggtggtgctgtttctgctcgcgcttttcattatataccgccataagcagaagggcaaagagtcctccatgccagccgtgacctatacgcctgcgatgcgcgttgtcaacgccgattacaccatcagtggtaccctcccgcacagtaacggcggaaatgcaaactctcattactttacaaatcctagttaccatacactcactcagtgtgctacctctccccatgtgaacaatcgggacaggatgaccgttacgaaaagcaaaaataaccagttgtttgtgaaccttaagaatgtgaatcccggcaagaggggtccggtgggtgactgcaccggaactctccccgctgactggaagcatggcgggtacctgaacgaactcggcgcgtttgggctcgaccgaagctacatgggtaaaagtcttaaggacctcggtaagaatagcgagtataatagctctaactgttccctttccagctccgagaatccgtacgctactataaaagaccccccggtgctcattcccaaatccagtgagtgcgggtacgtggagatgaaaagtcccgctcgaagagacagtccatacgcggaaatcaataactccaccagtgcgaaccgcaatgtgtacgaagtggagcccaccgtttccgttgtacaaggtgtattttcaaacaatgggaggcttagccaggacccctatgatcttccaaagaacagccacatcccgtgtcattatgatctgttgccggtgagggattctagctcttctcctaaacaagaggactcaggtggctctagttccaactcctccagttcttcagag

SEQ ID NO: 40 - protein, 283 aa, synthetic construct
AIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 41 - DNA, 147 bp, synthetic construct
  source 1..147: mol_type = other DNA
gttccagttctgtgccttttggccataatttttatccttacagctgcactttcatatgtcttgtgcaaacgacggagagggcaaagcccacagagcagcccagacctcccagtgcattatatacctgtggcacctgactcaaacaca

SEQ ID NO: 42 - protein, 49 aa, synthetic construct
VPVLCLLAIIFILTAALSYVLCKRRRGQSPQSSPDLPVHYIPVAPDSNT

SEQ ID NO: 43 - DNA, 276 bp, synthetic construct
  source 1..276: mol_type = other DNA
ggcgtaggtgcggttttggctgctggggccctgcttggcttggtggcgggagcgctttatctgcgagcacgaggaaagcctatgggatttggtttttctgcttttcaggccgaggacgacgccgatgacgactttagtccttggcaagaaggaactaatccaacgctggtttccgtgcctaatcccgtgtttggatctgatacgttttgcgagccatttgacgattctttgctggaggaagatttccccgacacccagaggatactcacagttaaa

SEQ ID NO: 44 - protein, 92 aa, synthetic construct
GVGAVLAAGALLGLVAGALYLRARGKPMGFGFSAFQAEDDADDDFSPWQEGTNPTLVSVPNPVFGSDTFCEPFDDSLLEEDFPDTQRILTVK

SEQ ID NO: 45 - DNA, 318 bp, synthetic construct
  source 1..318: mol_type = other DNA
tttattgccgtgggaattctcggtgtagtgcttcttgctatatttgtcgctttgttctttctgactaaaaagcgcaggcaaaggcagcggcttgctgtgagctctcggggagaaaacctcgttcaccaaatccaataccgagaaatgaactcctgtctcaacgccgacgatcttgacctgatgaactcatctgagaactcacacgagtccgccgatttcagcgcggcggaattgatctctgtcagcaaatttctgcctataagtggcatggaaaaagaagccatactctctcacacggaaaaggaaaatggcaacctt

SEQ ID NO: 46 - protein, 106 aa, synthetic construct
FIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 47 - DNA, 186 bp, synthetic construct
  source 1..186: mol_type = other DNA
ctcgcccttggtatcttgggcggtctgggcacagccgcgttgctcattggggttattttgtggcagagacgacagcgccgaggcgaagaaaggaaagcgccggagaaccaggaagaagaggaagaaagggctgagctgaaccagtctgaagaaccagaagccggggagagttccaccggcgggcca

SEQ ID NO: 48 - protein, 62 aa, synthetic construct
LALGILGGLGTAALLIGVILWQRRQRRGEERKAPENQEEEEERAELNQSEEPEAGESSTGGP

SEQ ID NO: 49 - DNA, 279 bp, synthetic construct
  source 1..279: mol_type = other DNA
tggcttatcattttggcctcacttcttgctcttgcacttatcctcgctgtgtgcattgcagtgaacagtagaaggcggtgcggtcagaagaagaaattggtaataaactcaggaaatggagcggtagaggataggaagccatctgggcttaacggagaagcaagcaagtctcaagagatggtacatttggtcaataaggagagttctgaaacacctgaccaatttatgaccgctgatgaaacgcgcaacctccaaaatgtagacatgaaaataggcgtg

SEQ ID NO: 50 - protein, 93 aa, synthetic construct
WLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 51 - DNA, 372 bp, synthetic construct
  source 1..372: mol_type = other DNA
catatagcttctatacttattccattgctcttgcttctgctgctggtattggtcgctggtgttgtcttttggtacaagcgccgcgtgcagggtgccaaggggttccaacaccaaagaatgaccaatggggcaatgaatgttgaaattggaaacccgacctacaaaatgtatgagggaggtgaacctgatgatgttggtggtctcttggacgcggattttgcgctcgaccccgataagccgactaatttcactcccgtgtatgccactctttatatggggggccatgggagtagacactcacttgcctccaccgatgaaaagagggaattgttgggtcgaggacccgaagacgagattggggacccacttgcg

SEQ ID NO: 52 - protein, 124 aa, synthetic construct
HIASILIPLLLLLLLVLVAGVVFWYKRRVQGAKGFQHQRMTNGAMNVEIGNPTYKMYEGGEPDDVGGLLDADFALDPDKPTNFTPVYATLYMGGHGSRHSLASTDEKRELLGRGPEDEIGDPLA

SEQ ID NO: 53 - DNA, 1200 bp, synthetic construct
  source 1..1200: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgactgaagatccaagtgcgaaaggcagctataaccagctatgagaaatcagatggtgtttacacgggcctgagcaccaggaaccaggagacttacgagactctgaagcatgagaaaccaccacagtag

SEQ ID NO: 54 - DNA, 2481 bp, synthetic construct
  source 1..2481: mol_type = other DNA
atggccttaccagtgaccgccttgctcctgccgctggccttgctgctccacgccgccaggccgggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 55 - DNA, 2679 bp, synthetic construct
  source 1..2679: mol_type = other DNA
atgaggctacccctgctcctggtttttgcctctgtcattccgggtgctgttctctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 56 - DNA, 3873 bp, synthetic construct
  source 1..3873: mol_type = other DNA
atgaggctacccctgctcctggtttttgcctctgtcattccgggtgctgttctctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattttggatgtttttgtggtttcatcctcatcggtttgatattgtacataagtctggcgataaggaagagagttcaagagacaaagttcggaaatgcctttacagaggaagacagtgagctcgttgtaaactacatcgcaaaaaaaagcttctgtagaagagcaatagagctcacgttgcactcactcggtgtgtccgaagaactccagaataaactggaagacgtcgttatcgatcggaacctcctcatacttggaaaaatactgggagaaggagagttcggcagtgtcatggaaggtaacttgaaacaagaggatggtacctcactcaaggtagctgtcaagacgatgaaacttgataacagttcacaaagggagatcgaagaatttctgtctgaggccgcctgtatgaaagacttctcacatcctaatgtcatcagacttcttggcgtttgtatcgagatgtctagccaaggaatcccaaaacctatggtcatattgcctttcatgaaatatggcgatctgcatacatatttgctctactctagacttgagacagggcccaaacatattcctctccagacattgctcaagtttatggtcgatattgccctgggtatggagtacttgagcaaccgaaattttctgcatcgggatcttgccgcacgcaactgcatgctgcgcgatgacatgaccgtctgcgtggctgattttgggctgtcaaaaaaaatatattctggagactactaccgacaagggcggattgcaaagatgcccgtcaaatggattgcgattgaaagtttggcggacagggtatatacttccaaatcagatgtttgggcttttggagtcactatgtgggaaatagctacacgcggtatgaccccgtaccccggagtacaaaatcatgaaatgtatgactatctccttcatggacacaggctgaagcagcccgaggactgcctggacgaactgtatgaaataatgtattcttgttggcgaaccgatcccttggaccggcctactttcagtgtccttagattgcaacttgagaaattgctcgagtctttgccggatgtgcgaaaccaggcagacgtgatctatgtcaatacccaacttttggaaagttctgagggcctcgcacagggttctacccttgccccgttggatcttaacatagacccagacagcataattgcttcttgtacacctcgcgctgccatatcagttgtaacagcggaggtccatgatagtaaacctcacgagggtcgctatatcctgaacggcgggtcagaagaatgggaagacctgacatcagcgccgagcgccgccgttactgctgagaaaaactctgtcctgcccggagagcgcttggttcggaacggggtaagttggagccatagctcaatgctccccctgggttcaagtctcccggacgagcttctttttgcggacgactcatctgaggggtccgaagttctgatg

SEQ ID NO: 57 - DNA, 3240 bp, synthetic construct
  source 1..3240: mol_type = other DNA
atgaggctacccctgctcctggtttttgcctctgtcattccgggtgctgttctctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgctatcgcggggatcattatattggtcttggtggtgctgtttctgctcgcgcttttcattatataccgccataagcagaagggcaaagagtcctccatgccagccgtgacctatacgcctgcgatgcgcgttgtcaacgccgattacaccatcagtggtaccctcccgcacagtaacggcggaaatgcaaactctcattactttacaaatcctagttaccatacactcactcagtgtgctacctctccccatgtgaacaatcgggacaggatgaccgttacgaaaagcaaaaataaccagttgtttgtgaaccttaagaatgtgaatcccggcaagaggggtccggtgggtgactgcaccggaactctccccgctgactggaagcatggcgggtacctgaacgaactcggcgcgtttgggctcgaccgaagctacatgggtaaaagtcttaaggacctcggtaagaatagcgagtataatagctctaactgttccctttccagctccgagaatccgtacgctactataaaagaccccccggtgctcattcccaaatccagtgagtgcgggtacgtggagatgaaaagtcccgctcgaagagacagtccatacgcggaaatcaataactccaccagtgcgaaccgcaatgtgtacgaagtggagcccaccgtttccgttgtacaaggtgtattttcaaacaatgggaggcttagccaggacccctatgatcttccaaagaacagccacatcccgtgtcattatgatctgttgccggtgagggattctagctcttctcctaaacaagaggactcaggtggctctagttccaactcctccagttcttcagag

SEQ ID NO: 58 - DNA, 2532 bp, synthetic construct
  source 1..2532: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 59 - DNA, 2913 bp, synthetic construct
  source 1..2913: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggaccatggcgatctggcgctccaactctggaagtaacacccttgaaaatggttacttcctcagtaggaacaaagagaaccattcccagccgacacagtcaagccttgaagattcagtcacccctacaaaggccgtaaaaacgacaggtgtcctgtcctctccgtgtccgcctaactggatcatctacgagaaaagttgttatctgtttagcatgagccttaacagttgggatggctcaaaaaggcagtgctggcaactggggagcaaccttttgaagatagacagttccaacgaactgggcttcatagtcaaacaggtgtcctctcaacctgataactcattctggatcgggctcagtcgaccccaaactgaggttccatggctttgggaagacggcagcactttctcttcaaatttgtttcaaataagaaccaccgctacgcaggagaatccgagtccgaactgtgtttggattcacgtctcagtcatttacgaccaactttgtagtgtccctagctattccatctgcgagaaaaagttcagtatgggatcctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 60 - DNA, 5736 bp, synthetic construct
  source 1..5736: mol_type = other DNA
atgaggctacccctgctcctggtttttgcctctgtcattccgggtgctgttctctccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattcatccctgggtggaacagataaggagttgaggttggtggatggagagaataagtgcagtggacgggttgaggtcaaggtgcaggaagagtggggtactgtttgtaacaacggctggagtatggaagcagtctccgtgatatgcaatcaacttggttgccctacagccataaaagctcctggatgggctaactcatctgccggaagcggtcgaatatggatggaccatgtatcctgtaggggcaatgagtcagcactgtgggactgcaagcatgacgggtggggaaaacactctaattgtacccaccagcaagatgccggtgtgacgtgtagcgacggcagcaacttggagatgcggttgacgcgcggcgggaacatgtgcagcggtcgcattgagataaagttccaagggcgctgggggaccgtttgtgacgacaacttcaatatagatcatgctagtgtgatatgcaggcagttggagtgcggtagcgccgtctcatttagtggttctagcaatttcggtgagggatccgggcctatatggttcgacgaccttatctgcaatggaaatgagagcgccctctggaactgcaagcaccaaggttggggtaagcataactgcgatcatgctgaggatgcgggtgtgatttgtagcaagggagccgacttgtcacttcgactcgtagatggcgtgacggaatgtagcggtcgccttgaggtacgcttccaaggtgaatggggcacaatctgtgatgacggttgggactcatatgacgctgcggttgcttgcaaacagctgggctgtccgacagcggtaacggcaattgggcgggtaaatgccagtaaggggtttggacatatatggttggacagtgtttcttgtcagggacacgaacctgcaatatggcaatgtaaacatcacgagtgggggaagcattactgcaatcataatgaagacgcaggcgtcacatgctctgacggatccgacctggaattgcggctgagaggggggggttcacggtgcgcaggaaccgtggaagttgagattcaacgcctgctcgggaaagtttgtgacagggggtggggccttaaggaagcagacgtagtctgccgacaactgggttgcgggagcgccctcaagacgtcctatcaagtttacagcaaaattcaagcaactaatacctggctgtttctttcctcctgcaatggtaacgaaacgagcctctgggattgtaaaaattggcaatggggaggccttacatgtgatcactatgaagaggccaaaatcacctgcagcgcgcaccgagagcctaggctggttggaggggatattccctgttcaggtagagttgaagtcaagcatggtgatacctgggggtccatatgcgactccgatttcagtttggaagcagcgagcgtgctgtgccgagagcttcaatgtgggacagtagtttccattcttggtggcgcccacttcggggaaggtaacggacagatttgggcggaggaattccaatgtgagggccatgaaagtcacctgagtctttgtcctgtagcgcctcggccggaaggtacttgttctcactccagagacgttggcgtggtttgttctaggtacactgagataaggctggtgaatggcaaaacgccttgtgaaggaagggtggagctcaaaacgcttggcgcctgggggtctctgtgcaactcccactgggacatagaggacgcacatgtcttgtgccagcaactcaaatgcggtgtcgcgctttcaacccctgggggcgctagattcgggaaaggtaacggccagatatggcgccatatgttccattgcaccggaactgaacagcatatgggagattgtcctgtgactgccttgggggcaagtctgtgtccctcagaacaagtggcttcagttatttgcagcggtaaccagagtcagacgctcagctcctgcaacagcagcagtctgggtccaacaagacccacaatacccgaggagtcagcggtcgcgtgcatcgaatccgggcaattgagactcgttaatggcgggggtcggtgcgcagggcgcgtggagatctaccacgagggtagttggggcacaatttgtgacgacagctgggacctttccgacgctcatgtcgtatgccgacaactggggtgcggtgaggccatcaatgccaccggaagcgcgcatttcggtgaaggcacgggcccaatttggcttgatgagatgaaatgtaatggaaaggagtcccgcatttggcaatgccatagccatggctggggtcaacaaaattgtcgacacaaagaagatgccggggtgatctgctcagaattcatgtccttgcggcttactagcgaagcgtcccgcgaggcctgtgctgggagactggaagttttttataacggggcttggggaacggttggtaagtcatcaatgagtgaaaccacagttggtgttgtgtgtagacaactcggttgcgccgacaagggaaagatcaacccggcgagccttgataaggccatgagcatccccatgtgggtggataacgttcagtgtccgaaaggtcccgataccttgtggcaatgcccaagttctccttgggagaagagactcgcctcaccaagtgaagagacgtggattacgtgtgataacaaaattaggctccaagaaggaccgaccagttgcagcggaagagttgagatatggcatggaggaagctggggaaccgtgtgcgatgacagctgggacctggacgacgcccaggtggtgtgccaacagctgggttgcgggcctgccctcaaagcatttaaggaagccgaattcggtcagggtactgggccaatctggctgaacgaggtaaagtgcaaaggtaacgaaagtagcctgtgggactgtccggcacgaaggtggggccacagcgagtgtggccataaggaagacgcggccgtgaactgtacagacatatccgtacaaaaaacgccccaaaaggcgacgaccgggcgatcatcaagacaatctagctttattgccgtgggaattctcggtgtagtgcttcttgctatatttgtcgctttgttctttctgactaaaaagcgcaggcaaaggcagcggcttgctgtgagctctcggggagaaaacctcgttcaccaaatccaataccgagaaatgaactcctgtctcaacgccgacgatcttgacctgatgaactcatctgagaactcacacgagtccgccgatttcagcgcggcggaattgatctctgtcagcaaatttctgcctataagtggcatggaaaaagaagccatactctctcacacggaaaaggaaaatggcaacctt

SEQ ID NO: 61 - protein, 827 aa, synthetic construct
MALPVTALLLPLALLLHAARPGSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSDTRKMDPSKPSSNVAGVVIIVILLILTGAGLAAYFFYKKRRVHLPQEGAFENTLYFNSQSSPGTSDMKDLVGNIEQNEHSVI

SEQ ID NO: 62 - protein, 893 aa, synthetic construct
MRLPLLLVFASVIPGAVLSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSFVPVFLPAKPTTTPAPRPPTPAPTIASQPLSLRPEACRPAAGGAVHTRGLDFACDIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 63 - protein, 1291 aa, synthetic construct
MRLPLLLVFASVIPGAVLSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSFVPVFLPAKPTTTPAPRPPTPAPTIASQPLSLRPEACRPAAGGAVHTRGLDFACDFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 64 - protein, 1080 aa, synthetic construct
MRLPLLLVFASVIPGAVLSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSFVPVFLPAKPTTTPAPRPPTPAPTIASQPLSLRPEACRPAAGGAVHTRGLDFACDAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 65 - protein, 844 aa, synthetic construct
MEYHPDLENLDEDGYTQLHFDSQSNTRIAVVSEKGSCAASPPWRLIAVILGILCLVILVIAVVLGFVPVFLPAKPTTTPAPRPPTPAPTIASQPLSLRPEACRPAAGGAVHTRGLDFACDGSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 66 - protein, 971 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMGSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 67 - protein, 1912 aa, synthetic construct
MRLPLLLVFASVIPGAVLSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSFVPVFLPAKPTTTPAPRPPTPAPTIASQPLSLRPEACRPAAGGAVHTRGLDFACDSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 68 - DNA, 1189 bp, synthetic construct
  source 1..1189: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatcg

SEQ ID NO: 69 - DNA, 1275 bp, synthetic construct
  source 1..1275: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 70 - DNA, 1104 bp, synthetic construct
  source 1..1104: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 71 - DNA, 1233 bp, synthetic construct
  source 1..1233: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 72 - DNA, 1062 bp, synthetic construct
  source 1..1062: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 73 - DNA, 1260 bp, synthetic construct
  source 1..1260: mol_type = other DNA
atgaggctacccctgctcctggtttttgcctctgtcattccgggtgctgttctcgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 74 - DNA, 2454 bp, synthetic construct
  source 1..2454: mol_type = other DNA
atgaggctacccctgctcctggtttttgcctctgtcattccgggtgctgttctcgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattttggatgtttttgtggtttcatcctcatcggtttgatattgtacataagtctggcgataaggaagagagttcaagagacaaagttcggaaatgcctttacagaggaagacagtgagctcgttgtaaactacatcgcaaaaaaaagcttctgtagaagagcaatagagctcacgttgcactcactcggtgtgtccgaagaactccagaataaactggaagacgtcgttatcgatcggaacctcctcatacttggaaaaatactgggagaaggagagttcggcagtgtcatggaaggtaacttgaaacaagaggatggtacctcactcaaggtagctgtcaagacgatgaaacttgataacagttcacaaagggagatcgaagaatttctgtctgaggccgcctgtatgaaagacttctcacatcctaatgtcatcagacttcttggcgtttgtatcgagatgtctagccaaggaatcccaaaacctatggtcatattgcctttcatgaaatatggcgatctgcatacatatttgctctactctagacttgagacagggcccaaacatattcctctccagacattgctcaagtttatggtcgatattgccctgggtatggagtacttgagcaaccgaaattttctgcatcgggatcttgccgcacgcaactgcatgctgcgcgatgacatgaccgtctgcgtggctgattttgggctgtcaaaaaaaatatattctggagactactaccgacaagggcggattgcaaagatgcccgtcaaatggattgcgattgaaagtttggcggacagggtatatacttccaaatcagatgtttgggcttttggagtcactatgtgggaaatagctacacgcggtatgaccccgtaccccggagtacaaaatcatgaaatgtatgactatctccttcatggacacaggctgaagcagcccgaggactgcctggacgaactgtatgaaataatgtattcttgttggcgaaccgatcccttggaccggcctactttcagtgtccttagattgcaacttgagaaattgctcgagtctttgccggatgtgcgaaaccaggcagacgtgatctatgtcaatacccaacttttggaaagttctgagggcctcgcacagggttctacccttgccccgttggatcttaacatagacccagacagcataattgcttcttgtacacctcgcgctgccatatcagttgtaacagcggaggtccatgatagtaaacctcacgagggtcgctatatcctgaacggcgggtcagaagaatgggaagacctgacatcagcgccgagcgccgccgttactgctgagaaaaactctgtcctgcccggagagcgcttggttcggaacggggtaagttggagccatagctcaatgctccccctgggttcaagtctcccggacgagcttctttttgcggacgactcatctgaggggtccgaagttctgatg

SEQ ID NO: 75 - DNA, 1821 bp, synthetic construct
  source 1..1821: mol_type = other DNA
atgaggctacccctgctcctggtttttgcctctgtcattccgggtgctgttctcgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgctatcgcggggatcattatattggtcttggtggtgctgtttctgctcgcgcttttcattatataccgccataagcagaagggcaaagagtcctccatgccagccgtgacctatacgcctgcgatgcgcgttgtcaacgccgattacaccatcagtggtaccctcccgcacagtaacggcggaaatgcaaactctcattactttacaaatcctagttaccatacactcactcagtgtgctacctctccccatgtgaacaatcgggacaggatgaccgttacgaaaagcaaaaataaccagttgtttgtgaaccttaagaatgtgaatcccggcaagaggggtccggtgggtgactgcaccggaactctccccgctgactggaagcatggcgggtacctgaacgaactcggcgcgtttgggctcgaccgaagctacatgggtaaaagtcttaaggacctcggtaagaatagcgagtataatagctctaactgttccctttccagctccgagaatccgtacgctactataaaagaccccccggtgctcattcccaaatccagtgagtgcgggtacgtggagatgaaaagtcccgctcgaagagacagtccatacgcggaaatcaataactccaccagtgcgaaccgcaatgtgtacgaagtggagcccaccgtttccgttgtacaaggtgtattttcaaacaatgggaggcttagccaggacccctatgatcttccaaagaacagccacatcccgtgtcattatgatctgttgccggtgagggattctagctcttctcctaaacaagaggactcaggtggctctagttccaactcctccagttcttcagag

SEQ ID NO: 76 - DNA, 1113 bp, synthetic construct
  source 1..1113: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctct

SEQ ID NO: 77 - DNA, 1494 bp, synthetic construct
  source 1..1494: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggaccatggcgatctggcgctccaactctggaagtaacacccttgaaaatggttacttcctcagtaggaacaaagagaaccattcccagccgacacagtcaagccttgaagattcagtcacccctacaaaggccgtaaaaacgacaggtgtcctgtcctctccgtgtccgcctaactggatcatctacgagaaaagttgttatctgtttagcatgagccttaacagttgggatggctcaaaaaggcagtgctggcaactggggagcaaccttttgaagatagacagttccaacgaactgggcttcatagtcaaacaggtgtcctctcaacctgataactcattctggatcgggctcagtcgaccccaaactgaggttccatggctttgggaagacggcagcactttctcttcaaatttgtttcaaataagaaccaccgctacgcaggagaatccgagtccgaactgtgtttggattcacgtctcagtcatttacgaccaactttgtagtgtccctagctattccatctgcgagaaaaagttcagtatgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctct

SEQ ID NO: 78 - DNA, 4317 bp, synthetic construct
  source 1..4317: mol_type = other DNA
atgaggctacccctgctcctggtttttgcctctgtcattccgggtgctgttctcgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattcatccctgggtggaacagataaggagttgaggttggtggatggagagaataagtgcagtggacgggttgaggtcaaggtgcaggaagagtggggtactgtttgtaacaacggctggagtatggaagcagtctccgtgatatgcaatcaacttggttgccctacagccataaaagctcctggatgggctaactcatctgccggaagcggtcgaatatggatggaccatgtatcctgtaggggcaatgagtcagcactgtgggactgcaagcatgacgggtggggaaaacactctaattgtacccaccagcaagatgccggtgtgacgtgtagcgacggcagcaacttggagatgcggttgacgcgcggcgggaacatgtgcagcggtcgcattgagataaagttccaagggcgctgggggaccgtttgtgacgacaacttcaatatagatcatgctagtgtgatatgcaggcagttggagtgcggtagcgccgtctcatttagtggttctagcaatttcggtgagggatccgggcctatatggttcgacgaccttatctgcaatggaaatgagagcgccctctggaactgcaagcaccaaggttggggtaagcataactgcgatcatgctgaggatgcgggtgtgatttgtagcaagggagccgacttgtcacttcgactcgtagatggcgtgacggaatgtagcggtcgccttgaggtacgcttccaaggtgaatggggcacaatctgtgatgacggttgggactcatatgacgctgcggttgcttgcaaacagctgggctgtccgacagcggtaacggcaattgggcgggtaaatgccagtaaggggtttggacatatatggttggacagtgtttcttgtcagggacacgaacctgcaatatggcaatgtaaacatcacgagtgggggaagcattactgcaatcataatgaagacgcaggcgtcacatgctctgacggatccgacctggaattgcggctgagaggggggggttcacggtgcgcaggaaccgtggaagttgagattcaacgcctgctcgggaaagtttgtgacagggggtggggccttaaggaagcagacgtagtctgccgacaactgggttgcgggagcgccctcaagacgtcctatcaagtttacagcaaaattcaagcaactaatacctggctgtttctttcctcctgcaatggtaacgaaacgagcctctgggattgtaaaaattggcaatggggaggccttacatgtgatcactatgaagaggccaaaatcacctgcagcgcgcaccgagagcctaggctggttggaggggatattccctgttcaggtagagttgaagtcaagcatggtgatacctgggggtccatatgcgactccgatttcagtttggaagcagcgagcgtgctgtgccgagagcttcaatgtgggacagtagtttccattcttggtggcgcccacttcggggaaggtaacggacagatttgggcggaggaattccaatgtgagggccatgaaagtcacctgagtctttgtcctgtagcgcctcggccggaaggtacttgttctcactccagagacgttggcgtggtttgttctaggtacactgagataaggctggtgaatggcaaaacgccttgtgaaggaagggtggagctcaaaacgcttggcgcctgggggtctctgtgcaactcccactgggacatagaggacgcacatgtcttgtgccagcaactcaaatgcggtgtcgcgctttcaacccctgggggcgctagattcgggaaaggtaacggccagatatggcgccatatgttccattgcaccggaactgaacagcatatgggagattgtcctgtgactgccttgggggcaagtctgtgtccctcagaacaagtggcttcagttatttgcagcggtaaccagagtcagacgctcagctcctgcaacagcagcagtctgggtccaacaagacccacaatacccgaggagtcagcggtcgcgtgcatcgaatccgggcaattgagactcgttaatggcgggggtcggtgcgcagggcgcgtggagatctaccacgagggtagttggggcacaatttgtgacgacagctgggacctttccgacgctcatgtcgtatgccgacaactggggtgcggtgaggccatcaatgccaccggaagcgcgcatttcggtgaaggcacgggcccaatttggcttgatgagatgaaatgtaatggaaaggagtcccgcatttggcaatgccatagccatggctggggtcaacaaaattgtcgacacaaagaagatgccggggtgatctgctcagaattcatgtccttgcggcttactagcgaagcgtcccgcgaggcctgtgctgggagactggaagttttttataacggggcttggggaacggttggtaagtcatcaatgagtgaaaccacagttggtgttgtgtgtagacaactcggttgcgccgacaagggaaagatcaacccggcgagccttgataaggccatgagcatccccatgtgggtggataacgttcagtgtccgaaaggtcccgataccttgtggcaatgcccaagttctccttgggagaagagactcgcctcaccaagtgaagagacgtggattacgtgtgataacaaaattaggctccaagaaggaccgaccagttgcagcggaagagttgagatatggcatggaggaagctggggaaccgtgtgcgatgacagctgggacctggacgacgcccaggtggtgtgccaacagctgggttgcgggcctgccctcaaagcatttaaggaagccgaattcggtcagggtactgggccaatctggctgaacgaggtaaagtgcaaaggtaacgaaagtagcctgtgggactgtccggcacgaaggtggggccacagcgagtgtggccataaggaagacgcggccgtgaactgtacagacatatccgtacaaaaaacgccccaaaaggcgacgaccgggcgatcatcaagacaatctagctttattgccgtgggaattctcggtgtagtgcttcttgctatatttgtcgctttgttctttctgactaaaaagcgcaggcaaaggcagcggcttgctgtgagctctcggggagaaaacctcgttcaccaaatccaataccgagaaatgaactcctgtctcaacgccgacgatcttgacctgatgaactcatctgagaactcacacgagtccgccgatttcagcgcggcggaattgatctctgtcagcaaatttctgcctataagtggcatggaaaaagaagccatactctctcacacggaaaaggaaaatggcaacctt

SEQ ID NO: 79 - DNA, 1089 bp, synthetic construct
  source 1..1089: mol_type = other DNA
atggccttaccagtgaccgccttgctcctgccgctggccttgctgctccacgccgccaggccgggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaatgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 80 - DNA, 1062 bp, synthetic construct
  source 1..1062: mol_type = other DNA
atggccttaccagtgaccgccttgctcctgccgctggccttgctgctccacgccgccaggccgggatcccaaattgttctctcccagtctccagcaatcctttctgcatctccaggggagaaggtcacaatgacttgcagggccagctcaagtttaagtttcatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttatttctgccatcagtggagtagtaacccgctcacgttcggtgctgggaccaagctggagctcaaacggggtggcggcggctctggcggcggtggctctggcggtggcggttccggcggtggtggtagccaggtgcaactgcggcagcctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagtctgacatctgaggactctgcggtctattactgtgcaagatcgcactacggtagtaactacgtagactactttgactactggggccaaggcaccactctcacagtctcctctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatc

SEQ ID NO: 81 - DNA, 2496 bp, synthetic construct
  source 1..2496: mol_type = other DNA
atggccttaccagtgaccgccttgctcctgccgctggccttgctgctccacgccgccaggccgggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaatggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattttggatgtttttgtggtttcatcctcatcggtttgatattgtacataagtctggcgataaggaagagagttcaagagacaaagttcggaaatgcctttacagaggaagacagtgagctcgttgtaaactacatcgcaaaaaaaagcttctgtagaagagcaatagagctcacgttgcactcactcggtgtgtccgaagaactccagaataaactggaagacgtcgttatcgatcggaacctcctcatacttggaaaaatactgggagaaggagagttcggcagtgtcatggaaggtaacttgaaacaagaggatggtacctcactcaaggtagctgtcaagacgatgaaacttgataacagttcacaaagggagatcgaagaatttctgtctgaggccgcctgtatgaaagacttctcacatcctaatgtcatcagacttcttggcgtttgtatcgagatgtctagccaaggaatcccaaaacctatggtcatattgcctttcatgaaatatggcgatctgcatacatatttgctctactctagacttgagacagggcccaaacatattcctctccagacattgctcaagtttatggtcgatattgccctgggtatggagtacttgagcaaccgaaattttctgcatcgggatcttgccgcacgcaactgcatgctgcgcgatgacatgaccgtctgcgtggctgattttgggctgtcaaaaaaaatatattctggagactactaccgacaagggcggattgcaaagatgcccgtcaaatggattgcgattgaaagtttggcggacagggtatatacttccaaatcagatgtttgggcttttggagtcactatgtgggaaatagctacacgcggtatgaccccgtaccccggagtacaaaatcatgaaatgtatgactatctccttcatggacacaggctgaagcagcccgaggactgcctggacgaactgtatgaaataatgtattcttgttggcgaaccgatcccttggaccggcctactttcagtgtccttagattgcaacttgagaaattgctcgagtctttgccggatgtgcgaaaccaggcagacgtgatctatgtcaatacccaacttttggaaagttctgagggcctcgcacagggttctacccttgccccgttggatcttaacatagacccagacagcataattgcttcttgtacacctcgcgctgccatatcagttgtaacagcggaggtccatgatagtaaacctcacgagggtcgctatatcctgaacggcgggtcagaagaatgggaagacctgacatcagcgccgagcgccgccgttactgctgagaaaaactctgtcctgcccggagagcgcttggttcggaacggggtaagttggagccatagctcaatgctccccctgggttcaagtctcccggacgagcttctttttgcggacgactcatctgaggggtccgaagttctgatg

SEQ ID NO: 82 - DNA, 1863 bp, synthetic construct
  source 1..1863: mol_type = other DNA
atggccttaccagtgaccgccttgctcctgccgctggccttgctgctccacgccgccaggccgggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaatggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatgctatcgcggggatcattatattggtcttggtggtgctgtttctgctcgcgcttttcattatataccgccataagcagaagggcaaagagtcctccatgccagccgtgacctatacgcctgcgatgcgcgttgtcaacgccgattacaccatcagtggtaccctcccgcacagtaacggcggaaatgcaaactctcattactttacaaatcctagttaccatacactcactcagtgtgctacctctccccatgtgaacaatcgggacaggatgaccgttacgaaaagcaaaaataaccagttgtttgtgaaccttaagaatgtgaatcccggcaagaggggtccggtgggtgactgcaccggaactctccccgctgactggaagcatggcgggtacctgaacgaactcggcgcgtttgggctcgaccgaagctacatgggtaaaagtcttaaggacctcggtaagaatagcgagtataatagctctaactgttccctttccagctccgagaatccgtacgctactataaaagaccccccggtgctcattcccaaatccagtgagtgcgggtacgtggagatgaaaagtcccgctcgaagagacagtccatacgcggaaatcaataactccaccagtgcgaaccgcaatgtgtacgaagtggagcccaccgtttccgttgtacaaggtgtattttcaaacaatgggaggcttagccaggacccctatgatcttccaaagaacagccacatcccgtgtcattatgatctgttgccggtgagggattctagctcttctcctaaacaagaggactcaggtggctctagttccaactcctccagttcttcagag

SEQ ID NO: 83 - DNA, 1140 bp, synthetic construct
  source 1..1140: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaat

SEQ ID NO: 84 - DNA, 1521 bp, synthetic construct
  source 1..1521: mol_type = other DNA
atggagtatcaccccgacctggagaatttggacgaggatggctacactcaactgcatttcgatagtcagtccaatacaaggatcgctgttgtgtctgaaaagggcagttgtgctgccagtccaccgtggcggttgattgccgtcatcctcggcattctgtgcttggttattctcgttatagcggtggtgcttgggaccatggcgatctggcgctccaactctggaagtaacacccttgaaaatggttacttcctcagtaggaacaaagagaaccattcccagccgacacagtcaagccttgaagattcagtcacccctacaaaggccgtaaaaacgacaggtgtcctgtcctctccgtgtccgcctaactggatcatctacgagaaaagttgttatctgtttagcatgagccttaacagttgggatggctcaaaaaggcagtgctggcaactggggagcaaccttttgaagatagacagttccaacgaactgggcttcatagtcaaacaggtgtcctctcaacctgataactcattctggatcgggctcagtcgaccccaaactgaggttccatggctttgggaagacggcagcactttctcttcaaatttgtttcaaataagaaccaccgctacgcaggagaatccgagtccgaactgtgtttggattcacgtctcagtcatttacgaccaactttgtagtgtccctagctattccatctgcgagaaaaagttcagtatgggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaat

SEQ ID NO: 85 - DNA, 4359 bp, synthetic construct
  source 1..4359: mol_type = other DNA
atggccttaccagtgaccgccttgctcctgccgctggccttgctgctccacgccgccaggccgggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcggcggtggctctggcggtggcggttccggcggtggtggtagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaatggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattcatccctgggtggaacagataaggagttgaggttggtggatggagagaataagtgcagtggacgggttgaggtcaaggtgcaggaagagtggggtactgtttgtaacaacggctggagtatggaagcagtctccgtgatatgcaatcaacttggttgccctacagccataaaagctcctggatgggctaactcatctgccggaagcggtcgaatatggatggaccatgtatcctgtaggggcaatgagtcagcactgtgggactgcaagcatgacgggtggggaaaacactctaattgtacccaccagcaagatgccggtgtgacgtgtagcgacggcagcaacttggagatgcggttgacgcgcggcgggaacatgtgcagcggtcgcattgagataaagttccaagggcgctgggggaccgtttgtgacgacaacttcaatatagatcatgctagtgtgatatgcaggcagttggagtgcggtagcgccgtctcatttagtggttctagcaatttcggtgagggatccgggcctatatggttcgacgaccttatctgcaatggaaatgagagcgccctctggaactgcaagcaccaaggttggggtaagcataactgcgatcatgctgaggatgcgggtgtgatttgtagcaagggagccgacttgtcacttcgactcgtagatggcgtgacggaatgtagcggtcgccttgaggtacgcttccaaggtgaatggggcacaatctgtgatgacggttgggactcatatgacgctgcggttgcttgcaaacagctgggctgtccgacagcggtaacggcaattgggcgggtaaatgccagtaaggggtttggacatatatggttggacagtgtttcttgtcagggacacgaacctgcaatatggcaatgtaaacatcacgagtgggggaagcattactgcaatcataatgaagacgcaggcgtcacatgctctgacggatccgacctggaattgcggctgagaggggggggttcacggtgcgcaggaaccgtggaagttgagattcaacgcctgctcgggaaagtttgtgacagggggtggggccttaaggaagcagacgtagtctgccgacaactgggttgcgggagcgccctcaagacgtcctatcaagtttacagcaaaattcaagcaactaatacctggctgtttctttcctcctgcaatggtaacgaaacgagcctctgggattgtaaaaattggcaatggggaggccttacatgtgatcactatgaagaggccaaaatcacctgcagcgcgcaccgagagcctaggctggttggaggggatattccctgttcaggtagagttgaagtcaagcatggtgatacctgggggtccatatgcgactccgatttcagtttggaagcagcgagcgtgctgtgccgagagcttcaatgtgggacagtagtttccattcttggtggcgcccacttcggggaaggtaacggacagatttgggcggaggaattccaatgtgagggccatgaaagtcacctgagtctttgtcctgtagcgcctcggccggaaggtacttgttctcactccagagacgttggcgtggtttgttctaggtacactgagataaggctggtgaatggcaaaacgccttgtgaaggaagggtggagctcaaaacgcttggcgcctgggggtctctgtgcaactcccactgggacatagaggacgcacatgtcttgtgccagcaactcaaatgcggtgtcgcgctttcaacccctgggggcgctagattcgggaaaggtaacggccagatatggcgccatatgttccattgcaccggaactgaacagcatatgggagattgtcctgtgactgccttgggggcaagtctgtgtccctcagaacaagtggcttcagttatttgcagcggtaaccagagtcagacgctcagctcctgcaacagcagcagtctgggtccaacaagacccacaatacccgaggagtcagcggtcgcgtgcatcgaatccgggcaattgagactcgttaatggcgggggtcggtgcgcagggcgcgtggagatctaccacgagggtagttggggcacaatttgtgacgacagctgggacctttccgacgctcatgtcgtatgccgacaactggggtgcggtgaggccatcaatgccaccggaagcgcgcatttcggtgaaggcacgggcccaatttggcttgatgagatgaaatgtaatggaaaggagtcccgcatttggcaatgccatagccatggctggggtcaacaaaattgtcgacacaaagaagatgccggggtgatctgctcagaattcatgtccttgcggcttactagcgaagcgtcccgcgaggcctgtgctgggagactggaagttttttataacggggcttggggaacggttggtaagtcatcaatgagtgaaaccacagttggtgttgtgtgtagacaactcggttgcgccgacaagggaaagatcaacccggcgagccttgataaggccatgagcatccccatgtgggtggataacgttcagtgtccgaaaggtcccgataccttgtggcaatgcccaagttctccttgggagaagagactcgcctcaccaagtgaagagacgtggattacgtgtgataacaaaattaggctccaagaaggaccgaccagttgcagcggaagagttgagatatggcatggaggaagctggggaaccgtgtgcgatgacagctgggacctggacgacgcccaggtggtgtgccaacagctgggttgcgggcctgccctcaaagcatttaaggaagccgaattcggtcagggtactgggccaatctggctgaacgaggtaaagtgcaaaggtaacgaaagtagcctgtgggactgtccggcacgaaggtggggccacagcgagtgtggccataaggaagacgcggccgtgaactgtacagacatatccgtacaaaaaacgccccaaaaggcgacgaccgggcgatcatcaagacaatctagctttattgccgtgggaattctcggtgtagtgcttcttgctatatttgtcgctttgttctttctgactaaaaagcgcaggcaaaggcagcggcttgctgtgagctctcggggagaaaacctcgttcaccaaatccaataccgagaaatgaactcctgtctcaacgccgacgatcttgacctgatgaactcatctgagaactcacacgagtccgccgatttcagcgcggcggaattgatctctgtcagcaaatttctgcctataagtggcatggaaaaagaagccatactctctcacacggaaaaggaaaatggcaacctt

SEQ ID NO: 86 - protein, 396 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDGVVIIVILLILTGAGLAAYFFYKKRRVHLPQEGAFENTLYFNSQSSPGTSDMKDLVGNIEQNEHSVI

SEQ ID NO: 87 - protein, 425 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 88 - protein, 368 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 89 - protein, 411 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDDTRKMDPSKPSSNVAGVVIIVILLILTGAGLAAYFFYKKRRVHLPQEGAFENTLYFNSQSSPGTSDMKDLVGNIEQNEHSVI

SEQ ID NO: 90 - protein, 354 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSDTRKMDPSKPSSNVAGVVIIVILLILTGAGLAAYFFYKKRRVHLPQEGAFENTLYFNSQSSPGTSDMKDLVGNIEQNEHSVI

SEQ ID NO: 91 - protein, 420 aa, synthetic construct
MRLPLLLVFASVIPGAVLDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 92 - protein, 818 aa, synthetic construct
MRLPLLLVFASVIPGAVLDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 93 - protein, 607 aa, synthetic construct
MRLPLLLVFASVIPGAVLDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 94 - protein, 371 aa, synthetic construct
MEYHPDLENLDEDGYTQLHFDSQSNTRIAVVSEKGSCAASPPWRLIAVILGILCLVILVIAVVLGFVPVFLPAKPTTTPAPRPPTPAPTIASQPLSLRPEACRPAAGGAVHTRGLDFACDGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 95 - protein, 498 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 96 - protein, 1439 aa, synthetic construct
MRLPLLLVFASVIPGAVLDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 97 - protein, 363 aa, synthetic construct
MALPVTALLLPLALLLHAARPGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNDTRKMDPSKPSSNVAGVVIIVILLILTGAGLAAYFFYKKRRVHLPQEGAFENTLYFNSQSSPGTSDMKDLVGNIEQNEHSVI

SEQ ID NO: 98 - protein, 354 aa, synthetic construct
MALPVTALLLPLALLLHAARPGSQIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKRGGGGSGGGGSGGGGSGGGGSQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTTLTVSSDTRKMDPSKPSSNVAGVVIIVILLILTGAGLAAYFFYKKRRVHLPQEGAFENTLYFNSQSSPGTSDMKDLVGNIEQNEHSVI

SEQ ID NO: 99 - protein, 832 aa, synthetic construct
MALPVTALLLPLALLLHAARPGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNGSFVPVFLPAKPTTTPAPRPPTPAPTIASQPLSLRPEACRPAAGGAVHTRGLDFACDFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 100 - protein, 621 aa, synthetic construct
MALPVTALLLPLALLLHAARPGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNGSFVPVFLPAKPTTTPAPRPPTPAPTIASQPLSLRPEACRPAAGGAVHTRGLDFACDAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 101 - protein, 380 aa, synthetic construct
MEYHPDLENLDEDGYTQLHFDSQSNTRIAVVSEKGSCAASPPWRLIAVILGILCLVILVIAVVLGFVPVFLPAKPTTTPAPRPPTPAPTIASQPLSLRPEACRPAAGGAVHTRGLDFACDGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 102 - protein, 507 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 103 - protein, 1453 aa, synthetic construct
MALPVTALLLPLALLLHAARPGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNGSFVPVFLPAKPTTTPAPRPPTPAPTIASQPLSLRPEACRPAAGGAVHTRGLDFACDSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 104 - DNA, 3027 bp, synthetic construct
  source 1..3027: mol_type = other DNA
tcgagcctcggcggcaccgataaggagctccggctcgttgacggcgaaaacaagtgctcaggcagagtggaagtgaaggttcaggaggaatgggggactgtctgcaacaatggctggtctatggaagcggtgtccgtgatctgcaatcagctgggctgtcctacagcaatcaaagcccccggctgggctaatagtagcgctgggtccggccgcatttggatggaccacgtgagctgtagaggaaacgagagcgccctgtgggattgcaagcacgacggctggggcaagcacagcaactgcacccaccagcaggacgcgggcgtgacctgcagcgatggcagcaacctggaaatgaggttgaccagaggcggcaatatgtgctctggccggatcgagatcaagttccagggcagatggggcaccgtgtgtgacgacaattttaatatcgatcacgcttcggttatctgcagacagctggaatgcggctctgccgtctctttcagcgggtcctccaatttcggcgaggggtctggaccaatctggttcgacgacctgatttgcaacggcaacgaatctgctctgtggaattgcaagcaccaggggtggggcaaacacaactgcgaccacgccgaggacgcaggcgttatctgctccaagggcgccgatctctctctgcggctggtggacggtgtcaccgaatgctctggtcggctggaagtacggtttcagggagagtggggcacaatctgtgatgatggctgggacagctacgacgccgccgtggcctgcaagcagctgggctgtcctaccgccgttaccgctatcggacgggtgaacgccagcaagggcttcggccatatctggctggacagcgtgagctgccagggccacgagcctgcaatctggcagtgtaagcaccacgaatggggcaagcactactgtaatcacaacgaagacgccggggtgacatgcagcgacggatccgacctggaattaagactacggggcgggggatccagatgcgccggaacggtcgaggtggagatacagcggttgctgggcaaggtgtgcgacagaggttggggtctgaaggaggctgacgtggtgtgtcgtcagctgggctgcggaagcgctctgaaaacctcttaccaagtgtactctaagatccaggccacaaacacttggctgttcctgagcagctgtaacgggaacgaaacctctctctgggactgcaagaattggcagtggggaggcttgacctgcgatcactacgaggaagctaaaatcacctgcagtgctcacagagagcctcgtttagtgggaggcgacatcccttgctctggccgggtagaagtgaagcacggtgacacttggggtagcatctgtgacagcgatttctccctggaagccgccagcgtgctgtgtagagaactgcagtgcggcaccgtcgtctcaatcctcgggggtgctcacttcggcgaggggaatggccagatctgggccgaggagttccagtgcgagggccacgagagccatctgagtctgtgccccgtggctcctagacctgagggaacctgttctcactccagagacgtcggggttgtgtgctctagatacacagagatccgactggtgaacggcaagaccccttgcgagggcagagttgagctgaagaccttgggcgcttggggcagcctgtgcaacagccactgggatatcgaagatgcccacgtattgtgtcagcagttaaagtgcggcgtggctctttctacacctggcggcgccagattcggcaagggcaacggccagatctggagacacatgttccactgcactggcacagagcagcacatgggcgattgtcctgtgaccgctctgggcgcttctctgtgcccctccgagcaggtggcctccgtaatctgctctggaaaccagagccagaccctctcctcctgcaactcttcttccctgggccccaccaggccgaccattcctgaagagtcagctgtggcgtgcatcgagagcggccagttaagactcgtgaacggcggaggacggtgtgcaggtagagtggagatctaccacgagggctcttggggcaccatatgcgacgacagctgggacctgtccgacgcgcacgtggtgtgtagacaactcggctgtggagaagccattaacgctactgggtctgctcacttcggcgaaggcacgggacccatctggctggacgagatgaagtgcaacggcaaagagagccggatatggcagtgtcactctcacggatggggccagcagaactgcagacacaaagaggatgccggagtcatctgctctgaattcatgagcctgcggctgacctccgaagcttctcgggaggcctgcgccggacggttagaggtgttctacaatggtgcttggggcactgtgggcaagtccagcatgtccgagactactgtcggggtggtgtgccggcagcttgggtgtgccgataaaggcaagatcaaccctgcctctctggacaaggccatgtccattccaatgtgggtggataacgtgcagtgccccaagggccctgacaccctgtggcaatgccctagcagcccttgggaaaagagattagcctctccttcagaagaaacatggatcacctgtgacaacaagatccggctgcaggagggcccaacaagctgttccgggagagtggaaatttggcatggcggcagctggggcaccgtgtgcgacgactcttgggacctggacgatgctcaggtggtgtgccagcaactgggctgtggccctgccctaaaggccttcaaggaggccgagttcggccagggcacgggccctatttggctgaacgaggtaaagtgcaagggcaatgaaagcagtttatgggattgccctgcaagaagatggggccacagcgaatgtggacataaggaagacgccgccgtgaattgtacagacatttcagtgcagaagacccctcagaaggccaccaccggcagaagcagccggcagtccagt

SEQ ID NO: 105 - protein, 1009 aa, synthetic construct
SSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSS

SEQ ID NO: 106 - DNA, 5268 bp, synthetic construct
  source 1..5268: mol_type = other DNA
atgggttggagctgcattatcttgtttcttgtcgccacggctacgggcgttcattcacaccatcaccaccatcatagcaccatcgaggagcaggcaaaaacttttcttgacaagttcaaccatgaggccgaagacttgttctatcaaagctcattggcgagctggaattataatacaaacatcacggaggaaaatgtacagaacatgaacaatgcaggggataaatggtccgcttttctgaaagagcaatccactctcgcacaaatgtatcccttgcaagagatacaaaacttgacagtgaagcttcagctccaggccctgcagcagaatgggtccagcgtcttgagcgaggataaatccaagcgccttaatacgattcttaacacgatgagcactatatacagtacgggcaaggtgtgcaaccccgacaatcctcaagagtgcttgcttctcgagccaggccttaacgaaatcatggcaaactcattggactataatgagcgcctctgggcgtgggaatcttggagatctgaggttggtaagcagcttcgacctttgtatgaagaatacgtggtattgaaaaacgaaatggcgcgagctaatcattacgaagactacggtgactactggcgaggagattatgaagtgaatggggtagacggctacgactactctcgagggcaactcatcgaagatgttgagcacacattcgaagaaatcaaaccactttatgagcatctccatgcgtacgtacgagcgaaactcatgaacgcgtaccccagttatataagtcccatcggttgcctccccgcgcatcttcttggagacatgtgggggagattctggaccaacctctatagtcttactgtacccttcgggcaaaagccgaatatagatgtgactgatgctatggtggaccaggcctgggacgcacaaaggatttttaaggaagcagaaaagttctttgtatctgtggggctccccaatatgactcaagggttctgggaaaactccatgctgacagatcctgggaacgtgcaaaaggccgtgtgtcaccctacagcgtgggaccttgggaaaggtgactttagaattctgatgtgtaccaaggtgactatggacgattttttgaccgctcatcatgagatgggacatatccagtacgatatggcttacgcagctcagcctttcctcctgaggaatggcgccaatgagggatttcatgaagccgtgggcgaaataatgtctctgagcgctgctactcctaagcatttgaaaagcataggcctcctctctcccgacttccaagaggacaacgagacagaaattaatttcctccttaaacaggcgctcaccatagtagggacattgcctttcacatacatgcttgagaaatggagatggatggttttcaaaggggagatccccaaagatcagtggatgaagaaatggtgggagatgaagcgggaaatagttggtgtggtggagccggtcccgcatgacgagacctattgcgatccagcatcactctttcacgtcagcaatgactactctttcattagatattatacccgcactctgtatcaatttcagttccaagaggcgttgtgccaagcggcaaaacatgagggcccccttcacaaatgtgacatatccaactccactgaagcaggccagaaattgtttaatatgctgagactgggtaagagtgaaccatggactcttgccctcgaaaacgtagtcggcgccaaaaatatgaacgttcgccccctgctgaattactttgaacccctctttacgtggctcaaagatcagaacaaaaattccttcgtggggtggtccacagactggtcaccttacgccgaccagagtataaaagtaaggatttcccttaagagtgccctgggtgacaaagcgtacgagtggaacgacaatgagatgtacctttttcgctctagcgttgcatacgctatgcggcagtacttcctgaaggttaagaatcaaatgattcttttcggtgaagaagatgtgcgagtagcaaatttgaagccgcggattagcttcaactttttcgtaaccgcacccaaaaacgtctcagatattatccctaggacggaagtcgagaaagcgatccggatgagtaggagtagaattaatgatgcctttcggctgaacgacaactcccttgaatttcttggcatccagccgacccttggcccgcccaatcagcctccagtgagcagctctctgggtgggacggataaagaattgagactcgttgacggcgagaataagtgctccggacgcgtcgaagtgaaggttcaagaggagtgggggaccgtgtgcaataatggctggagcatggaagccgtcagcgttatttgtaatcaactcggatgcccgactgctatcaaagcaccaggatgggccaattcttctgctgggagcggacgcatttggatggatcatgttagttgtcggggtaatgagagtgcgttgtgggactgcaaacatgatgggtggggtaaacactctaattgtacacatcaacaagatgcgggagtgacgtgtagtgacggctccaatctcgaaatgcgccttacaagaggaggaaatatgtgctctgggaggattgaaatcaaattccaaggccggtggggcacagtgtgcgatgataattttaacatagaccatgccagtgtgatctgccggcagcttgaatgtggttctgcagtcagtttcagcggctcatccaacttcggggagggctcagggcctatatggtttgatgacttgatttgcaacgggaatgagtcagcactgtggaattgtaaacaccagggatggggcaagcataactgtgaccatgccgaagatgccggcgtaatatgctccaaaggtgcggacctctctctccggcttgtggacggtgtcacggagtgctctggacggttggaggtccgctttcagggggagtggggtactatttgcgacgatggttgggactcctacgatgcggctgttgcatgcaaacaattgggatgtcctactgctgttacggcaatcggtcgggtaaacgcatcaaaggggtttgggcatatatggcttgacagcgtatcatgtcaaggtcatgaaccagctatctggcagtgtaaacatcatgagtggggaaaacactactgcaatcacaacgaggatgccggggtcacgtgctctgatggtagtgatctcgagttgaggcttcggggtggcggttcaagatgcgcaggcactgtcgaagtcgaaattcagcgactcctggggaaagtatgcgataggggctggggtctcaaagaggccgatgtcgtttgtagacagttgggttgcggctccgctcttaaaacatcctaccaagtttattctaaaatccaagcgactaatacttggctcttcttgtcttcctgtaatggtaatgagacgtcactctgggattgcaaaaattggcaatggggaggtctgacctgtgaccactacgaagaagctaagattacgtgtagtgcgcatcgagagcctcgcttggtaggaggagacattccttgctcaggccgcgtagaagtcaaacacggggatacttggggttctatctgtgattcagatttttcacttgaagctgcgtctgtgctgtgtagggaacttcaatgtggtacagtcgttagtattctcgggggcgcccattttggtgagggaaatgggcaaatttgggcagaagaattccaatgcgagggacacgagagtcatcttagcttgtgccccgtggcgccaaggccggaagggacatgctctcactcaagagatgtgggagtggtgtgctcaagatatacagagatcaggttggtgaacgggaaaactccttgtgagggtcgagtcgaacttaagacgttgggtgcctggggatcactttgcaatagccactgggacattgaagatgcccatgtgctctgccaacaactcaagtgtggagtcgctttgtccaccccaggcggcgctcgattcggtaagggaaacggtcaaatctggcggcacatgttccactgcactgggacggagcagcatatgggtgactgtccggtgacggctttgggcgccagcttgtgtccaagcgaacaggttgcctccgtgatctgcagtggcaatcagtctcaaacactgagcagctgcaacagttcaagcttggggccgactcggccgaccatacctgaggaaagtgcagtcgcctgcatcgaaagtgggcaattgcgcttggttaatggcggcgggcggtgcgctggccgagtagagatttatcatgaaggttcctgggggaccatctgtgatgactcatgggatcttagcgacgcccacgtggtatgtcgccagctgggttgtggcgaagcaattaatgcgacaggttctgcgcacttcggtgaaggaacggggccgatatggcttgacgagatgaaatgcaacggtaaagaatcaaggatttggcaatgtcacagccacggttgggggcaacagaactgtagacacaaggaagacgccggcgtcatatgttcagagtttatgtccttgagattgacgagcgaggccagtcgagaagcttgcgccgggcggcttgaagttttctacaatggagcctgggggaccgtgggtaaaagtagtatgagcgaaaccacagtaggagtagtttgtcgccaacttgggtgtgccgataagggcaagattaatcccgcttcccttgataaggcgatgtccataccgatgtgggtcgacaacgtgcaatgcccaaaaggacctgatacactttggcagtgccctagtagtccttgggagaagagattggccagtccgtctgaagaaacttggataacatgtgacaacaagatacgacttcaagagggacctacgtcatgttcaggtcgagtggaaatctggcacggaggatcatgggggacggtttgtgacgatagctgggatctggatgatgcccaggtagtctgccaacagctcggatgcggtccggcgttgaaggcgttcaaggaagccgagtttggccaaggcacaggaccaatttggcttaatgaagtgaaatgcaagggtaacgaaagctctctttgggactgtccggcacggcggtgggggcacagtgagtgtggccataaggaagacgcagcagtgaactgcacggatattagtgttcagaagaccccgcaaaaagcgacgaccgggcggagctcccgccagtccagt

SEQ ID NO: 107 - protein, 1756 aa, synthetic construct
MGWSCIILFLVATATGVHSHHHHHHSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSS

(19) United States
(12) Patent Application Publication
(10) Pub. No.: US 2025/0092112 A1
(43) Pub. Date: Mar. 20, 2025

(54) CHIMERIC BAIT RECEPTORS AND USES THEREOF

(71) Applicant: Hemogenyx Pharmaceuticals, LLC, Brooklyn, NY (US)

(72) Inventors: Vladislav Sandler, Brooklyn, NY (US); Ronen Ben Jehuda, Brooklyn, NY (US); Elina Shrestha, Brooklyn, NY (US); Carina Sirochinsky, Brooklyn, NY (US); Michael Haugbro, Brooklyn, NY (US); Raymond Liang, Brooklyn, NY (US)

(21) Appl. No.: 18/823,398

(22) Filed: Sep. 3, 2024

Related U.S. Application Data
(63) Continuation of application No. PCT/US2023/063514, filed on Mar. 1, 2023.
(60) Provisional application No. 63/315,388, filed on Mar. 1, 2022.

(51) Int. Cl. C07K 14/725; A61K 38/00; C07K 14/705; C07K 16/28; C12N 9/48

(52) U.S. Cl. CPC C07K 14/7051; C07K 14/70517; C07K 14/7056; C07K 14/70596; C07K 16/2863; C12N 9/48; A61K 38/00; C07K 2317/53; C07K 2317/622; C07K 2319/02; C07K 2319/03; C12Y 304/17023

(57) ABSTRACT

Provided herein are novel chimeric polypeptides that bind an antigenic peptide (e.g., a viral antigen or a tumor-associated antigen) and activate the endogenous phagocytic signaling pathway. Also provided are compositions and methods useful for producing such chimeric polypeptides, nucleic acids encoding same, phagocytic cells that have been modified to express such chimeric polypeptides, as well as methods for the treatment of various disorders, such as viral infections or cancers.

### CROSS REFERENCE TO RELATED APPLICATIONS

This application is a Continuation of PCT/US2023/063514 filed Mar. 1, 2023, which claims the benefit of U.S. Provisional Patent Application No. 63/315,388, filed Mar. 1, 2022, the entire contents of which are incorporated herein by reference.

### SEQUENCE LISTING

The instant application contains a Sequence Listing which has been submitted electronically in XML format and is hereby incorporated by reference in its entirety. Said XML file, created on Feb. 28, 2023, is named 405320-HGXR-001WO.xml and is 203,056 bytes in size.

### FIELD

The instant disclosure relates to novel chimeric polypeptides that bind an antigenic peptide (e.g., a viral antigen or a tumor-associated antigen) and activate the endogenous phagocytic signaling pathway. Also provided are compositions and methods useful for producing such chimeric polypeptides, nucleic acids encoding same, phagocytic cells that have been modified to express such chimeric polypeptides, as well as methods for the treatment of various disorders, such as viral infections or cancers.

### BACKGROUND

Many viruses bind to one or multiple specific receptors on host cells in order to carry out attachment, entry, and/or signaling. A single viral receptor can mediate all of the aforementioned functions or viruses can utilize distinct receptors to mediate each function, thereby lending even greater specificity in tissue tropism. In other cases, coordinated virus-receptor interactions are necessary to mediate specific functions, such as activation of signaling events. Studies have revealed common viral receptors including cell adhesion molecules (CAMs) such as integrins, selectins, cadherins, and immunoglobulin superfamily (IgSF) receptors and PtdSer receptors. Viruses including HIV, measles virus, reovirus, rhinovirus, adenovirus, poliovirus, and coxsackievirus B (CVB) utilize IgSF members as receptors, whereas integrins serve as receptors for reovirus, rotavirus, adenovirus, West Nile virus (WNV), human metapneuomovirus (hMPV), foot-and-mouth disease virus (FMDV), herpes simplex virus (HSV), human cytomegalovirus HCMV and human herpesvirus-8.

Other viruses bind to less common receptors on host cells to carry out attachment, entry, and/or signaling. For example, SARS-CoV-2 binds to human angiotensin converting enzyme 2 (ACE2), a membrane-bound enzyme which is part of the renin-angiotensin-aldosterone system.

The immune system constantly patrols human body, looking to eliminate cancerous cells and harmful microbes. Under normal circumstances, immune cells can identify these threats because they can recognize certain signals present at the surface of the target cells. Immunity provided by antibodies produced against viral antigens, either through previous infection or vaccination is currently the primary method for prevention of viral infection. However, frequent viral mutation could allow viruses to evade this immunity if the antibodies produced no longer recognize the viral antigen or bind viral antigen with less affinity.

Furthermore, cancer cells often find ways to evade the immune system and proliferate. The endogenous immune system is typically non-reactive to malignant cells or can be actively immunosuppressive with respect to the body's reaction to the presence of malignant cells. Despite many recent advances in cancer immunotherapy, a vast majority of current cellular therapeutics focus almost exclusively on T cells. This focus is largely due to the fact that T cells can be engineered to express a synthetic immunoreceptor containing an extracellular targeted antibody and intracellular signaling domain, known as chimeric antigen receptor (CAR), which give the T cells expressing such CAR the ability to target a tumor-associated antigen. For example, in recent years, T cells expressing a CAR directed against CD19 have been shown to have significant anti-leukemic efficacy, where complete remission has been achieved in 90% of acute lymphoblastic leukemia patients treated. These results are accompanied by robust T cell proliferation and clearly documented T cell infiltration into tumor sites in leukemic patients so treated. Despite the high response rates demonstrated in hematopoietic malignancies, CAR T cell efficacy in solid tumors, as well as in certain lymphoid tumors, may be limited. Possible explanations for this include the potentially impaired ability of T cells to infiltrate solid tumors, poor trafficking, immunosuppressive tumor microenvironment, and expression of few tumor specific antigens on solid tumor cells.

There is an ongoing need for new compositions and methods of treating infections, inflammatory diseases, immune diseases, and various cancers. Given the problems associated with natural and recombinant antibodies against viruses, there is an unmet need for antibody-independent innate immunity mechanisms to neutralize viruses. Targeting the viral receptor on the host cell provides an opportunity to prevent and/or treat viral infection. A need also exists for more effective compositions and methods to treat cancers by improving specificity for tumor cells and improving infiltration into tumor sites in both solid tumors and hematologic malignancies.

### SUMMARY

The instant disclosure provides chimeric polypeptides which include a portion that is capable of specifically binding to an antigen and a portion that targets the chimeric polypeptide for endocytosis upon binding to the antigen. As described in further detail below, the antigen may be a viral protein and the portion that is capable of specifically binding to the viral protein may be a binding region that the virus binds to on a host cell. In other embodiments, the antigen may be a tumor associated antigen (TAA) and the portion that is capable of specifically binding to the TAA may be an antigen binding portion, such as an antibody or fragment thereof. The portion of the chimeric polypeptides that targets the chimeric polypeptide for endocytosis may be an intracellular (i.e., cytoplasmic) signaling region of an endocytic receptor. In other embodiments, the portion of the chimeric polypeptides that targets the chimeric polypeptide for endocytosis may be a ligand for an endocytic receptor.

In an aspect, a chimeric bait receptor (CBR) is provided comprising a) an extracellular portion comprising a binding region that a virus specifically binds to, wherein the binding region is not an antibody; b) a transmembrane portion; and c) an intracellular portion comprising an intracellular signaling region of an endocytic receptor.

In some embodiments, the binding region is a portion of a host protein that confers attachment of the virus to a host cell when the binding region is exposed to the virus and the binding region is expressed in the host cell.

In some embodiments, the binding region comprises a portion of a protein selected from the group consisting of angiotensin converting enzyme 2 (ACE2), CD4, CCR5, CXCR4, T-cell Ig and mucin domain 1 (TIM-1), CD46, and SLAMF1. In some embodiments, the binding region comprises ACE2 or a fragment thereof. In some embodiments, the binding region comprises amino acids 19-358, 19-605 or 19-740 of SEQ ID NO: 2. In some embodiments, the binding region comprises an amino acid sequence at least 90, 91, 92, 93, 94, 95, 96, 97, 98, 99, or 100% an amino acid sequence selected from the group consisting of SEQ ID NO: 2, SEQ ID NO: 4, SEQ ID NO: 6, and SEQ ID NO: 8.

In some embodiments, the transmembrane portion comprises a transmembrane portion of a protein selected from the group consisting of CD8, mannose receptor, MER proto-oncogene tyrosine kinase (MERTK), dectin-1, and a scavenger receptor.

In some embodiments, the transmembrane portion comprises a transmembrane portion of a scavenger receptor. In some embodiments, the scavenger receptor is a member of a class of scavenger receptor selected from the group consisting of class A, B, C, D, E, F, G, H, I K, J, K and L scavenger receptor. In some embodiments, the scavenger receptor is selected from the group consisting of scavenger receptor class A type I/II (SR AI/II), macrophage receptor with collagenous structure (MARCO), SCARA5 receptor, scavenger receptor with C-type lectin (SRCL), CD36, scavenger receptor class B type I (SR-BI), CD68, lectin-like oxLDL receptor 1 (LOX-1), scavenger receptor expressed by endothelial cell (SREC), multiple EGF like portions 10 (MEGF10), scavenger receptor for phosphatidylserine and oxidized lipoprotein (SR-PSOX), link domain-containing scavenger receptor-1 (FEEL-1), CD163, receptor for advanced glycation end products (RAGE), CD44, and scavenger receptor class L type I (SR-L1).

In some embodiments, the intracellular signaling region of the endocytic receptor comprises an intracellular portion of the mannose receptor.

In some embodiments, the transmembrane portion comprises a transmembrane portion of the mannose receptor.

In some embodiments, the transmembrane portion and the intracellular portion of the mannose receptor comprises an amino acid sequence at least 90, 91, 92, 93, 94, 95, 96, 97, 98, 99, or 100% to the amino acid sequence of SEQ ID NO:10.

In some embodiments, the transmembrane portion and the intracellular portion of the mannose receptor comprises an amino acid sequence at least 90, 91, 92, 93, 94, 95, 96, 97, 98, 99, or 100% to the amino acid sequence of SEQ ID NO:12.

In some embodiments, the intracellular signaling region of the endocytic receptor comprises an intracellular signaling region of a phagocytic receptor.

In some embodiments, the transmembrane portion comprises a transmembrane portion of a phagocytic receptor.

In some embodiments, the intracellular signaling region of the phagocytic receptor comprises an intracellular portion from a protein selected from the group consisting of MERTK, dectin-1, and Fc gamma receptor (FcγR).

In some embodiments, the transmembrane portion and the intracellular signaling region of the phagocytic receptor comprises the transmembrane portion and the intracellular portion of MERTK. In some embodiments, the transmembrane portion and the intracellular portion of MERTK comprises an amino acid sequence at least 90, 91, 92, 93, 94, 95, 96, 97, 98, 99, or 100% to the amino acid sequence of SEQ ID NO:14.

In some embodiments, the transmembrane portion and the intracellular signaling region of the phagocytic receptor comprises the transmembrane portion and the intracellular portion of dectin-1. In some embodiments, the transmembrane portion and the intracellular portion of dectin-1 comprises an amino acid sequence at least 90, 91, 92, 93, 94, 95, 96, 97, 98, 99, or 100% to the amino acid sequence of SEQ ID NO:16. In some embodiments, the CBR comprises SEQ ID NO: 18.

In some embodiments, the intracellular signaling region of the phagocytic receptor comprises the intracellular portion of FcγR. In some embodiments, the intracellular portion of FcγR comprises an amino acid sequence at least 90, 91, 92, 93, 94, 95, 96, 97, 98, 99, or 100% to the amino acid sequence of SEQ ID NO:20.

In some embodiments, the transmembrane portion and the intracellular signaling region of the endocytic receptor comprises a transmembrane portion and an intracellular signaling region of a scavenger receptor. In some embodiments, the scavenger receptor is a member of a class of scavenger receptor selected from the group consisting of class A, B, C, D, E, F, G, H, I K, J, K and L scavenger receptor. In some embodiments, the scavenger receptor is selected from the group consisting of SR AI/II, MARCO, SCARA5 receptor, SRCL, CD36, SR-BI, CD68, LOX-1, SREC, MEGF10, SR-PSOX, FEEL-1, CD163, RAGE, CD44, and SR-L1.

In some embodiments, the transmembrane portion and the intracellular signaling region of the scavenger receptor comprises the transmembrane portion and the intracellular portion of SR AI/II. In some embodiments, the transmembrane portion and the intracellular portion of SR AI/II comprises an amino acid sequence at least 90, 91, 92, 93, 94, 95, 96, 97, 98, 99, or 100% to the amino acid sequence of SEQ ID NO:22.

In some embodiments, the transmembrane portion and the intracellular signaling region of the scavenger receptor comprises the transmembrane portion and the intracellular portion of MARCO. In some embodiments, the transmembrane portion and the intracellular portion of MARCO comprises an amino acid sequence at least 90, 91, 92, 93, 94, 95, 96, 97, 98, 99, or 100% to the amino acid sequence of SEQ ID NO:24.

In some embodiments, the transmembrane portion and the intracellular signaling region of the scavenger receptor comprises the transmembrane portion and the intracellular portion of SCARA5 receptor. In some embodiments, the transmembrane portion and the intracellular portion of SCARA5 receptor comprises an amino acid sequence at least 90, 91, 92, 93, 94, 95, 96, 97, 98, 99, or 100% to the amino acid sequence of SEQ ID NO:26.

In some embodiments, the transmembrane portion and the intracellular signaling region of the scavenger receptor comprises the transmembrane portion and the intracellular portion of SRCL. In some embodiments, the transmembrane portion and the intracellular portion of SRCL comprises an amino acid sequence at least 90, 91, 92, 93, 94, 95, 96, 97, 98, 99, or 100% to the amino acid sequence of SEQ ID NO:28.

In some embodiments, the transmembrane portion and the intracellular signaling region of the scavenger receptor comprises the transmembrane portion and the intracellular portion of CD36. In some embodiments, the transmembrane portion and the intracellular portion of CD36 comprises an amino acid sequence at least 90, 91, 92, 93, 94, 95, 96, 97, 98, 99, or 100% to the amino acid sequence of SEQ ID NO:30.

In some embodiments, the transmembrane portion and the intracellular signaling region of the scavenger receptor comprises the transmembrane portion and the intracellular portion of SR-BI. In some embodiments, the transmembrane portion and the intracellular portion of SR-BI comprises an amino acid sequence at least 90, 91, 92, 93, 94, 95, 96, 97, 98, 99, or 100% to the amino acid sequence of SEQ ID NO:32.

In some embodiments, the transmembrane portion and the intracellular signaling region of the scavenger receptor comprises the transmembrane portion and the intracellular portion of CD68. In some embodiments, the transmembrane portion and the intracellular portion of CD68 comprises an amino acid sequence at least 90, 91, 92, 93, 94, 95, 96, 97, 98, 99, or 100% to the amino acid sequence of SEQ ID NO:34.

In some embodiments, the transmembrane portion and the intracellular signaling region of the scavenger receptor comprises the transmembrane portion and the intracellular portion of LOX-1. In some embodiments, the transmembrane portion and the intracellular portion of LOX-1 comprises an amino acid sequence at least 90, 91, 92, 93, 94, 95, 96, 97, 98, 99, or 100% to the amino acid sequence of SEQ ID NO:36.

In some embodiments, the transmembrane portion and the intracellular signaling region of the scavenger receptor comprises the transmembrane portion and the intracellular portion of SREC. In some embodiments, the transmembrane portion and the intracellular portion of SREC comprises an amino acid sequence at least 90, 91, 92, 93, 94, 95, 96, 97, 98, 99, or 100% to the amino acid sequence of SEQ ID NO:38.

In some embodiments, the transmembrane portion and the intracellular signaling region of the scavenger receptor comprises the transmembrane portion and the intracellular portion of MEGF10. In some embodiments, the transmembrane portion and the intracellular portion of MEGF10 comprises an amino acid sequence at least 90, 91, 92, 93, 94, 95, 96, 97, 98, 99, or 100% to the amino acid sequence of SEQ ID NO:40.

In some embodiments, the transmembrane portion and the intracellular signaling region of the scavenger receptor comprises the transmembrane portion and the intracellular portion of SR-PSOX. In some embodiments, the transmembrane portion and the intracellular portion of SR-PSOX comprises an amino acid sequence at least 90, 91, 92, 93, 94, 95, 96, 97, 98, 99, or 100% to the amino acid sequence of SEQ ID NO:42.

In some embodiments, the transmembrane portion and the intracellular signaling region of the scavenger receptor comprises the transmembrane portion and the intracellular portion of FEEL-1. In some embodiments, the transmembrane portion and the intracellular portion of FEEL-1 comprises an amino acid sequence at least 90, 91, 92, 93, 94, 95, 96, 97, 98, 99, or 100% to the amino acid sequence of SEQ ID NO:44.

In some embodiments, the transmembrane portion and the intracellular signaling region of the scavenger receptor comprises the transmembrane portion and the intracellular portion of CD163. In some embodiments, the transmembrane portion and the intracellular portion of CD163 comprises an amino acid sequence at least 90, 91, 92, 93, 94, 95, 96, 97, 98, 99, or 100% to the amino acid sequence of SEQ ID NO:46.

In some embodiments, the transmembrane portion and the intracellular signaling region of the scavenger receptor comprises the transmembrane portion and the intracellular portion of RAGE. In some embodiments, the transmembrane portion and the intracellular portion of RAGE comprises an amino acid sequence at least 90, 91, 92, 93, 94, 95, 96, 97, 98, 99, or 100% to the amino acid sequence of SEQ ID NO:48.

In some embodiments, the transmembrane portion and the intracellular signaling region of the scavenger receptor comprises the transmembrane portion and the intracellular portion of CD44. In some embodiments, the transmembrane portion and the intracellular portion of CD44 comprises an amino acid sequence at least 90, 91, 92, 93, 94, 95, 96, 97, 98, 99, or 100% to the amino acid sequence of SEQ ID NO:50.

In some embodiments, the transmembrane portion and the intracellular signaling region of the scavenger receptor comprises the transmembrane portion and the intracellular portion of SR-L1. In some embodiments, the transmembrane portion and the intracellular portion of SR-L1 comprises an amino acid sequence at least 90, 91, 92, 93, 94, 95, 96, 97, 98, 99, or 100% to the amino acid sequence of SEQ ID NO:52.

In some embodiments, the CBR further comprises an N-terminal signal peptide. In some embodiments, the N-terminal signal peptide comprises a CD8 signal peptide or a mannose receptor signal peptide.

In some embodiments, the CBR comprises an amino acid sequence at least 90, 91, 92, 93, 94, 95, 96, 97, 98, 99, or 100% identical to an amino acid sequence selected from the group consisting of SEQ ID NOs: 61-67.

Also provided are nucleic acids encoding one or more of the CBRs described herein.

In some embodiments, the nucleic acid comprises a nucleotide sequence at least 75, 80, 85, 90, 91, 92, 93, 94, 95, 96, 97, 98, 99 or 100% identical to a nucleotide sequence selected from the group consisting of SEQ ID NOs: 54-60.

Recombinant vectors encoding one or more CBRs of the instant disclosure are also provided, as are cells comprising one of more CBRs, nucleic acids encoding one or more CBRs, and/or vectors capable of expressing one or more CBRs. In some embodiments, the host cell is a phagocytic cell, such as, for example, a macrophage, a dendritic cell, a mast cell, a monocyte, a neutrophil, a microglial cell or an astrocyte. A population of cells comprising two or more cells described herein is also provided.

A pharmaceutical composition comprising a therapeutically effective amount of one or more CBR, one or more nucleic acid encoding a CBR, one or more vectors capable of expressing a CBR, a host cell or the population of host cells comprising a CBR, and a pharmaceutically acceptable carrier is also provided.

A method of treating or preventing a viral infection in a subject in need thereof is also provided comprising administering to the subject a therapeutically effective amount of one or more CBRs, one or more nucleic acids encoding a CBR, one or more vectors capable of expressing a CBR, a host cell or the population of host cells comprising a CBR, or a pharmaceutical composition containing any of the above is also provided.

In some embodiments, the subject is a human.

In some embodiments, the viral infection is caused by a virus selected from the group consisting of a Togaviridae virus, a Coronaviridae virus, a Flaviviridae virus, an Orthomyxoviridae virus, a Filoviridae virus, a Paramyxoviridae virus, a Retroviridae virus and a Bunyaviradae virus.

In some embodiments, the virus is a coronavirus. In some embodiments, the coronavirus is SARS-CoV-2.

In an aspect, a chimeric phagocytic receptor (CPR) is provided comprising a) an extracellular portion comprising an antigen binding portion, b) a transmembrane portion; and c) an intracellular portion comprising an intracellular signaling region of an endocytic receptor, wherein the CPR does not comprise a recruitment portion, wherein the recruitment portion is not the endocytic receptor intracellular region, and wherein the recruitment portion binds to a cytosolic protein of a phagocytic signaling pathway.

In some embodiments, the antigen binding portion comprises an antibody. In some embodiments, the antibody comprises a single-chain variable fragment (scFv) or a single portion antibody (sdAb) variable portion.

In some embodiments, the antigen binding portion specifically binds to an antigen selected from the group consisting of CD19, CD22, HER2 (ERBB2/neu), Mesothelin, PSCA, CD123, CD30, CD171, CD138, CS-1, CLECL1, CD33, CD10, CD79b, EGFRvIII, GD2, GD3, BCMA, PSMA, RORI, FLT3 (CD135), TAG72, CD38, CD44v6, CEA, EPCAM, B7H3 (CD276), KIT (CD 117), CD213A2, IL-1 IRa, PRSS21, VEGFR2, FSHR, TROP2, CD24, MUC-16, PDGFR-beta, SSEA-4, CD20, MUC1, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, Tn-glycopeptides and IGLL1.

In some embodiments, the transmembrane portion comprises a transmembrane portion of a protein selected from the group consisting of CD8, mannose receptor, MER proto-oncogene tyrosine kinase (MERTK), dectin-1, and a scavenger receptor.

In some embodiments, the transmembrane portion comprises a transmembrane portion of a scavenger receptor. In some embodiments, the scavenger receptor is a member of a class of scavenger receptor selected from the group consisting of class A, B, C, D, E, F, G, H, I K, J, K and L scavenger receptor. In some embodiments, the scavenger receptor is selected from the group consisting of scavenger receptor class A type I/II (SR AI/II), macrophage receptor with collagenous structure (MARCO), SCARA5 receptor, scavenger receptor with C-type lectin (SRCL), CD36, scavenger receptor class B type I (SR-BI), CD68, lectin-like oxLDL receptor 1 (LOX-1), scavenger receptor expressed by endothelial cell (SREC), multiple EGF like portions 10 (MEGF10), scavenger receptor for phosphatidylserine and oxidized lipoprotein (SR-PSOX), link domain-containing scavenger receptor-1 (FEEL-1), CD163, receptor for advanced glycation endproducts (RAGE), CD44, and scavenger receptor class L type I (SR-L1).

In some embodiments, the intracellular signaling region of the endocytic receptor comprises an intracellular portion of the mannose receptor.

In some embodiments, the transmembrane portion comprises a transmembrane portion of the mannose receptor.

In some embodiments, the transmembrane portion and the intracellular portion of the mannose receptor comprises an amino acid sequence at least 90, 91, 92, 93, 94, 95, 96, 97, 98, 99, or 100% to the amino acid sequence of SEQ ID NO:10.

In some embodiments, the transmembrane portion and the intracellular portion of the mannose receptor comprises an amino acid sequence at least 90, 91, 92, 93, 94, 95, 96, 97, 98, 99, or 100% to the amino acid sequence of SEQ ID NO:12.

In some embodiments, the intracellular signaling region of the endocytic receptor comprises an intracellular signaling region of a phagocytic receptor.

In some embodiments, the transmembrane portion comprises a transmembrane portion of a phagocytic receptor.

In some embodiments, the intracellular signaling region of the phagocytic receptor comprises an intracellular portion from a protein selected from the group consisting of MERTK, dectin-1, and Fc gamma receptor (FcγR).

In some embodiments, the transmembrane portion and the intracellular signaling region of the phagocytic receptor comprises the transmembrane portion and the intracellular portion of MERTK. In some embodiments, the transmembrane portion and the intracellular portion of MERTK comprises an amino acid sequence at least 90, 91, 92, 93, 94, 95, 96, 97, 98, 99, or 100% to the amino acid sequence of SEQ ID NO:14.

In some embodiments, the transmembrane portion and the intracellular signaling region of the phagocytic receptor comprises the transmembrane portion and the intracellular portion of dectin-1. In some embodiments, the transmembrane portion and the intracellular portion of dectin-1 comprises an amino acid sequence at least 90, 91, 92, 93, 94, 95, 96, 97, 98, 99, or 100% to the amino acid sequence of SEQ ID NO:16. In some embodiments, the CBR comprises an amino acid sequence at least 90, 91, 92, 93, 94, 95, 96, 97, 98, 99, or 100% to the amino acid sequence of SEQ ID NO: 18.

In some embodiments, the intracellular signaling region of the phagocytic receptor comprises the intracellular portion of FcγR. In some embodiments, the intracellular portion of FcγR comprises an amino acid sequence at least 90, 91, 92, 93, 94, 95, 96, 97, 98, 99, or 100% to the amino acid sequence of SEQ ID NO:20.

In some embodiments, the transmembrane portion and the intracellular signaling region of the endocytic receptor comprises a transmembrane portion and an intracellular signaling region of a scavenger receptor. In some embodiments, the scavenger receptor is a member of a class of scavenger receptor selected from selected from the group consisting of class A, B, C, D, E, F, G, H, I K, J, K and L scavenger receptor. In some embodiments, the scavenger receptor is selected from the group consisting of SR AI/II, MARCO, SCARA5 receptor, SRCL, CD36, SR-BI, CD68, LOX-1, SREC, MEGF10, SR-PSOX, FEEL-1, CD163, RAGE, CD44, and SR-L1.

In some embodiments, the transmembrane portion and the intracellular signaling region of the scavenger receptor comprises the transmembrane portion and the intracellular portion of SR AI/II. In some embodiments, the transmembrane portion and the intracellular portion of SR AI/II comprises an amino acid sequence at least 90, 91, 92, 93, 94, 95, 96, 97, 98, 99, or 100 to the amino acid sequence of SEQ ID NO:22.

In some embodiments, the transmembrane portion and the intracellular signaling region of the scavenger receptor comprises the transmembrane portion and the intracellular portion of MARCO. In some embodiments, the transmembrane portion and the intracellular portion of MARCO comprises an amino acid sequence at least 90, 91, 92, 93, 94, 95, 96, 97, 98, 99, or 100% to the amino acid sequence of SEQ ID NO:24.

In some embodiments, the transmembrane portion and the intracellular signaling region of the scavenger receptor comprises the transmembrane portion and the intracellular portion of SCARA5 receptor. In some embodiments, the transmembrane portion and the intracellular portion of SCARA5 receptor comprises an amino acid sequence at least 90, 91, 92, 93, 94, 95, 96, 97, 98, 99, or 100% to the amino acid sequence of SEQ ID NO:26.

In some embodiments, the transmembrane portion and the intracellular signaling region of the scavenger receptor comprises the transmembrane portion and the intracellular portion of SRCL. In some embodiments, the transmembrane portion and the intracellular portion of SRCL comprises an amino acid sequence at least 90, 91, 92, 93, 94, 95, 96, 97, 98, 99, or 100% to the amino acid sequence of SEQ ID NO:28.

In some embodiments, the transmembrane portion and the intracellular signaling region of the scavenger receptor comprises the transmembrane portion and the intracellular portion of CD36. In some embodiments, the transmembrane portion and the intracellular portion of CD36 comprises an amino acid sequence at least 90, 91, 92, 93, 94, 95, 96, 97, 98, 99, or 100% to the amino acid sequence of SEQ ID NO:30.

In some embodiments, the transmembrane portion and the intracellular signaling region of the scavenger receptor comprises the transmembrane portion and the intracellular portion of SR-BI. In some embodiments, the transmembrane portion and the intracellular portion of SR-BI comprises an amino acid sequence at least 90, 91, 92, 93, 94, 95, 96, 97, 98, 99, or 100% to the amino acid sequence of SEQ ID NO:32.

In some embodiments, the transmembrane portion and the intracellular signaling region of the scavenger receptor comprises the transmembrane portion and the intracellular portion of CD68. In some embodiments, the transmembrane portion and the intracellular portion of CD68 comprises an amino acid sequence at least 90, 91, 92, 93, 94, 95, 96, 97, 98, 99, or 100% to the amino acid sequence of SEQ ID NO:34.

In some embodiments, the transmembrane portion and the intracellular signaling region of the scavenger receptor comprises the transmembrane portion and the intracellular portion of LOX-1. In some embodiments, the transmembrane portion and the intracellular portion of LOX-1 comprises an amino acid sequence at least 90, 91, 92, 93, 94, 95, 96, 97, 98, 99, or 100% to the amino acid sequence of SEQ ID NO:36.

In some embodiments, the transmembrane portion and the intracellular signaling region of the scavenger receptor comprises the transmembrane portion and the intracellular portion of SREC. In some embodiments, the transmembrane portion and the intracellular portion of SREC comprises an amino acid sequence at least 90, 91, 92, 93, 94, 95, 96, 97, 98, 99, or 100% to the amino acid sequence of SEQ ID NO:38.

In some embodiments, the transmembrane portion and the intracellular signaling region of the scavenger receptor comprises the transmembrane portion and the intracellular portion of MEGF10. In some embodiments, the transmembrane portion and the intracellular portion of MEGF10 comprises an amino acid sequence at least 90, 91, 92, 93, 94, 95, 96, 97, 98, 99, or 100% to the amino acid sequence of SEQ ID NO:40.

In some embodiments, the transmembrane portion and the intracellular signaling region of the scavenger receptor comprises the transmembrane portion and the intracellular portion of SR-PSOX. In some embodiments, the intracellular portion of SR-PSOX comprises an amino acid sequence at least 90, 91, 92, 93, 94, 95, 96, 97, 98, 99, or 100% to the amino acid sequence of SEQ ID NO:42.

In some embodiments, the transmembrane portion and the intracellular signaling region of the scavenger receptor comprises the transmembrane portion and the intracellular portion of FEEL-1. In some embodiments, the transmembrane portion and the intracellular portion of FEEL-1 comprises an amino acid sequence at least 90, 91, 92, 93, 94, 95, 96, 97, 98, 99, or 100% to the amino acid sequence of SEQ ID NO:44.

In some embodiments, the transmembrane portion and the intracellular signaling region of the scavenger receptor comprises the transmembrane portion and the intracellular portion of CD163. In some embodiments, the transmembrane portion and the intracellular portion of CD163 comprises an amino acid sequence at least 90, 91, 92, 93, 94, 95, 96, 97, 98, 99, or 100% to the amino acid sequence of SEQ ID NO:46.

In some embodiments, the transmembrane portion and the intracellular signaling region of the scavenger receptor comprises the transmembrane portion and the intracellular portion of RAGE. In some embodiments, the transmembrane portion and the intracellular portion of RAGE comprises an amino acid sequence at least 90, 91, 92, 93, 94, 95, 96, 97, 98, 99, or 100% to the amino acid sequence of SEQ ID NO:48.

In some embodiments, the transmembrane portion and the intracellular signaling region of the scavenger receptor comprises the transmembrane portion and the intracellular portion of CD44. In some embodiments, the transmembrane portion and the intracellular portion of CD44 comprises an amino acid sequence at least 90, 91, 92, 93, 94, 95, 96, 97, 98, 99, or 100% to the amino acid sequence of SEQ ID NO:50.

In some embodiments, the transmembrane portion and the intracellular signaling region of the scavenger receptor comprises the transmembrane portion and the intracellular portion of SR-L1. In some embodiments, the transmembrane portion and the intracellular portion of SR-L1 comprises an amino acid sequence at least 90, 91, 92, 93, 94, 95, 96, 97, 98, 99, or 100% to the amino acid sequence of SEQ ID NO:52.

In some embodiments, the recruitment portion is selected from the group consisting of a) a p85-recruitment portion that binds a p85 regulatory subunit of phosphoinositide 3-kinase (PI3K); b) an SH3 portion derived from Crk, Cdc25, Phospholipase, Ras, Vav, GRB2, FAK, Pyk2, TRIP10 or Gads; and c) a proline-rich peptide sequence from C3G, p41, PEP, p4′7, HPK1, SLP-1, CD3.epsilon., PAK, AIP4, or Sos, wherein the proline-rich peptide sequence binds to an SH3 portion-containing protein. In some embodiments, the recruitment portion is the p85-recruitment portion, and wherein the p85-recruitment portion is derived from CD19, Gab2, IREM-1, PDGF receptor, CSFR-1, c-Kit, ErbB3, or CD7.

In some embodiments, the CPR further comprises an N-terminal signal peptide. In some embodiments, the N-terminal signal peptide comprises a CD8 signal peptide or a mannose receptor signal peptide.

In some embodiments, the CPR comprises an amino acid sequence at least 90, 91, 92, 93, 94, 95, 96, 97, 98, 99, or 100% identical to an amino acid sequence selected from the group consisting of SEQ ID NOs: 86-103.

Also provided are nucleic acids encoding one or more of the CPRs described herein.

In some embodiments, the nucleic acid comprises a nucleotide sequence at least 75, 80, 85, 90, 91, 92, 93, 94, 95, 96, 97, 98, 99 or 100% identical to a nucleotide sequence selected from the group consisting of SEQ ID NOs: 68-85.

Recombinant vectors encoding one or more CPRs of the instant disclosure are also provided, as are cells comprising one of more CPRs, nucleic acids encoding one or more CPRs, and/or vectors capable of expressing one or more CPRs. In some embodiments, the host cell is a phagocytic cell, such as, for example, a macrophage, a dendritic cell, a mast cell, a monocyte, a neutrophil, a microglial cell or an astrocyte. A population of cells comprising two or more cells described herein is also provided.

A pharmaceutical composition comprising a therapeutically effective amount of one or more CPR, one or more nucleic acid encoding a CPR, one or more vectors capable of expressing a CPR, a host cell or the population of host cells comprising a CPR, and a pharmaceutically acceptable carrier is also provided.

A method of treating cancer in a subject in need thereof is also provided comprising administering to the subject a therapeutically effective amount of one or more CPR, one or more nucleic acid encoding a CPR, one or more vectors capable of expressing a CPR, a host cell or the population of host cells comprising a CPR, or a pharmaceutical composition containing any of the above. In some embodiments, the subject is a human.

In an aspect, a bait macrophage engager (BME) is provided comprising a) a binding region that a virus specifically binds to, wherein the binding region is not an antibody; and b) a ligand for an endocytic receptor.

In an aspect, a bait macrophage engager (BME) is provided comprising a) a binding region that a virus specifically binds to, wherein the binding region is a portion of a host protein that confers attachment of the virus to a host cell when the binding region is exposed to the virus and the binding region is expressed in the host cell; and b) a ligand for an endocytic receptor.

In some embodiments, the binding region comprises a portion of a protein selected from the group consisting of angiotensin converting enzyme 2 (ACE2), CD4, CCR5, CXCR4, T-cell Ig and mucin domain 1 (TIM-1), CD46, and SLAMF1.

In some embodiments, the binding region comprises ACE2 or a fragment thereof. In some embodiments, the binding region comprises amino acids 19-358, 19-605 or 19-740 of SEQ ID NO: 2. In some embodiments, the binding region comprises an amino acid sequence at least 90, 91, 92, 93, 94, 95, 96, 97, 98, 99, or 100% an amino acid sequence selected from the group consisting of SEQ ID NO: 2, SEQ ID NO: 4, SEQ ID NO: 6, and SEQ ID NO: 8.

In some embodiments, the endocytic receptor is a mannose receptor.

In some embodiments, the endocytic receptor is a phagocytic receptor. In some embodiments, the phagocytic receptor is selected from the group consisting of MERTK, dectin-1, and Fc gamma receptor (FcγR).

In some embodiments, the endocytic receptor is a scavenger receptor. In some embodiments, the scavenger receptor is a member of a class of scavenger receptor selected from the group consisting of class A, B, C, D, E, F, G, H, I K, J, K and L scavenger receptor. In some embodiments, the scavenger receptor is selected from the group consisting of SR AIII, MARCO, SCARA5 receptor, SRCL, CD36, SR-BI, CD68, LOX-1, SREC, MEGF10, SR-PSOX, FEEL-1, CD163, RAGE, CD44, and SR-L1.

In some embodiments, the ligand is selected from the group consisting of soluble CD163 (sCD163), mannose, growth arrest specific factor 6 (Gas6), Protein S (Pros1), Low Density Cholesterol (LDL), acetylated LDL (AcLDL), oxidised LDL (OxLDL) polyanions, ferritin, ferritin light chain, beta-glucans, N-acetylgalactosamine, GAL-type ligands (beta-D-galactopyranose), L-fucose, D-fucose, diacylated lipopeptides, High Density Cholesterol (HDL), lectins, selectins, Clq, hemoglobin, haptoglobin, amyloid-beta peptide, hyaluronic acid (HA aka hyaluronan), and microtubule-associated protein Tau (MAPT).

In some embodiments, the ligand is soluble CD163. In some embodiments, the ligand comprises an amino acid sequence at least 90, 91, 92, 93, 94, 95, 96, 97, 98, 99, or 100% to the amino acid sequence of SEQ ID NO:105.

In some embodiments, the BME comprises an amino acid sequence at least 90, 91, 92, 93, 94, 95, 96, 97, 98, 99, or 100% identical to the amino acid sequence of SEQ ID NO: 107.

In some embodiments, the BME further comprises an IgG Fc.

Nucleic acids encoding one or more of the BMEs described herein are also provided.

In some embodiments, the nucleic acid comprises a nucleotide sequence at least 75, 80, 85, 90, 91, 92, 93, 94, 95, 96, 97, 98, 99 or 100% identical to the nucleotide sequence of SEQ ID NO: 106.

Recombinant vectors encoding one or more BMEs of the instant disclosure are also provided, as are cells comprising one of more BMEs, nucleic acids encoding one or more BMEs, and/or vectors capable of expressing one or more BMEs. A population of cells comprising two or more cells described herein is also provided.

A pharmaceutical composition comprising a therapeutically effective amount of one or more BME, and a pharmaceutically acceptable carrier is also provided.

A method of treating or preventing a viral infection in a subject in need thereof is also provided comprising administering to the subject a therapeutically effective amount of one or more BME or a pharmaceutical composition thereof.

In some embodiments, the subject is a human.

In some embodiments, the viral infection is caused by a virus selected from the group consisting of a Togaviridae virus, a Coronaviridae virus, a Flaviviridae virus, an Orthomyxoviridae virus, a Filoviridae virus, a Paramyxoviridae virus, a Retroviridae virus, and a Bunyaviradae virus.

In some embodiments, the virus is a coronavirus. In some embodiments, the coronavirus is SARS-CoV-2.

In an aspect, an antigen macrophage engager (AME) is provided comprising a) an antibody that binds to an antigen expressed on a surface of a cancer cell; and b) a ligand for an endocytic receptor.

In some embodiments, the antibody comprises a single-chain variable fragment (scFv) or a single portion antibody (sdAb) variable portion.

In some embodiments, the antigen binding portion specifically binds to an antigen selected from the group consisting of CD19, CD22, HER2 (ERBB2/neu), Mesothelin, PSCA, CD123, CD30, CD171, CD138, CS-1, CLECL1, CD33, CD79b, EGFRvIII, GD2, GD3, BCMA, PSMA, RORI, FLT3, TAG72, CD38, CD44v6, CEA, EPCAM, B7H3 (CD276), KIT (CD 117), CD213A2, IL-1 IRa, PRSS21, VEGFR2, FSHR, TROP2, CD24, MUC-16, PDGFR-beta, SSEA-4, CD20, MUC1, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, Tn-glycopeptides, and IGLL1.

In some embodiments, the endocytic receptor is a mannose receptor.

In some embodiments, the endocytic receptor is a phagocytic receptor. In some embodiments, the intracellular signaling region of the phagocytic receptor comprises an intracellular portion from a protein selected from the group consisting of MERTK, and Fc gamma receptor (FcγR).

In some embodiments, the endocytic receptor is a scavenger receptor. In some embodiments, the scavenger receptor is a member of a class of scavenger receptor selected from the group consisting of class A, B, C, D, E, F, G, H, I K, J, K and L scavenger receptor. In some embodiments, the scavenger receptor is selected from the group consisting of SR AI/II, MARCO, SCARA5 receptor, SRCL, CD36, SR-BI, CD68, LOX-1, SREC, MEGF10, SR-PSOX, FEEL-1, CD163, RAGE, CD44, and SR-L1.

In some embodiments, the ligand is selected from the group consisting of soluble CD163 (sCD163), mannose, growth arrest specific factor 6 (Gas6), Protein S (Pros1), Low Density Cholesterol (LDL), acetylated LDL (AcLDL), oxidised LDL (OxLDL) polyanions, ferritin, ferritin light chain, beta-glucans, N-acetylgalactosamine, GAL-type ligands (beta-D-galactopyranose), L-fucose, D-fucose, diacylated lipopeptides, High Density Cholesterol (HDL), lectins, selectins, Clq, hemoglobin, haptoglobin, amyloid-beta peptide, hyaluronic acid (HA aka hyaluronan), and microtubule-associated protein Tau (MAPT).

In some embodiments, the ligand is soluble CD163. In some embodiments, the ligand comprises an amino acid sequence at least 90, 91, 92, 93, 94, 95, 96, 97, 98, 99, or 100% to the amino acid sequence of SEQ ID NO:105.

Nucleic acids encoding one or more of the AMEs described herein are also provided.

Recombinant vectors encoding one or more AMEs of the instant disclosure are also provided, as are cells comprising one of more AMEs, nucleic acids encoding one or more AMEs, and/or vectors capable of expressing one or more AMEs. A population of cells comprising two or more cells described herein is also provided.

A pharmaceutical composition comprising a therapeutically effective amount of one or more AME, and a pharmaceutically acceptable carrier is also provided.

A method of treating cancer in a subject in need thereof is also provided comprising administering to the subject a therapeutically effective amount of one or more AMEs described herein or a pharmaceutical composition thereof. In some embodiments, the subject is a human.

### BRIEF DESCRIPTION OF THE DRAWINGS

The accompanying drawings which are incorporated in and constitute a part of this specification, illustrate exemplary embodiments of the present disclosure and, together with the description, serve to explain principles of the present disclosure.

FIG. 1 is a schematic comparing antibody recognition of viruses (left panel) to chimeric bait receptor (CBR) recognition of viruses (right panel). Mutated viruses (labeled “Mut”) are not recognized by antibody. However, both wild-type viruses and mutated viruses are recognized by the extracellular “bait” portion (e.g., ACE2) of chimeric bait receptor.

FIG. 2 is a set of fluorescent microscope images demonstrating phagocytosis of SARS-CoV-2 spike protein-coated or non-coated beads by either untransduced (UTD) Thp1 cells or by Thp1 cells transduced with mannose receptor-based F4-AC construct containing ACE2 (19-740 AA) bait.

FIG. 3A-3B shows (3A) Flow cytometry plots of SARS-CoV-2 Spike-coated or non-coated beads phagocytosis by either untransduced (UTD) Thp1 cells or by Thp1 cells that were transduced with mannose receptor-based F4-AC construct containing ACE2 (19-740 AA) bait. (3B) Summary of flow cytometry results. Mean±SEM (n=3). One-way ANOVA followed by Tukey's post-hoc analysis. ***p<0.0001.

FIG. 4A-4B shows (4A) Flow cytometry plots of B.1.1.7 or WT SARS-CoV-2 Spike-coated or non-coated beads phagocytosis by either untransduced (UTD) Thp1 cells or by Thp1 cells that were transduced with mannose receptor-based F4-AC construct containing ACE2 (19-740 AA) bait. (4B) Summary of flow cytometry results. Mean±SEM (n=3). One-way ANOVA followed by Tukey's post-hoc analysis. ***p<0.0001.

FIG. 5A-5B shows (5A) Flow cytometry plots of B.1.617.2 or WT SARS-CoV-2 Spike-coated or non-coated beads phagocytosis by either untransduced (UTD) Thp1 cells or by Thp1 cells that were transduced with mannose receptor-based F4-AC construct containing ACE2 (19-740 AA) bait. (5B) Summary of flow cytometry results. Mean±SEM (n=3). One-way ANOVA followed by Tukey's post-hoc analysis. ***p<0.0001.

FIG. 6 is a schematic of a neutralization assay. Lentivirus carrying GFP transfer plasmid was pseudotyped with Spike envelope protein, to generate Spike-LV. Spike-LV particles were pre-incubated with Thp1 effector cells expressing the CBR construct. After 30 min to 2 hr pre-incubation, cells were spun down, and supernatant was collected and incubated with HEK 293T cells expressing hACE2 receptor on the cell surface (ACE2-293). GFP⁺ frequency was determined by flow cytometer to test neutralization effect.

FIG. 7A-7B shows results from a neutralization assay. (7A) Flow cytometry plots of transduced ACE2-293 target cells after the virus was pre-incubated with effector cells for 30 minutes. (7B) Summary of flow cytometry results. Mean±SEM (n=2). One-way ANOVA for each MOI separately followed by Holm-Sidak post-hoc analysis compared to mean of control (No effector cells w/ virus). For MOI 0.1 the **/*** are compared to control (No effector cells w/ virus).

FIG. 8 is a set of schematics for various chimeric bait receptors (CBRs) and chimeric phagocytic receptors (CPRs) provided by the instant disclosure. The CBR constructs include ACE2 as bait for virus (e.g., SARS-COV-2) and CPR constructs include anti-FLT3 scFv. The extracellular bait (e.g., ACE2) or antigen-binding portion (e.g., anti-FLT3 scFv) is fused to the intracellular signaling domain of one of the following phagocytic receptors: MERTK, MEGF10, Dectin-1, or CD163.

FIG. 9A-9C shows (9A) representative flow cytometry plots of Spike-coated and non-coated beads phagocytosis by either untransduced (UTD) Thp1 cells or by Thp1 cells that were transduced with MEGF10-based B4-AC construct containing ACE2 (19-740 AA) bait. (9B) Summary of flow cytometry results. Mean±SEM (n=3). Two-way ANOVA followed by Tukey post-hoc analysis. (9C) Microscope images of UTD and B4-AC Thp1 cells, showing cell clustering.

FIG. 10A-10B shows results from a neutralization assay. (10A) Representative flow cytometry plots of transduced ACE2-293 target cells after the virus was pre-incubated with effector cells for 2 hr. (10B) Summary of flow cytometry results. Mean±SEM (n=2). One-way ANOVA followed by Holm-Sidakpost-hoc analysis.

FIG. 11 is a set of schematics for various exemplary chimeric phagocytic receptor (CPR) constructs provided by the instant disclosure. The CPR constructs include either anti-CD19 scFv or anti-CD20 scFv fused to the intracellular signaling domain of one of the following phagocytic receptors: Mannose Receptor (F4), MERTK, MEGF10, Dectin-1, or CD163.

FIG. 12 is a schematic of an exemplary bait macrophage engager (BME) construct provided by the instant disclosure in which soluble CD163 (sCD163) is fused to ACE2 (19-740).

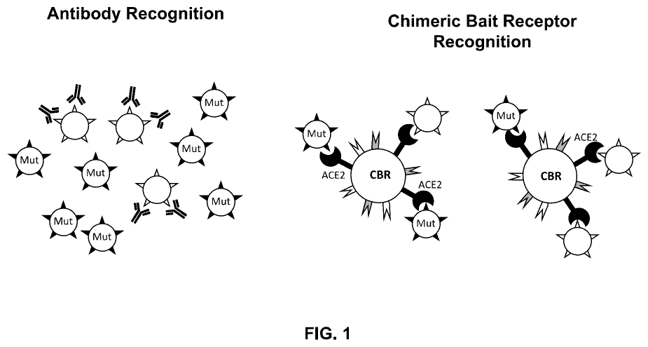

### DETAILED DESCRIPTION

The instant disclosure provides novel chimeric bait receptors (CBRs) to program the immune cells that are responsible for innate immunity (e.g., macrophage) to eliminate viral infections by destroying the viruses that cause them. The CBRs provided herein contain a portion of the protein to which a virus binds to infect human cells, which acts as bait for the virus, allowing the immune cells to then destroy it. Major advantages of these CBRs compared to other existing approaches of combatting viral infections include: durability (e.g., insensitive to mutations of the targeted virus due to use of bait); efficiency (e.g., CBRs are made from parts of naturally occurring proteins/receptors that are responsible for phagocytosis or endocytosis in macrophage and endow immune cells with the ability to destroy invading pathogens); and versatility (CBRs are modular synthetic receptors that can be reconfigured to attack almost any virus, bacteria or mammalian cells, including malignant ones).

The CBRs described herein comprise an extracellular portion comprising a binding region that a virus specifically binds to (“viral bait”); a transmembrane portion; and an intracellular portion comprising an intracellular signaling region of an endocytic receptor. Typically, the viral binding region is not an antibody but instead is a receptor or protein on a host cell that the virus binds to infect the host cell. Upon binding of the virus to the extracellular viral bait, the intracellular signaling region is activated, leading to endocytosis of the virus. As such, CBRs are particularly useful in methods of treating or preventing a viral infection (e.g., SARS-CoV-2 infection) in a subject in need thereof.

The CBRs of the instant disclosure can be reconfigured to replace the extracellular viral bait with an extracellular portion comprising an antigen binding portion. These reconfigured CBRs are referred to herein as chimeric phagocytic receptors (CPRs) and are also provided by the instant disclosure. These CPRs can be used to target any antigen, such as a tumor associated antigen (TAA), to destroy any cells expressing the antigen at the surface. Thus, these CPRs are particularly useful in methods of treating cancer in a subject in need thereof.

Also provided herein are bait macrophage engagers (BMEs) and antigen macrophage engagers (AMEs), which are similar to CBRs and CPRs, respectively, in which the transmembrane portion and intracellular portion are replaced with a ligand for an endocytic receptor. In other words, BMEs comprise a binding region that a virus specifically binds to (“viral bait”) and a ligand for an endocytic receptor and AMEs comprise an antigen binding portion and a ligand for an endocytic receptor. BMEs and AMEs function by inducing endocytosis of the virus or antigen-presenting cell upon binding of the ligand portion to an endocytic receptor. As such, BMEs are particularly useful in methods of treating or preventing a viral infection (e.g., SARS-CoV-2 infection) in a subject in need thereof and AMEs are particularly useful in methods of treating cancer in a subject in need thereof.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the term “extracellular” with respect to a recombinant transmembrane protein refers to the portion or portions of the recombinant transmembrane protein that are located outside of a cell.

As used herein, the term “transmembrane” with respect to a recombinant transmembrane protein refers to the portion or portions of the recombinant transmembrane protein that are embedded in the plasma membrane of a cell.

As used herein, the term “intracellular” with respect to a recombinant transmembrane protein refers to the portion or portions of the recombinant transmembrane protein that are located in the cytoplasm of a cell. The terms “cytoplasmic” and “intracellular” are interchangeable.

“Binding affinity” generally refers to the strength of the sum total of non-covalent interactions between a single binding site of a molecule (e.g., a CBR) and its binding partner (e.g., a viral protein). Unless indicated otherwise, as used herein, “binding affinity” refers to intrinsic binding affinity which reflects a 1:1 interaction between members of a binding pair (e.g., a CBR and a viral protein). The affinity of a molecule X for its partner Y can generally be represented by the dissociation constant (K_{D}). Affinity can be measured and/or expressed in a number of ways known in the art, including, but not limited to, equilibrium dissociation constant (K_{D}) and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant and k_{off} refers to the dissociation rate constant. The kₒₙ and k_{off} can be determined by techniques known to one of ordinary skill in the art, such as use of BIAcore® or KinExA. As used herein, a “lower affinity” refers to a larger K_{D}.

For example, “specifically binds to” may be used to refer to the ability of a receptor to preferentially bind to a particular ligand (e.g., an antigen or a viral protein) as such binding is understood by one skilled in the art. For example, an antibody or antibody fragment that specifically binds to an antigen can bind to other antigens, generally with lower affinity as determined by, e.g., BIAcore®, or other immunoassays known in the art (see, e.g., Savage et al., (1999) Immunity. 10(4):485-92, which is incorporated by reference herein in its entirety).

As used herein, an “epitope” is a term in the art and refers to a localized region of an antigen (e.g., a peptide or a peptide-MHC complex) to which a CPR can bind. In certain embodiments, the epitope to which a CPR binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), flow cytometry analysis, mutagenesis mapping (e.g., site-directed mutagenesis mapping), and/or structural modeling. For X-ray crystallography, crystallization may be accomplished using any of the known methods in the art (e.g., Giegé R et al., (1994) Acta Crystallogr D Biol Crystallogr 50(Pt 4): 339-350; McPherson A, (1990) Eur J Biochem 189: 1-23; Chayen N E, (1997) Structure 5: 1269-1274; McPherson A, (1976) J Biol Chem 251: 6300-6303, each of which is herein incorporated by reference in its entirety). TCR:antigen crystals may be studied using well-known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H. W., et al.; U.S. 2004/0014194); and BUSTER (Bricogne G, (1993) Acta Crystallogr D Biol Crystallogr 49(Pt 1): 37-60; Bricogne G, (1997) Meth Enzymol 276A: 361-423, ed Carter C W; and Roversi P et al., (2000) Acta Crystallogr D Biol Crystallogr 56(Pt 10): 1316-1323), each of which is herein incorporated by reference in its entirety. Mutagenesis mapping studies may be accomplished using any method known to one of skill in the art. See, e.g., Champe M et al., (1995) J Biol Chem 270: 1388-1394 and Cunningham B C & Wells J A, (1989) Science 244: 1081-1085, each of which is herein incorporated by reference in its entirety, for a description of mutagenesis techniques, including alanine scanning mutagenesis techniques. In a specific embodiment, the epitope of an antigen is determined using alanine scanning mutagenesis studies. In a specific embodiment, the epitope of an antigen is determined using hydrogen/deuterium exchange coupled with mass spectrometry. In certain embodiments, the antigen is a peptide-MHC complex. In certain embodiments, the antigen is a peptide presented by an MHC molecule.

As used herein, the terms “treat,” “treating,” and “treatment” refer to therapeutic or preventative measures described herein. In some embodiments, the methods of “treatment” employ administration of a CBR or a CPR or a cell expressing a CBR or a CPR to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “operably linked” refers to a linkage of polynucleotide sequence elements or amino acid sequence elements in a functional relationship. For example, a polynucleotide sequence is operably linked when it is placed into a functional relationship with another polynucleotide sequence. In some embodiments, a transcription regulatory polynucleotide sequence e.g., a promoter, enhancer, or other expression control element is operably linked to a polynucleotide sequence that encodes a protein if it affects the transcription of the polynucleotide sequence that encodes the protein. Operably linked elements may be contiguous or non-contiguous. In addition, in the context of a polypeptide, “operably linked” refers to a physical linkage (e.g., directly or indirectly linked) between amino acid sequences (e.g., different segments, regions, or domains) to provide for a described activity of the polypeptide. In the present disclosure, various segments, regions, or domains of the chimeric polypeptides of the disclosure may be operably linked to retain proper folding, processing, targeting, expression, binding, and other functional properties of the chimeric polypeptides in the cell. Unless stated otherwise, various regions, domains, and segments of the chimeric polypeptides of the disclosure are operably linked to each other. Operably linked regions, domains, and segments of the chimeric polypeptides of the disclosure may be contiguous or non-contiguous (e.g., linked to one another through a linker).

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

The term “functional fragment” as used herein in reference to a protein or polypeptide refers to a fragment of a reference protein that retains at least one particular function. Not all functions of the reference protein need be retained by a functional fragment of the protein. In some instances, one or more functions are selectively reduced or eliminated.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. As used herein, the term “modification,” with reference to an amino acid sequence, refers to an amino acid sequence that comprises at least one substitution, alteration, inversion, addition, or deletion of an amino acid residue compared to a reference amino acid sequence.

As used herein, the term “derived from,” with reference to a polynucleotide sequence, refers to a polynucleotide sequence that has at least 85% sequence identity to a reference naturally occurring nucleic acid sequence from which it is derived. The term “derived from,” with reference to an amino acid sequence, refers to an amino acid sequence that has at least 85% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

The term “recombinant” or “engineered” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reach on(s), or to which linkers have been attached, or that has been integrated into a vector, such as a cloning vector or expression vector.

As used herein, the term “host protein” refers to a protein associated with a cell that a virus binds to in the process of infecting the cell. For example, for SARS-CoV-2 at least one host protein includes human angiotensin converting enzyme 2 (ACE2). In some embodiments, the host protein can be an antibody or other immune system protein that specifically binds to a virus in a process to clear the virus from an organism. In some embodiments, the host protein cannot be an antibody or other immune system protein that specifically binds to a virus in a process to clear the virus from an organism.

As used herein, the term “vector” refers to a nucleic acid molecule or sequence capable of transferring or transporting another nucleic acid molecule. The transferred nucleic acid molecule is generally linked to, e.g., inserted into, the vector nucleic acid molecule.

### Phagocytosis

Phagocytosis generally refers to an engulfment process of cells or large particles (>0.5 μm) wherein tethering of a target cell or particle, engulfment of the target cell or particle, and degradation of the internalized target cell or particle occurs. In certain embodiments, phagocytosis includes formation of a phagosome that encompasses the internalized target cell or particle and phagosome fusion with a lysosome to form a phagolysosome, wherein the contents therein are degraded. As such, “phagocytosis” includes the process of “efferocytosis”, which specifically refers to the phagocytosis of apoptotic or necrotic cells in a non-inflammatory manner.

There are two principle types of phagocytosis, which are influenced by the target, cell-type and surrounding milieu. Anti-microbe phagocytosis clears and degrades disease-causing microbes, induces pro-inflammatory signaling through cytokine and chemokine secretion, and recruits immune cells to mount an effective inflammatory response. This type of phagocytosis is often referred to as “inflammatory phagocytosis” (or “immunogenic phagocytosis”). However, in some instances, such as with certain persistent infections, anti-inflammatory responses may follow microbial uptake. Anti-microbe phagocytosis is commonly performed by professional phagocytes of the myeloid lineage, such as immature dendritic cells (DCs) and macrophages and by tissue-resident immune cells.

Phagocytosis of damaged, self-derived apoptotic cells or cell debris (e.g., efferocytosis), in contrast, is typically a non-inflammatory (also referred to as a “non-immunogenic”) process. Billions of damaged, dying, and unwanted cells undergo apoptosis each day. Unwanted cells include, for example, excess cells generated during development, senescent cells, infected cells (intracellular bacteria or viruses), transformed or malignant cells, and cells irreversibly damaged by cytotoxic agents. Phagocytes execute specific, swift removal of apoptotic cells without causing damage to the surrounding tissues or inducing a pro-inflammatory immune response. Steps for apoptotic cell clearance include: (1) release of “find me” signals from apoptotic cells to recruit phagocytes to the location of apoptotic cells; (2) “eat me” signals exposed on the surface of apoptotic cells are bound by phagocytes via specific receptors; (3) cytoskeletal rearrangement to engulf the apoptotic cell; and (4) the ingested apoptotic cell is digested and specific phagocytic responses are elicited (e.g., secretion of anti-inflammatory cytokines).

The terms “phagocytic cells” and “phagocytes” are used interchangeably herein to refer to a cell that is capable of phagocytosis, e.g., ingesting microorganisms and foreign particles, for example, capable of engulfing a large particulate mass, for example from about 0.1 μm in diameter up to about 2 mm or about 1 mm in diameter; from about 0.5 μm in diameter to about 1 mm in diameter, etc., particularly including up to the size of a microbial cell or mammalian cell, e.g., a tumor cell. Phagocytosis, as described above, encompasses the engulfment of cells, pathogens, and various particles by surrounding it with the effector cell membrane. As such, phagocytes protect the body by ingesting harmful foreign particles, bacteria, and dead or dying cells. These cells are essential for fighting infections and for subsequent immunity.

There are several categories of phagocytic cells. Exemplary phagocytic cells include macrophages, mononuclear cells (histiocytes and monocytes), polymorph nuclear leukocytes, (neutrophils) and dendritic cells. Phagocytic cells of humans and other jawed vertebrates are divided into “professional” and “non-professional” groups based on the efficiency with which they participate in phagocytosis. The professional phagocytes include many types of white blood cells (such as neutrophils, monocytes, macrophages, mast cells, and dendritic cells). The main difference between professional and non-professional phagocytes is that the professional phagocytes have molecules called receptors on their surfaces that can detect harmful objects, such as bacteria, that are not normally found in the body. As such, professional phagocytes are capable of recognizing a wide variety of phagocytic targets, and of ingesting them at a higher rate than non-phagocytic cells.

Dendritic cell (DC) refers to any member of a diverse population of morphologically similar cell types found in lymphoid or non-lymphoid tissues. DCs are referred to as “professional” antigen presenting cells and have a high capacity for sensitizing MHC-restricted T cells. DCs may be recognized by function, by phenotype and/or by gene expression pattern, particularly by cell surface phenotype. These cells are characterized by their distinctive morphology, high levels of surface MHC-class II expression and ability to present antigen to CD4+ and/or CD8+ T cells, particularly to naive T cells.

Neutrophils and macrophages are representative of fully differentiated phagocytic cells. While neutrophils leaving the bone marrow are fully differentiated, macrophages differentiate from circulating monocytes in extravascular tissues. Monocytes display a lower phagocytic response, compared to neutrophils and macrophages, and must respond to activation and differentiation signals to achieve optimal phagocytic capacity. The process of monocyte-to-macrophage differentiation is well-characterized and can be performed in vitro or in vivo.

Macrophages are of particular interest. These immune cells can make their way inside tumors and travel to cancers that the rest of the immune system cannot reach. Macrophages are critical effectors of the innate immune system, responsible for engulfing debris and pathogens. Accumulating evidence suggests that macrophages are abundant in the tumor microenvironment of numerous cancers where they can adopt a classically activated (M1, anti-tumor) or an alternatively activated (M2, pro-tumor) phenotype. Macrophages are potent effectors of the innate immune system and are capable of at least three distinct anti-tumor functions: phagocytosis, cellular cytotoxicity, and antigen presentation to orchestrate an adaptive immune response. While T cells require antigen-dependent activation via the T cell receptor or the chimeric immunoreceptor, macrophages can be activated in a variety of ways. Direct macrophage activation is antigen-independent, relying on mechanisms such as pathogen associated molecular pattern recognition by Toll-like receptors (TLRs). Macrophages are uniquely capable of penetrating solid tumors, while other immune cells, like T cells, are physically excluded or inactivated. This suggests that engineered macrophages may augment existing T cell-based therapies.

### Chimeric Polypeptides

The instant disclosure provides chimeric polypeptides which include a portion that is capable of specifically binding to an antigen and a portion that targets the chimeric polypeptide for endocytosis upon binding to the antigen. As described in further detail below, the antigen may be a viral protein and the portion that is capable of specifically binding to the viral protein may be a binding region that the virus binds to on a host cell. In other embodiments, the antigen may be a tumor associated antigen (TAA) and the portion that is capable of specifically binding to the TAA may be an antigen binding portion, such as an antibody or fragment thereof. The portion of the chimeric polypeptides that targets the chimeric polypeptide for endocytosis may be an intracellular (i.e., cytoplasmic) signaling region of an endocytic receptor. In other embodiments, the portion of the chimeric polypeptides that targets the chimeric polypeptide for endocytosis may be a ligand for an endocytic receptor.

In one aspect, the instant disclosure provides a chimeric bait receptor (CBR), which includes a) an extracellular portion that is capable of specifically binding to a viral protein, b) a transmembrane portion, and c) an intracellular portion containing an intracellular signaling region of an endocytic receptor. In some embodiments, the extracellular portion is not an antibody. In some embodiments, the extracellular portion specifically binds to a viral protein that binds to and confers attachment of the virus to a host cell. In some embodiments, the intracellular portion contains an intracellular signaling region of an endocytic receptor selected from a phagocytic receptor or a scavenger receptor. The transmembrane portion may be any transmembrane portion capable of expressing the CBR at the surface of a cell, particularly of a phagocytic cell. In some embodiments, the transmembrane portion may be a transmembrane portion of an endocytic receptor. In some embodiments, the transmembrane portion and the intracellular portion of a CBR may be derived from the same endocytic receptor. In other embodiments, the transmembrane portion and the intracellular portion of a CBR may be derived from different endocytic receptors. In other embodiments, the transmembrane portion is not derived from an endocytic receptor.

As proof of concept, exemplary CBRs of the present disclosure were developed to program macrophage to neutralize SARS-CoV-2 virus by phagocytosis. The exemplary CBR contains a portion of angiotensin converting enzyme 2 (ACE2), the receptor SARS-CoV-2 virus binds to in order to infect cells. When the CBR is expressed by macrophage, the ACE2 portion is presented on the cell surface as a bait for the virus. Macrophage expressing CBR against SARS-CoV-2 were shown to selectively phagocytose 1) beads with SARS-CoV-2 spike envelop protein from multiple variant strains attached; and 2) live lentivirus that carry (pseudo-typed) spike envelop protein of SARS-COV-2 virus.

Accordingly, in some embodiments, the extracellular portion of CBRs of the present disclosure contains a portion of ACE2 sufficient for binding to SARS-CoV-2 spike protein. In some embodiments, CBRs of the present disclosure contain a) a portion of ACE2 sufficient for binding to SARS-CoV-2 spike protein, b) a transmembrane portion of a protein selected from the group consisting of CD8, mannose receptor, MER proto-oncogene tyrosine kinase (MERTK), dectin-1, multiple EGF like portions 10 (MEGF10), and CD163, and c) an intracellular signaling region of an endocytic receptor selected from the group consisting of mannose receptor, MERTK, dectin-1, MEGF10, and CD163.

In some embodiments, CBRs of the present disclosure contain a) a portion of ACE2 sufficient for binding to SARS-CoV-2 spike protein, b) a transmembrane portion of a mannose receptor, and c) an intracellular signaling region of a mannose receptor.

In some embodiments, CBRs of the present disclosure contain a) a portion of ACE2 sufficient for binding to SARS-CoV-2 spike protein, b) a transmembrane portion of a MERTK, and c) an intracellular signaling region of a MERTK.

In some embodiments, CBRs of the present disclosure contain a) a portion of ACE2 sufficient for binding to SARS-CoV-2 spike protein, b) a transmembrane portion of a dectin-1, and c) an intracellular signaling region of a dectin-1.

In some embodiments, CBRs of the present disclosure contain a) a portion of ACE2 sufficient for binding to SARS-CoV-2 spike protein, b) a transmembrane portion of a MEGF10, and c) an intracellular signaling region of a MEGF10.

In some embodiments, CBRs of the present disclosure contain a) a portion of ACE2 sufficient for binding to SARS-CoV-2 spike protein, b) a transmembrane portion of a CD163, and c) an intracellular signaling region of a CD163.

In some embodiments, CBRs of the present disclosure include an amino acid sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 61-67.

In another aspect, the instant disclosure provides a chimeric phagocytic receptor (CPR), which includes a) an extracellular portion containing an antigen binding portion, b) a transmembrane portion, and c) an intracellular portion containing an intracellular signaling region of an endocytic receptor. In some embodiments, the extracellular portion is an antibody or fragment thereof (such as a single-chain variable fragment (scFv) or a single portion antibody (sdAb) variable portion). In some embodiments, the extracellular portion specifically binds to a tumor associated antigen (TAA). In some embodiments, the intracellular portion contains an intracellular signaling region of an endocytic receptor selected from a phagocytic receptor or a scavenger receptor. The transmembrane portion may be any transmembrane portion capable of expressing the CBR at the surface of a cell, particularly of a phagocytic cell. In some embodiments, the transmembrane portion may be a transmembrane portion of an endocytic receptor. In some embodiments, the transmembrane portion and the intracellular portion of a CBR may be derived from the same endocytic receptor. In other embodiments, the transmembrane portion and the intracellular portion of a CBR may be derived from different endocytic receptors. In other embodiments, the transmembrane portion is not derived from an endocytic receptor.

In some embodiments, CPRs of the instant disclosure do not comprise a recruitment portion, which portion is not the endocytic receptor intracellular region, and which portion binds to a cytosolic protein of a phagocytic signaling pathway.

Exemplary CPRs of the present disclosure were designed to target cancer cells for phagocytosis. The exemplary CPRs contain scFv that specifically bind to an antigen selected from FLT3, CD19, or CD20. Similar to the CBRs described above, when a CPR is expressed by macrophage, the scFv is presented on the cell surface as a bait for cancer cells expressing the tumor associated antigen(s) (such as FLT3, CD19, or CD20). Macrophage expressing CPR thus present an alternative to conventional chimeric antigen receptor (CAR)-T and/or T cell receptor (TCR)-T cells currently in development as cancer therapeutics. For example, in recent years, T cells expressing a CAR directed against CD19 have been shown to have significant anti-leukemic efficacy, where complete remission has been achieved in 90% of acute lymphoblastic leukemia patients treated. These results are accompanied by robust T cell proliferation and clearly documented T cell infiltration into tumor sites in leukemic patients so treated. Despite the high response rates demonstrated in hematopoietic malignancies, CAR-T cell efficacy in solid tumors as well as in certain lymphoid tumors may be limited. Possible explanations for this include the potentially impaired ability of T cells to infiltrate solid tumors, poor trafficking, immunosuppressive tumor microenvironment, and expression of few tumor specific antigens on solid tumor cells.

In contrast, macrophages are uniquely capable of enriching in the tumor microenvironment, where T cells are often excluded. Accordingly, use of CPR expressing macrophage of the instant disclosure provides a distinct and potentially synergistic approach to existing CAR-T therapy. Additionally, CPR expressing macrophage can act as bait for metastatic cancer cells in order to spread around the body. Use of CPRs to treat cancer is thus provided by the instant disclosure and will be described further herein.

In some embodiments, the extracellular portion of CPRs of the present disclosure contain an antigen binding portion that specifically binds to a tumor associated antigen including, but not limited to, an antigen selected from the group consisting of CD19, CD22, HER2 (ERBB2/neu), Mesothelin, PSCA, CD123, CD30, CD171, CD138, CS-1, CLECL1, CD33, CD10, CD79b, EGFRvIII, GD2, GD3, BCMA, PSMA, RORI, FLT3 (CD135), TAG72, CD38, CD44v6, CEA, EPCAM, B7H3 (CD276), KIT (CD 117), CD213A2, IL-1 IRa, PRSS21, VEGFR2, FSHR, TROP2, CD24, MUC-16, PDGFR-beta, SSEA-4, CD20, MUC1, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, Tn-glycopeptides (e.g., O-glycans comprised of a single N-acetylgalactosamine—GalNAc, known as Tn-antigen), and IGLL 1. In certain embodiments, the extracellular portion of CPRs of the present disclosure contain an antigen binding portion that specifically binds to an antigen selected from the group consisting of CD19, CD20, and FLT3. In certain embodiments, the antigen binding portion is an scFv.

In some embodiments, CPRs of the present disclosure contain a) an antigen binding portion that specifically binds to an antigen selected from the group consisting of CD19, CD20, and FLT3, b) a transmembrane portion of a protein selected from the group consisting of CD8, mannose receptor, MER proto-oncogene tyrosine kinase (MERTK), dectin-1, multiple EGF like portions 10 (MEGF 10), and CD163, and c) an intracellular signaling region of an endocytic receptor selected from the group consisting of mannose receptor, MERTK, dectin-1, MEGF10, and CD163.

In some embodiments, CPRs of the present disclosure contain a) an antigen binding portion that specifically binds to an antigen selected from the group consisting of CD19, CD20, and FLT3, b) a transmembrane portion of a mannose receptor, and c) an intracellular signaling region of a mannose receptor.

In some embodiments, CPRs of the present disclosure contain a) an antigen binding portion that specifically binds to an antigen selected from the group consisting of CD19, CD20, and FLT3, b) a transmembrane portion of a MERTK, and c) an intracellular signaling region of a MERTK.

In some embodiments, CPRs of the present disclosure contain a) an antigen binding portion that specifically binds to an antigen selected from the group consisting of CD19, CD20, and FLT3, b) a transmembrane portion of a dectin-1, and c) an intracellular signaling region of a dectin-1.

In some embodiments, CPRs of the present disclosure contain a) an antigen binding portion that specifically binds to an antigen selected from the group consisting of CD19, CD20, and FLT3, b) a transmembrane portion of a MEGF10, and c) an intracellular signaling region of a MEGF10.

In some embodiments, CPRs of the present disclosure contain a) an antigen binding portion that specifically binds to an antigen selected from the group consisting of CD19, CD20, and FLT3, b) a transmembrane portion of a CD163, and c) an intracellular signaling region of a CD163.

In some embodiments, CPRs of the present disclosure include an amino acid sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 86-103.

In another aspect, the instant disclosure provides a bait macrophage engager (BME), which includes a) a binding region that a virus specifically binds to, and b) a ligand for an endocytic receptor. In some embodiments, the binding region is not an antibody. In some embodiments, the binding region specifically binds to a viral protein that binds to and confers attachment of the virus to a host cell. In some embodiments, the ligand is a ligand for an endocytic receptor selected from a phagocytic receptor or a scavenger receptor. In some embodiments, the ligand is one or more of the following: soluble CD163 (sCD163), mannose, growth arrest specific factor 6 (Gas6), Protein S (Pros1), Low Density Cholesterol (LDL), acetylated LDL (AcLDL), oxidised LDL (OxLDL) polyanions, ferritin, ferritin light chain, beta-glucans, N-acetylgalactosamine, GAL-type ligands (beta-D-galactopyranose), L-fucose, D-fucose, diacylated lipopeptides, High Density Cholesterol (IDL), lectins, selectins, C1q, hemoglobin, haptoglobin, amyloid-beta peptide, hyaluronic acid (HA aka hyaluronan), microtubule-associated protein Tau (MAPT), or a fragment of any ligand described herein.

An exemplary BME of the present disclosure was developed to neutralize SARS-CoV-2 virus by phagocytosis. The exemplary BME contains a portion of ACE2 fused to soluble CD163 (sCD163). sCD163 is a natural scavenger involved in iron recycling by recruiting macrophages. Thus, a BME containing ACE2 and sCD163 can be used as a substitution for neutralizing antibodies for SARS-CoV-2. Instead of recruiting immune cells via Fc, the BME will recruit macrophages via CD163.

Accordingly, in some embodiments, the binding region of BMEs of the present disclosure contains a portion of ACE2 sufficient for binding to SARS-CoV-2 spike protein. In some embodiments, BMEs of the present disclosure contain a) a portion of ACE2 sufficient for binding to SARS-CoV-2 spike protein, and b) a ligand for an endocytic receptor selected from the group consisting of mannose receptor, MERTK, dectin-1, MEGF10, and CD163.

In some embodiments, BMEs of the present disclosure contain a) a portion of ACE2 sufficient for binding to SARS-CoV-2 spike protein, and b) a ligand for a mannose receptor, such as mannose or a fragment thereof.

In some embodiments, BMEs of the present disclosure contain a) a portion of ACE2 sufficient for binding to SARS-CoV-2 spike protein, and b) a ligand for a MERTK, such as growth arrest specific factor 6 (Gas6) or Protein S (Pros1), or fragments thereof.

In some embodiments, BMEs of the present disclosure contain a) a portion of ACE2 sufficient for binding to SARS-CoV-2 spike protein, and b) a ligand for a dectin-1, such as beta-glucans.

In some embodiments, BMEs of the present disclosure contain a) a portion of ACE2 sufficient for binding to SARS-CoV-2 spike protein, and b) a ligand for a MEGF10, such as Clq or a fragment thereof.

In some embodiments, BMEs of the present disclosure contain a) a portion of ACE2 sufficient for binding to SARS-CoV-2 spike protein, and b) a ligand for a CD163, such as sCD163 of a fragment thereof.

In some embodiments, BMEs of the present disclosure include an amino acid sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 107.

In another aspect, the instant disclosure provides an antigen macrophage engager (AME), which includes a) an antibody that binds to an antigen expressed on a surface of a cancer cell, and b) a ligand for an endocytic receptor. In some embodiments, the antibody comprises, or alternatively consists of, a single-chain variable fragment (scFv) or a single portion antibody (sdAb) variable portion). In some embodiments, the ligand is a ligand for an endocytic receptor selected from a phagocytic receptor or a scavenger receptor.

Exemplary AMEs of the present disclosure target cancer cells for phagocytosis. The exemplary AMEs contain scFv that specifically bind to an antigen selected from FLT3, CD19, or CD20. Similar to the CPRs described above, the scFv acts as a bait for cancer cells expressing the tumor associated antigen(s) (such as FLT3, CD19, or CD20). Upon binding of the ligand portion of the AME to its respective endocytic receptor, phagocytosis of the AME and attached cancer cell will occur. Thus, AMEs can also be used as cancer therapeutics.

In some embodiments, the AMEs of the present disclosure contain an antibody or antigen binding portion thereof that specifically binds to a tumor associated antigen including, but not limited to, an antigen selected from the group consisting of CD19, CD22, HER2 (ERBB2/neu), Mesothelin, PSCA, CD123, CD30, CD171, CD138, CS-1, CLECL1, CD33, CD10, CD79b, EGFRvIII, GD2, GD3, BCMA, PSMA, RORI, FLT3 (CD135), TAG72, CD38, CD44v6, CEA, EPCAM, B7H3 (CD276), KIT (CD 117), CD213A2, IL-1 IRa, PRSS21, VEGFR2, FSHR, TROP2, CD24, MUC-16, PDGFR-beta, SSEA-4, CD20, MUC1, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, Tn-glycopeptides, and IGLL1. In certain embodiments, the AMEs of the present disclosure contain an antibody or antigen binding portion thereof that specifically binds to an antigen selected from the group consisting of CD19, CD20, and FLT3. In certain embodiments, the antibody or antigen binding portion thereof is an scFv.

In some embodiments, AMEs of the present disclosure contain a) an antigen binding portion that specifically binds to an antigen selected from the group consisting of CD19, CD20, and FLT3, b) a transmembrane portion of a protein selected from the group consisting of CD8, mannose receptor, MER proto-oncogene tyrosine kinase (MERTK), multiple EGF like portions 10 (MEGF10), and CD163, and c) a ligand for an endocytic receptor selected from the group consisting of mannose receptor, MERTK, dectin-1, MEGF10, and CD163.

In some embodiments, AMEs of the present disclosure contain a) an antigen binding portion that specifically binds to an antigen selected from the group consisting of CD19, CD20, and FLT3, and b) a ligand for a mannose receptor, such as mannose or a fragment thereof.

In some embodiments, AMEs of the present disclosure contain a) an antigen binding portion that specifically binds to an antigen selected from the group consisting of CD19, CD20, and FLT3, and b) a ligand for a MERTK, such as growth arrest specific factor 6 (Gas6) or Protein S (Pros1), or fragments thereof.

In some embodiments, AMEs of the present disclosure contain a) an antigen binding portion that specifically binds to an antigen selected from the group consisting of CD19, CD20, and FLT3, and b) a ligand for a MEGF10, such as Clq or a fragment thereof.

In some embodiments, AMEs of the present disclosure contain a) an antigen binding portion that specifically binds to an antigen selected from the group consisting of CD19, CD20, and FLT3, and b) a ligand for a CD163, such as sCD163 of a fragment thereof.

In some embodiments, the chimeric polypeptides of the present disclosure contain a portion of the extracellular domain of an endocytic receptors. For example, a CBR of the present disclosure may comprise a binding region that a virus specifically binds to fused to a portion of an endocytic receptor including a portion of the extracellular domain, the transmembrane domain and the intracellular domain of the endocytic receptor. Similarly, a CPR of the present disclosure may comprise an antigen binding portion fused to a portion of an endocytic receptor including a portion of the extracellular domain, the transmembrane domain and the intracellular domain of the endocytic receptor. In some embodiments, the chimeric polypeptides of the present disclosure include a full-length endocytic receptor (e.g., the entire extracellular domain, transmembrane domain and intracellular domain of an endocytic receptor). For example, a CBR of the present disclosure may comprise a binding region that a virus specifically binds to fused to a full-length endocytic receptor. Similarly, a CPR of the present disclosure may comprise an antigen binding portion fused to a full-length endocytic receptor.

Non-limiting examples of sequences that may be used to generate chimeric polypeptides of the present disclosure are provided in Table 1.

TABLE 1
Examples of sequences useful for generation of chimeric polypeptides

| Name | Nucleotide Sequence | |
|---|---|---|
| ACE2 | SEQ ID NO: 1: | SEQ ID NO: 2: |
| (full | GGCACTCATACATACACTCTGGCAATGAGGACACTGAGCTCGCTTCTGA | MSSSSWLLLSLVAVTAAQSTIEEQAKTFLDKFNHEAEDLFYQSS |
| length) | AATTTGACAAGATAACCACTAAAATCTCTTTGAATTCTATGTTGTTGTG | LASWNYNTNITEENVQNMNNAGDKWSAFLKEQSTLAQMYPLQEI |
| | ATCCCATGGCTACAGAGGATCAGGAGTTGACATAGATACTCTTTGGATT | QNLTVKLQLQALQQNGSSVLSEDKSKRLNTILNTMSTIYSTGKV |
| | TCATACCATGTGGAGGCTTTCTTACTTCCACGTGACCTTGACTGAGTTT | CNPDNPQECLLLEPGLNEIMANSLDYNERLWAWESWRSEVGKQL |
| | TGAATAGCGCCCAACCCAAGTTCAAAGGCTGATAAGAGAGAAAATCTCA | RPLYEEYVVLKNEMARANHYEDYGDYWRGDYEVNGVDGYDYSRG |
| | TGAGGAGGTTTTAGTCTAGGGAAAGTCATTCAGTGGATGTGATCTTGGC | QLIEDVEHTFEEIKPLYEHLHAYVRAKLMNAYPSYISPIGCLPA |
| | TCACAGGGGACGATGTCAAGCTCTTCCTGGCTCCTTCTCAGCCTTGTTG | HLLGDMWGRFWTNLYSLTVPFGQKPNIDVTDAMVDQAWDAQRIF |
| | CTGTAACTGCTGCTCAGTCCACCATTGAGGAACAGGCCAAGACATTTTT | KEAEKFFVSVGLPNMTQGFWENSMLTDPGNVQKAVCHPTAWDLG |
| | GGACAAGTTTAACCACGAAGCCGAAGACCTGTTCTATCAAAGTTCACTT | KGDFRILMCTKVTMDDFLTAHHEMGHIQYDMAYAAQPFLLRNGA |
| | GCTTCTTGGAATTATAACACCAATATTACTGAAGAGAATGTCCAAAACA | NEGFHEAVGEIMSLSAATPKHLKSIGLLSPDFQEDNETEINFLL |
| | TGAATAATGCTGGGGACAAATGGTCTGCCTTTTTAAAGGAACAGTCCAC | KQALTIVGTLPFTYMLEKWRWMVFKGEIPKDQWMKKWWEMKREI |
| | ACTTGCCCAAATGTATCCACTACAAGAAATTCAGAATCTCACAGTCAAG | VGVVEPVPHDETYCDPASLFHVSNDYSFIRYYTRTLYQFQFQEA |
| | CTTCAGCTGCAGGCTCTTCAGCAAAATGGGTCTTCAGTGCTCTCAGAAG | LCQAAKHEGPLHKCDISNSTEAGQKLFNMLRLGKSEPWTLALEN |
| | ACAAGAGCAAACGGTTGAACACAATTCTAAATACAATGAGCACCATCTA | VVGAKNMNVRPLLNYFEPLFTWLKDONKNSFVGWSTDWSPYADQ |
| | CAGTACTGGAAAAGTTTGTAACCCAGATAATCCACAAGAATGCTTATTA | SIKVRISLKSALGDKAYEWNDNEMYLFRSSVAYAMRQYFLKVKN |
| | CTTGAACCAGGTTTGAATGAAATAATGGCAAACAGTTTAGACTACAATG | QMILFGEEDVRVANLKPRISFNFFVTAPKNVSDIIPRTEVEKAI |
| | AGAGGCTCTGGGCTTGGGAAAGCTGGAGATCTGAGGTCGGCAAGCAGCT | RMSRSRINDAFRLNDNSLEFLGIQPTLGPPNQPPVSIWLIVFGV |
| | GAGGCCATTATATGAAGAGTATGTGGTCTTGAAAAATGAGATGGCAAGA | VMGVIVVGIVILIFTGIRDRKKKNKARSGENPYASIDISKGENN |
| | GCAAATCATTATGAGGACTATGGGGATTATTGGAGAGGAGACTATGAAG | PGFQNTDDVQTSF |
| | TAAATGGGGTAGATGGCTATGACTACAGCCGCGGCCAGTTGATTGAAGA | |
| | TGTGGAACATACCTTTGAAGAGATTAAACCATTATATGAACATCTTCAT | |
| | GCCTATGTGAGGGCAAAGTTGATGAATGCCTATCCTTCCTATATCAGTC | |
| | CAATTGGATGCCTCCCTGCTCATTTGCTTGGTGATATGTGGGGTAGATT | |
| | TTGGACAAATCTGTACTCTTTGACAGTTCCCTTTGGACAGAAACCAAAC | |
| | ATAGATGTTACTGATGCAATGGTGGACCAGGCCTGGGATGCACAGAGAA | |
| | TATTCAAGGAGGCCGAGAAGTTCTTTGTATCTGTTGGTCTTCCTAATAT | |
| | GACTCAAGGATTCTGGGAAAATTCCATGCTAACGGACCCAGGAAATGTT | |
| | CAGAAAGCAGTCTGCCATCCCACAGCTTGGGACCTGGGGAAGGGCGACT | |
| | TCAGGATCCTTATGTGCACAAAGGTGACAATGGACGACTTCCTGACAGC | |
| | TCATCATGAGATGGGGCATATCCAGTATGATATGGCATATGCTGCACAA | |
| | CCTTTTCTGCTAAGAAATGGAGCTAATGAAGGATTCCATGAAGCTGTTG | |
| | GGGAAATCATGTCACTTTCTGCAGCCACACCTAAGCATTTAAAATCCAT | |
| | TGGTCTTCTGTCACCCGATTTTCAAGAAGACAATGAAACAGAAATAAAC | |
| | TTCCTGCTCAAACAAGCACTCACGATTGTTGGGACTCTGCCATTTACTT | |
| | ACATGTTAGAGAAGTGGAGGTGGATGGTCTTTAAAGGGGAAATTCCCAA | |
| | AGACCAGTGGATGAAAAAGTGGTGGGAGATGAAGCGAGAGATAGTTGGG | |
| | GTGGTGGAACCTGTGCCCCATGATGAAACATACTGTGACCCCGCATCTC | |
| | TGTTCCATGTTTCTAATGATTACTCATTCATTCGATATTACACAAGGAC | |
| | CCTTTACCAATTCCAGTTTCAAGAAGCACTTTGTCAAGCAGCTAAACAT | |
| | GAAGGCCCTCTGCACAAATGTGACATCTCAAACTCTACAGAAGCTGGAC | |
| | AGAAACTGTTCAATATGCTGAGGCTTGGAAAATCAGAACCCTGGACCCT | |
| | AGCATTGGAAAATGTTGTAGGAGCAAAGAACATGAATGTAAGGCCACTG | |
| | CTCAACTACTTTGAGCCCTTATTTACCTGGCTGAAAGACCAGAACAAGA | |
| | ATTCTTTTGTGGGATGGAGTACCGACTGGAGTCCATATGCAGACCAAAG | |
| | CATCAAAGTGAGGATAAGCCTAAAATCAGCTCTTGGAGATAAAGCATAT | |
| | GAATGGAACGACAATGAAATGTACCTGTTCCGATCATCTGTTGCATATG | |
| | CTATGAGGCAGTACTTTTTAAAAGTAAAAAATCAGATGATTCTTTTTGG | |
| | GGAGGAGGATGTGCGAGTGGCTAATTTGAAACCAAGAATCTCCTTTAAT | |
| | TTCTTTGTCACTGCACCTAAAAATGTGTCTGATATCATTCCTAGAACTG | |
| | AAGTTGAAAAGGCCATCAGGATGTCCCGGAGCCGTATCAATGATGCTTT | |
| | CCGTCTGAATGACAACAGCCTAGAGTTTCTGGGGATACAGCCAACACTT | |
| | GGACCTCCTAACCAGCCCCCTGTTTCCATATGGCTGATTGTTTTTGGAG | |
| | TTGTGATGGGAGTGATAGTGGTTGGCATTGTCATCCTGATCTTCACTGG | |
| | GATCAGAGATCGGAAGAAGAAAAATAAAGCAAGAAGTGGAGAAAATCCT | |
| | TATGCCTCCATCGATATTAGCAAAGGAGAAAATAATCCAGGATTCCAAA | |
| | ACACTGATGATGTTCAGACCTCCTTTTAGAAAAATCTATGTTTTTCCTC | |
| | TTGAGGTGATTTTGTTGTATGTAAATGTTAATTTCATGGTATAGAAAAT | |
| | ATAAGATGATAAAGATATCATTAAATGTCAAAACTATGACTCTGTTCAG | |
| | AAAAAAAATTGTCCAAAGACAACATGGCCAAGGAGAGAGCATCTTCATT | |
| | GACATTGCTTTCAGTATTTATTTCTGTCTCTGGATTTGACTTCTGTTCT | |
| | GTTTCTTAATAAGGATTTTGTATTAGAGTATATTAGGGAAAGTGTGTAT | |
| | TTGGTCTCACAGGCTGTTCAGGGATAATCTAAATGTAAATGTCTGTTGA | |
| | ATTTCTGAAGTTGAAAACAAGGATATATCATTGGAGCAAGTGTTGGATC | |
| | TTGTATGGAATATGGATGGATCACTTGTAAGGACAGTGCCTGGGAACTG | |
| | GTGTAGCTGCAAGGATTGAGAATGGCATGCATTAGCTCACTTTCATTTA | |
| | ATCCATTGTCAAGGATGACATGCTTTCTTCACAGTAACTCAGTTCAAGT | |
| | ACTATGGTGATTTGCCTACAGTGATGTTTGGAATCGATCATGCTTTCTT | |
| | CAAGGTGACAGGTCTAAAGAGAGAAGAATCCAGGGAACAGGTAGAGGAC | |
| | ATTGCTTTTTCACTTCCAAGGTGCTTGATCAACATCTCCCTGACAACAC | |
| | AAAACTAGAGCCAGGGGCCTCCGTGAACTCCCAGAGCATGCCTGATAGA | |
| | AACTCATTTCTACTGTTCTCTAACTGTGGAGTGAATGGAAATTCCAACT | |
| | GTATGTTCACCCTCTGAAGTGGGTACCCAGTCTCTTAAATCTTTTGTAT | |
| | TTGCTCACAGTGTTTGAGCAGTGCTGAGCACAAAGCAGACACTCAATAA | |
| | ATGCTAGATTTACACACTC | |
| ACE2 | SEQ ID NO: 3: | SEQ ID NO: 4: |
| (19-358) | CTGGCCTTACCAGTGACCGCCTTGCTCCTGCCGCTGGCCTTGCTGCTCC | STIEEQAKTFLDKFNHEAEDLFYQSSLASWNYNTNITEENVQNM |
| | ACGCCGCCAGGCCGGGATCCTCCACCATTGAGGAACAGGCCAAGACATT | NNAGDKWSAFLKEQSTLAQMYPLQEIQNLTVKLQLQALQONGSS |
| | TTTGGACAAGTTTAACCACGAAGCCGAAGACCTGTTCTATCAAAGTTCA | VLSEDKSKRLNTILNTMSTIYSTGKVCNPDNPQECLLLEPGLNE |
| | CTTGCTTCTTGGAATTATAACACCAATATTACTGAAGAGAATGTCCAAA | IMANSLDYNERLWAWESWRSEVGKQLRPLYEEYVVLKNEMARAN |
| | ACATGAATAATGCTGGGGACAAATGGTCTGCCTTTTTAAAGGAACAGTC | HYEDYGDYWRGDYEVNGVDGYDYSRGQLIEDVEHTFEEIKPLYE |
| | CACACTTGCCCAAATGTATCCACTACAAGAAATTCAGAATCTCACAGTC | HLHAYVRAKLMNAYPSYISPIGCLPAHLLGDMWGRFWTNLYSLT |
| | AAGCTTCAGCTGCAGGCTCTTCAGCAAAATGGGTCTTCAGTGCTCTCAG | VPFGQKPNIDVTDAMVDQAWDAQRIFKEAEKFFVSVGLPNMTQG |
| | AAGACAAGAGCAAACGGTTGAACACAATTCTAAATACAATGAGCACCAT | FWENSMLTDPGNVQKAVCHPTAWDLGKGDFRI |
| | CTACAGTACTGGAAAAGTTTGTAACCCAGATAATCCACAAGAATGCTTA | |
| | TTACTTGAACCAGGTTTGAATGAAATAATGGCAAACAGTTTAGACTACA | |
| | ATGAGAGGCTCTGGGCTTGGGAAAGCTGGAGATCTGAGGTCGGCAAGCA | |
| | GCTGAGGCCATTATATGAAGAGTATGTGGTCTTGAAAAATGAGATGGCA | |
| | AGAGCAAATCATTATGAGGACTATGGGGATTATTGGAGAGGAGACTATG | |
| | AAGTAAATGGGGTAGATGGCTATGACTACAGCCGCGGCCAGTTGATTGA | |
| | AGATGTGGAACATACCTTTGAAGAGATTAAACCATTATATGAACATCTT | |
| | CATGCCTATGTGAGGGCAAAGTTGATGAATGCCTATCCTTCCTATATCA | |
| | GTCCAATTGGATGCCTCCCTGCTCATTTGCTTGGTGATATGTGGGGTAG | |
| | ATTTTGGACAAATCTGTACTCTTTGACAGTTCCCTTTGGACAGAAACCA | |
| | AACATAGATGTTACTGATGCAATGGTGGACCAGGCCTGGGATGCACAGA | |
| | GAATATTCAAGGAGGCCGAGAAGTTCTTTGTATCTGTTGGTCTTCCTAA | |
| | TATGACTCAAGGATTCTGGGAAAATTCCATGCTAACGGACCCAGGAAAT | |
| | GTTCAGAAAGCAGTCTGCCATCCCACAGCTTGGGACCTGGGGAAGGGCG | |
| | ACTTCAGGATCGGATCCTTCGTGCCGGTCTTCCTGCCAGCGAAGCCCAC | |
| | CACGACGCCAGCGCCGCGACCACCAACACCGGCGCCCACCATCGCGTCG | |
| | CAGCCCCTGTCCCTGCGCCCAGAGGCGTGCCGGCCAGCGGCGGGGGGCG | |
| | CAGTGCACACGAGGGGGCTGGACTTCGCCTGTGATATCTACATCTGGGC | |
| | GCCCTTGGCCGGGACTTGTGGGGTCCTTCTCCTGTCACTGGTTATCACC | |
| | CTTTACTGCAACCACAGGAACAGAGTGAAGTTCAGCAGGAGCGCAGACG | |
| | CCCCCGCGTACCAGCAGGGCCAGAACCAGCTCTATAACGAGCTCAATCT | |
| | AGGACGAAGAGAGGAGTACGATGTTTTGGACAAGAGACGTGGCCGGGAC | |
| | CCTGAGATGGGGGGAAAGCCGAGAAGGAAGAACCCTCAGGAAGGCCTGT | |
| | ACAATGAACTGCAGAAAGATAAGATGGCGGAGGCCTACAGTGAGATTGG | |
| | GATGAAAGGCGAGCGCCGGAGGGGCAAGGGGCACGATGGCCTTTACCAG | |
| | GGTCTCAGTACAGCCACCAAGGACACCTACGACGCCCTTCACATGCAGG | |
| | CCCTGCCCCCTCGC | |
| ACE2 | SEQ ID NO: 5: | SEQ ID NO: 6: |
| (19-605) | ATGGCCTTACCAGTGACCGCCTTGCTCCTGCCGCTGGCCTTGCTGCTCC | STIEEQAKTFLDKFNHEAEDLFYQSSLASWNYNTNITEENVQNM |
| | ACGCCGCCAGGCCGGGATCCTCCACCATTGAGGAACAGGCCAAGACATT | NNAGDKWSAFLKEQSTLAQMYPLQEIQNLTVKLQLQALQQNGSS |
| | TTTGGACAAGTTTAACCACGAAGCCGAAGACCTGTTCTATCAAAGTTCA | VLSEDKSKRLNTILNTMSTIYSTGKVCNPDNPQECLLLEPGLNE |
| | CTTGCTTCTTGGAATTATAACACCAATATTACTGAAGAGAATGTCCAAA | IMANSLDYNERLWAWESWRSEVGKQLRPLYEEYVVLKNEMARAN |
| | ACATGAATAATGCTGGGGACAAATGGTCTGCCTTTTTAAAGGAACAGTC | HYEDYGDYWRGDYEVNGVDGYDYSRGQLIEDVEHTFEEIKPLYE |
| | CACACTTGCCCAAATGTATCCACTACAAGAAATTCAGAATCTCACAGTC | HLHAYVRAKLMNAYPSYISPIGCLPAHLLGDMWGRFWINLYSLT |
| | AAGCTTCAGCTGCAGGCTCTTCAGCAAAATGGGTCTTCAGTGCTCTCAG | VPFGQKPNIDVTDAMVDQAWDAQRIFKEAEKFFVSVGLPNMTQG |
| | AAGACAAGAGCAAACGGTTGAACACAATTCTAAATACAATGAGCACCAT | FWENSMLTDPGNVQKAVCHPTAWDLGKGDFRILMCTKVTMDDEL |
| | CTACAGTACTGGAAAAGTTTGTAACCCAGATAATCCACAAGAATGCTTA | TAHHEMGHIQYDMAYAAQPFLLRNGANEGFHEAVGEIMSLSAAT |
| | TTACTTGAACCAGGTTTGAATGAAATAATGGCAAACAGTTTAGACTACA | PKHLKSIGLLSPDFQEDNETEINFLLKQALTIVGTLPFTYMLEK |
| | ATGAGAGGCTCTGGGCTTGGGAAAGCTGGAGATCTGAGGTCGGCAAGCA | WRWMVFKGEIPKDQWMKKWWEMKREIVGVVEPVPHDETYCDPAS |
| | GCTGAGGCCATTATATGAAGAGTATGTGGTCTTGAAAAATGAGATGGCA | LFHVSNDYSFIRYYTRTLYQFQFQEALCQAAKHEGPLHKCDISN |
| | AGAGCAAATCATTATGAGGACTATGGGGATTATTGGAGAGGAGACTATG | STEAGQKLENMLRLGKSEPWTLALENVVGAKNMNVRPLLNYFEP |
| | AAGTAAATGGGGTAGATGGCTATGACTACAGCCGCGGCCAGTTGATTGA | LFTWLKDQNKNSFVG |
| | AGATGTGGAACATACCTTTGAAGAGATTAAACCATTATATGAACATCTT | |
| | CATGCCTATGTGAGGGCAAAGTTGATGAATGCCTATCCTTCCTATATCA | |
| | GTCCAATTGGATGCCTCCCTGCTCATTTGCTTGGTGATATGTGGGGTAG | |
| | ATTTTGGACAAATCTGTACTCTTTGACAGTTCCCTTTGGACAGAAACCA | |
| | AACATAGATGTTACTGATGCAATGGTGGACCAGGCCTGGGATGCACAGA | |
| | GAATATTCAAGGAGGCCGAGAAGTTCTTTGTATCTGTTGGTCTTCCTAA | |
| | TATGACTCAAGGATTCTGGGAAAATTCCATGCTAACGGACCCAGGAAAT | |
| | GTTCAGAAAGCAGTCTGCCATCCCACAGCTTGGGACCTGGGGAAGGGCG | |
| | ACTTCAGGATCCTTATGTGCACAAAGGTGACAATGGACGACTTCCTGAC | |
| | AGCTCATCATGAGATGGGGCATATCCAGTATGATATGGCATATGCTGCA | |
| | CAACCTTTTCTGCTAAGAAATGGAGCTAATGAAGGATTCCATGAAGCTG | |
| | TTGGGGAAATCATGTCACTTTCTGCAGCCACACCTAAGCATTTAAAATC | |
| | CATTGGTCTTCTGTCACCCGATTTTCAAGAAGACAATGAAACAGAAATA | |
| | AACTTCCTGCTCAAACAAGCACTCACGATTGTTGGGACTCTGCCATTTA | |
| | CTTACATGTTAGAGAAGTGGAGGTGGATGGTCTTTAAAGGGGAAATTCC | |
| | CAAAGACCAGTGGATGAAAAAGTGGTGGGAGATGAAGCGAGAGATAGTT | |
| | GGGGTGGTGGAACCTGTGCCCCATGATGAAACATACTGTGACCCCGCAT | |
| | CTCTGTTCCATGTTTCTAATGATTACTCATTCATTCGATATTACACAAG | |
| | GACCCTTTACCAATTCCAGTTTCAAGAAGCACTTTGTCAAGCAGCTAAA | |
| | CATGAAGGCCCTCTGCACAAATGTGACATCTCAAACTCTACAGAAGCTG | |
| | GACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCA | |
| | CTGCTCAACTACTTTGAGCCCTTATTTACCTGGCTGAAAGACCAGAACA | |
| | AGAATTCTTTTGTGGGAGGATCCTTCGTGCCGGTCTTCCTGCCAGCGAA | |
| | GCCCACCACGACGCCAGCGCCGCGACCACCAACACCGGCGCCCACCATC | |
| | GCGTCGCAGCCCCTGTCCCTGCGCCCAGAGGCGTGCCGGCCAGCGGCGG | |
| | GGGGCGCAGTGCACACGAGGGGGCTGGACTTCGCCTGTGATATCTACAT | |
| | CTGGGCGCCCTTGGCCGGGACTTGTGGGGTCCTTCTCCTGTCACTGGTT | |
| | ATCACCCTTTACTGCAACCACAGGAACAGAGTGAAGTTCAGCAGGAGCG | |
| | CAGACGCCCCCGCGTACCAGCAGGGCCAGAACCAGCTCTATAACGAGCT | |
| | CAATCTAGGACGAAGAGAGGAGTACGATGTTTTGGACAAGAGACGTGGC | |
| | CGGGACCCTGAGATGGGGGGAAAGCCGAGAAGGAAGAACCCTCAGGAAG | |
| | GCCTGTACAATGAACTGCAGAAAGATAAGATGGCGGAGGCCTACAGTGA | |
| | GATTGGGATGAAAGGCGAGCGCCGGAGGGGCAAGGGGCACGATGGCCTT | |
| | TACCAGGGTCTCAGTACAGCCACCAAGGACACCTACGACGCCCTTCACA | |
| | TGCAGGCCCTGCCCCCTCGC | |
| ACE2 | SEQ ID NO: 7: | SEQ ID NO: 8: |
| (19-740) | ATGGCCTTACCAGTGACCGCCTTGCTCCTGCCGCTGGCCTTGCTGCTCC | STIEEQAKTFLDKFNHEAEDLFYQSSLASWNYNTNITEENVQNM |
| | ACGCCGCCAGGCCGGGATCCTCCACCATTGAGGAACAGGCCAAGACATT | NNAGDKWSAFLKEQSTLAQMYPLQEIQNLTVKLQLQALQQNGSS |
| | TTTGGACAAGTTTAACCACGAAGCCGAAGACCTGTTCTATCAAAGTTCA | VLSEDKSKRLNTILNTMSTIYSTGKVCNPDNPQECLLLEPGLNE |
| | CTTGCTTCTTGGAATTATAACACCAATATTACTGAAGAGAATGTCCAAA | IMANSLDYNERLWAWESWRSEVGKOLRPLYEEYVVLKNEMARAN |
| | ACATGAATAATGCTGGGGACAAATGGTCTGCCTTTTTAAAGGAACAGTC | HYEDYGDYWRGDYEVNGVDGYDYSRGQLIEDVEHTFEEIKPLYE |
| | CACACTTGCCCAAATGTATCCACTACAAGAAATTCAGAATCTCACAGTC | HLHAYVRAKLMNAYPSYISPIGCLPAHLLGDMWGRFWTNLYSLT |
| | AAGCTTCAGCTGCAGGCTCTTCAGCAAAATGGGTCTTCAGTGCTCTCAG | VPFGQKPNIDVTDAMVDQAWDAQRIFKEAEKFFVSVGLPNMTQG |
| | AAGACAAGAGCAAACGGTTGAACACAATTCTAAATACAATGAGCACCAT | FWENSMLTDPGNVQKAVCHPTAWDLGKGDFRILMCTKVTMDDEL |
| | CTACAGTACTGGAAAAGTTTGTAACCCAGATAATCCACAAGAATGCTTA | TAHHEMGHIQYDMAYAAQPFLLRNGANEGFHEAVGEIMSLSAAT |
| | TTACTTGAACCAGGTTTGAATGAAATAATGGCAAACAGTTTAGACTACA | PKHLKSIGLLSPDFQEDNETEINFLLKQALTIVGTLPFTYMLEK |
| | ATGAGAGGCTCTGGGCTTGGGAAAGCTGGAGATCTGAGGTCGGCAAGCA | WRWMVFKGEIPKDQWMKKWWEMKREIVGVVEPVPHDETYCDPAS |
| | GCTGAGGCCATTATATGAAGAGTATGTGGTCTTGAAAAATGAGATGGCA | LFHVSNDYSFIRYYTRTLYQFQFQEALCQAAKHEGPLHKCDISN |
| | AGAGCAAATCATTATGAGGACTATGGGGATTATTGGAGAGGAGACTATG | STEAGQKLFNMLRLGKSEPWTLALENVVGAKNMNVRPLLNYFEP |
| | AAGTAAATGGGGTAGATGGCTATGACTACAGCCGCGGCCAGTTGATTGA | LFTWLKDQNKNSFVGWSTDWSPYADQSIKVRISLKSALGDKAYE |
| | AGATGTGGAACATACCTTTGAAGAGATTAAACCATTATATGAACATCTT | WNDNEMYLFRSSVAYAMRQYFLKVKNQMILFGEEDVRVANLKPR |
| | CATGCCTATGTGAGGGCAAAGTTGATGAATGCCTATCCTTCCTATATCA | ISFNFFVTAPKNVSDIIPRTEVEKAIRMSRSRINDAFRLNDNSL |
| | GTCCAATTGGATGCCTCCCTGCTCATTTGCTTGGTGATATGTGGGGTAG | EFLGIQPTLGPPNQPPVS |
| | ATTTTGGACAAATCTGTACTCTTTGACAGTTCCCTTTGGACAGAAACCA | |
| | AACATAGATGTTACTGATGCAATGGTGGACCAGGCCTGGGATGCACAGA | |
| | GAATATTCAAGGAGGCCGAGAAGTTCTTTGTATCTGTTGGTCTTCCTAA | |
| | TATGACTCAAGGATTCTGGGAAAATTCCATGCTAACGGACCCAGGAAAT | |
| | GTTCAGAAAGCAGTCTGCCATCCCACAGCTTGGGACCTGGGGAAGGGCG | |
| | ACTTCAGGATCCTTATGTGCACAAAGGTGACAATGGACGACTTCCTGAC | |
| | AGCTCATCATGAGATGGGGCATATCCAGTATGATATGGCATATGCTGCA | |
| | CAACCTTTTCTGCTAAGAAATGGAGCTAATGAAGGATTCCATGAAGCTG | |
| | TTGGGGAAATCATGTCACTTTCTGCAGCCACACCTAAGCATTTAAAATC | |
| | CATTGGTCTTCTGTCACCCGATTTTCAAGAAGACAATGAAACAGAAATA | |
| | AACTTCCTGCTCAAACAAGCACTCACGATTGTTGGGACTCTGCCATTTA | |
| | CTTACATGTTAGAGAAGTGGAGGTGGATGGTCTTTAAAGGGGAAATTCC | |
| | CAAAGACCAGTGGATGAAAAAGTGGTGGGAGATGAAGCGAGAGATAGTT | |
| | GGGGTGGTGGAACCTGTGCCCCATGATGAAACATACTGTGACCCCGCAT | |
| | CTCTGTTCCATGTTTCTAATGATTACTCATTCATTCGATATTACACAAG | |
| | GACCCTTTACCAATTCCAGTTTCAAGAAGCACTTTGTCAAGCAGCTAAA | |
| | CATGAAGGCCCTCTGCACAAATGTGACATCTCAAACTCTACAGAAGCTG | |
| | GACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCA | |
| | CTGCTCAACTACTTTGAGCCCTTATTTACCTGGCTGAAAGACCAGAACA | |
| | AGAATTCTTTTGTGGGATGGAGTACCGACTGGAGTCCATATGCAGACCA | |
| | AAGCATCAAAGTGAGGATAAGCCTAAAATCAGCTCTTGGAGATAAAGCA | |
| | TATGAATGGAACGACAATGAAATGTACCTGTTCCGATCATCTGTTGCAT | |
| | ATGCTATGAGGCAGTACTTTTTAAAAGTAAAAAATCAGATGATTCTTTT | |
| | TGGGGAGGAGGATGTGCGAGTGGCTAATTTGAAACCAAGAATCTCCTTT | |
| | AATTTCTTTGTCACTGCACCTAAAAATGTGTCTGATATCATTCCTAGAA | |
| | CTGAAGTTGAAAAGGCCATCAGGATGTCCCGGAGCCGTATCAATGATGC | |
| | TTTCCGTCTGAATGACAACAGCCTAGAGTTTCTGGGGATACAGCCAACA | |
| | CTTGGACCTCCTAACCAGCCCCCTGTTTCCATCTACATCTGGGCGCCCT | |
| | TGGCCGGGACTTGTGGGGTCCTTCTCCTGTCACTGGTTATCACCCTTTA | |
| | CTGCAACCACAGGAACAGAGTGAAGTTCAGCAGGAGCGCAGACGCCCCC | |
| | GCGTACCAGCAGGGCCAGAACCAGCTCTATAACGAGCTCAATCTAGGAC | |
| | GAAGAGAGGAGTACGATGTTTTGGACAAGAGACGTGGCCGGGACCCTGA | |
| | GATGGGGGGAAAGCCGAGAAGGAAGAACCCTCAGGAAGGCCTGTACAAT | |
| | GAACTGCAGAAAGATAAGATGGCGGAGGCCTACAGTGAGATTGGGATGA | |
| | AAGGCGAGCGCCGGAGGGGCAAGGGGCACGATGGCCTTTACCAGGGTCT | |
| | CAGTACAGCCACCAAGGACACCTACGACGCCCTTCACATGCAGGCCCTG | |
| | CCCCCTCGC | |
| Mannose | SEQ ID NO: 9: | SEQ ID NO: 10: |
| Receptor | GACACAAGGAAGATGGACCCTTCTAAACCGTCTTCCAACGTGGCCGGAG | DTRKMDPSKPSSNVAGVVIIVILLILTGAGLAAYFFYKKRRVHL |
| (82 aa | TAGTCATCATTGTGATCCTCCTGATTTTAACGGGTGCTGGCCTTGCCGC | PQEGAFENTLYFNSQSSPGTSDMKDLVGNIEQNEHSVI |
| fragment) | CTATTTCTTTTATAAGAAAAGACGTGTGCACCTACCTCAAGAGGGCGCC | |
| | TTTGAAAACACTCTGTATTTTAACAGTCAGTCAAGCCCAGGAACTAGTG | |
| | ATATGAAAGATCTCGTGGGCAATATTGAACAGAATGAACACTCGGTCAT | |
| | C | |
| Mannose | SEQ ID NO: 11: | SEQ ID NO: 12: |
| Receptor | ATTGATGCTAAACCTACTCATGAATTACTTACAACAAAAGCTGACACAA | IDAKPTHELLTTKADTRKMDPSKPSSNVAGVVIIVILLILTGAG |
| (96 aa | GGAAGATGGACCCTTCTAAACCGTCTTCCAACGTGGCCGGAGTAGTCAT | LAAYFFYKKRRVHLPQEGAFENTLYFNSQSSPGTSDMKDLVGNI |
| fragment) | CATTGTGATCCTCCTGATTTTAACGGGTGCTGGCCTTGCCGCCTATTTC | EQNEHSVI |
| | TTTTATAAGAAAAGACGTGTGCACCTACCTCAAGAGGGCGCCTTTGAAA | |
| | ACACTCTGTATTTTAACAGTCAGTCAAGCCCAGGAACTAGTGATATGAA | |
| | AGATCTCGTGGGCAATATTGAACAGAATGAACACTCGGTCATC | |
| MERTK | SEQ ID NO: 13: | SEQ ID NO: 14: |
| fragment | TTTGGATGTTTTTGTGGTTTCATCCTCATCGGTTTGATATTGTACATAA | FGCFCGFILIGLILYISLAIRKRVQETKFGNAFTEEDSELVVNY |
| | GTCTGGCGATAAGGAAGAGAGTTCAAGAGACAAAGTTCGGAAATGCCTT | IAKKSFCRRAIELTLHSLGVSEELQNKLEDVVIDRNLLILGKIL |
| | TACAGAGGAAGACAGTGAGCTCGTTGTAAACTACATCGCAAAAAAAAGC | GEGEFGSVMEGNLKQEDGTSLKVAVKTMKLDNSSQREIEEFLSE |
| | TTCTGTAGAAGAGCAATAGAGCTCACGTTGCACTCACTCGGTGTGTCCG | AACMKDFSHPNVIRLLGVCIEMSSQGIPKPMVILPFMKYGDLHT |
| | AAGAACTCCAGAATAAACTGGAAGACGTCGTTATCGATCGGAACCTCCT | YLLYSRLETGPKHIPLQTLLKFMVDIALGMEYLSNRNFLHRDLA |
| | CATACTTGGAAAAATACTGGGAGAAGGAGAGTTCGGCAGTGTCATGGAA | ARNCMLRDDMTVCVADFGLSKKIYSGDYYRQGRIAKMPVKWIAI |
| | GGTAACTTGAAACAAGAGGATGGTACCTCACTCAAGGTAGCTGTCAAGA | ESLADRVYTSKSDVWAFGVIMWEIATRGMTPYPGVQNHEMYDYL |
| | CGATGAAACTTGATAACAGTTCACAAAGGGAGATCGAAGAATTTCTGTC | LHGHRLKQPEDCLDELYEIMYSCWRTDPLDRPTFSVLRLQLEKL |
| | TGAGGCCGCCTGTATGAAAGACTTCTCACATCCTAATGTCATCAGACTT | LESLPDVRNQADVIYVNTOLLESSEGLAQGSTLAPLDLNIDPDS |
| | CTTGGCGTTTGTATCGAGATGTCTAGCCAAGGAATCCCAAAACCTATGG | IIASCTPRAAISVVTAEVHDSKPHEGRYILNGGSEEWEDLTSAP |
| | TCATATTGCCTTTCATGAAATATGGCGATCTGCATACATATTTGCTCTA | SAAVTAEKNSVLPGERLVRNGVSWSHSSMLPLGSSLPDELLFAD |
| | CTCTAGACTTGAGACAGGGCCCAAACATATTCCTCTCCAGACATTGCTC | DSSEGSEVLM |
| | AAGTTTATGGTCGATATTGCCCTGGGTATGGAGTACTTGAGCAACCGAA | |
| | ATTTTCTGCATCGGGATCTTGCCGCACGCAACTGCATGCTGCGCGATGA | |
| | CATGACCGTCTGCGTGGCTGATTTTGGGCTGTCAAAAAAAATATATTCT | |
| | GGAGACTACTACCGACAAGGGCGGATTGCAAAGATGCCCGTCAAATGGA | |
| | TTGCGATTGAAAGTTTGGCGGACAGGGTATATACTTCCAAATCAGATGT | |
| | TTGGGCTTTTGGAGTCACTATGTGGGAAATAGCTACACGCGGTATGACC | |
| | CCGTACCCCGGAGTACAAAATCATGAAATGTATGACTATCTCCTTCATG | |
| | GACACAGGCTGAAGCAGCCCGAGGACTGCCTGGACGAACTGTATGAAAT | |
| | AATGTATTCTTGTTGGCGAACCGATCCCTTGGACCGGCCTACTTTCAGT | |
| | GTCCTTAGATTGCAACTTGAGAAATTGCTCGAGTCTTTGCCGGATGTGC | |
| | GAAACCAGGCAGACGTGATCTATGTCAATACCCAACTTTTGGAAAGTTC | |
| | TGAGGGCCTCGCACAGGGTTCTACCCTTGCCCCGTTGGATCTTAACATA | |
| | GACCCAGACAGCATAATTGCTTCTTGTACACCTCGCGCTGCCATATCAG | |
| | TTGTAACAGCGGAGGTCCATGATAGTAAACCTCACGAGGGTCGCTATAT | |
| | CCTGAACGGCGGGTCAGAAGAATGGGAAGACCTGACATCAGCGCCGAGC | |
| | GCCGCCGTTACTGCTGAGAAAAACTCTGTCCTGCCCGGAGAGCGCTTGG | |
| | TTCGGAACGGGGTAAGTTGGAGCCATAGCTCAATGCTCCCCCTGGGTTC | |
| | AAGTCTCCCGGACGAGCTTCTTTTTGCGGACGACTCATCTGAGGGGTCC | |
| | GAAGTTCTGATG | |
| Dectin-1 | SEQ ID NO: 15: | SEQ ID NO: 16: |
| fragment | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLG |
| | AAAGGGCAGTTGTGCTGCCAGTCCACCGTGGCGGTTGATTGCCGTCATC | |
| | CTCGGCATTCTGTGCTTGGTTATTCTCGTTATAGCGGTGGTGCTTGGG | |
| Dectin-1 | SEQ ID NO: 17: | SEQ ID NO: 18: |
| full | ATGGAGTATCACCCCGACCTGGAGAATTTGGACGAGGATGGCTACACTC | MEYHPDLENLDEDGYTQLHFDSQSNTRIAVVSEKGSCAASPPWR |
| length | AACTGCATTTCGATAGTCAGTCCAATACAAGGATCGCTGTTGTGTCTGA | LIAVILGILCLVILVIAVVLGTMAIWRSNSGSNTLENGYELSRN |
| | AAAGGGCAGTTGTGCTGCCAGTCCACCGTGGCGGTTGATTGCCGTCATC | KENHSQPTQSSLEDSVTPTKAVKTTGVLSSPCPPNWIIYEKSCY |
| | CTCGGCATTCTGTGCTTGGTTATTCTCGTTATAGCGGTGGTGCTTGGGA | LFSMSLNSWDGSKRQCWOLGSNLLKIDSSNELGFIVKQVSSQPD |
| | CCATGGCGATCTGGCGCTCCAACTCTGGAAGTAACACCCTTGAAAATGG | NSFWIGLSRPQTEVPWLWEDGSTFSSNLFQIRTTATQENPSPNC |
| | TTACTTCCTCAGTAGGAACAAAGAGAACCATTCCCAGCCGACACAGTCA | VWIHVSVIYDQLCSVPSYSICEKKFSM |
| | AGCCTTGAAGATTCAGTCACCCCTACAAAGGCCGTAAAAACGACAGGTG | |
| | TCCTGTCCTCTCCGTGTCCGCCTAACTGGATCATCTACGAGAAAAGTTG | |
| | TTATCTGTTTAGCATGAGCCTTAACAGTTGGGATGGCTCAAAAAGGCAG | |
| | TGCTGGCAACTGGGGAGCAACCTTTTGAAGATAGACAGTTCCAACGAAC | |
| | TGGGCTTCATAGTCAAACAGGTGTCCTCTCAACCTGATAACTCATTCTG | |
| | GATCGGGCTCAGTCGACCCCAAACTGAGGTTCCATGGCTTTGGGAAGAC | |
| | GGCAGCACTTTCTCTTCAAATTTGTTTCAAATAAGAACCACCGCTACGC | |
| | AGGAGAATCCGAGTCCGAACTGTGTTTGGATTCACGTCTCAGTCATTTA | |
| | CGACCAACTTTGTAGTGTCCCTAGCTATTCCATCTGCGAGAAAAAGTTC | |
| | AGTATG | |
| FcyR | SEQ ID NO: 19: | SEQ ID NO: 20: |
| intra- | CGACTGAAGATCCAAGTGCGAAAGGCAGCTATAACCAGCTATGAGAAAT | RLKIQVRKAAITSYEKSDGVYTGLSTRNQETYETLKHEKPPQ |
| cellular | CAGATGGTGTTTACACGGGCCTGAGCACCAGGAACCAGGAGACTTACGA | |
| | GACTCTGAAGCATGAGAAACCACCACAG | |
| SR AI/II | SEQ ID NO: 21: | SEQ ID NO: 22: |
| fragment | ATGGAACAATGGGATCATTTTCACAACCAACAGGAGGACACTGACAGTT | MEQWDHFHNQQEDTDSCSESVKFDARSMTALLPPNPKNSPSLQE |
| | GTTCCGAGTCCGTAAAATTTGATGCTCGGTCCATGACGGCTCTCCTCCC | KLKSFKAALIALYLLVFAVLIPLIGIVAAQLL |
| | CCCCAATCCGAAAAATTCCCCCTCTCTTCAAGAAAAGCTGAAGAGTTTC | |
| | AAGTTCAGTTTGGCTGTAGTCGTCATTTATCTGATACTCTTGACCGCTG | |
| | GGGCTGGGCTGCTGGTG | |
| MARCO | SEQ ID NO: 23: | SEQ ID NO: 24: |
| fragment | ATGAGGAATAAGAAAATACTCAAGGAAGACGAACTTCTGAGTGAAACTC | MRNKKILKEDELLSETQQAAFHQIAMEPFEINVPKPKRRNGVNF |
| | AACAGGCTGCATTCCATCAAATCGCAATGGAGCCTTTTGAAATTAACGT | SLAVVVIYLILLTAGAGLLV |
| | CCCGAAGCCCAAAAGACGGAACGGTGTCAACTTCTCCTTGGCAGTTGTT | |
| | GTAATTTATCTTATCCTGCTTACTGCCGGAGCGGGGCTGCTGGTT | |
| SCARA5 | SEQ ID NO: 25: | SEQ ID NO: 26: |
| fragment | ATGGAAAATAAAGCAATGTACCTCCACACCGTATCAGACTGTGACACGT | MENKAMYLHTVSDCDTSSICEDSFDGRSLSKLNLCEDGPCHKRR |
| | CCTCTATCTGTGAGGATTCTTTTGACGGTCGAAGTCTTAGTAAGTTGAA | ASICCTQLGSLSALKHAVLGLYLLVFLILVGIFILAV |
| | TCTGTGCGAGGATGGGCCCTGTCACAAGAGGAGAGCCTCAATCTGCTGC | |
| | ACACAACTGGGGTCACTTAGTGCTCTGAAGCATGCTGTTTTGGGGTTGT | |
| | ATCTTCTGGTGTTCCTCATCCTGGTCGGTATATTTATATTGGCGGTA | |
| SRCL | SEQ ID NO: 27: | SEQ ID NO: 28: |
| fragment | ATGAAGGATGATTTCGCAGAAGAAGAAGAAGTACAATCCTTCGGGTACA | MKDDFAEEEEVQSFGYKRFGIQEGTQCTKCKNNWALKFSIILLY |
| | AGCGCTTCGGGATTCAAGAGGGTACACAATGCACAAAATGCAAGAACAA | ILCALLTITVAILG |
| | TTGGGCCCTTAAGTTCTCAATAATACTCCTTTACATACTTTGTGCGCTT | |
| | CTTACGATAACTGTAGCGATCCTGGGA | |
| CD36 | SEQ ID NO: 29: | SEQ ID NO: 30: |
| fragment | GGGTGCGATCGGAACTGCGGACTTATCGCTGGCGCGGTTATTGGGGCAG | GCDRNCGLIAGAVIGAVLAVFGGILMPVLLGLIEMILLSVGVVM |
| | TGCTCGCTGTGTTTGGTGGCATTCTCATGCCAGTCCTTCTCGGACTGAT | FVAFMISYCACRSKTIK |
| | TGAAATGATCCTTTTGTCTGTTGGAGTTGTAATGTTCGTAGCCTTTATG | |
| | ATCTCATACTGCGCGTGCAGATCAAAGACTATCAAG | |
| SR-B1 | SEQ ID NO: 31: | SEQ ID NO: 32: |
| fragment | ATGGGATGCTCTGCAAAGGCAAGATGGGCAGCAGGGGCGTTGGGCGTCG | MGCSAKARWAAGALGVAGLLCAVLGAVMIVMVAQYVLLALGCVL |
| | CGGGCCTGCTGTGTGCTGTTCTTGGGGCTGTTATGATAGTTATGGTCGC | LLVPVICQIRSQVGAGORAARADSHSLACWGKGASDRTLWPTAA |
| | GCAGTATGTGCTCTTGGCACTGGGGTGTGTCCTCCTGCTTGTTCCAGTT | WSPPPAAVLRLCRSGSGHCWGLRSTLASFACRVATTLPVLEGLG |
| | ATATGCCAGATACGAAGTCAAGTTGGGGGGGGTCAAAGGGCAGCTAGAG | PSLGGGTGS |
| | CAGATTCCCATAGCCTCGCGTGTTGGGGTAAAGGTGCATCCGACCGAAC | |
| | CCTTTGGCCTACTGCTGCGTGGTCACCTCCACCTGCTGCGGTGCTTCGC | |
| | CTGTGTCGCTCAGGTAGCGGTCACTGTTGGGGCCTTCGAAGTACATTGG | |
| | CGTCTTTCGCGTGCCGCGTGGCAACCACCTTGCCGGTCCTGGAAGGGCT | |
| | GGGCCCTTCTCTCGGAGGAGGTACTGGTAGC | |
| CD68 | SEQ ID NO: 33: | SEQ ID NO: 34: |
| fragment | ATTTTGCTGCCGCTGATAATCGGACTTATTCTCTTGGGGCTCCTGGCTC | ILLPLIIGLILLGLLALVLIAFCIIRRRPSAYQAL |
| | TCGTTTTGATCGCGTTCTGCATCATAAGGCGGCGCCCCAGCGCATACCA | |
| | GGCTCTG | |
| LOX-1 | SEQ ID NO: 35: | SEQ ID NO: 36: |
| fragment | ATGACCTTTGACGACCTGAAGATACAGACGGTGAAAGATCAACCTGATG | MTFDDLKIQTVKDQPDEKSNGKKAKGLQFLYSPWWCLAAATLGV |
| | AGAAATCAAATGGGAAAAAAGCGAAAGGTTTGCAGTTTCTTTATTCCCC | LCLGLVVTIMVLG |
| | CTGGTGGTGCCTTGCAGCGGCAACCCTTGGTGTTCTTTGTCTGGGGCTT | |
| | GTTGTGACGATCATGGTGCTGGGC | |
| SREC | SEQ ID NO: 37: | SEQ ID NO: 38: |
| fragment | GCACTGATAGCTGGCTCCCTTGTGCCCTTGCTCCTCCTTTTCCTTGGCT | ALIAGSLVPLLLLFLGLACCACCCWAPRSDLKDRPARDGATVSR |
| | TGGCTTGTTGCGCGTGTTGCTGCTGGGCCCCGCGGTCAGATCTTAAGGA | MKLQVWGTLTSLGSTLPCRSLSSHKLPWVTVSHHDPEVPENHSF |
| | TAGGCCTGCAAGGGATGGCGCAACAGTGAGTAGGATGAAACTGCAGGTG | IEPPSAGWATDDSFSSDPESGEADEVPAYCVPPQEGMVPVAQAG |
| | TGGGGAACCTTGACATCCCTCGGCTCAACTCTGCCTTGCAGGTCCTTGA | SSEASLAAGAFPPPEDASTPFAIPRTSSLARAKRPSVSFAEGTK |
| | GCAGCCATAAATTGCCTTGGGTTACGGTTAGTCATCATGACCCGGAGGT | FAPQSRRSSGELSSPLRKPKRLSRGAQSGPEGREAEESTGPEEA |
| | CCCTTTTAACCATTCTTTCATAGAACCACCGAGCGCCGGATGGGCGACT | EAPESFPAAASPGDSATGHRRPPLGGRTVAEHVEAIEGSVQESS |
| | GATGACTCTTTCAGTTCTGATCCAGAAAGTGGTGAGGCGGACGAGGTAC | GPVTTIYMLAGKPRGSEGPVRSVFRHFGSFQKGQAEAKVKRAIP |
| | CCGCGTATTGTGTACCCCCACAAGAGGGGATGGTTCCTGTCGCTCAGGC | KPPRQALNRKKGSPGLASGSVGQSPNSAPKAGLPGATGPMAVRP |
| | CGGTAGCAGCGAGGCGTCTTTGGCCGCAGGCGCTTTTCCTCCGCCTGAG | EEAVRGLGAGTESSRRAQEPVSGCGSPEQDPQKQAEEERQEEPE |
| | GACGCCTCCACTCCTTTTGCAATTCCGAGAACCTCAAGCCTCGCCCGGG | YENVVPISRPPEP |
| | CTAAGAGACCTTCCGTCTCCTTCGCCGAGGGAACGAAGTTCGCACCACA | |
| | AAGCAGAAGGAGCTCAGGTGAGCTCTCATCTCCACTCAGAAAACCTAAA | |
| | AGACTGTCCCGAGGTGCCCAAAGTGGTCCAGAAGGACGCGAGGCAGAAG | |
| | AAAGTACCGGCCCGGAGGAAGCTGAAGCGCCAGAATCATTCCCGGCAGC | |
| | GGCCTCCCCGGGAGATTCAGCCACCGGACACAGACGACCACCGTTGGGT | |
| | GGCCGCACCGTAGCCGAACATGTCGAAGCCATCGAAGGCTCCGTACAAG | |
| | AGTCTTCTGGTCCAGTGACTACAATATATATGCTCGCAGGGAAGCCGCG | |
| | AGGTTCAGAGGGTCCGGTTAGATCTGTTTTTCGCCACTTTGGCAGCTTC | |
| | CAGAAAGGACAGGCAGAGGCGAAGGTAAAAAGGGCTATTCCGAAGCCGC | |
| | CCCGCCAGGCACTCAATCGGAAAAAGGGAAGCCCAGGTCTTGCTAGTGG | |
| | GAGTGTGGGTCAATCTCCCAATAGTGCTCCGAAAGCAGGTCTGCCAGGC | |
| | GCGACCGGTCCCATGGCGGTCAGACCAGAAGAGGCTGTGAGAGGCTTGG | |
| | GAGCGGGAACTGAGAGTTCACGGCGCGCGCAGGAGCCAGTGTCTGGCTG | |
| | CGGAAGTCCAGAGCAAGACCCCCAAAAACAGGCAGAAGAGGAAAGACAA | |
| | GAGGAGCCCGAATATGAAAATGTTGTCCCTATATCACGGCCTCCTGAGC | |
| | CC | |
| MEGF10 | SEQ ID NO: 39: | SEQ ID NO: 40: |
| fragment | GCTATCGCGGGGATCATTATATTGGTCTTGGTGGTGCTGTTTCTGCTCG | AIAGIIILVLVVLFLLALFIIYRHKQKGKESSMPAVTYTPAMRV |
| | CGCTTTTCATTATATACCGCCATAAGCAGAAGGGCAAAGAGTCCTCCAT | VNADYTISGTLPHSNGGNANSHYFTNPSYHTLTQCATSPHVNNR |
| | GCCAGCCGTGACCTATACGCCTGCGATGCGCGTTGTCAACGCCGATTAC | DRMTVTKSKNNQLFVNLKNVNPGKRGPVGDCTGTLPADWKHGGY |
| | ACCATCAGTGGTACCCTCCCGCACAGTAACGGCGGAAATGCAAACTCTC | LNELGAFGLDRSYMGKSLKDLGKNSEYNSSNCSLSSSENPYATI |
| | ATTACTTTACAAATCCTAGTTACCATACACTCACTCAGTGTGCTACCTC | KDPPVLIPKSSECGYVEMKSPARRDSPYAEINNSTSANRNVYEV |
| | TCCCCATGTGAACAATCGGGACAGGATGACCGTTACGAAAAGCAAAAAT | EPTVSVVQGVFSNNGRLSQDPYDLPKNSHIPCHYDLLPVRDSSS |
| | AACCAGTTGTTTGTGAACCTTAAGAATGTGAATCCCGGCAAGAGGGGTC | SPKQEDSGGSSSNSSSSSE |
| | CGGTGGGTGACTGCACCGGAACTCTCCCCGCTGACTGGAAGCATGGCGG | |
| | GTACCTGAACGAACTCGGCGCGTTTGGGCTCGACCGAAGCTACATGGGT | |
| | AAAAGTCTTAAGGACCTCGGTAAGAATAGCGAGTATAATAGCTCTAACT | |
| | GTTCCCTTTCCAGCTCCGAGAATCCGTACGCTACTATAAAAGACCCCCC | |
| | GGTGCTCATTCCCAAATCCAGTGAGTGCGGGTACGTGGAGATGAAAAGT | |
| | CCCGCTCGAAGAGACAGTCCATACGCGGAAATCAATAACTCCACCAGTG | |
| | CGAACCGCAATGTGTACGAAGTGGAGCCCACCGTTTCCGTTGTACAAGG | |
| | TGTATTTTCAAACAATGGGAGGCTTAGCCAGGACCCCTATGATCTTCCA | |
| | AAGAACAGCCACATCCCGTGTCATTATGATCTGTTGCCGGTGAGGGATT | |
| | CTAGCTCTTCTCCTAAACAAGAGGACTCAGGTGGCTCTAGITCCAACTC | |
| | CTCCAGTTCTTCAGAG | |
| SR-PSOX | SEQ ID NO: 41: | SEQ ID NO: 42: |
| fragment | GTTCCAGTTCTGTGCCTTTTGGCCATAATTTTTATCCTTACAGCTGCAC | VPVLCLLAIIFILTAALSYVLCKRRRGQSPQSSPDLPVHYIPVA |
| | TTTCATATGTCTTGTGCAAACGACGGAGAGGGCAAAGCCCACAGAGCAG | PDSNT |
| | CCCAGACCTCCCAGTGCATTATATACCTGTGGCACCTGACTCAAACACA | |
| FEEL-1 | SEQ ID NO: 43: | SEQ ID NO: 44: |
| fragment | GGCGTAGGTGCGGTTTTGGCTGCTGGGGCCCTGCTTGGCTTGGTGGCGG | GVGAVLAAGALLGLVAGALYLRARGKPMGFGFSAFQAEDDADDD |
| | GAGCGCTTTATCTGCGAGCACGAGGAAAGCCTATGGGATTTGGTTTTTC | FSPWQEGTNPTLVSVPNPVFGSDTFCEPFDDSLLEEDFPDTQRI |
| | TGCTTTTCAGGCCGAGGACGACGCCGATGACGACTTTAGTCCTTGGCAA | LTVK |
| | GAAGGAACTAATCCAACGCTGGTTTCCGTGCCTAATCCCGTGTTTGGAT | |
| | CTGATACGTTTTGCGAGCCATTTGACGATTCTTTGCTGGAGGAAGATTT | |
| | CCCCGACACCCAGAGGATACTCACAGTTAAA | |
| CD163 | SEQ ID NO: 45: | SEQ ID NO: 46: |
| TM/IC | TTTATTGCCGTGGGAATTCTCGGTGTAGTGCTTCTTGCTATATTTGTCG | FIAVGILGVVLLAIFVALFFLTKKRRQRQRLAVSSRGENLVHQI |
| fragment | CTTTGTTCTTTCTGACTAAAAAGCGCAGGCAAAGGCAGCGGCTTGCTGT | QYREMNSCLNADDLDLMNSSENSHESADFSAAELISVSKFLPIS |
| | GAGCTCTCGGGGAGAAAACCTCGTTCACCAAATCCAATACCGAGAAATG | GMEKEAILSHTEKENGNL |
| | AACTCCTGTCTCAACGCCGACGATCTTGACCTGATGAACTCATCTGAGA | |
| | ACTCACACGAGTCCGCCGATTTCAGCGCGGCGGAATTGATCTCTGTCAG | |
| | CAAATTTCTGCCTATAAGTGGCATGGAAAAAGAAGCCATACTCTCTCAC | |
| | ACGGAAAAGGAAAATGGCAACCTT | |
| RAGE | SEQ ID NO: 47: | SEQ ID NO: 48: |
| fragment | CTCGCCCTTGGTATCTTGGGCGGTCTGGGCACAGCCGCGTTGCTCATTG | LALGILGGLGTAALLIGVILWQRRQRRGEERKAPENQEEEEERA |
| | GGGTTATTTTGTGGCAGAGACGACAGCGCCGAGGCGAAGAAAGGAAAGC | ELNQSEEPEAGESSTGGP |
| | GCCGGAGAACCAGGAAGAAGAGGAAGAAAGGGCTGAGCTGAACCAGTCT | |
| | GAAGAACCAGAAGCCGGGGAGAGTTCCACCGGCGGGCCA | |
| CD44 | SEQ ID NO: 49: | SEQ ID NO: 50: |
| fragment | TGGCTTATCATTTTGGCCTCACTTCTTGCTCTTGCACTTATCCTCGCTG | WLIILASLLALALILAVCIAVNSRRRCGQKKKLVINSGNGAVED |
| | TGTGCATTGCAGTGAACAGTAGAAGGCGGTGCGGTCAGAAGAAGAAATT | RKPSGLNGEASKSQEMVHLVNKESSETPDQFMTADETRNLQNVD |
| | GGTAATAAACTCAGGAAATGGAGCGGTAGAGGATAGGAAGCCATCTGGG | MKIGV |
| | CTTAACGGAGAAGCAAGCAAGTCTCAAGAGATGGTACATTTGGTCAATA | |
| | AGGAGAGTTCTGAAACACCTGACCAATTTATGACCGCTGATGAAACGCG | |
| | CAACCTCCAAAATGTAGACATGAAAATAGGCGTG | |
| SR-L1 | SEQ ID NO: 51: | SEQ ID NO: 52: |
| fragment | CATATAGCTTCTATACTTATTCCATTGCTCTTGCTTCTGCTGCTGGTAT | HIASILIPLLLLLLLVLVAGVVFWYKRRVQGAKGFQHQRMTNGA |
| | TGGTCGCTGGTGTTGTCTTTTGGTACAAGCGCCGCGTGCAGGGTGCCAA | MNVEIGNPTYKMYEGGEPDDVGGLLDADFALDPDKPTNFTPVYA |
| | GGGGTTCCAACACCAAAGAATGACCAATGGGGCAATGAATGTTGAAATT | TLYMGGHGSRHSLASTDEKRELLGRGPEDEIGDPLA |
| | GGAAACCCGACCTACAAAATGTATGAGGGAGGTGAACCTGATGATGTTG | |
| | GTGGTCTCTTGGACGCGGATTTTGCGCTCGACCCCGATAAGCCGACTAA | |
| | TTTCACTCCCGTGTATGCCACTCTTTATATGGGGGGCCATGGGAGTAGA | |
| | CACTCACTTGCCTCCACCGATGAAAAGAGGGAATTGTTGGGTCGAGGAC | |
| | CCGAAGACGAGATTGGGGACCCACTTGCG | |
| CD163 | SEQ ID NO: 104: | SEQ ID NO: 105: |
| soluble | TCGAGCCTCGGCGGCACCGATAAGGAGCTCCGGCTCGTTGACGGCGAAA | SSLGGTDKELRLVDGENKCSGRVEVKVQEEWGTVCNNGWSMEAV |
| | ACAAGTGCTCAGGCAGAGTGGAAGTGAAGGTTCAGGAGGAATGGGGGAC | SVICNQLGCPTAIKAPGWANSSAGSGRIWMDHVSCRGNESALWD |
| | TGTCTGCAACAATGGCTGGTCTATGGAAGCGGTGTCCGTGATCTGCAAT | CKHDGWGKHSNCTHQQDAGVTCSDGSNLEMRLTRGGNMCSGRIE |
| | CAGCTGGGCTGTCCTACAGCAATCAAAGCCCCCGGCTGGGCTAATAGTA | IKFQGRWGTVCDDNFNIDHASVICRQLECGSAVSFSGSSNFGEG |
| | GCGCTGGGTCCGGCCGCATTTGGATGGACCACGTGAGCTGTAGAGGAAA | SGPIWFDDLICNGNESALWNCKHQGWGKHNCDHAEDAGVICSKG |
| | CGAGAGCGCCCTGTGGGATTGCAAGCACGACGGCTGGGGCAAGCACAGC | ADLSLRLVDGVTECSGRLEVRFQGEWGTICDDGWDSYDAAVACK |
| | AACTGCACCCACCAGCAGGACGCGGGCGTGACCTGCAGCGATGGCAGCA | QLGCPTAVTAIGRVNASKGFGHIWLDSVSCQGHEPAIWQCKHHE |
| | ACCTGGAAATGAGGTTGACCAGAGGCGGCAATATGTGCTCTGGCCGGAT | WGKHYCNHNEDAGVTCSDGSDLELRLRGGGSRCAGTVEVEIQRL |
| | CGAGATCAAGTTCCAGGGCAGATGGGGCACCGTGTGTGACGACAATTTT | LGKVCDRGWGLKEADVVCRQLGCGSALKTSYQVYSKIQATNTWL |
| | AATATCGATCACGCTTCGGTTATCTGCAGACAGCTGGAATGCGGCTCTG | FLSSCNGNETSLWDCKNWOWGGLTCDHYEEAKITCSAHREPRLV |
| | CCGTCTCTTTCAGCGGGTCCTCCAATTTCGGCGAGGGGTCTGGACCAAT | GGDIPCSGRVEVKHGDTWGSICDSDFSLEAASVLCRELQCGTVV |
| | CTGGTTCGACGACCTGATTTGCAACGGCAACGAATCTGCTCTGTGGAAT | SILGGAHFGEGNGQIWAEEFQCEGHESHLSLCPVAPRPEGTCSH |
| | TGCAAGCACCAGGGGTGGGGCAAACACAACTGCGACCACGCCGAGGACG | SRDVGVVCSRYTEIRLVNGKTPCEGRVELKTLGAWGSLCNSHWD |
| | CAGGCGTTATCTGCTCCAAGGGCGCCGATCTCTCTCTGCGGCTGGTGGA | IEDAHVLCQQLKCGVALSTPGGARFGKGNGQIWRHMFHCTGTEQ |
| | CGGTGTCACCGAATGCTCTGGTCGGCTGGAAGTACGGTTTCAGGGAGAG | HMGDCPVTALGASLCPSEQVASVICSGNQSQTLSSCNSSSLGPT |
| | TGGGGCACAATCTGTGATGATGGCTGGGACAGCTACGACGCCGCCGTGG | RPTIPEESAVACIESGQLRLVNGGGRCAGRVEIYHEGSWGTICD |
| | CCTGCAAGCAGCTGGGCTGTCCTACCGCCGTTACCGCTATCGGACGGGT | DSWDLSDAHVVCRQLGCGEAINATGSAHFGEGTGPIWLDEMKCN |
| | GAACGCCAGCAAGGGCTTCGGCCATATCTGGCTGGACAGCGTGAGCTGC | GKESRIWQCHSHGWGQQNCRHKEDAGVICSEFMSLRLTSEASRE |
| | CAGGGCCACGAGCCTGCAATCTGGCAGTGTAAGCACCACGAATGGGGCA | ACAGRLEVFYNGAWGTVGKSSMSETTVGVVCRQLGCADKGKINP |
| | AGCACTACTGTAATCACAACGAAGACGCCGGGGTGACATGCAGCGACGG | ASLDKAMSIPMWVDNVQCPKGPDTLWQCPSSPWEKRLASPSEET |
| | ATCCGACCTGGAATTAAGACTACGGGGCGGGGGATCCAGATGCGCCGGA | WITCDNKIRLQEGPTSCSGRVEIWHGGSWGTVCDDSWDLDDAQV |
| | ACGGTCGAGGTGGAGATACAGCGGTTGCTGGGCAAGGTGTGCGACAGAG | VCQQLGCGPALKAFKEAEFGQGTGPIWLNEVKCKGNESSLWDCP |
| | GTTGGGGTCTGAAGGAGGCTGACGTGGTGTGTCGTCAGCTGGGCTGCGG | ARRWGHSECGHKEDAAVNCTDISVQKTPQKATTGRSSROSS |
| | AAGCGCTCTGAAAACCTCTTACCAAGTGTACTCTAAGATCCAGGCCACA | |
| | AACACTTGGCTGTTCCTGAGCAGCTGTAACGGGAACGAAACCTCTCTCT | |
| | GGGACTGCAAGAATTGGCAGTGGGGAGGCTTGACCTGCGATCACTACGA | |
| | GGAAGCTAAAATCACCTGCAGTGCTCACAGAGAGCCTCGTTTAGTGGGA | |
| | GGCGACATCCCTTGCTCTGGCCGGGTAGAAGTGAAGCACGGTGACACTT | |
| | GGGGTAGCATCTGTGACAGCGATTTCTCCCTGGAAGCCGCCAGCGTGCT | |
| | GTGTAGAGAACTGCAGTGCGGCACCGTCGTCTCAATCCTCGGGGGTGCT | |
| | CACTTCGGCGAGGGGAATGGCCAGATCTGGGCCGAGGAGTTCCAGTGCG | |
| | AGGGCCACGAGAGCCATCTGAGTCTGTGCCCCGTGGCTCCTAGACCTGA | |
| | GGGAACCTGTTCTCACTCCAGAGACGTCGGGGTTGTGTGCTCTAGATAC | |
| | ACAGAGATCCGACTGGTGAACGGCAAGACCCCTTGCGAGGGCAGAGTTG | |
| | AGCTGAAGACCTTGGGCGCTTGGGGCAGCCTGTGCAACAGCCACTGGGA | |
| | TATCGAAGATGCCCACGTATTGTGTCAGCAGTTAAAGTGCGGCGTGGCT | |
| | CTTTCTACACCTGGCGGCGCCAGATTCGGCAAGGGCAACGGCCAGATCT | |
| | GGAGACACATGTTCCACTGCACTGGCACAGAGCAGCACATGGGCGATTG | |
| | TCCTGTGACCGCTCTGGGCGCTTCTCTGTGCCCCTCCGAGCAGGTGGCC | |
| | TCCGTAATCTGCTCTGGAAACCAGAGCCAGACCCTCTCCTCCTGCAACT | |
| | CTTCTTCCCTGGGCCCCACCAGGCCGACCATTCCTGAAGAGTCAGCTGT | |
| | GGCGTGCATCGAGAGCGGCCAGTTAAGACTCGTGAACGGCGGAGGACGG | |
| | TGTGCAGGTAGAGTGGAGATCTACCACGAGGGCTCTTGGGGCACCATAT | |
| | GCGACGACAGCTGGGACCTGTCCGACGCGCACGTGGTGTGTAGACAACT | |
| | CGGCTGTGGAGAAGCCATTAACGCTACTGGGTCTGCTCACTTCGGCGAA | |
| | GGCACGGGACCCATCTGGCTGGACGAGATGAAGTGCAACGGCAAAGAGA | |
| | GCCGGATATGGCAGTGTCACTCTCACGGATGGGGCCAGCAGAACTGCAG | |
| | ACACAAAGAGGATGCCGGAGTCATCTGCTCTGAATTCATGAGCCTGCGG | |
| | CTGACCTCCGAAGCTTCTCGGGAGGCCTGCGCCGGACGGTTAGAGGTGT | |
| | TCTACAATGGTGCTTGGGGCACTGTGGGCAAGTCCAGCATGTCCGAGAC | |
| | TACTGTCGGGGTGGTGTGCCGGCAGCTTGGGTGTGCCGATAAAGGCAAG | |
| | ATCAACCCTGCCTCTCTGGACAAGGCCATGTCCATTCCAATGTGGGTGG | |
| | ATAACGTGCAGTGCCCCAAGGGCCCTGACACCCTGTGGCAATGCCCTAG | |
| | CAGCCCTTGGGAAAAGAGATTAGCCTCTCCTTCAGAAGAAACATGGATC | |
| | ACCTGTGACAACAAGATCCGGCTGCAGGAGGGCCCAACAAGCTGTTCCG | |
| | GGAGAGTGGAAATTTGGCATGGCGGCAGCTGGGGCACCGTGTGCGACGA | |
| | CTCTTGGGACCTGGACGATGCTCAGGTGGTGTGCCAGCAACTGGGCTGT | |
| | GGCCCTGCCCTAAAGGCCTTCAAGGAGGCCGAGTTCGGCCAGGGCACGG | |
| | GCCCTATTTGGCTGAACGAGGTAAAGTGCAAGGGCAATGAAAGCAGTTT | |
| | ATGGGATTGCCCTGCAAGAAGATGGGGCCACAGCGAATGTGGACATAAG | |
| | GAAGACGCCGCCGTGAATTGTACAGACATTTCAGTGCAGAAGACCCCTC | |
| | AGAAGGCCACCACCGGCAGAAGCAGCCGGCAGTCCAGT | |

For SEQ ID NOS: 9-52, italicized text indicates extracellular domain; underlined text indicates transmembrane domain; and normal text indicates intracellular domain.

Exemplary chimeric polypeptide constructs are provided in Table 2.

TABLE 2
Exemplary chimeric polypeptide constructs

| Name | Nucleotide Sequence | Amino Acid Sequence |
|---|---|---|
| | SEQ ID NO: 54: | SEQ ID NO: 61: |
| F4-ACE2 | ATGGCCTTACCAGTGACCGCCTTGCTCCTGCCGCTGGCCTTGCTGCTCC | MALPVTALLLPLALLLHAARPGSSTIEEQAKTFLDKFNHEAEDL |
| CBR | ACGCCGCCAGGCCGGGATCCTCCACCATTGAGGAACAGGCCAAGACATT | FYQSSLASWNYNTNITEENVQNMNNAGDKWSAFLKEQSTLAQMY |
| | TTTGGACAAGTTTAACCACGAAGCCGAAGACCTGTTCTATCAAAGTTCA | PLQEIQNLTVKLQLQALQQNGSSVLSEDKSKRLNTILNTMSTIY |
| | CTTGCTTCTTGGAATTATAACACCAATATTACTGAAGAGAATGTCCAAA | STGKVCNPDNPQECLLLEPGLNEIMANSLDYNERLWAWESWRSE |
| | ACATGAATAATGCTGGGGACAAATGGTCTGCCTTTTTAAAGGAACAGTC | VGKQLRPLYEEYVVLKNEMARANHYEDYGDYWRGDYEVNGVDGY |
| | CACACTTGCCCAAATGTATCCACTACAAGAAATTCAGAATCTCACAGTC | DYSRGQLIEDVEHTFEEIKPLYEHLHAYVRAKLMNAYPSYISPI |
| | AAGCTTCAGCTGCAGGCTCTTCAGCAAAATGGGTCTTCAGTGCTCTCAG | GCLPAHLLGDMWGRFWTNLYSLTVPFGQKPNIDVTDAMVDQAWD |
| | AAGACAAGAGCAAACGGTTGAACACAATTCTAAATACAATGAGCACCAT | AQRIFKEAEKFFVSVGLPNMTQGFWENSMLTDPGNVQKAVCHPT |
| | CTACAGTACTGGAAAAGTTTGTAACCCAGATAATCCACAAGAATGCTTA | AWDLGKGDFRILMCTKVTMDDFLTAHHEMGHIQYDMAYAAQPFL |
| | TTACTTGAACCAGGTTTGAATGAAATAATGGCAAACAGTTTAGACTACA | LRNGANEGFHEAVGEIMSLSAATPKHLKSIGLLSPDFQEDNETE |
| | ATGAGAGGCTCTGGGCTTGGGAAAGCTGGAGATCTGAGGTCGGCAAGCA | INFLLKQALTIVGTLPFTYMLEKWRWMVFKGEIPKDQWMKKWWE |
| | GCTGAGGCCATTATATGAAGAGTATGTGGTCTTGAAAAATGAGATGGCA | MKREIVGVVEPVPHDETYCDPASLFHVSNDYSFIRYYTRTLYQF |
| | AGAGCAAATCATTATGAGGACTATGGGGATTATTGGAGAGGAGACTATG | QFQEALCQAAKHEGPLHKCDISNSTEAGQKLFNMLRLGKSEPWT |
| | AAGTAAATGGGGTAGATGGCTATGACTACAGCCGCGGCCAGTTGATTGA | LALENVVGAKNMNVRPLLNYFEPLFTWLKDONKNSFVGWSTDWS |
| | AGATGTGGAACATACCTTTGAAGAGATTAAACCATTATATGAACATCTT | PYADQSIKVRISLKSALGDKAYEWNDNEMYLFRSSVAYAMRQYF |
| | CATGCCTATGTGAGGGCAAAGTTGATGAATGCCTATCCTTCCTATATCA | LKVKNQMILFGEEDVRVANLKPRISENFFVTAPKNVSDIIPRTE |
| | GTCCAATTGGATGCCTCCCTGCTCATTTGCTTGGTGATATGTGGGGTAG | VEKAIRMSRSRINDAFRLNDNSLEFLGIQPTLGPPNQPPVSDTR |
| | ATTTTGGACAAATCTGTACTCTTTGACAGTTCCCTTTGGACAGAAACCA | KMDPSKPSSNVAGVVIIVILLILTGAGLAAYFFYKKRRVHLPQE |
| | AACATAGATGTTACTGATGCAATGGTGGACCAGGCCTGGGATGCACAGA | GAFENTLYFNSQSSPGTSDMKDLVGNIEQNEHSVI |
| | GAATATTCAAGGAGGCCGAGAAGTTCTTTGTATCTGTTGGTCTTCCTAA | |
| | TATGACTCAAGGATTCTGGGAAAATTCCATGCTAACGGACCCAGGAAAT | |
| | GTTCAGAAAGCAGTCTGCCATCCCACAGCTTGGGACCTGGGGAAGGGCG | |
| | ACTTCAGGATCCTTATGTGCACAAAGGTGACAATGGACGACTTCCTGAC | |
| | AGCTCATCATGAGATGGGGCATATCCAGTATGATATGGCATATGCTGCA | |
| | CAACCTTTTCTGCTAAGAAATGGAGCTAATGAAGGATTCCATGAAGCTG | |
| | TTGGGGAAATCATGTCACTTTCTGCAGCCACACCTAAGCATTTAAAATC | |
| | CATTGGTCTTCTGTCACCCGATTTTCAAGAAGACAATGAAACAGAAATA | |
| | AACTTCCTGCTCAAACAAGCACTCACGATTGTTGGGACTCTGCCATTTA | |
| | CTTACATGTTAGAGAAGTGGAGGTGGATGGTCTTTAAAGGGGAAATTCC | |
| | CAAAGACCAGTGGATGAAAAAGTGGTGGGAGATGAAGCGAGAGATAGTT | |
| | GGGGTGGTGGAACCTGTGCCCCATGATGAAACATACTGTGACCCCGCAT | |
| | CTCTGTTCCATGTTTCTAATGATTACTCATTCATTCGATATTACACAAG | |
| | GACCCTTTACCAATTCCAGTTTCAAGAAGCACTTTGTCAAGCAGCTAAA | |
| | CATGAAGGCCCTCTGCACAAATGTGACATCTCAAACTCTACAGAAGCTG | |
| | GACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCA | |
| | CTGCTCAACTACTTTGAGCCCTTATTTACCTGGCTGAAAGACCAGAACA | |
| | AGAATTCTTTTGTGGGATGGAGTACCGACTGGAGTCCATATGCAGACCA | |
| | AAGCATCAAAGTGAGGATAAGCCTAAAATCAGCTCTTGGAGATAAAGCA | |
| | TATGAATGGAACGACAATGAAATGTACCTGTTCCGATCATCTGTTGCAT | |
| | ATGCTATGAGGCAGTACTTTTTAAAAGTAAAAAATCAGATGATTCTTTT | |
| | TGGGGAGGAGGATGTGCGAGTGGCTAATTTGAAACCAAGAATCTCCTTT | |
| | AATTTCTTTGTCACTGCACCTAAAAATGTGTCTGATATCATTCCTAGAA | |
| | CTGAAGTTGAAAAGGCCATCAGGATGTCCCGGAGCCGTATCAATGATGC | |
| | TTTCCGTCTGAATGACAACAGCCTAGAGTTTCTGGGGATACAGCCAACA | |
| | CTTGGACCTCCTAACCAGCCCCCTGTTTCCGACACAAGGAAGATGGACC | |
| | CTTCTAAACCGTCTTCCAACGTGGCCGGAGTAGTCATCATTGTGATCCT | |
| | CCTGATTTTAACGGGTGCTGGCCTTGCCGCCTATTTCTTTTATAAGAAA | |
| | AGACGTGTGCACCTACCTCAAGAGGGCGCCTTTGAAAACACTCTGTATT | |
| | TTAACAGTCAGTCAAGCCCAGGAACTAGTGATATGAAAGATCTCGTGGG | |
| | CAATATTGAACAGAATGAACACTCGGTCATC | |
| F5-ACE2 | SEQ ID NO: 55: | SEQ ID NO: 62: |
| CBR | ATGAGGCTACCCCTGCTCCTGGTTTTTGCCTCTGTCATTCCGGGTGCTG | MRLPLLLVFASVIPGAVLSTIEEQAKTFLDKFNHEAEDLFYQSS |
| | TTCTCTCCACCATTGAGGAACAGGCCAAGACATTTTTGGACAAGTTTAA | LASWNYNTNITEENVONMNNAGDKWSAFLKEQSTLAQMYPLQEI |
| | CCACGAAGCCGAAGACCTGTTCTATCAAAGTTCACTTGCTTCTTGGAAT | QNLTVKLQLQALQQNGSSVLSEDKSKRLNTILNTMSTIYSTGKV |
| | TATAACACCAATATTACTGAAGAGAATGTCCAAAACATGAATAATGCTG | CNPDNPQECLLLEPGLNEIMANSLDYNERLWAWESWRSEVGKQL |
| | GGGACAAATGGTCTGCCTTTTTAAAGGAACAGTCCACACTTGCCCAAAT | RPLYEEYVVLKNEMARANHYEDYGDYWRGDYEVNGVDGYDYSRG |
| | GTATCCACTACAAGAAATTCAGAATCTCACAGTCAAGCTTCAGCTGCAG | QLIEDVEHTFEEIKPLYEHLHAYVRAKLMNAYPSYISPIGCLPA |
| | GCTCTTCAGCAAAATGGGTCTTCAGTGCTCTCAGAAGACAAGAGCAAAC | HLLGDMWGRFWTNLYSLTVPFGQKPNIDVTDAMVDQAWDAQRIF |
| | GGTTGAACACAATTCTAAATACAATGAGCACCATCTACAGTACTGGAAA | KEAEKFFVSVGLPNMTQGFWENSMLTDPGNVQKAVCHPTAWDLG |
| | AGTTTGTAACCCAGATAATCCACAAGAATGCTTATTACTTGAACCAGGT | KGDFRILMCTKVTMDDFLTAHHEMGHIQYDMAYAAQPFLLRNGA |
| | TTGAATGAAATAATGGCAAACAGTTTAGACTACAATGAGAGGCTCTGGG | NEGFHEAVGEIMSLSAATPKHLKSIGLLSPDFQEDNETEINFLL |
| | CTTGGGAAAGCTGGAGATCTGAGGTCGGCAAGCAGCTGAGGCCATTATA | KQALTIVGTLPFTYMLEKWRWMVFKGEIPKDQWMKKWWEMKREI |
| | TGAAGAGTATGTGGTCTTGAAAAATGAGATGGCAAGAGCAAATCATTAT | VGVVEPVPHDETYCDPASLFHVSNDYSFIRYYTRTLYQFQFQEA |
| | GAGGACTATGGGGATTATTGGAGAGGAGACTATGAAGTAAATGGGGTAG | LCQAAKHEGPLHKCDISNSTEAGQKLENMLRLGKSEPWTLALEN |
| | ATGGCTATGACTACAGCCGCGGCCAGTTGATTGAAGATGTGGAACATAC | VVGAKNMNVRPLLNYFEPLFTWLKDONKNSFVGWSTDWSPYADQ |
| | CTTTGAAGAGATTAAACCATTATATGAACATCTTCATGCCTATGTGAGG | SIKVRISLKSALGDKAYEWNDNEMYLFRSSVAYAMRQYFLKVKN |
| | GCAAAGTTGATGAATGCCTATCCTTCCTATATCAGTCCAATTGGATGCC | QMILFGEEDVRVANLKPRISENFFVTAPKNVSDIIPRTEVEKAI |
| | TCCCTGCTCATTTGCTTGGTGATATGTGGGGTAGATTTTGGACAAATCT | RMSRSRINDAFRLNDNSLEFLGIQPTLGPPNQPPVSGSFVPVEL |
| | GTACTCTTTGACAGTTCCCTTTGGACAGAAACCAAACATAGATGTTACT | PAKPTTTPAPRPPTPAPTIASQPLSLRPEACRPAAGGAVHTRGL |
| | GATGCAATGGTGGACCAGGCCTGGGATGCACAGAGAATATTCAAGGAGG | DFACDIDAKPTHELLTTKADTRKMDPSKPSSNVAGVVIIVILLI |
| | CCGAGAAGTTCTTTGTATCTGTTGGTCTTCCTAATATGACTCAAGGATT | LTGAGLAAYFFYKKRRVHLPQEGAFENTLYFNSQSSPGTSDMKD |
| | CTGGGAAAATTCCATGCTAACGGACCCAGGAAATGTTCAGAAAGCAGTC | LVGNIEQNEHSVI |
| | TGCCATCCCACAGCTTGGGACCTGGGGAAGGGCGACTTCAGGATCCTTA | |
| | TGTGCACAAAGGTGACAATGGACGACTTCCTGACAGCTCATCATGAGAT | |
| | GGGGCATATCCAGTATGATATGGCATATGCTGCACAACCTTTTCTGCTA | |
| | AGAAATGGAGCTAATGAAGGATTCCATGAAGCTGTTGGGGAAATCATGT | |
| | CACTTTCTGCAGCCACACCTAAGCATTTAAAATCCATTGGTCTTCTGTC | |
| | ACCCGATTTTCAAGAAGACAATGAAACAGAAATAAACTTCCTGCTCAAA | |
| | CAAGCACTCACGATTGTTGGGACTCTGCCATTTACTTACATGTTAGAGA | |
| | AGTGGAGGTGGATGGTCTTTAAAGGGGAAATTCCCAAAGACCAGTGGAT | |
| | GAAAAAGTGGTGGGAGATGAAGCGAGAGATAGTTGGGGTGGTGGAACCT | |
| | GTGCCCCATGATGAAACATACTGTGACCCCGCATCTCTGTTCCATGTTT | |
| | CTAATGATTACTCATTCATTCGATATTACACAAGGACCCTTTACCAATT | |
| | CCAGTTTCAAGAAGCACTTTGTCAAGCAGCTAAACATGAAGGCCCTCTG | |
| | CACAAATGTGACATCTCAAACTCTACAGAAGCTGGACAGAAACTGTTCA | |
| | ATATGCTGAGGCTTGGAAAATCAGAACCCTGGACCCTAGCATTGGAAAA | |
| | TGTTGTAGGAGCAAAGAACATGAATGTAAGGCCACTGCTCAACTACTTT | |
| | GAGCCCTTATTTACCTGGCTGAAAGACCAGAACAAGAATTCTTTTGTGG | |
| | GATGGAGTACCGACTGGAGTCCATATGCAGACCAAAGCATCAAAGTGAG | |
| | GATAAGCCTAAAATCAGCTCTTGGAGATAAAGCATATGAATGGAACGAC | |
| | AATGAAATGTACCTGTTCCGATCATCTGTTGCATATGCTATGAGGCAGT | |
| | ACTTTTTAAAAGTAAAAAATCAGATGATTCTTTTTGGGGAGGAGGATGT | |
| | GCGAGTGGCTAATTTGAAACCAAGAATCTCCTTTAATTTCTTTGTCACT | |
| | GCACCTAAAAATGTGTCTGATATCATTCCTAGAACTGAAGTTGAAAAGG | |
| | CCATCAGGATGTCCCGGAGCCGTATCAATGATGCTTTCCGTCTGAATGA | |
| | CAACAGCCTAGAGTTTCTGGGGATACAGCCAACACTTGGACCTCCTAAC | |
| | CAGCCCCCTGTTTCCGGATCCTTCGTGCCGGTCTTCCTGCCAGCGAAGC | |
| | CCACCACGACGCCAGCGCCGCGACCACCAACACCGGCGCCCACCATCGC | |
| | GTCGCAGCCCCTGTCCCTGCGCCCAGAGGCGTGCCGGCCAGCGGCGGGG | |
| | GGCGCAGTGCACACGAGGGGGCTGGACTTCGCCTGTGATATTGATGCTA | |
| | AACCTACTCATGAATTACTTACAACAAAAGCTGACACAAGGAAGATGGA | |
| | CCCTTCTAAACCGTCTTCCAACGTGGCCGGAGTAGTCATCATTGTGATC | |
| | CTCCTGATTTTAACGGGTGCTGGCCTTGCCGCCTATTTCTTTTATAAGA | |
| | AAAGACGTGTGCACCTACCTCAAGAGGGCGCCTTTGAAAACACTCTGTA | |
| | TTTTAACAGTCAGTCAAGCCCAGGAACTAGTGATATGAAAGATCTCGTG | |
| | GGCAATATTGAACAGAATGAACACTCGGTCATC | |
| B2-AC-MER | SEQ ID NO: 56: | SEQ ID NO: 63: |
| CBR | ATGAGGCTACCCCTGCTCCTGGTTTTTGCCTCTGTCATTCCGGGTGCTG | MRLPLLLVFASVIPGAVLSTIEEQAKTFLDKFNHEAEDLFYQSS |
| | TTCTCTCCACCATTGAGGAACAGGCCAAGACATTTTTGGACAAGTTTAA | LASWNYNTNITEENVQNMNNAGDKWSAFLKEQSTLAQMYPLQEI |
| | CCACGAAGCCGAAGACCTGTTCTATCAAAGTTCACTTGCTTCTTGGAAT | QNLTVKLQLQALQQNGSSVLSEDKSKRLNTILNTMSTIYSTGKV |
| | TATAACACCAATATTACTGAAGAGAATGTCCAAAACATGAATAATGCTG | CNPDNPQECLLLEPGLNEIMANSLDYNERLWAWESWRSEVGKQL |
| | GGGACAAATGGTCTGCCTTTTTAAAGGAACAGTCCACACTTGCCCAAAT | RPLYEEYVVLKNEMARANHYEDYGDYWRGDYEVNGVDGYDYSRG |
| | GTATCCACTACAAGAAATTCAGAATCTCACAGTCAAGCTTCAGCTGCAG | QLIEDVEHTFEEIKPLYEHLHAYVRAKLMNAYPSYISPIGCLPA |
| | GCTCTTCAGCAAAATGGGTCTTCAGTGCTCTCAGAAGACAAGAGCAAAC | HLLGDMWGRFWTNLYSLTVPFGQKPNIDVTDAMVDQAWDAQRIF |
| | GGTTGAACACAATTCTAAATACAATGAGCACCATCTACAGTACTGGAAA | KEAEKFFVSVGLPNMTQGFWENSMLTDPGNVQKAVCHPTAWDLG |
| | AGTTTGTAACCCAGATAATCCACAAGAATGCTTATTACTTGAACCAGGT | KGDFRILMCTKVTMDDFLTAHHEMGHIQYDMAYAAQPFLLRNGA |
| | TTGAATGAAATAATGGCAAACAGTTTAGACTACAATGAGAGGCTCTGGG | NEGFHEAVGEIMSLSAATPKHLKSIGLLSPDFQEDNETEINFLL |
| | CTTGGGAAAGCTGGAGATCTGAGGTCGGCAAGCAGCTGAGGCCATTATA | KQALTIVGTLPFTYMLEKWRWMVFKGEIPKDQWMKKWWEMKREI |
| | TGAAGAGTATGTGGTCTTGAAAAATGAGATGGCAAGAGCAAATCATTAT | VGVVEPVPHDETYCDPASLFHVSNDYSFIRYYTRTLYQFQFQEA |
| | GAGGACTATGGGGATTATTGGAGAGGAGACTATGAAGTAAATGGGGTAG | LCQAAKHEGPLHKCDISNSTEAGQKLFNMLRLGKSEPWTLALEN |
| | ATGGCTATGACTACAGCCGCGGCCAGTTGATTGAAGATGTGGAACATAC | VVGAKNMNVRPLLNYFEPLFTWLKDONKNSFVGWSTDWSPYADQ |
| | CTTTGAAGAGATTAAACCATTATATGAACATCTTCATGCCTATGTGAGG | SIKVRISLKSALGDKAYEWNDNEMYLFRSSVAYAMRQYFLKVKN |
| | GCAAAGTTGATGAATGCCTATCCTTCCTATATCAGTCCAATTGGATGCC | QMILFGEEDVRVANLKPRISENFFVTAPKNVSDIIPRTEVEKAI |
| | TCCCTGCTCATTTGCTTGGTGATATGTGGGGTAGATTTTGGACAAATCT | RMSRSRINDAFRLNDNSLEFLGIQPTLGPPNQPPVSGSFVPVFL |
| | GTACTCTTTGACAGTTCCCTTTGGACAGAAACCAAACATAGATGTTACT | PAKPTTTPAPRPPTPAPTIASQPLSLRPEACRPAAGGAVHTRGL |
| | GATGCAATGGTGGACCAGGCCTGGGATGCACAGAGAATATTCAAGGAGG | DFACDFGCFCGFILIGLILYISLAIRKRVQETKFGNAFTEEDSE |
| | CCGAGAAGTTCTTTGTATCTGTTGGTCTTCCTAATATGACTCAAGGATT | LVVNYIAKKSFCRRAIELTLHSLGVSEELQNKLEDVVIDRNLLI |
| | CTGGGAAAATTCCATGCTAACGGACCCAGGAAATGTTCAGAAAGCAGTC | LGKILGEGEFGSVMEGNLKQEDGTSLKVAVKTMKLDNSSQREIE |
| | TGCCATCCCACAGCTTGGGACCTGGGGAAGGGCGACTTCAGGATCCTTA | EFLSEAACMKDFSHPNVIRLLGVCIEMSSQGIPKPMVILPFMKY |
| | TGTGCACAAAGGTGACAATGGACGACTTCCTGACAGCTCATCATGAGAT | GDLHTYLLYSRLETGPKHIPLQTLLKFMVDIALGMEYLSNRNFL |
| | GGGGCATATCCAGTATGATATGGCATATGCTGCACAACCTTTTCTGCTA | HRDLAARNCMLRDDMTVCVADFGLSKKIYSGDYYRQGRIAKMPV |
| | AGAAATGGAGCTAATGAAGGATTCCATGAAGCTGTTGGGGAAATCATGT | KWIAIESLADRVYTSKSDVWAFGVTMWEIATRGMTPYPGVQNHE |
| | CACTTTCTGCAGCCACACCTAAGCATTTAAAATCCATTGGTCTTCTGTC | MYDYLLHGHRLKQPEDCLDELYEIMYSCWRTDPLDRPTFSVLRL |
| | ACCCGATTTTCAAGAAGACAATGAAACAGAAATAAACTTCCTGCTCAAA | QLEKLLESLPDVRNQADVIYVNTOLLESSEGLAQGSTLAPLDLN |
| | CAAGCACTCACGATTGTTGGGACTCTGCCATTTACTTACATGTTAGAGA | IDPDSIIASCTPRAAISVVTAEVHDSKPHEGRYILNGGSEEWED |
| | AGTGGAGGTGGATGGTCTTTAAAGGGGAAATTCCCAAAGACCAGTGGAT | LTSAPSAAVTAEKNSVLPGERLVRNGVSWSHSSMLPLGSSLPDE |
| | GAAAAAGTGGTGGGAGATGAAGCGAGAGATAGTTGGGGTGGTGGAACCT | LLFADDSSEGSEVLM |
| | GTGCCCCATGATGAAACATACTGTGACCCCGCATCTCTGTTCCATGTTT | |
| | CTAATGATTACTCATTCATTCGATATTACACAAGGACCCTTTACCAATT | |
| | CCAGTTTCAAGAAGCACTTTGTCAAGCAGCTAAACATGAAGGCCCTCTG | |
| | CACAAATGTGACATCTCAAACTCTACAGAAGCTGGACAGAAACTGTTCA | |
| | ATATGCTGAGGCTTGGAAAATCAGAACCCTGGACCCTAGCATTGGAAAA | |
| | TGTTGTAGGAGCAAAGAACATGAATGTAAGGCCACTGCTCAACTACTTT | |
| | GAGCCCTTATTTACCTGGCTGAAAGACCAGAACAAGAATTCTTTTGTGG | |
| | GATGGAGTACCGACTGGAGTCCATATGCAGACCAAAGCATCAAAGTGAG | |
| | GATAAGCCTAAAATCAGCTCTTGGAGATAAAGCATATGAATGGAACGAC | |
| | AATGAAATGTACCTGTTCCGATCATCTGTTGCATATGCTATGAGGCAGT | |
| | ACTTTTTAAAAGTAAAAAATCAGATGATTCTTTTTGGGGAGGAGGATGT | |
| | GCGAGTGGCTAATTTGAAACCAAGAATCTCCTTTAATTTCTTTGTCACT | |
| | GCACCTAAAAATGTGTCTGATATCATTCCTAGAACTGAAGTTGAAAAGG | |
| | CCATCAGGATGTCCCGGAGCCGTATCAATGATGCTTTCCGTCTGAATGA | |
| | CAACAGCCTAGAGTTTCTGGGGATACAGCCAACACTTGGACCTCCTAAC | |
| | CAGCCCCCTGTTTCCGGATCCTTCGTGCCGGTCTTCCTGCCAGCGAAGC | |
| | CCACCACGACGCCAGCGCCGCGACCACCAACACCGGCGCCCACCATCGC | |
| | GTCGCAGCCCCTGTCCCTGCGCCCAGAGGCGTGCCGGCCAGCGGCGGGG | |
| | GGCGCAGTGCACACGAGGGGGCTGGACTTCGCCTGTGATTTTGGATGTT | |
| | TTTGTGGTTTCATCCTCATCGGTTTGATATTGTACATAAGTCTGGCGAT | |
| | AAGGAAGAGAGTTCAAGAGACAAAGTTCGGAAATGCCTTTACAGAGGAA | |
| | GACAGTGAGCTCGTTGTAAACTACATCGCAAAAAAAAGCTTCTGTAGAA | |
| | GAGCAATAGAGCTCACGTTGCACTCACTCGGTGTGTCCGAAGAACTCCA | |
| | GAATAAACTGGAAGACGTCGTTATCGATCGGAACCTCCTCATACTTGGA | |
| | AAAATACTGGGAGAAGGAGAGTTCGGCAGTGTCATGGAAGGTAACTTGA | |
| | AACAAGAGGATGGTACCTCACTCAAGGTAGCTGTCAAGACGATGAAACT | |
| | TGATAACAGTTCACAAAGGGAGATCGAAGAATTTCTGTCTGAGGCCGCC | |
| | TGTATGAAAGACTTCTCACATCCTAATGTCATCAGACTTCTTGGCGTTT | |
| | GTATCGAGATGTCTAGCCAAGGAATCCCAAAACCTATGGTCATATTGCC | |
| | TTTCATGAAATATGGCGATCTGCATACATATTTGCTCTACTCTAGACTT | |
| | GAGACAGGGCCCAAACATATTCCTCTCCAGACATTGCTCAAGTTTATGG | |
| | TCGATATTGCCCTGGGTATGGAGTACTTGAGCAACCGAAATTTTCTGCA | |
| | TCGGGATCTTGCCGCACGCAACTGCATGCTGCGCGATGACATGACCGTC | |
| | TGCGTGGCTGATTTTGGGCTGTCAAAAAAAATATATTCTGGAGACTACT | |
| | ACCGACAAGGGCGGATTGCAAAGATGCCCGTCAAATGGATTGCGATTGA | |
| | AAGTTTGGCGGACAGGGTATATACTTCCAAATCAGATGTTTGGGCTTTT | |
| | GGAGTCACTATGTGGGAAATAGCTACACGCGGTATGACCCCGTACCCCG | |
| | GAGTACAAAATCATGAAATGTATGACTATCTCCTTCATGGACACAGGCT | |
| | GAAGCAGCCCGAGGACTGCCTGGACGAACTGTATGAAATAATGTATTCT | |
| | TGTTGGCGAACCGATCCCTTGGACCGGCCTACTTTCAGTGTCCTTAGAT | |
| | TGCAACTTGAGAAATTGCTCGAGTCTTTGCCGGATGTGCGAAACCAGGC | |
| | AGACGTGATCTATGTCAATACCCAACTTTTGGAAAGTTCTGAGGGCCTC | |
| | GCACAGGGTTCTACCCTTGCCCCGTTGGATCTTAACATAGACCCAGACA | |
| | GCATAATTGCTTCTTGTACACCTCGCGCTGCCATATCAGTTGTAACAGC | |
| | GGAGGTCCATGATAGTAAACCTCACGAGGGTCGCTATATCCTGAACGGC | |
| | GGGTCAGAAGAATGGGAAGACCTGACATCAGCGCCGAGCGCCGCCGTTA | |
| | CTGCTGAGAAAAACTCTGTCCTGCCCGGAGAGCGCTTGGTTCGGAACGG | |
| | GGTAAGTTGGAGCCATAGCTCAATGCTCCCCCTGGGTTCAAGTCTCCCG | |
| | GACGAGCTTCTTTTTGCGGACGACTCATCTGAGGGGTCCGAAGTTCTGA | |
| | TG | |
| B4-AC-MEG | SEQ ID NO: 57: | SEQ ID NO: 64: |
| CBR | ATGAGGCTACCCCTGCTCCTGGTTTTTGCCTCTGTCATTCCGGGTGCTG | MRLPLLLVFASVIPGAVLSTIEEQAKTFLDKFNHEAEDLFYQSS |
| | TTCTCTCCACCATTGAGGAACAGGCCAAGACATTTTTGGACAAGTTTAA | LASWNYNTNITEENVQNMNNAGDKWSAFLKEQSTLAQMYPLQEI |
| | CCACGAAGCCGAAGACCTGTTCTATCAAAGTTCACTTGCTTCTTGGAAT | QNLTVKLQLQALQQNGSSVLSEDKSKRLNTILNTMSTIYSTGKV |
| | TATAACACCAATATTACTGAAGAGAATGTCCAAAACATGAATAATGCTG | CNPDNPQECLLLEPGLNEIMANSLDYNERLWAWESWRSEVGKQL |
| | GGGACAAATGGTCTGCCTTTTTAAAGGAACAGTCCACACTTGCCCAAAT | RPLYEEYVVLKNEMARANHYEDYGDYWRGDYEVNGVDGYDYSRG |
| | GTATCCACTACAAGAAATTCAGAATCTCACAGTCAAGCTTCAGCTGCAG | QLIEDVEHTFEEIKPLYEHLHAYVRAKLMNAYPSYISPIGCLPA |
| | GCTCTTCAGCAAAATGGGTCTTCAGTGCTCTCAGAAGACAAGAGCAAAC | HLLGDMWGRFWTNLYSLTVPFGQKPNIDVTDAMVDQAWDAQRIF |
| | GGTTGAACACAATTCTAAATACAATGAGCACCATCTACAGTACTGGAAA | KEAEKFFVSVGLPNMTQGFWENSMLTDPGNVQKAVCHPTAWDLG |
| | AGTTTGTAACCCAGATAATCCACAAGAATGCTTATTACTTGAACCAGGT | KGDFRILMCTKVTMDDFLTAHHEMGHIQYDMAYAAQPFLLRNGA |
| | TTGAATGAAATAATGGCAAACAGTTTAGACTACAATGAGAGGCTCTGGG | NEGFHEAVGEIMSLSAATPKHLKSIGLLSPDFQEDNETEINFLL |
| | CTTGGGAAAGCTGGAGATCTGAGGTCGGCAAGCAGCTGAGGCCATTATA | KQALTIVGTLPFTYMLEKWRWMVFKGEIPKDQWMKKWWEMKREI |
| | TGAAGAGTATGTGGTCTTGAAAAATGAGATGGCAAGAGCAAATCATTAT | VGVVEPVPHDETYCDPASLFHVSNDYSFIRYYTRTLYQFQFQEA |
| | GAGGACTATGGGGATTATTGGAGAGGAGACTATGAAGTAAATGGGGTAG | LCQAAKHEGPLHKCDISNSTEAGQKLFNMLRLGKSEPWTLALEN |
| | ATGGCTATGACTACAGCCGCGGCCAGTTGATTGAAGATGTGGAACATAC | VVGAKNMNVRPLLNYFEPLFTWLKDONKNSFVGWSTDWSPYADQ |
| | CTTTGAAGAGATTAAACCATTATATGAACATCTTCATGCCTATGTGAGG | SIKVRISLKSALGDKAYEWNDNEMYLFRSSVAYAMRQYFLKVKN |
| | GCAAAGTTGATGAATGCCTATCCTTCCTATATCAGTCCAATTGGATGCC | QMILFGEEDVRVANLKPRISFNFFVTAPKNVSDIIPRTEVEKAI |
| | TCCCTGCTCATTTGCTTGGTGATATGTGGGGTAGATTTTGGACAAATCT | RMSRSRINDAFRLNDNSLEFLGIQPTLGPPNQPPVSGSFVPVFL |
| | GTACTCTTTGACAGTTCCCTTTGGACAGAAACCAAACATAGATGTTACT | PAKPTTTPAPRPPTPAPTIASQPLSLRPEACRPAAGGAVHTRGL |
| | GATGCAATGGTGGACCAGGCCTGGGATGCACAGAGAATATTCAAGGAGG | DFACDAIAGIIILVLVVLFLLALFIIYRHKQKGKESSMPAVTYT |
| | CCGAGAAGTTCTTTGTATCTGTTGGTCTTCCTAATATGACTCAAGGATT | PAMRVVNADYTISGTLPHSNGGNANSHYFTNPSYHTLTQCATSP |
| | CTGGGAAAATTCCATGCTAACGGACCCAGGAAATGTTCAGAAAGCAGTC | HVNNRDRMTVTKSKNNQLFVNLKNVNPGKRGPVGDCTGTLPADW |
| | TGCCATCCCACAGCTTGGGACCTGGGGAAGGGCGACTTCAGGATCCTTA | KHGGYLNELGAFGLDRSYMGKSLKDLGKNSEYNSSNCSLSSSEN |
| | TGTGCACAAAGGTGACAATGGACGACTTCCTGACAGCTCATCATGAGAT | PYATIKDPPVLIPKSSECGYVEMKSPARRDSPYAEINNSTSANR |
| | GGGGCATATCCAGTATGATATGGCATATGCTGCACAACCTTTTCTGCTA | NVYEVEPTVSVVQGVFSNNGRLSQDPYDLPKNSHIPCHYDLLPV |
| | AGAAATGGAGCTAATGAAGGATTCCATGAAGCTGTTGGGGAAATCATGT | RDSSSSPKQEDSGGSSSNSSSSSE |
| | CACTTTCTGCAGCCACACCTAAGCATTTAAAATCCATTGGTCTTCTGTC | |
| | ACCCGATTTTCAAGAAGACAATGAAACAGAAATAAACTTCCTGCTCAAA | |
| | CAAGCACTCACGATTGTTGGGACTCTGCCATTTACTTACATGTTAGAGA | |
| | AGTGGAGGTGGATGGTCTTTAAAGGGGAAATTCCCAAAGACCAGTGGAT | |
| | GAAAAAGTGGTGGGAGATGAAGCGAGAGATAGTTGGGGTGGTGGAACCT | |
| | GTGCCCCATGATGAAACATACTGTGACCCCGCATCTCTGTTCCATGTTT | |
| | CTAATGATTACTCATTCATTCGATATTACACAAGGACCCTTTACCAATT | |
| | CCAGTTTCAAGAAGCACTTTGTCAAGCAGCTAAACATGAAGGCCCTCTG | |
| | CACAAATGTGACATCTCAAACTCTACAGAAGCTGGACAGAAACTGTTCA | |
| | ATATGCTGAGGCTTGGAAAATCAGAACCCTGGACCCTAGCATTGGAAAA | |
| | TGTTGTAGGAGCAAAGAACATGAATGTAAGGCCACTGCTCAACTACTTT | |
| | GAGCCCTTATTTACCTGGCTGAAAGACCAGAACAAGAATTCTTTTGTGG | |
| | GATGGAGTACCGACTGGAGTCCATATGCAGACCAAAGCATCAAAGTGAG | |
| | GATAAGCCTAAAATCAGCTCTTGGAGATAAAGCATATGAATGGAACGAC | |
| | AATGAAATGTACCTGTTCCGATCATCTGTTGCATATGCTATGAGGCAGT | |
| | ACTTTTTAAAAGTAAAAAATCAGATGATTCTTTTTGGGGAGGAGGATGT | |
| | GCGAGTGGCTAATTTGAAACCAAGAATCTCCTTTAATTTCTTTGTCACT | |
| | GCACCTAAAAATGTGTCTGATATCATTCCTAGAACTGAAGTTGAAAAGG | |
| | CCATCAGGATGTCCCGGAGCCGTATCAATGATGCTTTCCGTCTGAATGA | |
| | CAACAGCCTAGAGTTTCTGGGGATACAGCCAACACTTGGACCTCCTAAC | |
| | CAGCCCCCTGTTTCCGGATCCTTCGTGCCGGTCTTCCTGCCAGCGAAGC | |
| | CCACCACGACGCCAGCGCCGCGACCACCAACACCGGCGCCCACCATCGC | |
| | GTCGCAGCCCCTGTCCCTGCGCCCAGAGGCGTGCCGGCCAGCGGCGGGG | |
| | GGCGCAGTGCACACGAGGGGGCTGGACTTCGCCTGTGATGCTATCGCGG | |
| | GGATCATTATATTGGTCTTGGTGGTGCTGTTTCTGCTCGCGCTTTTCAT | |
| | TATATACCGCCATAAGCAGAAGGGCAAAGAGTCCTCCATGCCAGCCGTG | |
| | ACCTATACGCCTGCGATGCGCGTTGTCAACGCCGATTACACCATCAGTG | |
| | GTACCCTCCCGCACAGTAACGGCGGAAATGCAAACTCTCATTACTTTAC | |
| | AAATCCTAGTTACCATACACTCACTCAGTGTGCTACCTCTCCCCATGTG | |
| | AACAATCGGGACAGGATGACCGTTACGAAAAGCAAAAATAACCAGTTGT | |
| | TTGTGAACCTTAAGAATGTGAATCCCGGCAAGAGGGGTCCGGTGGGTGA | |
| | CTGCACCGGAACTCTCCCCGCTGACTGGAAGCATGGCGGGTACCTGAAC | |
| | GAACTCGGCGCGTTTGGGCTCGACCGAAGCTACATGGGTAAAAGTCTTA | |
| | AGGACCTCGGTAAGAATAGCGAGTATAATAGCTCTAACTGTTCCCTTTC | |
| | CAGCTCCGAGAATCCGTACGCTACTATAAAAGACCCCCCGGTGCTCATT | |
| | CCCAAATCCAGTGAGTGCGGGTACGTGGAGATGAAAAGTCCCGCTCGAA | |
| | GAGACAGTCCATACGCGGAAATCAATAACTCCACCAGTGCGAACCGCAA | |
| | TGTGTACGAAGTGGAGCCCACCGTTTCCGTTGTACAAGGTGTATTTTCA | |
| | AACAATGGGAGGCTTAGCCAGGACCCCTATGATCTTCCAAAGAACAGCC | |
| | ACATCCCGTGTCATTATGATCTGTTGCCGGTGAGGGATTCTAGCTCTTC | |
| | TCCTAAACAAGAGGACTCAGGTGGCTCTAGTTCCAACTCCTCCAGTTCT | |
| | TCAGAG | |
| B6-AC-Dec | SEQ ID NO: 58: | SEQ ID NO: 65: |
| CBR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGFVPVFLPAKPTTTPAPRPPTPAP |
| | AAAGGGCAGTTGTGCTGCCAGTCCACCGTGGCGGTTGATTGCCGTCATC | TIASQPLSLRPEACRPAAGGAVHTRGLDFACDGSSTIEEQAKTE |
| | CTCGGCATTCTGTGCTTGGTTATTCTCGTTATAGCGGTGGTGCTTGGGT | LDKFNHEAEDLFYQSSLASWNYNTNITEENVONMNNAGDKWSAF |
| | TCGTGCCGGTCTTCCTGCCAGCGAAGCCCACCACGACGCCAGCGCCGCG | LKEQSTLAQMYPLQEIQNLTVKLQLQALQQNGSSVLSEDKSKRL |
| | ACCACCAACACCGGCGCCCACCATCGCGTCGCAGCCCCTGTCCCTGCGC | NTILNTMSTIYSTGKVCNPDNPQECLLLEPGLNEIMANSLDYNE |
| | CCAGAGGCGTGCCGGCCAGCGGCGGGGGGCGCAGTGCACACGAGGGGGC | RLWAWESWRSEVGKQLRPLYEEYVVLKNEMARANHYEDYGDYWR |
| | TGGACTTCGCCTGTGATGGATCCTCCACCATTGAGGAACAGGCCAAGAC | GDYEVNGVDGYDYSRGQLIEDVEHTFEEIKPLYEHLHAYVRAKL |
| | ATTTTTGGACAAGTTTAACCACGAAGCCGAAGACCTGTTCTATCAAAGT | MNAYPSYISPIGCLPAHLLGDMWGRFWTNLYSLTVPFGQKPNID |
| | TCACTTGCTTCTTGGAATTATAACACCAATATTACTGAAGAGAATGTCC | VTDAMVDQAWDAQRIFKEAEKFFVSVGLPNMTQGFWENSMLTDP |
| | AAAACATGAATAATGCTGGGGACAAATGGTCTGCCTTTTTAAAGGAACA | GNVQKAVCHPTAWDLGKGDFRILMCTKVTMDDFLTAHHEMGHIQ |
| | GTCCACACTTGCCCAAATGTATCCACTACAAGAAATTCAGAATCTCACA | YDMAYAAQPFLLRNGANEGFHEAVGEIMSLSAATPKHLKSIGLL |
| | GTCAAGCTTCAGCTGCAGGCTCTTCAGCAAAATGGGTCTTCAGTGCTCT | SPDFQEDNETEINFLLKQALTIVGTLPFTYMLEKWRWMVFKGEI |
| | CAGAAGACAAGAGCAAACGGTTGAACACAATTCTAAATACAATGAGCAC | PKDQWMKKWWEMKREIVGVVEPVPHDETYCDPASLFHVSNDYSF |
| | CATCTACAGTACTGGAAAAGTTTGTAACCCAGATAATCCACAAGAATGC | IRYYTRTLYQFQFQEALCQAAKHEGPLHKCDISNSTEAGQKLEN |
| | TTATTACTTGAACCAGGTTTGAATGAAATAATGGCAAACAGTTTAGACT | MLRLGKSEPWTLALENVVGAKNMNVRPLLNYFEPLFTWLKDONK |
| | ACAATGAGAGGCTCTGGGCTTGGGAAAGCTGGAGATCTGAGGTCGGCAA | NSFVGWSTDWSPYADQSIKVRISLKSALGDKAYEWNDNEMYLFR |
| | GCAGCTGAGGCCATTATATGAAGAGTATGTGGTCTTGAAAAATGAGATG | SSVAYAMRQYFLKVKNQMILFGEEDVRVANLKPRISENFFVTAP |
| | GCAAGAGCAAATCATTATGAGGACTATGGGGATTATTGGAGAGGAGACT | KNVSDIIPRTEVEKAIRMSRSRINDAFRLNDNSLEFLGIQPTLG |
| | ATGAAGTAAATGGGGTAGATGGCTATGACTACAGCCGCGGCCAGTTGAT | PPNQPPVS |
| | TGAAGATGTGGAACATACCTTTGAAGAGATTAAACCATTATATGAACAT | |
| | CTTCATGCCTATGTGAGGGCAAAGTTGATGAATGCCTATCCTTCCTATA | |
| | TCAGTCCAATTGGATGCCTCCCTGCTCATTTGCTTGGTGATATGTGGGG | |
| | TAGATTTTGGACAAATCTGTACTCTTTGACAGTTCCCTTTGGACAGAAA | |
| | CCAAACATAGATGTTACTGATGCAATGGTGGACCAGGCCTGGGATGCAC | |
| | AGAGAATATTCAAGGAGGCCGAGAAGTTCTTTGTATCTGTTGGTCTTCC | |
| | TAATATGACTCAAGGATTCTGGGAAAATTCCATGCTAACGGACCCAGGA | |
| | AATGTTCAGAAAGCAGTCTGCCATCCCACAGCTTGGGACCTGGGGAAGG | |
| | GCGACTTCAGGATCCTTATGTGCACAAAGGTGACAATGGACGACTTCCT | |
| | GACAGCTCATCATGAGATGGGGCATATCCAGTATGATATGGCATATGCT | |
| | GCACAACCTTTTCTGCTAAGAAATGGAGCTAATGAAGGATTCCATGAAG | |
| | CTGTTGGGGAAATCATGTCACTTTCTGCAGCCACACCTAAGCATTTAAA | |
| | ATCCATTGGTCTTCTGTCACCCGATTTTCAAGAAGACAATGAAACAGAA | |
| | ATAAACTTCCTGCTCAAACAAGCACTCACGATTGTTGGGACTCTGCCAT | |
| | TTACTTACATGTTAGAGAAGTGGAGGTGGATGGTCTTTAAAGGGGAAAT | |
| | TCCCAAAGACCAGTGGATGAAAAAGTGGTGGGAGATGAAGCGAGAGATA | |
| | GTTGGGGTGGTGGAACCTGTGCCCCATGATGAAACATACTGTGACCCCG | |
| | CATCTCTGTTCCATGTTTCTAATGATTACTCATTCATTCGATATTACAC | |
| | AAGGACCCTTTACCAATTCCAGTTTCAAGAAGCACTTTGTCAAGCAGCT | |
| | AAACATGAAGGCCCTCTGCACAAATGTGACATCTCAAACTCTACAGAAG | |
| | CTGGACAGAAACTGTTCAATATGCTGAGGCTTGGAAAATCAGAACCCTG | |
| | GACCCTAGCATTGGAAAATGTTGTAGGAGCAAAGAACATGAATGTAAGG | |
| | CCACTGCTCAACTACTTTGAGCCCTTATTTACCTGGCTGAAAGACCAGA | |
| | ACAAGAATTCTTTTGTGGGATGGAGTACCGACTGGAGTCCATATGCAGA | |
| | CCAAAGCATCAAAGTGAGGATAAGCCTAAAATCAGCTCTTGGAGATAAA | |
| | GCATATGAATGGAACGACAATGAAATGTACCTGTTCCGATCATCTGTTG | |
| | CATATGCTATGAGGCAGTACTTTTTAAAAGTAAAAAATCAGATGATTCT | |
| | TTTTGGGGAGGAGGATGTGCGAGTGGCTAATTTGAAACCAAGAATCTCC | |
| | TTTAATTTCTTTGTCACTGCACCTAAAAATGTGTCTGATATCATTCCTA | |
| | GAACTGAAGTTGAAAAGGCCATCAGGATGTCCCGGAGCCGTATCAATGA | |
| | TGCTTTCCGTCTGAATGACAACAGCCTAGAGTTTCTGGGGATACAGCCA | |
| | ACACTTGGACCTCCTAACCAGCCCCCTGTTTCC | |
| B8-AC- | SEQ ID NO: 59: | SEQ ID NO: 66: |
| DecFull CBR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGTMAIWRSNSGSNTLENGYELSRN |
| | AAAGGGCAGTTGTGCTGCCAGTCCACCGTGGCGGTTGATTGCCGTCATC | KENHSQPTQSSLEDSVTPTKAVKTTGVLSSPCPPNWIIYEKSCY |
| | CTCGGCATTCTGTGCTTGGTTATTCTCGTTATAGCGGTGGTGCTTGGGA | LFSMSLNSWDGSKRQCWQLGSNLLKIDSSNELGFIVKQVSSQPD |
| | CCATGGCGATCTGGCGCTCCAACTCTGGAAGTAACACCCTTGAAAATGG | NSFWIGLSRPQTEVPWLWEDGSTFSSNLFQIRTTATQENPSPNC |
| | TTACTTCCTCAGTAGGAACAAAGAGAACCATTCCCAGCCGACACAGTCA | VWIHVSVIYDQLCSVPSYSICEKKFSMGSSTIEEQAKTFLDKEN |
| | AGCCTTGAAGATTCAGTCACCCCTACAAAGGCCGTAAAAACGACAGGTG | HEAEDLFYQSSLASWNYNTNITEENVONMNNAGDKWSAFLKEQS |
| | TCCTGTCCTCTCCGTGTCCGCCTAACTGGATCATCTACGAGAAAAGTTG | TLAQMYPLQEIQNLTVKLQLQALQQNGSSVLSEDKSKRLNTILN |
| | TTATCTGTTTAGCATGAGCCTTAACAGTTGGGATGGCTCAAAAAGGCAG | TMSTIYSTGKVCNPDNPQECLLLEPGLNEIMANSLDYNERLWAW |
| | TGCTGGCAACTGGGGAGCAACCTTTTGAAGATAGACAGTTCCAACGAAC | ESWRSEVGKQLRPLYEEYVVLKNEMARANHYEDYGDYWRGDYEV |
| | TGGGCTTCATAGTCAAACAGGTGTCCTCTCAACCTGATAACTCATTCTG | NGVDGYDYSRGQLIEDVEHTFEEIKPLYEHLHAYVRAKLMNAYP |
| | GATCGGGCTCAGTCGACCCCAAACTGAGGTTCCATGGCTTTGGGAAGAC | SYISPIGCLPAHLLGDMWGRFWTNLYSLTVPFGQKPNIDVTDAM |
| | GGCAGCACTTTCTCTTCAAATTTGTTTCAAATAAGAACCACCGCTACGC | VDQAWDAQRIFKEAEKFFVSVGLPNMTQGFWENSMLTDPGNVQK |
| | AGGAGAATCCGAGTCCGAACTGTGTTTGGATTCACGTCTCAGTCATTTA | AVCHPTAWDLGKGDFRILMCTKVTMDDELTAHHEMGHIQYDMAY |
| | CGACCAACTTTGTAGTGTCCCTAGCTATTCCATCTGCGAGAAAAAGTTC | AAQPFLLRNGANEGFHEAVGEIMSLSAATPKHLKSIGLLSPDFQ |
| | AGTATGGGATCCTCCACCATTGAGGAACAGGCCAAGACATTTTTGGACA | EDNETEINFLLKQALTIVGTLPFTYMLEKWRWMVFKGEIPKDOW |
| | AGTTTAACCACGAAGCCGAAGACCTGTTCTATCAAAGTTCACTTGCTTC | MKKWWEMKREIVGVVEPVPHDETYCDPASLFHVSNDYSFIRYYT |
| | TTGGAATTATAACACCAATATTACTGAAGAGAATGTCCAAAACATGAAT | RTLYQFQFQEALCQAAKHEGPLHKCDISNSTEAGQKLENMLRLG |
| | AATGCTGGGGACAAATGGTCTGCCTTTTTAAAGGAACAGTCCACACTTG | KSEPWTLALENVVGAKNMNVRPLLNYFEPLFTWLKDONKNSFVG |
| | CCCAAATGTATCCACTACAAGAAATTCAGAATCTCACAGTCAAGCTTCA | WSTDWSPYADQSIKVRISLKSALGDKAYEWNDNEMYLFRSSVAY |
| | GCTGCAGGCTCTTCAGCAAAATGGGTCTTCAGTGCTCTCAGAAGACAAG | AMRQYFLKVKNQMILFGEEDVRVANLKPRISFNFFVTAPKNVSD |
| | AGCAAACGGTTGAACACAATTCTAAATACAATGAGCACCATCTACAGTA | IIPRTEVEKAIRMSRSRINDAFRLNDNSLEFLGIQPTLGPPNQP |
| | CTGGAAAAGTTTGTAACCCAGATAATCCACAAGAATGCTTATTACTTGA | PVS |
| | ACCAGGTTTGAATGAAATAATGGCAAACAGTTTAGACTACAATGAGAGG | |
| | CTCTGGGCTTGGGAAAGCTGGAGATCTGAGGTCGGCAAGCAGCTGAGGC | |
| | CATTATATGAAGAGTATGTGGTCTTGAAAAATGAGATGGCAAGAGCAAA | |
| | TCATTATGAGGACTATGGGGATTATTGGAGAGGAGACTATGAAGTAAAT | |
| | GGGGTAGATGGCTATGACTACAGCCGCGGCCAGTTGATTGAAGATGTGG | |
| | AACATACCTTTGAAGAGATTAAACCATTATATGAACATCTTCATGCCTA | |
| | TGTGAGGGCAAAGTTGATGAATGCCTATCCTTCCTATATCAGTCCAATT | |
| | GGATGCCTCCCTGCTCATTTGCTTGGTGATATGTGGGGTAGATTTTGGA | |
| | CAAATCTGTACTCTTTGACAGTTCCCTTTGGACAGAAACCAAACATAGA | |
| | TGTTACTGATGCAATGGTGGACCAGGCCTGGGATGCACAGAGAATATTC | |
| | AAGGAGGCCGAGAAGTTCTTTGTATCTGTTGGTCTTCCTAATATGACTC | |
| | AAGGATTCTGGGAAAATTCCATGCTAACGGACCCAGGAAATGTTCAGAA | |
| | AGCAGTCTGCCATCCCACAGCTTGGGACCTGGGGAAGGGCGACTTCAGG | |
| | ATCCTTATGTGCACAAAGGTGACAATGGACGACTTCCTGACAGCTCATC | |
| | ATGAGATGGGGCATATCCAGTATGATATGGCATATGCTGCACAACCTTT | |
| | TCTGCTAAGAAATGGAGCTAATGAAGGATTCCATGAAGCTGTTGGGGAA | |
| | ATCATGTCACTTTCTGCAGCCACACCTAAGCATTTAAAATCCATTGGTC | |
| | TTCTGTCACCCGATTTTCAAGAAGACAATGAAACAGAAATAAACTTCCT | |
| | GCTCAAACAAGCACTCACGATTGTTGGGACTCTGCCATTTACTTACATG | |
| | TTAGAGAAGTGGAGGTGGATGGTCTTTAAAGGGGAAATTCCCAAAGACC | |
| | AGTGGATGAAAAAGTGGTGGGAGATGAAGCGAGAGATAGTTGGGGTGGT | |
| | GGAACCTGTGCCCCATGATGAAACATACTGTGACCCCGCATCTCTGTTC | |
| | CATGTTTCTAATGATTACTCATTCATTCGATATTACACAAGGACCCTTT | |
| | ACCAATTCCAGTTTCAAGAAGCACTTTGTCAAGCAGCTAAACATGAAGG | |
| | CCCTCTGCACAAATGTGACATCTCAAACTCTACAGAAGCTGGACAGAAA | |
| | CTGTTCAATATGCTGAGGCTTGGAAAATCAGAACCCTGGACCCTAGCAT | |
| | TGGAAAATGTTGTAGGAGCAAAGAACATGAATGTAAGGCCACTGCTCAA | |
| | CTACTTTGAGCCCTTATTTACCTGGCTGAAAGACCAGAACAAGAATTCT | |
| | TTTGTGGGATGGAGTACCGACTGGAGTCCATATGCAGACCAAAGCATCA | |
| | AAGTGAGGATAAGCCTAAAATCAGCTCTTGGAGATAAAGCATATGAATG | |
| | GAACGACAATGAAATGTACCTGTTCCGATCATCTGTTGCATATGCTATG | |
| | AGGCAGTACTTTTTAAAAGTAAAAAATCAGATGATTCTTTTTGGGGAGG | |
| | AGGATGTGCGAGTGGCTAATTTGAAACCAAGAATCTCCTTTAATTTCTT | |
| | TGTCACTGCACCTAAAAATGTGTCTGATATCATTCCTAGAACTGAAGTT | |
| | GAAAAGGCCATCAGGATGTCCCGGAGCCGTATCAATGATGCTTTCCGTC | |
| | TGAATGACAACAGCCTAGAGTTTCTGGGGATACAGCCAACACTTGGACC | |
| | TCCTAACCAGCCCCCTGTTTCC | |
| B10-AC-163 | SEQ ID NO: 60: | SEQ ID NO: 67: |
| CBR | ATGAGGCTACCCCTGCTCCTGGTTTTTGCCTCTGTCATTCCGGGTGCTG | MRLPLLLVFASVIPGAVLSTIEEQAKTFLDKFNHEAEDLFYQSS |
| | TTCTCTCCACCATTGAGGAACAGGCCAAGACATTTTTGGACAAGTTTAA | LASWNYNTNITEENVONMNNAGDKWSAFLKEQSTLAQMYPLQEI |
| | CCACGAAGCCGAAGACCTGTTCTATCAAAGTTCACTTGCTTCTTGGAAT | QNLTVKLQLQALQQNGSSVLSEDKSKRLNTILNTMSTIYSTGKV |
| | TATAACACCAATATTACTGAAGAGAATGTCCAAAACATGAATAATGCTG | CNPDNPQECLLLEPGLNEIMANSLDYNERLWAWESWRSEVGKQL |
| | GGGACAAATGGTCTGCCTTTTTAAAGGAACAGTCCACACTTGCCCAAAT | RPLYEEYVVLKNEMARANHYEDYGDYWRGDYEVNGVDGYDYSRG |
| | GTATCCACTACAAGAAATTCAGAATCTCACAGTCAAGCTTCAGCTGCAG | QLIEDVEHTFEEIKPLYEHLHAYVRAKLMNAYPSYISPIGCLPA |
| | GCTCTTCAGCAAAATGGGTCTTCAGTGCTCTCAGAAGACAAGAGCAAAC | HLLGDMWGRFWTNLYSLTVPFGQKPNIDVTDAMVDQAWDAQRIF |
| | GGTTGAACACAATTCTAAATACAATGAGCACCATCTACAGTACTGGAAA | KEAEKFFVSVGLPNMTQGFWENSMLTDPGNVQKAVCHPTAWDLG |
| | AGTTTGTAACCCAGATAATCCACAAGAATGCTTATTACTTGAACCAGGT | KGDFRILMCTKVTMDDFLTAHHEMGHIQYDMAYAAQPFLLRNGA |
| | TTGAATGAAATAATGGCAAACAGTTTAGACTACAATGAGAGGCTCTGGG | NEGFHEAVGEIMSLSAATPKHLKSIGLLSPDFQEDNETEINFLL |
| | CTTGGGAAAGCTGGAGATCTGAGGTCGGCAAGCAGCTGAGGCCATTATA | KQALTIVGTLPFTYMLEKWRWMVFKGEIPKDQWMKKWWEMKREI |
| | TGAAGAGTATGTGGTCTTGAAAAATGAGATGGCAAGAGCAAATCATTAT | VGVVEPVPHDETYCDPASLFHVSNDYSFIRYYTRTLYQFQFQEA |
| | GAGGACTATGGGGATTATTGGAGAGGAGACTATGAAGTAAATGGGGTAG | LCQAAKHEGPLHKCDISNSTEAGQKLENMLRLGKSEPWTLALEN |
| | ATGGCTATGACTACAGCCGCGGCCAGTTGATTGAAGATGTGGAACATAC | VVGAKNMNVRPLLNYFEPLFTWLKDONKNSFVGWSTDWSPYADQ |
| | CTTTGAAGAGATTAAACCATTATATGAACATCTTCATGCCTATGTGAGG | SIKVRISLKSALGDKAYEWNDNEMYLFRSSVAYAMRQYFLKVKN |
| | GCAAAGTTGATGAATGCCTATCCTTCCTATATCAGTCCAATTGGATGCC | QMILFGEEDVRVANLKPRISFNFFVTAPKNVSDIIPRTEVEKAI |
| | TCCCTGCTCATTTGCTTGGTGATATGTGGGGTAGATTTTGGACAAATCT | RMSRSRINDAFRLNDNSLEFLGIQPTLGPPNQPPVSGSFVPVFL |
| | GTACTCTTTGACAGTTCCCTTTGGACAGAAACCAAACATAGATGTTACT | PAKPTTTPAPRPPTPAPTIASQPLSLRPEACRPAAGGAVHTRGL |
| | GATGCAATGGTGGACCAGGCCTGGGATGCACAGAGAATATTCAAGGAGG | DFACDSSLGGTDKELRLVDGENKCSGRVEVKVQEEWGTVCNNGW |
| | CCGAGAAGTTCTTTGTATCTGTTGGTCTTCCTAATATGACTCAAGGATT | SMEAVSVICNQLGCPTAIKAPGWANSSAGSGRIWMDHVSCRGNE |
| | CTGGGAAAATTCCATGCTAACGGACCCAGGAAATGTTCAGAAAGCAGTC | SALWDCKHDGWGKHSNCTHQQDAGVTCSDGSNLEMRLTRGGNMC |
| | TGCCATCCCACAGCTTGGGACCTGGGGAAGGGCGACTTCAGGATCCTTA | SGRIEIKFQGRWGTVCDDNFNIDHASVICRQLECGSAVSFSGSS |
| | TGTGCACAAAGGTGACAATGGACGACTTCCTGACAGCTCATCATGAGAT | NFGEGSGPIWFDDLICNGNESALWNCKHQGWGKHNCDHAEDAGV |
| | GGGGCATATCCAGTATGATATGGCATATGCTGCACAACCTTTTCTGCTA | ICSKGADLSLRLVDGVTECSGRLEVRFQGEWGTICDDGWDSYDA |
| | AGAAATGGAGCTAATGAAGGATTCCATGAAGCTGTTGGGGAAATCATGT | AVACKQLGCPTAVTAIGRVNASKGFGHIWLDSVSCQGHEPAIWQ |
| | CACTTTCTGCAGCCACACCTAAGCATTTAAAATCCATTGGTCTTCTGTC | CKHHEWGKHYCNHNEDAGVTCSDGSDLELRLRGGGSRCAGTVEV |
| | ACCCGATTTTCAAGAAGACAATGAAACAGAAATAAACTTCCTGCTCAAA | EIQRLLGKVCDRGWGLKEADVVCRQLGCGSALKTSYQVYSKIQA |
| | CAAGCACTCACGATTGTTGGGACTCTGCCATTTACTTACATGTTAGAGA | TNTWLFLSSCNGNETSLWDCKNWQWGGLTCDHYEEAKITCSAHR |
| | AGTGGAGGTGGATGGTCTTTAAAGGGGAAATTCCCAAAGACCAGTGGAT | EPRLVGGDIPCSGRVEVKHGDTWGSICDSDFSLEAASVLCRELQ |
| | GAAAAAGTGGTGGGAGATGAAGCGAGAGATAGTTGGGGTGGTGGAACCT | CGTVVSILGGAHFGEGNGQIWAEEFQCEGHESHLSLCPVAPRPE |
| | GTGCCCCATGATGAAACATACTGTGACCCCGCATCTCTGTTCCATGTTT | GTCSHSRDVGVVCSRYTEIRLVNGKTPCEGRVELKTLGAWGSLC |
| | CTAATGATTACTCATTCATTCGATATTACACAAGGACCCTTTACCAATT | NSHWDIEDAHVLCQQLKCGVALSTPGGARFGKGNGQIWRHMFHC |
| | CCAGTTTCAAGAAGCACTTTGTCAAGCAGCTAAACATGAAGGCCCTCTG | TGTEQHMGDCPVTALGASLCPSEQVASVICSGNQSQTLSSCNSS |
| | CACAAATGTGACATCTCAAACTCTACAGAAGCTGGACAGAAACTGTTCA | SLGPTRPTIPEESAVACIESGOLRLVNGGGRCAGRVEIYHEGSW |
| | ATATGCTGAGGCTTGGAAAATCAGAACCCTGGACCCTAGCATTGGAAAA | GTICDDSWDLSDAHVVCRQLGCGEAINATGSAHFGEGTGPIWLD |
| | TGTTGTAGGAGCAAAGAACATGAATGTAAGGCCACTGCTCAACTACTTT | EMKCNGKESRIWQCHSHGWGQQNCRHKEDAGVICSEFMSLRLTS |
| | GAGCCCTTATTTACCTGGCTGAAAGACCAGAACAAGAATTCTTTTGTGG | EASREACAGRLEVFYNGAWGTVGKSSMSETTVGVVCRQLGCADK |
| | GATGGAGTACCGACTGGAGTCCATATGCAGACCAAAGCATCAAAGTGAG | GKINPASLDKAMSIPMWVDNVQCPKGPDTLWQCPSSPWEKRLAS |
| | GATAAGCCTAAAATCAGCTCTTGGAGATAAAGCATATGAATGGAACGAC | PSEETWITCDNKIRLQEGPTSCSGRVEIWHGGSWGTVCDDSWDL |
| | AATGAAATGTACCTGTTCCGATCATCTGTTGCATATGCTATGAGGCAGT | DDAQVVCQQLGCGPALKAFKEAEFGQGTGPIWLNEVKCKGNESS |
| | ACTTTTTAAAAGTAAAAAATCAGATGATTCTTTTTGGGGAGGAGGATGT | LWDCPARRWGHSECGHKEDAAVNCTDISVQKTPQKATTGRSSRQ |
| | GCGAGTGGCTAATTTGAAACCAAGAATCTCCTTTAATTTCTTTGTCACT | SSFIAVGILGVVLLAIFVALFFLTKKRRORQRLAVSSRGENLVH |
| | GCACCTAAAAATGTGTCTGATATCATTCCTAGAACTGAAGTTGAAAAGG | QIQYREMNSCLNADDLDLMNSSENSHESADFSAAELISVSKFLP |
| | CCATCAGGATGTCCCGGAGCCGTATCAATGATGCTTTCCGTCTGAATGA | ISGMEKEAILSHTEKENGNL |
| | CAACAGCCTAGAGTTTCTGGGGATACAGCCAACACTTGGACCTCCTAAC | |
| | CAGCCCCCTGTTTCCGGATCCTTCGTGCCGGTCTTCCTGCCAGCGAAGC | |
| | CCACCACGACGCCAGCGCCGCGACCACCAACACCGGCGCCCACCATCGC | |
| | GTCGCAGCCCCTGTCCCTGCGCCCAGAGGCGTGCCGGCCAGCGGCGGGG | |
| | GGCGCAGTGCACACGAGGGGGCTGGACTTCGCCTGTGATTCATCCCTGG | |
| | GTGGAACAGATAAGGAGTTGAGGTTGGTGGATGGAGAGAATAAGTGCAG | |
| | TGGACGGGTTGAGGTCAAGGTGCAGGAAGAGTGGGGTACTGTTTGTAAC | |
| | AACGGCTGGAGTATGGAAGCAGTCTCCGTGATATGCAATCAACTTGGTT | |
| | GCCCTACAGCCATAAAAGCTCCTGGATGGGCTAACTCATCTGCCGGAAG | |
| | CGGTCGAATATGGATGGACCATGTATCCTGTAGGGGCAATGAGTCAGCA | |
| | CTGTGGGACTGCAAGCATGACGGGTGGGGAAAACACTCTAATTGTACCC | |
| | ACCAGCAAGATGCCGGTGTGACGTGTAGCGACGGCAGCAACTTGGAGAT | |
| | GCGGTTGACGCGCGGCGGGAACATGTGCAGCGGTCGCATTGAGATAAAG | |
| | TTCCAAGGGCGCTGGGGGACCGTTTGTGACGACAACTTCAATATAGATC | |
| | ATGCTAGTGTGATATGCAGGCAGTTGGAGTGCGGTAGCGCCGTCTCATT | |
| | TAGTGGTTCTAGCAATTTCGGTGAGGGATCCGGGCCTATATGGTTCGAC | |
| | GACCTTATCTGCAATGGAAATGAGAGCGCCCTCTGGAACTGCAAGCACC | |
| | AAGGTTGGGGTAAGCATAACTGCGATCATGCTGAGGATGCGGGTGTGAT | |
| | TTGTAGCAAGGGAGCCGACTTGTCACTTCGACTCGTAGATGGCGTGACG | |
| | GAATGTAGCGGTCGCCTTGAGGTACGCTTCCAAGGTGAATGGGGCACAA | |
| | TCTGTGATGACGGTTGGGACTCATATGACGCTGCGGTTGCTTGCAAACA | |
| | GCTGGGCTGTCCGACAGCGGTAACGGCAATTGGGCGGGTAAATGCCAGT | |
| | AAGGGGTTTGGACATATATGGTTGGACAGTGTTTCTTGTCAGGGACACG | |
| | AACCTGCAATATGGCAATGTAAACATCACGAGTGGGGGAAGCATTACTG | |
| | CAATCATAATGAAGACGCAGGCGTCACATGCTCTGACGGATCCGACCTG | |
| | GAATTGCGGCTGAGAGGGGGGGGTTCACGGTGCGCAGGAACCGTGGAAG | |
| | TTGAGATTCAACGCCTGCTCGGGAAAGTTTGTGACAGGGGGTGGGGCCT | |
| | TAAGGAAGCAGACGTAGTCTGCCGACAACTGGGTTGCGGGAGCGCCCTC | |
| | AAGACGTCCTATCAAGTTTACAGCAAAATTCAAGCAACTAATACCTGGC | |
| | TGTTTCTTTCCTCCTGCAATGGTAACGAAACGAGCCTCTGGGATTGTAA | |
| | AAATTGGCAATGGGGAGGCCTTACATGTGATCACTATGAAGAGGCCAAA | |
| | ATCACCTGCAGCGCGCACCGAGAGCCTAGGCTGGTTGGAGGGGATATTC | |
| | CCTGTTCAGGTAGAGTTGAAGTCAAGCATGGTGATACCTGGGGGTCCAT | |
| | ATGCGACTCCGATTTCAGTTTGGAAGCAGCGAGCGTGCTGTGCCGAGAG | |
| | CTTCAATGTGGGACAGTAGTTTCCATTCTTGGTGGCGCCCACTTCGGGG | |
| | AAGGTAACGGACAGATTTGGGCGGAGGAATTCCAATGTGAGGGCCATGA | |
| | AAGTCACCTGAGTCTTTGTCCTGTAGCGCCTCGGCCGGAAGGTACTTGT | |
| | TCTCACTCCAGAGACGTTGGCGTGGTTTGTTCTAGGTACACTGAGATAA | |
| | GGCTGGTGAATGGCAAAACGCCTTGTGAAGGAAGGGTGGAGCTCAAAAC | |
| | GCTTGGCGCCTGGGGGTCTCTGTGCAACTCCCACTGGGACATAGAGGAC | |
| | GCACATGTCTTGTGCCAGCAACTCAAATGCGGTGTCGCGCTTTCAACCC | |
| | CTGGGGGCGCTAGATTCGGGAAAGGTAACGGCCAGATATGGCGCCATAT | |
| | GTTCCATTGCACCGGAACTGAACAGCATATGGGAGATTGTCCTGTGACT | |
| | GCCTTGGGGGCAAGTCTGTGTCCCTCAGAACAAGTGGCTTCAGTTATTT | |
| | GCAGCGGTAACCAGAGTCAGACGCTCAGCTCCTGCAACAGCAGCAGTCT | |
| | GGGTCCAACAAGACCCACAATACCCGAGGAGTCAGCGGTCGCGTGCATC | |
| | GAATCCGGGCAATTGAGACTCGTTAATGGCGGGGGTCGGTGCGCAGGGC | |
| | GCGTGGAGATCTACCACGAGGGTAGTTGGGGCACAATTTGTGACGACAG | |
| | CTGGGACCTTTCCGACGCTCATGTCGTATGCCGACAACTGGGGTGCGGT | |
| | GAGGCCATCAATGCCACCGGAAGCGCGCATTTCGGTGAAGGCACGGGCC | |
| | CAATTTGGCTTGATGAGATGAAATGTAATGGAAAGGAGTCCCGCATTTG | |
| | GCAATGCCATAGCCATGGCTGGGGTCAACAAAATTGTCGACACAAAGAA | |
| | GATGCCGGGGTGATCTGCTCAGAATTCATGTCCTTGCGGCTTACTAGCG | |
| | AAGCGTCCCGCGAGGCCTGTGCTGGGAGACTGGAAGTTTTTTATAACGG | |
| | GGCTTGGGGAACGGTTGGTAAGTCATCAATGAGTGAAACCACAGTTGGT | |
| | GTTGTGTGTAGACAACTCGGTTGCGCCGACAAGGGAAAGATCAACCCGG | |
| | CGAGCCTTGATAAGGCCATGAGCATCCCCATGTGGGTGGATAACGTTCA | |
| | GTGTCCGAAAGGTCCCGATACCTTGTGGCAATGCCCAAGTTCTCCTTGG | |
| | GAGAAGAGACTCGCCTCACCAAGTGAAGAGACGTGGATTACGTGTGATA | |
| | ACAAAATTAGGCTCCAAGAAGGACCGACCAGTTGCAGCGGAAGAGTTGA | |
| | GATATGGCATGGAGGAAGCTGGGGAACCGTGTGCGATGACAGCTGGGAC | |
| | CTGGACGACGCCCAGGTGGTGTGCCAACAGCTGGGTTGCGGGCCTGCCC | |
| | TCAAAGCATTTAAGGAAGCCGAATTCGGTCAGGGTACTGGGCCAATCTG | |
| | GCTGAACGAGGTAAAGTGCAAAGGTAACGAAAGTAGCCTGTGGGACTGT | |
| | CCGGCACGAAGGTGGGGCCACAGCGAGTGTGGCCATAAGGAAGACGCGG | |
| | CCGTGAACTGTACAGACATATCCGTACAAAAAACGCCCCAAAAGGCGAC | |
| | GACCGGGCGATCATCAAGACAATCTAGCTTTATTGCCGTGGGAATTCTC | |
| | GGTGTAGTGCTTCTTGCTATATTTGTCGCTTTGTTCTTTCTGACTAAAA | |
| | AGCGCAGGCAAAGGCAGCGGCTTGCTGTGAGCTCTCGGGGAGAAAACCT | |
| | CGTTCACCAAATCCAATACCGAGAAATGAACTCCTGTCTCAACGCCGAC | |
| | GATCTTGACCTGATGAACTCATCTGAGAACTCACACGAGTCCGCCGATT | |
| | TCAGCGCGGCGGAATTGATCTCTGTCAGCAAATTTCTGCCTATAAGTGG | |
| | CATGGAAAAAGAAGCCATACTCTCTCACACGGAAAAGGAAAATGGCAAC | |
| | CTT | |
| MR CPR | SEQ ID NO: 68: | SEQ ID NO: 86: |
| | ATGGCCTTACCAGTGACCGCCTTGCTCCTGCCGCTGGCCTTGCTGCTCC | MALPVTALLLPLALLLHAARPGSDIQMTQSPSSLSASVGDRVTI |
| | ACGCCGCCAGGCCGGGATCCGACATCCAGATGACCCAGAGCCCCTCCTC | TCRASQEISGYLSWLQQKPGKAIKRLIYAASTLQSGVPSRFSGS |
| | TTTATCCGCCTCTGTGGGCGACAGAGTGACCATCACTTGTCGTGCCAGC | RSGSDYTLTISSLOPEDFATYYCLQYASYPFTFGQGTKLEIKGG |
| | CAAGAAATCTCCGGCTATCTGTCTTGGCTGCAGCAGAAGCCCGGTAAGG | GGSGGGGSGGGSGGGGSQVTLKESGPTLVKPTQTLTLTCTFSGF |
| | CTATCAAGAGACTCATCTACGCCGCCAGCACTTTACAGAGCGGAGTGCC | SLSTSTMGVGWIRQPPGKALEWLAHILWNDSKRYNPSLKSRLTI |
| | TAGCAGATTTAGCGGCTCTCGTAGCGGCAGCGATTATACTTTAACCATC | TKDTSKKQVVLTMTNMDPVDTATYYCARIVYYSTYVGYFDVWGQ |
| | TCCTCTTTACAGCCCGAAGACTTTGCTACCTACTACTGTTTACAGTACG | GTTVTVSSGSFVPVFLPAKPTTTPAPRPPTPAPTIASQPLSLRP |
| | CCAGCTACCCCTTCACCTTCGGTCAAGGTACCAAGCTGGAGATCAAAGG | EACRPAAGGAVHTRGLDFACDGVVIIVILLILTGAGLAAYFFYK |
| | TGGCGGCGGCTCTGGCGGCGGTGGCTCTGGCGGTGGCTCCGGCGGTGGT | KRRVHLPQEGAFENTLYFNSQSSPGTSDMKDLVGNIEQNEHSVI |
| | GGTAGCCAAGTTACCCTCAAGGAGAGCGGCCCCACTTTAGTGAAGCCTA | |
| | CCCAGACTTTAACTTTAACTTGTACCTTCAGCGGCTTCTCTTTAAGCAC | |
| | CTCCACAATGGGCGTGGGCTGGATCAGACAGCCTCCCGGCAAGGCTCTG | |
| | GAGTGGCTGGCCCACATCCTCTGGAACGACAGCAAGAGGTACAACCCCT | |
| | CTTTAAAGTCTCGTCTGACCATCACCAAGGACACCTCCAAGAAGCAAGT | |
| | TGTGCTGACCATGACCAATATGGACCCCGTGGACACCGCCACCTATTAC | |
| | TGCGCTCGTATCGTGTACTACTCCACCTACGTGGGCTACTTCGACGTCT | |
| | GGGGACAAGGTACCACCGTGACCGTGAGCTCTGGATCCTTCGTGCCGGT | |
| | CTTCCTGCCAGCGAAGCCCACCACGACGCCAGCGCCGCGACCACCAACA | |
| | CCGGCGCCCACCATCGCGTCGCAGCCCCTGTCCCTGCGCCCAGAGGCGT | |
| | GCCGGCCAGCGGCGGGGGGCGCAGTGCACACGAGGGGGCTGGACTTCGC | |
| | CTGTGATGGAGTAGTCATCATTGTGATCCTCCTGATTTTAACGGGTGCT | |
| | GGCCTTGCCGCCTATTTCTTTTATAAGAAAAGACGTGTGCACCTACCTC | |
| | AAGAGGGCGCCTTTGAAAACACTCTGTATTTTAACAGTCAGTCAAGCCC | |
| | AGGAACTAGTGATATGAAAGATCTCGTGGGCAATATTGAACAGAATGAA | |
| | CACTCGGTCATCG | |
| F1-FLT3- | SEQ ID NO: 69: | SEQ ID NO: 87: |
| scFv CPR | ATGGCCTTACCAGTGACCGCCTTGCTCCTGCCGCTGGCCTTGCTGCTCC | MALPVTALLLPLALLLHAARPGSDIQMTQSPSSLSASVGDRVTI |
| | ACGCCGCCAGGCCGGGATCCGACATCCAGATGACCCAGAGCCCCTCCTC | TCRASQEISGYLSWLQQKPGKAIKRLIYAASTLQSGVPSRFSGS |
| | TTTATCCGCCTCTGTGGGCGACAGAGTGACCATCACTTGTCGTGCCAGC | RSGSDYTLTISSLOPEDFATYYCLQYASYPFTFGQGTKLEIKGG |
| | CAAGAAATCTCCGGCTATCTGTCTTGGCTGCAGCAGAAGCCCGGTAAGG | GGSGGGGSGGGSGGGGSQVTLKESGPTLVKPTQTLTLTCTFSGF |
| | CTATCAAGAGACTCATCTACGCCGCCAGCACTTTACAGAGCGGAGTGCC | SLSTSTMGVGWIRQPPGKALEWLAHILWNDSKRYNPSLKSRLTI |
| | TAGCAGATTTAGCGGCTCTCGTAGCGGCAGCGATTATACTTTAACCATC | TKDTSKKQVVLTMTNMDPVDTATYYCARIVYYSTYVGYFDVWGQ |
| | TCCTCTTTACAGCCCGAAGACTTTGCTACCTACTACTGTTTACAGTACG | GTTVTVSSGSFVPVFLPAKPTTTPAPRPPTPAPTIASQPLSLRP |
| | CCAGCTACCCCTTCACCTTCGGTCAAGGTACCAAGCTGGAGATCAAAGG | EACRPAAGGAVHTRGLDFACDIDAKPTHELLTTKADTRKMDPSK |
| | TGGCGGCGGCTCTGGCGGCGGTGGCTCTGGCGGTGGCTCCGGCGGTGGT | PSSNVAGVVIIVILLILTGAGLAAYFFYKKRRVHLPQEGAFENT |
| | GGTAGCCAAGTTACCCTCAAGGAGAGCGGCCCCACTTTAGTGAAGCCTA | LYFNSQSSPGTSDMKDLVGNIEQNEHSVI |
| | CCCAGACTTTAACTTTAACTTGTACCTTCAGCGGCTTCTCTTTAAGCAC | |
| | CTCCACAATGGGCGTGGGCTGGATCAGACAGCCTCCCGGCAAGGCTCTG | |
| | GAGTGGCTGGCCCACATCCTCTGGAACGACAGCAAGAGGTACAACCCCT | |
| | CTTTAAAGTCTCGTCTGACCATCACCAAGGACACCTCCAAGAAGCAAGT | |
| | TGTGCTGACCATGACCAATATGGACCCCGTGGACACCGCCACCTATTAC | |
| | TGCGCTCGTATCGTGTACTACTCCACCTACGTGGGCTACTTCGACGTCT | |
| | GGGGACAAGGTACCACCGTGACCGTGAGCTCTGGATCCTTCGTGCCGGT | |
| | CTTCCTGCCAGCGAAGCCCACCACGACGCCAGCGCCGCGACCACCAACA | |
| | CCGGCGCCCACCATCGCGTCGCAGCCCCTGTCCCTGCGCCCAGAGGCGT | |
| | GCCGGCCAGCGGCGGGGGGCGCAGTGCACACGAGGGGGCTGGACTTCGC | |
| | CTGTGATATTGATGCTAAACCTACTCATGAATTACTTACAACAAAAGCT | |
| | GACACAAGGAAGATGGACCCTTCTAAACCGTCTTCCAACGTGGCCGGAG | |
| | TAGTCATCATTGTGATCCTCCTGATTTTAACGGGTGCTGGCCTTGCCGC | |
| | CTATTTCTTTTATAAGAAAAGACGTGTGCACCTACCTCAAGAGGGCGCC | |
| | TTTGAAAACACTCTGTATTTTAACAGTCAGTCAAGCCCAGGAACTAGTG | |
| | ATATGAAAGATCTCGTGGGCAATATTGAACAGAATGAACACTCGGTCAT | |
| | C | |
| F2-FLT3- | SEQ ID NO: 70: | SEQ ID NO: 88: |
| scFv CPR | ATGGCCTTACCAGTGACCGCCTTGCTCCTGCCGCTGGCCTTGCTGCTCC | MALPVTALLLPLALLLHAARPGSDIQMTQSPSSLSASVGDRVTI |
| | ACGCCGCCAGGCCGGGATCCGACATCCAGATGACCCAGAGCCCCTCCTC | TCRASQEISGYLSWLQQKPGKAIKRLIYAASTLQSGVPSRFSGS |
| | TTTATCCGCCTCTGTGGGCGACAGAGTGACCATCACTTGTCGTGCCAGC | RSGSDYTLTISSLOPEDFATYYCLQYASYPFTFGQGTKLEIKGG |
| | CAAGAAATCTCCGGCTATCTGTCTTGGCTGCAGCAGAAGCCCGGTAAGG | GGSGGGGSGGGSGGGGSQVTLKESGPTLVKPTQTLTLTCTFSGF |
| | CTATCAAGAGACTCATCTACGCCGCCAGCACTTTACAGAGCGGAGTGCC | SLSTSTMGVGWIRQPPGKALEWLAHILWNDSKRYNPSLKSRLTI |
| | TAGCAGATTTAGCGGCTCTCGTAGCGGCAGCGATTATACTTTAACCATC | TKDTSKKQVVLTMTNMDPVDTATYYCARIVYYSTYVGYFDVWGQ |
| | TCCTCTTTACAGCCCGAAGACTTTGCTACCTACTACTGTTTACAGTACG | GTTVTVSSIDAKPTHELLTTKADTRKMDPSKPSSNVAGVVIIVI |
| | CCAGCTACCCCTTCACCTTCGGTCAAGGTACCAAGCTGGAGATCAAAGG | LLILTGAGLAAYFFYKKRRVHLPQEGAFENTLYFNSQSSPGTSD |
| | TGGCGGCGGCTCTGGCGGCGGTGGCTCTGGCGGTGGCTCCGGCGGTGGT | MKDLVGNIEQNEHSVI |
| | GGTAGCCAAGTTACCCTCAAGGAGAGCGGCCCCACTTTAGTGAAGCCTA | |
| | CCCAGACTTTAACTTTAACTTGTACCTTCAGCGGCTTCTCTTTAAGCAC | |
| | CTCCACAATGGGCGTGGGCTGGATCAGACAGCCTCCCGGCAAGGCTCTG | |
| | GAGTGGCTGGCCCACATCCTCTGGAACGACAGCAAGAGGTACAACCCCT | |
| | CTTTAAAGTCTCGTCTGACCATCACCAAGGACACCTCCAAGAAGCAAGT | |
| | TGTGCTGACCATGACCAATATGGACCCCGTGGACACCGCCACCTATTAC | |
| | TGCGCTCGTATCGTGTACTACTCCACCTACGTGGGCTACTTCGACGTCT | |
| | GGGGACAAGGTACCACCGTGACCGTGAGCTCTATTGATGCTAAACCTAC | |
| | TCATGAATTACTTACAACAAAAGCTGACACAAGGAAGATGGACCCTTCT | |
| | AAACCGTCTTCCAACGTGGCCGGAGTAGTCATCATTGTGATCCTCCTGA | |
| | TTTTAACGGGTGCTGGCCTTGCCGCCTATTTCTTTTATAAGAAAAGACG | |
| | TGTGCACCTACCTCAAGAGGGCGCCTTTGAAAACACTCTGTATTTTAAC | |
| | AGTCAGTCAAGCCCAGGAACTAGTGATATGAAAGATCTCGTGGGCAATA | |
| | TTGAACAGAATGAACACTCGGTCATC | |
| F3-FLT3- | SEQ ID NO: 71: | SEQ ID NO: 89: |
| scFv CPR | ATGGCCTTACCAGTGACCGCCTTGCTCCTGCCGCTGGCCTTGCTGCTCC | MALPVTALLLPLALLLHAARPGSDIQMTQSPSSLSASVGDRVTI |
| | ACGCCGCCAGGCCGGGATCCGACATCCAGATGACCCAGAGCCCCTCCTC | TCRASQEISGYLSWLOQKPGKAIKRLIYAASTLQSGVPSRESGS |
| | TTTATCCGCCTCTGTGGGCGACAGAGTGACCATCACTTGTCGTGCCAGC | RSGSDYTLTISSLOPEDFATYYCLQYASYPFTFGQGTKLEIKGG |
| | CAAGAAATCTCCGGCTATCTGTCTTGGCTGCAGCAGAAGCCCGGTAAGG | GGSGGGGSGGGSGGGGSQVTLKESGPTLVKPTQTLTLTCTFSGF |
| | CTATCAAGAGACTCATCTACGCCGCCAGCACTTTACAGAGCGGAGTGCC | SLSTSTMGVGWIRQPPGKALEWLAHILWNDSKRYNPSLKSRLTI |
| | TAGCAGATTTAGCGGCTCTCGTAGCGGCAGCGATTATACTTTAACCATC | TKDTSKKQVVLTMTNMDPVDTATYYCARIVYYSTYVGYFDVWGQ |
| | TCCTCTTTACAGCCCGAAGACTTTGCTACCTACTACTGTTTACAGTACG | GTTVTVSSGSFVPVFLPAKPTTTPAPRPPTPAPTIASQPLSLRP |
| | CCAGCTACCCCTTCACCTTCGGTCAAGGTACCAAGCTGGAGATCAAAGG | EACRPAAGGAVHTRGLDFACDDTRKMDPSKPSSNVAGVVIIVIL |
| | TGGCGGCGGCTCTGGCGGCGGTGGCTCTGGCGGTGGCTCCGGCGGTGGT | LILTGAGLAAYFFYKKRRVHLPQEGAFENTLYFNSQSSPGTSDM |
| | GGTAGCCAAGTTACCCTCAAGGAGAGCGGCCCCACTTTAGTGAAGCCTA | KDLVGNIEQNEHSVI |
| | CCCAGACTTTAACTTTAACTTGTACCTTCAGCGGCTTCTCTTTAAGCAC | |
| | CTCCACAATGGGCGTGGGCTGGATCAGACAGCCTCCCGGCAAGGCTCTG | |
| | GAGTGGCTGGCCCACATCCTCTGGAACGACAGCAAGAGGTACAACCCCT | |
| | CTTTAAAGTCTCGTCTGACCATCACCAAGGACACCTCCAAGAAGCAAGT | |
| | TGTGCTGACCATGACCAATATGGACCCCGTGGACACCGCCACCTATTAC | |
| | TGCGCTCGTATCGTGTACTACTCCACCTACGTGGGCTACTTCGACGTCT | |
| | GGGGACAAGGTACCACCGTGACCGTGAGCTCTGGATCCTTCGTGCCGGT | |
| | CTTCCTGCCAGCGAAGCCCACCACGACGCCAGCGCCGCGACCACCAACA | |
| | CCGGCGCCCACCATCGCGTCGCAGCCCCTGTCCCTGCGCCCAGAGGCGT | |
| | GCCGGCCAGCGGCGGGGGGCGCAGTGCACACGAGGGGGCTGGACTTCGC | |
| | CTGTGATGACACAAGGAAGATGGACCCTTCTAAACCGTCTTCCAACGTG | |
| | GCCGGAGTAGTCATCATTGTGATCCTCCTGATTTTAACGGGTGCTGGCC | |
| | TTGCCGCCTATTTCTTTTATAAGAAAAGACGTGTGCACCTACCTCAAGA | |
| | GGGCGCCTTTGAAAACACTCTGTATTTTAACAGTCAGTCAAGCCCAGGA | |
| | ACTAGTGATATGAAAGATCTCGTGGGCAATATTGAACAGAATGAACACT | |
| | CGGTCATC | |
| F4-FLT3- | SEQ ID NO: 72: | SEQ ID NO: 90: |
| scFv CPR | ATGGCCTTACCAGTGACCGCCTTGCTCCTGCCGCTGGCCTTGCTGCTCC | MALPVTALLLPLALLLHAARPGSDIQMTQSPSSLSASVGDRVTI |
| | ACGCCGCCAGGCCGGGATCCGACATCCAGATGACCCAGAGCCCCTCCTC | TCRASQEISGYLSWLQQKPGKAIKRLIYAASTLQSGVPSRFSGS |
| | TTTATCCGCCTCTGTGGGCGACAGAGTGACCATCACTTGTCGTGCCAGC | RSGSDYTLTISSLOPEDFATYYCLQYASYPFTFGQGTKLEIKGG |
| | CAAGAAATCTCCGGCTATCTGTCTTGGCTGCAGCAGAAGCCCGGTAAGG | GGSGGGGSGGGSGGGGSQVTLKESGPTLVKPTQTLTLTCTFSGF |
| | CTATCAAGAGACTCATCTACGCCGCCAGCACTTTACAGAGCGGAGTGCC | SLSTSTMGVGWIRQPPGKALEWLAHILWNDSKRYNPSLKSRLTI |
| | TAGCAGATTTAGCGGCTCTCGTAGCGGCAGCGATTATACTTTAACCATC | TKDTSKKQVVLTMTNMDPVDTATYYCARIVYYSTYVGYFDVWGQ |
| | TCCTCTTTACAGCCCGAAGACTTTGCTACCTACTACTGTTTACAGTACG | GTTVTVSSDTRKMDPSKPSSNVAGVVIIVILLILTGAGLAAYFF |
| | CCAGCTACCCCTTCACCTTCGGTCAAGGTACCAAGCTGGAGATCAAAGG | YKKRRVHLPQEGAFENTLYFNSQSSPGTSDMKDLVGNIEQNEHS |
| | TGGCGGCGGCTCTGGCGGCGGTGGCTCTGGCGGTGGCTCCGGCGGTGGT | VI |
| | GGTAGCCAAGTTACCCTCAAGGAGAGCGGCCCCACTTTAGTGAAGCCTA | |
| | CCCAGACTTTAACTTTAACTTGTACCTTCAGCGGCTTCTCTTTAAGCAC | |
| | CTCCACAATGGGCGTGGGCTGGATCAGACAGCCTCCCGGCAAGGCTCTG | |
| | GAGTGGCTGGCCCACATCCTCTGGAACGACAGCAAGAGGTACAACCCCT | |
| | CTTTAAAGTCTCGTCTGACCATCACCAAGGACACCTCCAAGAAGCAAGT | |
| | TGTGCTGACCATGACCAATATGGACCCCGTGGACACCGCCACCTATTAC | |
| | TGCGCTCGTATCGTGTACTACTCCACCTACGTGGGCTACTTCGACGTCT | |
| | GGGGACAAGGTACCACCGTGACCGTGAGCTCTGACACAAGGAAGATGGA | |
| | CCCTTCTAAACCGTCTTCCAACGTGGCCGGAGTAGTCATCATTGTGATC | |
| | CTCCTGATTTTAACGGGTGCTGGCCTTGCCGCCTATTTCTTTTATAAGA | |
| | AAAGACGTGTGCACCTACCTCAAGAGGGCGCCTTTGAAAACACTCTGTA | |
| | TTTTAACAGTCAGTCAAGCCCAGGAACTAGTGATATGAAAGATCTCGTG | |
| | GGCAATATTGAACAGAATGAACACTCGGTCATC | |
| F5-FLT3- | SEQ ID NO: 73: | SEQ ID NO: 91: |
| scFv CPR | ATGAGGCTACCCCTGCTCCTGGTTTTTGCCTCTGTCATTCCGGGTGCTG | MRLPLLLVFASVIPGAVLDIQMTQSPSSLSASVGDRVTITCRAS |
| | TTCTCGACATCCAGATGACCCAGAGCCCCTCCTCTTTATCCGCCTCTGT | QEISGYLSWLQQKPGKAIKRLIYAASTLQSGVPSRFSGSRSGSD |
| | GGGCGACAGAGTGACCATCACTTGTCGTGCCAGCCAAGAAATCTCCGGC | YTLTISSLQPEDFATYYCLQYASYPFTFGQGTKLEIKGGGGSGG |
| | TATCTGTCTTGGCTGCAGCAGAAGCCCGGTAAGGCTATCAAGAGACTCA | GGSGGGSGGGGSQVTLKESGPTLVKPTQTLTLTCTFSGFSLSTS |
| | TCTACGCCGCCAGCACTTTACAGAGCGGAGTGCCTAGCAGATTTAGCGG | TMGVGWIRQPPGKALEWLAHILWNDSKRYNPSLKSRLTITKDTS |
| | CTCTCGTAGCGGCAGCGATTATACTTTAACCATCTCCTCTTTACAGCCC | KKQVVLTMTNMDPVDTATYYCARIVYYSTYVGYFDVWGQGTTVT |
| | GAAGACTTTGCTACCTACTACTGTTTACAGTACGCCAGCTACCCCTTCA | VSSGSFVPVFLPAKPTTTPAPRPPTPAPTIASQPLSLRPEACRP |
| | CCTTCGGTCAAGGTACCAAGCTGGAGATCAAAGGTGGCGGCGGCTCTGG | AAGGAVHTRGLDFACDIDAKPTHELLTTKADTRKMDPSKPSSNV |
| | CGGCGGTGGCTCTGGCGGTGGCTCCGGCGGTGGTGGTAGCCAAGTTACC | AGVVIIVILLILTGAGLAAYFFYKKRRVHLPQEGAFENTLYENS |
| | CTCAAGGAGAGCGGCCCCACTTTAGTGAAGCCTACCCAGACTTTAACTT | QSSPGTSDMKDLVGNIEQNEHSVI |
| | TAACTTGTACCTTCAGCGGCTTCTCTTTAAGCACCTCCACAATGGGCGT | |
| | GGGCTGGATCAGACAGCCTCCCGGCAAGGCTCTGGAGTGGCTGGCCCAC | |
| | ATCCTCTGGAACGACAGCAAGAGGTACAACCCCTCTTTAAAGTCTCGTC | |
| | TGACCATCACCAAGGACACCTCCAAGAAGCAAGTTGTGCTGACCATGAC | |
| | CAATATGGACCCCGTGGACACCGCCACCTATTACTGCGCTCGTATCGTG | |
| | TACTACTCCACCTACGTGGGCTACTTCGACGTCTGGGGACAAGGTACCA | |
| | CCGTGACCGTGAGCTCTGGATCCTTCGTGCCGGTCTTCCTGCCAGCGAA | |
| | GCCCACCACGACGCCAGCGCCGCGACCACCAACACCGGCGCCCACCATC | |
| | GCGTCGCAGCCCCTGTCCCTGCGCCCAGAGGCGTGCCGGCCAGCGGCGG | |
| | GGGGCGCAGTGCACACGAGGGGGCTGGACTTCGCCTGTGATATTGATGC | |
| | TAAACCTACTCATGAATTACTTACAACAAAAGCTGACACAAGGAAGATG | |
| | GACCCTTCTAAACCGTCTTCCAACGTGGCCGGAGTAGTCATCATTGTGA | |
| | TCCTCCTGATTTTAACGGGTGCTGGCCTTGCCGCCTATTTCTTTTATAA | |
| | GAAAAGACGTGTGCACCTACCTCAAGAGGGCGCCTTTGAAAACACTCTG | |
| | TATTTTAACAGTCAGTCAAGCCCAGGAACTAGTGATATGAAAGATCTCG | |
| | TGGGCAATATTGAACAGAATGAACACTCGGTCATC | |
| B1-sc-MER | SEQ ID NO: 74: | SEQ ID NO: 92: |
| CPR | ATGAGGCTACCCCTGCTCCTGGTTTTTGCCTCTGTCATTCCGGGTGCTG | MRLPLLLVFASVIPGAVLDIQMTQSPSSLSASVGDRVTITCRAS |
| | TTCTCGACATCCAGATGACCCAGAGCCCCTCCTCTTTATCCGCCTCTGT | QEISGYLSWLQQKPGKAIKRLIYAASTLQSGVPSRFSGSRSGSD |
| | GGGCGACAGAGTGACCATCACTTGTCGTGCCAGCCAAGAAATCTCCGGC | YTLTISSLOPEDFATYYCLQYASYPFTFGQGTKLEIKGGGGSGG |
| | TATCTGTCTTGGCTGCAGCAGAAGCCCGGTAAGGCTATCAAGAGACTCA | GGSGGGSGGGGSQVTLKESGPTLVKPTQTLTLTCTFSGFSLSTS |
| | TCTACGCCGCCAGCACTTTACAGAGCGGAGTGCCTAGCAGATTTAGCGG | TMGVGWIRQPPGKALEWLAHILWNDSKRYNPSLKSRLTITKDTS |
| | CTCTCGTAGCGGCAGCGATTATACTTTAACCATCTCCTCTTTACAGCCC | KKQVVLTMTNMDPVDTATYYCARIVYYSTYVGYFDVWGQGTTVT |
| | GAAGACTTTGCTACCTACTACTGTTTACAGTACGCCAGCTACCCCTTCA | VSSGSFVPVFLPAKPTTTPAPRPPTPAPTIASQPLSLRPEACRP |
| | CCTTCGGTCAAGGTACCAAGCTGGAGATCAAAGGTGGCGGCGGCTCTGG | AAGGAVHTRGLDFACDFGCFCGFILIGLILYISLAIRKRVQETK |
| | CGGCGGTGGCTCTGGCGGTGGCTCCGGCGGTGGTGGTAGCCAAGTTACC | FGNAFTEEDSELVVNYIAKKSFCRRAIELTLHSLGVSEELQNKL |
| | CTCAAGGAGAGCGGCCCCACTTTAGTGAAGCCTACCCAGACTTTAACTT | EDVVIDRNLLILGKILGEGEFGSVMEGNLKQEDGTSLKVAVKTM |
| | TAACTTGTACCTTCAGCGGCTTCTCTTTAAGCACCTCCACAATGGGCGT | KLDNSSQREIEEFLSEAACMKDFSHPNVIRLLGVCIEMSSQGIP |
| | GGGCTGGATCAGACAGCCTCCCGGCAAGGCTCTGGAGTGGCTGGCCCAC | KPMVILPFMKYGDLHTYLLYSRLETGPKHIPLQTLLKFMVDIAL |
| | ATCCTCTGGAACGACAGCAAGAGGTACAACCCCTCTTTAAAGTCTCGTC | GMEYLSNRNFLHRDLAARNCMLRDDMTVCVADFGLSKKIYSGDY |
| | TGACCATCACCAAGGACACCTCCAAGAAGCAAGTTGTGCTGACCATGAC | YRQGRIAKMPVKWIAIESLADRVYTSKSDVWAFGVTMWEIATRG |
| | CAATATGGACCCCGTGGACACCGCCACCTATTACTGCGCTCGTATCGTG | MTPYPGVQNHEMYDYLLHGHRLKQPEDCLDELYEIMYSCWRTDP |
| | TACTACTCCACCTACGTGGGCTACTTCGACGTCTGGGGACAAGGTACCA | LDRPTFSVLRLQLEKLLESLPDVRNQADVIYVNTQLLESSEGLA |
| | CCGTGACCGTGAGCTCTGGATCCTTCGTGCCGGTCTTCCTGCCAGCGAA | QGSTLAPLDLNIDPDSIIASCTPRAAISVVTAEVHDSKPHEGRY |
| | GCCCACCACGACGCCAGCGCCGCGACCACCAACACCGGCGCCCACCATC | ILNGGSEEWEDLTSAPSAAVTAEKNSVLPGERLVRNGVSWSHSS |
| | GCGTCGCAGCCCCTGTCCCTGCGCCCAGAGGCGTGCCGGCCAGCGGCGG | MLPLGSSLPDELLFADDSSEGSEVLM |
| | GGGGCGCAGTGCACACGAGGGGGCTGGACTTCGCCTGTGATTTTGGATG | |
| | TTTTTGTGGTTTCATCCTCATCGGTTTGATATTGTACATAAGTCTGGCG | |
| | ATAAGGAAGAGAGTTCAAGAGACAAAGTTCGGAAATGCCTTTACAGAGG | |
| | AAGACAGTGAGCTCGTTGTAAACTACATCGCAAAAAAAAGCTTCTGTAG | |
| | AAGAGCAATAGAGCTCACGTTGCACTCACTCGGTGTGTCCGAAGAACTC | |
| | CAGAATAAACTGGAAGACGTCGTTATCGATCGGAACCTCCTCATACTTG | |
| | GAAAAATACTGGGAGAAGGAGAGTTCGGCAGTGTCATGGAAGGTAACTT | |
| | GAAACAAGAGGATGGTACCTCACTCAAGGTAGCTGTCAAGACGATGAAA | |
| | CTTGATAACAGTTCACAAAGGGAGATCGAAGAATTTCTGTCTGAGGCCG | |
| | CCTGTATGAAAGACTTCTCACATCCTAATGTCATCAGACTTCTTGGCGT | |
| | TTGTATCGAGATGTCTAGCCAAGGAATCCCAAAACCTATGGTCATATTG | |
| | CCTTTCATGAAATATGGCGATCTGCATACATATTTGCTCTACTCTAGAC | |
| | TTGAGACAGGGCCCAAACATATTCCTCTCCAGACATTGCTCAAGTTTAT | |
| | GGTCGATATTGCCCTGGGTATGGAGTACTTGAGCAACCGAAATTTTCTG | |
| | CATCGGGATCTTGCCGCACGCAACTGCATGCTGCGCGATGACATGACCG | |
| | TCTGCGTGGCTGATTTTGGGCTGTCAAAAAAAATATATTCTGGAGACTA | |
| | CTACCGACAAGGGCGGATTGCAAAGATGCCCGTCAAATGGATTGCGATT | |
| | GAAAGTTTGGCGGACAGGGTATATACTTCCAAATCAGATGTTTGGGCTT | |
| | TTGGAGTCACTATGTGGGAAATAGCTACACGCGGTATGACCCCGTACCC | |
| | CGGAGTACAAAATCATGAAATGTATGACTATCTCCTTCATGGACACAGG | |
| | CTGAAGCAGCCCGAGGACTGCCTGGACGAACTGTATGAAATAATGTATT | |
| | CTTGTTGGCGAACCGATCCCTTGGACCGGCCTACTTTCAGTGTCCTTAG | |
| | ATTGCAACTTGAGAAATTGCTCGAGTCTTTGCCGGATGTGCGAAACCAG | |
| | GCAGACGTGATCTATGTCAATACCCAACTTTTGGAAAGTTCTGAGGGCC | |
| | TCGCACAGGGTTCTACCCTTGCCCCGTTGGATCTTAACATAGACCCAGA | |
| | CAGCATAATTGCTTCTTGTACACCTCGCGCTGCCATATCAGTTGTAACA | |
| | GCGGAGGTCCATGATAGTAAACCTCACGAGGGTCGCTATATCCTGAACG | |
| | GCGGGTCAGAAGAATGGGAAGACCTGACATCAGCGCCGAGCGCCGCCGT | |
| | TACTGCTGAGAAAAACTCTGTCCTGCCCGGAGAGCGCTTGGTTCGGAAC | |
| | GGGGTAAGTTGGAGCCATAGCTCAATGCTCCCCCTGGGTTCAAGTCTCC | |
| | CGGACGAGCTTCTTTTTGCGGACGACTCATCTGAGGGGTCCGAAGTTCT | |
| | GATG | |
| B3-sc-MEG | SEQ ID NO: 75: | SEQ ID NO: 93: |
| CPR | ATGAGGCTACCCCTGCTCCTGGTTTTTGCCTCTGTCATTCCGGGTGCTG | MRLPLLLVFASVIPGAVLDIQMTQSPSSLSASVGDRVTITCRAS |
| | TTCTCGACATCCAGATGACCCAGAGCCCCTCCTCTTTATCCGCCTCTGT | QEISGYLSWLQQKPGKAIKRLIYAASTLQSGVPSRFSGSRSGSD |
| | GGGCGACAGAGTGACCATCACTTGTCGTGCCAGCCAAGAAATCTCCGGC | YTLTISSLOPEDFATYYCLQYASYPFTFGQGTKLEIKGGGGSGG |
| | TATCTGTCTTGGCTGCAGCAGAAGCCCGGTAAGGCTATCAAGAGACTCA | GGSGGGSGGGGSQVTLKESGPTLVKPTQTLTLTCTFSGFSLSTS |
| | TCTACGCCGCCAGCACTTTACAGAGCGGAGTGCCTAGCAGATTTAGCGG | TMGVGWIRQPPGKALEWLAHILWNDSKRYNPSLKSRLTITKDTS |
| | CTCTCGTAGCGGCAGCGATTATACTTTAACCATCTCCTCTTTACAGCCC | KKQVVLTMTNMDPVDTATYYCARIVYYSTYVGYFDVWGQGTTVT |
| | GAAGACTTTGCTACCTACTACTGTTTACAGTACGCCAGCTACCCCTTCA | VSSGSFVPVFLPAKPTTTPAPRPPTPAPTIASQPLSLRPEACRP |
| | CCTTCGGTCAAGGTACCAAGCTGGAGATCAAAGGTGGCGGCGGCTCTGG | AAGGAVHTRGLDFACDAIAGIIILVLVVLFLLALFIIYRHKQKG |
| | CGGCGGTGGCTCTGGCGGTGGCTCCGGCGGTGGTGGTAGCCAAGTTACC | KESSMPAVTYTPAMRVVNADYTISGTLPHSNGGNANSHYFTNPS |
| | CTCAAGGAGAGCGGCCCCACTTTAGTGAAGCCTACCCAGACTTTAACTT | YHTLTQCATSPHVNNRDRMTVTKSKNNQLFVNLKNVNPGKRGPV |
| | TAACTTGTACCTTCAGCGGCTTCTCTTTAAGCACCTCCACAATGGGCGT | GDCTGTLPADWKHGGYLNELGAFGLDRSYMGKSLKDLGKNSEYN |
| | GGGCTGGATCAGACAGCCTCCCGGCAAGGCTCTGGAGTGGCTGGCCCAC | SSNCSLSSSENPYATIKDPPVLIPKSSECGYVEMKSPARRDSPY |
| | ATCCTCTGGAACGACAGCAAGAGGTACAACCCCTCTTTAAAGTCTCGTC | AEINNSTSANRNVYEVEPTVSVVQGVFSNNGRLSQDPYDLPKNS |
| | TGACCATCACCAAGGACACCTCCAAGAAGCAAGTTGTGCTGACCATGAC | HIPCHYDLLPVRDSSSSPKQEDSGGSSSNSSSSSE |
| | CAATATGGACCCCGTGGACACCGCCACCTATTACTGCGCTCGTATCGTG | |
| | TACTACTCCACCTACGTGGGCTACTTCGACGTCTGGGGACAAGGTACCA | |
| | CCGTGACCGTGAGCTCTGGATCCTTCGTGCCGGTCTTCCTGCCAGCGAA | |
| | GCCCACCACGACGCCAGCGCCGCGACCACCAACACCGGCGCCCACCATC | |
| | GCGTCGCAGCCCCTGTCCCTGCGCCCAGAGGCGTGCCGGCCAGCGGCGG | |
| | GGGGCGCAGTGCACACGAGGGGGCTGGACTTCGCCTGTGATGCTATCGC | |
| | GGGGATCATTATATTGGTCTTGGTGGTGCTGTTTCTGCTCGCGCTTTTC | |
| | ATTATATACCGCCATAAGCAGAAGGGCAAAGAGTCCTCCATGCCAGCCG | |
| | TGACCTATACGCCTGCGATGCGCGTTGTCAACGCCGATTACACCATCAG | |
| | TGGTACCCTCCCGCACAGTAACGGCGGAAATGCAAACTCTCATTACTTT | |
| | ACAAATCCTAGTTACCATACACTCACTCAGTGTGCTACCTCTCCCCATG | |
| | TGAACAATCGGGACAGGATGACCGTTACGAAAAGCAAAAATAACCAGTT | |
| | GTTTGTGAACCTTAAGAATGTGAATCCCGGCAAGAGGGGTCCGGTGGGT | |
| | GACTGCACCGGAACTCTCCCCGCTGACTGGAAGCATGGCGGGTACCTGA | |
| | ACGAACTCGGCGCGTTTGGGCTCGACCGAAGCTACATGGGTAAAAGTCT | |
| | TAAGGACCTCGGTAAGAATAGCGAGTATAATAGCTCTAACTGTTCCCTT | |
| | TCCAGCTCCGAGAATCCGTACGCTACTATAAAAGACCCCCCGGTGCTCA | |
| | TTCCCAAATCCAGTGAGTGCGGGTACGTGGAGATGAAAAGTCCCGCTCG | |
| | AAGAGACAGTCCATACGCGGAAATCAATAACTCCACCAGTGCGAACCGC | |
| | AATGTGTACGAAGTGGAGCCCACCGTTTCCGTTGTACAAGGTGTATTTT | |
| | CAAACAATGGGAGGCTTAGCCAGGACCCCTATGATCTTCCAAAGAACAG | |
| | CCACATCCCGTGTCATTATGATCTGTTGCCGGTGAGGGATTCTAGCTCT | |
| | TCTCCTAAACAAGAGGACTCAGGTGGCTCTAGTTCCAACTCCTCCAGTT | |
| | CTTCAGAG | |
| B5-sc-Dec | SEQ ID NO: 76: | SEQ ID NO: 94: |
| CPR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGFVPVFLPAKPTTTPAPRPPTPAP |
| | AAAGGGCAGTTGTGCTGCCAGTCCACCGTGGCGGTTGATTGCCGTCATC | TIASQPLSLRPEACRPAAGGAVHTRGLDFACDGSDIQMTQSPSS |
| | CTCGGCATTCTGTGCTTGGTTATTCTCGTTATAGCGGTGGTGCTTGGGT | LSASVGDRVTITCRASQEISGYLSWLQQKPGKAIKRLIYAASTL |
| | TCGTGCCGGTCTTCCTGCCAGCGAAGCCCACCACGACGCCAGCGCCGCG | QSGVPSRFSGSRSGSDYTLTISSLOPEDFATYYCLQYASYPFTF |
| | ACCACCAACACCGGCGCCCACCATCGCGTCGCAGCCCCTGTCCCTGCGC | GQGTKLEIKGGGGSGGGGSGGGSGGGGSQVTLKESGPTLVKPTQ |
| | CCAGAGGCGTGCCGGCCAGCGGCGGGGGGCGCAGTGCACACGAGGGGGC | TLTLTCTFSGFSLSTSTMGVGWIRQPPGKALEWLAHILWNDSKR |
| | TGGACTTCGCCTGTGATGGATCCGACATCCAGATGACCCAGAGCCCCTC | YNPSLKSRLTITKDTSKKQVVLTMTNMDPVDTATYYCARIVYYS |
| | CTCTTTATCCGCCTCTGTGGGCGACAGAGTGACCATCACTTGTCGTGCC | TYVGYFDVWGQGTTVTVSS |
| | AGCCAAGAAATCTCCGGCTATCTGTCTTGGCTGCAGCAGAAGCCCGGTA | |
| | AGGCTATCAAGAGACTCATCTACGCCGCCAGCACTTTACAGAGCGGAGT | |
| | GCCTAGCAGATTTAGCGGCTCTCGTAGCGGCAGCGATTATACTTTAACC | |
| | ATCTCCTCTTTACAGCCCGAAGACTTTGCTACCTACTACTGTTTACAGT | |
| | ACGCCAGCTACCCCTTCACCTTCGGTCAAGGTACCAAGCTGGAGATCAA | |
| | AGGTGGCGGCGGCTCTGGCGGCGGTGGCTCTGGCGGTGGCTCCGGCGGT | |
| | GGTGGTAGCCAAGTTACCCTCAAGGAGAGCGGCCCCACTTTAGTGAAGC | |
| | CTACCCAGACTTTAACTTTAACTTGTACCTTCAGCGGCTTCTCTTTAAG | |
| | CACCTCCACAATGGGCGTGGGCTGGATCAGACAGCCTCCCGGCAAGGCT | |
| | CTGGAGTGGCTGGCCCACATCCTCTGGAACGACAGCAAGAGGTACAACC | |
| | CCTCTTTAAAGTCTCGTCTGACCATCACCAAGGACACCTCCAAGAAGCA | |
| | AGTTGTGCTGACCATGACCAATATGGACCCCGTGGACACCGCCACCTAT | |
| | TACTGCGCTCGTATCGTGTACTACTCCACCTACGTGGGCTACTTCGACG | |
| | TCTGGGGACAAGGTACCACCGTGACCGTGAGCTCT | |
| B7-sc- | SEQ ID NO: 77: | SEQ ID NO: 95: |
| DecFull CPR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGTMAIWRSNSGSNTLENGYELSRN |
| | AAAGGGCAGTTGTGCTGCCAGTCCACCGTGGCGGTTGATTGCCGTCATC | KENHSQPTQSSLEDSVTPTKAVKTTGVLSSPCPPNWIIYEKSCY |
| | CTCGGCATTCTGTGCTTGGTTATTCTCGTTATAGCGGTGGTGCTTGGGA | LFSMSLNSWDGSKRQCWQLGSNLLKIDSSNELGFIVKQVSSQPD |
| | CCATGGCGATCTGGCGCTCCAACTCTGGAAGTAACACCCTTGAAAATGG | NSFWIGLSRPQTEVPWLWEDGSTFSSNLFQIRTTATQENPSPNC |
| | TTACTTCCTCAGTAGGAACAAAGAGAACCATTCCCAGCCGACACAGTCA | VWIHVSVIYDQLCSVPSYSICEKKFSMGSDIQMTQSPSSLSASV |
| | AGCCTTGAAGATTCAGTCACCCCTACAAAGGCCGTAAAAACGACAGGTG | GDRVTITCRASQEISGYLSWLQQKPGKAIKRLIYAASTLQSGVP |
| | TCCTGTCCTCTCCGTGTCCGCCTAACTGGATCATCTACGAGAAAAGTTG | SRFSGSRSGSDYTLTISSLOPEDFATYYCLQYASYPFTFGQGTK |
| | TTATCTGTTTAGCATGAGCCTTAACAGTTGGGATGGCTCAAAAAGGCAG | LEIKGGGGSGGGGSGGGSGGGGSQVTLKESGPTLVKPTQTLTLT |
| | TGCTGGCAACTGGGGAGCAACCTTTTGAAGATAGACAGTTCCAACGAAC | CTFSGFSLSTSTMGVGWIRQPPGKALEWLAHILWNDSKRYNPSL |
| | TGGGCTTCATAGTCAAACAGGTGTCCTCTCAACCTGATAACTCATTCTG | KSRLTITKDTSKKQVVLTMTNMDPVDTATYYCARIVYYSTYVGY |
| | GATCGGGCTCAGTCGACCCCAAACTGAGGTTCCATGGCTTTGGGAAGAC | FDVWGQGTTVTVSS |
| | GGCAGCACTTTCTCTTCAAATTTGTTTCAAATAAGAACCACCGCTACGC | |
| | AGGAGAATCCGAGTCCGAACTGTGTTTGGATTCACGTCTCAGTCATTTA | |
| | CGACCAACTTTGTAGTGTCCCTAGCTATTCCATCTGCGAGAAAAAGTTC | |
| | AGTATGGGATCCGACATCCAGATGACCCAGAGCCCCTCCTCTTTATCCG | |
| | CCTCTGTGGGCGACAGAGTGACCATCACTTGTCGTGCCAGCCAAGAAAT | |
| | CTCCGGCTATCTGTCTTGGCTGCAGCAGAAGCCCGGTAAGGCTATCAAG | |
| | AGACTCATCTACGCCGCCAGCACTTTACAGAGCGGAGTGCCTAGCAGAT | |
| | TTAGCGGCTCTCGTAGCGGCAGCGATTATACTTTAACCATCTCCTCTTT | |
| | ACAGCCCGAAGACTTTGCTACCTACTACTGTTTACAGTACGCCAGCTAC | |
| | CCCTTCACCTTCGGTCAAGGTACCAAGCTGGAGATCAAAGGTGGCGGCG | |
| | GCTCTGGCGGCGGTGGCTCTGGCGGTGGCTCCGGCGGTGGTGGTAGCCA | |
| | AGTTACCCTCAAGGAGAGCGGCCCCACTTTAGTGAAGCCTACCCAGACT | |
| | TTAACTTTAACTTGTACCTTCAGCGGCTTCTCTTTAAGCACCTCCACAA | |
| | TGGGCGTGGGCTGGATCAGACAGCCTCCCGGCAAGGCTCTGGAGTGGCT | |
| | GGCCCACATCCTCTGGAACGACAGCAAGAGGTACAACCCCTCTTTAAAG | |
| | TCTCGTCTGACCATCACCAAGGACACCTCCAAGAAGCAAGTTGTGCTGA | |
| | CCATGACCAATATGGACCCCGTGGACACCGCCACCTATTACTGCGCTCG | |
| | TATCGTGTACTACTCCACCTACGTGGGCTACTTCGACGTCTGGGGACAA | |
| | GGTACCACCGTGACCGTGAGCTCT | |
| B9-sc-163 | SEQ ID NO: 78: | SEQ ID NO: 96: |
| CPR | ATGAGGCTACCCCTGCTCCTGGTTTTTGCCTCTGTCATTCCGGGTGCTG | MRLPLLLVFASVIPGAVLDIQMTQSPSSLSASVGDRVTITCRAS |
| | TTCTCGACATCCAGATGACCCAGAGCCCCTCCTCTTTATCCGCCTCTGT | QEISGYLSWLQQKPGKAIKRLIYAASTLQSGVPSRFSGSRSGSD |
| | GGGCGACAGAGTGACCATCACTTGTCGTGCCAGCCAAGAAATCTCCGGC | YTLTISSLQPEDFATYYCLQYASYPFTFGQGTKLEIKGGGGSGG |
| | TATCTGTCTTGGCTGCAGCAGAAGCCCGGTAAGGCTATCAAGAGACTCA | GGSGGGSGGGGSQVTLKESGPTLVKPTQTLTLTCTFSGFSLSTS |
| | TCTACGCCGCCAGCACTTTACAGAGCGGAGTGCCTAGCAGATTTAGCGG | TMGVGWIRQPPGKALEWLAHILWNDSKRYNPSLKSRLTITKDTS |
| | CTCTCGTAGCGGCAGCGATTATACTTTAACCATCTCCTCTTTACAGCCC | KKQVVLTMTNMDPVDTATYYCARIVYYSTYVGYFDVWGQGTTVT |
| | GAAGACTTTGCTACCTACTACTGTTTACAGTACGCCAGCTACCCCTTCA | VSSGSFVPVFLPAKPTTTPAPRPPTPAPTIASQPLSLRPEACRP |
| | CCTTCGGTCAAGGTACCAAGCTGGAGATCAAAGGTGGCGGCGGCTCTGG | AAGGAVHTRGLDFACDSSLGGTDKELRLVDGENKCSGRVEVKVQ |
| | CGGCGGTGGCTCTGGCGGTGGCTCCGGCGGTGGTGGTAGCCAAGTTACC | EEWGTVCNNGWSMEAVSVICNQLGCPTAIKAPGWANSSAGSGRI |
| | CTCAAGGAGAGCGGCCCCACTTTAGTGAAGCCTACCCAGACTTTAACTT | WMDHVSCRGNESALWDCKHDGWGKHSNCTHQQDAGVTCSDGSNL |
| | TAACTTGTACCTTCAGCGGCTTCTCTTTAAGCACCTCCACAATGGGCGT | EMRLTRGGNMCSGRIEIKFQGRWGTVCDDNFNIDHASVICRQLE |
| | GGGCTGGATCAGACAGCCTCCCGGCAAGGCTCTGGAGTGGCTGGCCCAC | CGSAVSFSGSSNFGEGSGPIWFDDLICNGNESALWNCKHQGWGK |
| | ATCCTCTGGAACGACAGCAAGAGGTACAACCCCTCTTTAAAGTCTCGTC | HNCDHAEDAGVICSKGADLSLRLVDGVTECSGRLEVRFQGEWGT |
| | TGACCATCACCAAGGACACCTCCAAGAAGCAAGTTGTGCTGACCATGAC | ICDDGWDSYDAAVACKQLGCPTAVTAIGRVNASKGFGHIWLDSV |
| | CAATATGGACCCCGTGGACACCGCCACCTATTACTGCGCTCGTATCGTG | SCQGHEPAIWQCKHHEWGKHYCNHNEDAGVTCSDGSDLELRLRG |
| | TACTACTCCACCTACGTGGGCTACTTCGACGTCTGGGGACAAGGTACCA | GGSRCAGTVEVEIQRLLGKVCDRGWGLKEADVVCRQLGCGSALK |
| | CCGTGACCGTGAGCTCTGGATCCTTCGTGCCGGTCTTCCTGCCAGCGAA | TSYQVYSKIQATNTWLFLSSCNGNETSLWDCKNWOWGGLTCDHY |
| | GCCCACCACGACGCCAGCGCCGCGACCACCAACACCGGCGCCCACCATC | EEAKITCSAHREPRLVGGDIPCSGRVEVKHGDTWGSICDSDFSL |
| | GCGTCGCAGCCCCTGTCCCTGCGCCCAGAGGCGTGCCGGCCAGCGGCGG | EAASVLCRELQCGTVVSILGGAHFGEGNGQIWAEEFQCEGHESH |
| | GGGGCGCAGTGCACACGAGGGGGCTGGACTTCGCCTGTGATTCATCCCT | LSLCPVAPRPEGTCSHSRDVGVVCSRYTEIRLVNGKTPCEGRVE |
| | GGGTGGAACAGATAAGGAGTTGAGGTTGGTGGATGGAGAGAATAAGTGC | LKTLGAWGSLCNSHWDIEDAHVLCQQLKCGVALSTPGGARFGKG |
| | AGTGGACGGGTTGAGGTCAAGGTGCAGGAAGAGTGGGGTACTGTTTGTA | NGQIWRHMFHCTGTEQHMGDCPVTALGASLCPSEQVASVICSGN |
| | ACAACGGCTGGAGTATGGAAGCAGTCTCCGTGATATGCAATCAACTTGG | QSQTLSSCNSSSLGPTRPTIPEESAVACIESGQLRLVNGGGRCA |
| | TTGCCCTACAGCCATAAAAGCTCCTGGATGGGCTAACTCATCTGCCGGA | GRVEIYHEGSWGTICDDSWDLSDAHVVCRQLGCGEAINATGSAH |
| | AGCGGTCGAATATGGATGGACCATGTATCCTGTAGGGGCAATGAGTCAG | FGEGTGPIWLDEMKCNGKESRIWQCHSHGWGQQNCRHKEDAGVI |
| | CACTGTGGGACTGCAAGCATGACGGGTGGGGAAAACACTCTAATTGTAC | CSEFMSLRLTSEASREACAGRLEVFYNGAWGTVGKSSMSETTVG |
| | CCACCAGCAAGATGCCGGTGTGACGTGTAGCGACGGCAGCAACTTGGAG | VVCRQLGCADKGKINPASLDKAMSIPMWVDNVQCPKGPDTLWQC |
| | ATGCGGTTGACGCGCGGCGGGAACATGTGCAGCGGTCGCATTGAGATAA | PSSPWEKRLASPSEETWITCDNKIRLQEGPTSCSGRVEIWHGGS |
| | AGTTCCAAGGGCGCTGGGGGACCGTTTGTGACGACAACTTCAATATAGA | WGTVCDDSWDLDDAQVVCQQLGCGPALKAFKEAEFGQGTGPIWL |
| | TCATGCTAGTGTGATATGCAGGCAGTTGGAGTGCGGTAGCGCCGTCTCA | NEVKCKGNESSLWDCPARRWGHSECGHKEDAAVNCTDISVQKTP |
| | TTTAGTGGTTCTAGCAATTTCGGTGAGGGATCCGGGCCTATATGGTTCG | QKATTGRSSRQSSFIAVGILGVVLLAIFVALFFLTKKRRORQRL |
| | ACGACCTTATCTGCAATGGAAATGAGAGCGCCCTCTGGAACTGCAAGCA | AVSSRGENLVHQIQYREMNSCLNADDLDLMNSSENSHESADFSA |
| | CCAAGGTTGGGGTAAGCATAACTGCGATCATGCTGAGGATGCGGGTGTG | AELISVSKFLPISGMEKEAILSHTEKENGNL |
| | ATTTGTAGCAAGGGAGCCGACTTGTCACTTCGACTCGTAGATGGCGTGA | |
| | CGGAATGTAGCGGTCGCCTTGAGGTACGCTTCCAAGGTGAATGGGGCAC | |
| | AATCTGTGATGACGGTTGGGACTCATATGACGCTGCGGTTGCTTGCAAA | |
| | CAGCTGGGCTGTCCGACAGCGGTAACGGCAATTGGGCGGGTAAATGCCA | |
| | GTAAGGGGTTTGGACATATATGGTTGGACAGTGTTTCTTGTCAGGGACA | |
| | CGAACCTGCAATATGGCAATGTAAACATCACGAGTGGGGGAAGCATTAC | |
| | TGCAATCATAATGAAGACGCAGGCGTCACATGCTCTGACGGATCCGACC | |
| | TGGAATTGCGGCTGAGAGGGGGGGGTTCACGGTGCGCAGGAACCGTGGA | |
| | AGTTGAGATTCAACGCCTGCTCGGGAAAGTTTGTGACAGGGGGTGGGGC | |
| | CTTAAGGAAGCAGACGTAGTCTGCCGACAACTGGGTTGCGGGAGCGCCC | |
| | TCAAGACGTCCTATCAAGTTTACAGCAAAATTCAAGCAACTAATACCTG | |
| | GCTGTTTCTTTCCTCCTGCAATGGTAACGAAACGAGCCTCTGGGATTGT | |
| | AAAAATTGGCAATGGGGAGGCCTTACATGTGATCACTATGAAGAGGCCA | |
| | AAATCACCTGCAGCGCGCACCGAGAGCCTAGGCTGGTTGGAGGGGATAT | |
| | TCCCTGTTCAGGTAGAGTTGAAGTCAAGCATGGTGATACCTGGGGGTCC | |
| | ATATGCGACTCCGATTTCAGTTTGGAAGCAGCGAGCGTGCTGTGCCGAG | |
| | AGCTTCAATGTGGGACAGTAGTTTCCATTCTTGGTGGCGCCCACTTCGG | |
| | GGAAGGTAACGGACAGATTTGGGCGGAGGAATTCCAATGTGAGGGCCAT | |
| | GAAAGTCACCTGAGTCTTTGTCCTGTAGCGCCTCGGCCGGAAGGTACTT | |
| | GTTCTCACTCCAGAGACGTTGGCGTGGTTTGTTCTAGGTACACTGAGAT | |
| | AAGGCTGGTGAATGGCAAAACGCCTTGTGAAGGAAGGGTGGAGCTCAAA | |
| | ACGCTTGGCGCCTGGGGGTCTCTGTGCAACTCCCACTGGGACATAGAGG | |
| | ACGCACATGTCTTGTGCCAGCAACTCAAATGCGGTGTCGCGCTTTCAAC | |
| | CCCTGGGGGCGCTAGATTCGGGAAAGGTAACGGCCAGATATGGCGCCAT | |
| | ATGTTCCATTGCACCGGAACTGAACAGCATATGGGAGATTGTCCTGTGA | |
| | CTGCCTTGGGGGCAAGTCTGTGTCCCTCAGAACAAGTGGCTTCAGTTAT | |
| | TTGCAGCGGTAACCAGAGTCAGACGCTCAGCTCCTGCAACAGCAGCAGT | |
| | CTGGGTCCAACAAGACCCACAATACCCGAGGAGTCAGCGGTCGCGTGCA | |
| | TCGAATCCGGGCAATTGAGACTCGTTAATGGCGGGGGTCGGTGCGCAGG | |
| | GCGCGTGGAGATCTACCACGAGGGTAGTTGGGGCACAATTTGTGACGAC | |
| | AGCTGGGACCTTTCCGACGCTCATGTCGTATGCCGACAACTGGGGTGCG | |
| | GTGAGGCCATCAATGCCACCGGAAGCGCGCATTTCGGTGAAGGCACGGG | |
| | CCCAATTTGGCTTGATGAGATGAAATGTAATGGAAAGGAGTCCCGCATT | |
| | TGGCAATGCCATAGCCATGGCTGGGGTCAACAAAATTGTCGACACAAAG | |
| | AAGATGCCGGGGTGATCTGCTCAGAATTCATGTCCTTGCGGCTTACTAG | |
| | CGAAGCGTCCCGCGAGGCCTGTGCTGGGAGACTGGAAGTTTTTTATAAC | |
| | GGGGCTTGGGGAACGGTTGGTAAGTCATCAATGAGTGAAACCACAGTTG | |
| | GTGTTGTGTGTAGACAACTCGGTTGCGCCGACAAGGGAAAGATCAACCC | |
| | GGCGAGCCTTGATAAGGCCATGAGCATCCCCATGTGGGTGGATAACGTT | |
| | CAGTGTCCGAAAGGTCCCGATACCTTGTGGCAATGCCCAAGTTCTCCTT | |
| | GGGAGAAGAGACTCGCCTCACCAAGTGAAGAGACGTGGATTACGTGTGA | |
| | TAACAAAATTAGGCTCCAAGAAGGACCGACCAGTTGCAGCGGAAGAGTT | |
| | GAGATATGGCATGGAGGAAGCTGGGGAACCGTGTGCGATGACAGCTGGG | |
| | ACCTGGACGACGCCCAGGTGGTGTGCCAACAGCTGGGTTGCGGGCCTGC | |
| | CCTCAAAGCATTTAAGGAAGCCGAATTCGGTCAGGGTACTGGGCCAATC | |
| | TGGCTGAACGAGGTAAAGTGCAAAGGTAACGAAAGTAGCCTGTGGGACT | |
| | GTCCGGCACGAAGGTGGGGCCACAGCGAGTGTGGCCATAAGGAAGACGC | |
| | GGCCGTGAACTGTACAGACATATCCGTACAAAAAACGCCCCAAAAGGCG | |
| | ACGACCGGGCGATCATCAAGACAATCTAGCTTTATTGCCGTGGGAATTC | |
| | TCGGTGTAGTGCTTCTTGCTATATTTGTCGCTTTGTTCTTTCTGACTAA | |
| | AAAGCGCAGGCAAAGGCAGCGGCTTGCTGTGAGCTCTCGGGGAGAAAAC | |
| | CTCGTTCACCAAATCCAATACCGAGAAATGAACTCCTGTCTCAACGCCG | |
| | ACGATCTTGACCTGATGAACTCATCTGAGAACTCACACGAGTCCGCCGA | |
| | TTTCAGCGCGGCGGAATTGATCTCTGTCAGCAAATTTCTGCCTATAAGT | |
| | GGCATGGAAAAAGAAGCCATACTCTCTCACACGGAAAAGGAAAATGGCA | |
| | ACCTT | |
| F4-sc19 CPR | SEQ ID NO: 79: | SEQ ID NO: 97: |
| | ATGGCCTTACCAGTGACCGCCTTGCTCCTGCCGCTGGCCTTGCTGCTCC | MALPVTALLLPLALLLHAARPGSEVKLQESGPGLVAPSQSLSVT |
| | ACGCCGCCAGGCCGGGATCCGAGGTGAAACTGCAGGAGTCAGGACCTGG | CTVSGVSLPDYGVSWIRQPPRKGLEWLGVIWGSETTYYNSALKS |
| | CCTGGTGGCGCCCTCACAGAGCCTGTCCGTCACATGCACTGTCTCAGGG | RLTIIKDNSKSQVFLKMNSLQTDDTAIYYCAKHYYYGGSYAMDY |
| | GTCTCATTACCCGACTATGGTGTAAGCTGGATTCGCCAGCCTCCACGAA | WGQGTSVTVSSGGGGSGGGGSGGGGSDIQMTQTTSSLSASLGDR |
| | AGGGTCTGGAGTGGCTGGGAGTAATATGGGGTAGTGAAACCACATACTA | VTISCRASQDISKYLNWYQQKPDGTVKLLIYHTSRLHSGVPSRF |
| | TAATTCAGCTCTCAAATCCAGACTGACCATCATCAAGGACAACTCCAAG | SGSGSGTDYSLTISNLEQEDIATYFCQQGNTLPYTFGGGTKLEI |
| | AGCCAAGTTTTCTTAAAAATGAACAGTCTGCAAACTGATGACACAGCCA | TRADAAPTVSIFPPSSNDTRKMDPSKPSSNVAGVVIIVILLILT |
| | TTTACTACTGTGCCAAACATTATTACTACGGTGGTAGCTATGCTATGGA | GAGLAAYFFYKKRRVHLPQEGAFENTLYFNSQSSPGTSDMKDLV |
| | CTACTGGGGTCAAGGAACCTCAGTCACCGTCTCCTCAGGCGGCGGTGGC | GNIEQNEHSVI |
| | TCTGGCGGTGGCGGTTCCGGCGGTGGTGGTAGCGACATCCAGATGACAC | |
| | AGACTACATCCTCCCTGTCTGCCTCTCTGGGAGACAGAGTCACCATCAG | |
| | TTGCAGGGCAAGTCAGGACATTAGTAAATATTTAAATTGGTATCAGCAG | |
| | AAACCAGATGGAACTGTTAAACTCCTGATCTACCATACATCAAGATTAC | |
| | ACTCAGGAGTCCCATCAAGGTTCAGTGGCAGTGGGTCTGGAACAGATTA | |
| | TTCTCTCACCATTAGCAACCTGGAGCAAGAAGATATTGCCACTTACTTT | |
| | TGCCAACAGGGTAATACGCTTCCGTACACGTTCGGAGGGGGGACTAAGT | |
| | TGGAAATAACACGGGCTGATGCTGCACCAACTGTATCCATCTTCCCACC | |
| | ATCCAGTAATGACACAAGGAAGATGGACCCTTCTAAACCGTCTTCCAAC | |
| | GTGGCCGGAGTAGTCATCATTGTGATCCTCCTGATTTTAACGGGTGCTG | |
| | GCCTTGCCGCCTATTTCTTTTATAAGAAAAGACGTGTGCACCTACCTCA | |
| | AGAGGGCGCCTTTGAAAACACTCTGTATTTTAACAGTCAGTCAAGCCCA | |
| | GGAACTAGTGATATGAAAGATCTCGTGGGCAATATTGAACAGAATGAAC | |
| | ACTCGGTCATC | |
| F4-sc20 CPR | SEQ ID NO: 80: | SEQ ID NO: 98: |
| | ATGGCCTTACCAGTGACCGCCTTGCTCCTGCCGCTGGCCTTGCTGCTCC | MALPVTALLLPLALLLHAARPGSQIVLSQSPAILSASPGEKVTM |
| | ACGCCGCCAGGCCGGGATCCCAAATTGTTCTCTCCCAGTCTCCAGCAAT | TCRASSSLSFMHWYQQKPGSSPKPWIYATSNLASGVPARFSGSG |
| | CCTTTCTGCATCTCCAGGGGAGAAGGTCACAATGACTTGCAGGGCCAGC | SGTSYSLTISRVEAEDAATYFCHQWSSNPLTFGAGTKLELKRGG |
| | TCAAGTTTAAGTTTCATGCACTGGTACCAGCAGAAGCCAGGATCCTCCC | GGSGGGGSGGGGSGGGGSQVQLRQPGAELVKPGASVKMSCKASG |
| | CCAAACCCTGGATTTATGCCACATCCAACCTGGCTTCTGGAGTCCCTGC | YTFTSYNMHWVKQTPGQGLEWIGAIYPGNGDTSYNQKFKGKATL |
| | TCGCTTCAGTGGCAGTGGGTCTGGGACCTCTTACTCTCTCACAATCAGC | TADKSSSTAYMQLSSLTSEDSAVYYCARSHYGSNYVDYFDYWGQ |
| | AGAGTGGAGGCTGAAGATGCTGCCACTTATTTCTGCCATCAGTGGAGTA | GTTLTVSSDTRKMDPSKPSSNVAGVVIIVILLILTGAGLAAYFF |
| | GTAACCCGCTCACGTTCGGTGCTGGGACCAAGCTGGAGCTCAAACGGGG | YKKRRVHLPQEGAFENTLYFNSQSSPGTSDMKDLVGNIEQNEHS |
| | TGGCGGCGGCTCTGGCGGCGGTGGCTCTGGCGGTGGCGGTTCCGGCGGT | VI |
| | GGTGGTAGCCAGGTGCAACTGCGGCAGCCTGGGGCTGAGCTGGTGAAGC | |
| | CTGGGGCCTCAGTGAAGATGTCCTGCAAGGCTTCTGGCTACACATTTAC | |
| | CAGTTACAATATGCACTGGGTAAAGCAGACACCTGGACAGGGCCTGGAA | |
| | TGGATTGGAGCTATTTATCCAGGAAATGGTGATACTTCCTACAATCAGA | |
| | AGTTCAAAGGCAAGGCCACATTGACTGCAGACAAATCCTCCAGCACAGC | |
| | CTACATGCAGCTCAGCAGTCTGACATCTGAGGACTCTGCGGTCTATTAC | |
| | TGTGCAAGATCGCACTACGGTAGTAACTACGTAGACTACTTTGACTACT | |
| | GGGGCCAAGGCACCACTCTCACAGTCTCCTCTGACACAAGGAAGATGGA | |
| | CCCTTCTAAACCGTCTTCCAACGTGGCCGGAGTAGTCATCATTGTGATC | |
| | CTCCTGATTTTAACGGGTGCTGGCCTTGCCGCCTATTTCTTTTATAAGA | |
| | AAAGACGTGTGCACCTACCTCAAGAGGGCGCCTTTGAAAACACTCTGTA | |
| | TTTTAACAGTCAGTCAAGCCCAGGAACTAGTGATATGAAAGATCTCGTG | |
| | GGCAATATTGAACAGAATGAACACTCGGTCATC | |
| C1-sc19- | SEQ ID NO: 81: | SEQ ID NO: 99: |
| MER CPR | ATGGCCTTACCAGTGACCGCCTTGCTCCTGCCGCTGGCCTTGCTGCTCC | MALPVTALLLPLALLLHAARPGSEVKLQESGPGLVAPSQSLSVT |
| | ACGCCGCCAGGCCGGGATCCGAGGTGAAACTGCAGGAGTCAGGACCTGG | CTVSGVSLPDYGVSWIRQPPRKGLEWLGVIWGSETTYYNSALKS |
| | CCTGGTGGCGCCCTCACAGAGCCTGTCCGTCACATGCACTGTCTCAGGG | RLTIIKDNSKSQVFLKMNSLOTDDTAIYYCAKHYYYGGSYAMDY |
| | GTCTCATTACCCGACTATGGTGTAAGCTGGATTCGCCAGCCTCCACGAA | WGQGTSVTVSSGGGGSGGGGSGGGGSDIQMTQTTSSLSASLGDR |
| | AGGGTCTGGAGTGGCTGGGAGTAATATGGGGTAGTGAAACCACATACTA | VTISCRASQDISKYLNWYQQKPDGTVKLLIYHTSRLHSGVPSRF |
| | TAATTCAGCTCTCAAATCCAGACTGACCATCATCAAGGACAACTCCAAG | SGSGSGTDYSLTISNLEQEDIATYFCQQGNTLPYTFGGGTKLEI |
| | AGCCAAGTTTTCTTAAAAATGAACAGTCTGCAAACTGATGACACAGCCA | TRADAAPTVSIFPPSSNGSFVPVFLPAKPTTTPAPRPPTPAPTI |
| | TTTACTACTGTGCCAAACATTATTACTACGGTGGTAGCTATGCTATGGA | ASQPLSLRPEACRPAAGGAVHTRGLDFACDFGCFCGFILIGLIL |
| | CTACTGGGGTCAAGGAACCTCAGTCACCGTCTCCTCAGGCGGCGGTGGC | YISLAIRKRVQETKFGNAFTEEDSELVVNYIAKKSFCRRAIELT |
| | TCTGGCGGTGGCGGTTCCGGCGGTGGTGGTAGCGACATCCAGATGACAC | LHSLGVSEELQNKLEDVVIDRNLLILGKILGEGEFGSVMEGNLK |
| | AGACTACATCCTCCCTGTCTGCCTCTCTGGGAGACAGAGTCACCATCAG | QEDGTSLKVAVKTMKLDNSSQREIEEFLSEAACMKDFSHPNVIR |
| | TTGCAGGGCAAGTCAGGACATTAGTAAATATTTAAATTGGTATCAGCAG | LLGVCIEMSSQGIPKPMVILPFMKYGDLHTYLLYSRLETGPKHI |
| | AAACCAGATGGAACTGTTAAACTCCTGATCTACCATACATCAAGATTAC | PLQTLLKFMVDIALGMEYLSNRNFLHRDLAARNCMLRDDMTVCV |
| | ACTCAGGAGTCCCATCAAGGTTCAGTGGCAGTGGGTCTGGAACAGATTA | ADFGLSKKIYSGDYYRQGRIAKMPVKWIAIESLADRVYTSKSDV |
| | TTCTCTCACCATTAGCAACCTGGAGCAAGAAGATATTGCCACTTACTTT | WAFGVTMWEIATRGMTPYPGVQNHEMYDYLLHGHRLKQPEDCLD |
| | TGCCAACAGGGTAATACGCTTCCGTACACGTTCGGAGGGGGGACTAAGT | ELYEIMYSCWRTDPLDRPTFSVLRLQLEKLLESLPDVRNQADVI |
| | TGGAAATAACACGGGCTGATGCTGCACCAACTGTATCCATCTTCCCACC | YVNTQLLESSEGLAQGSTLAPLDLNIDPDSIIASCTPRAAISVV |
| | ATCCAGTAATGGATCCTTCGTGCCGGTCTTCCTGCCAGCGAAGCCCACC | TAEVHDSKPHEGRYILNGGSEEWEDLTSAPSAAVTAEKNSVLPG |
| | ACGACGCCAGCGCCGCGACCACCAACACCGGCGCCCACCATCGCGTCGC | ERLVRNGVSWSHSSMLPLGSSLPDELLFADDSSEGSEVLM |
| | AGCCCCTGTCCCTGCGCCCAGAGGCGTGCCGGCCAGCGGCGGGGGGCGC | |
| | AGTGCACACGAGGGGGCTGGACTTCGCCTGTGATTTTGGATGTTTTTGT | |
| | GGTTTCATCCTCATCGGTTTGATATTGTACATAAGTCTGGCGATAAGGA | |
| | AGAGAGTTCAAGAGACAAAGTTCGGAAATGCCTTTACAGAGGAAGACAG | |
| | TGAGCTCGTTGTAAACTACATCGCAAAAAAAAGCTTCTGTAGAAGAGCA | |
| | ATAGAGCTCACGTTGCACTCACTCGGTGTGTCCGAAGAACTCCAGAATA | |
| | AACTGGAAGACGTCGTTATCGATCGGAACCTCCTCATACTTGGAAAAAT | |
| | ACTGGGAGAAGGAGAGTTCGGCAGTGTCATGGAAGGTAACTTGAAACAA | |
| | GAGGATGGTACCTCACTCAAGGTAGCTGTCAAGACGATGAAACTTGATA | |
| | ACAGTTCACAAAGGGAGATCGAAGAATTTCTGTCTGAGGCCGCCTGTAT | |
| | GAAAGACTTCTCACATCCTAATGTCATCAGACTTCTTGGCGTTTGTATC | |
| | GAGATGTCTAGCCAAGGAATCCCAAAACCTATGGTCATATTGCCTTTCA | |
| | TGAAATATGGCGATCTGCATACATATTTGCTCTACTCTAGACTTGAGAC | |
| | AGGGCCCAAACATATTCCTCTCCAGACATTGCTCAAGTTTATGGTCGAT | |
| | ATTGCCCTGGGTATGGAGTACTTGAGCAACCGAAATTTTCTGCATCGGG | |
| | ATCTTGCCGCACGCAACTGCATGCTGCGCGATGACATGACCGTCTGCGT | |
| | GGCTGATTTTGGGCTGTCAAAAAAAATATATTCTGGAGACTACTACCGA | |
| | CAAGGGCGGATTGCAAAGATGCCCGTCAAATGGATTGCGATTGAAAGTT | |
| | TGGCGGACAGGGTATATACTTCCAAATCAGATGTTTGGGCTTTTGGAGT | |
| | CACTATGTGGGAAATAGCTACACGCGGTATGACCCCGTACCCCGGAGTA | |
| | CAAAATCATGAAATGTATGACTATCTCCTTCATGGACACAGGCTGAAGC | |
| | AGCCCGAGGACTGCCTGGACGAACTGTATGAAATAATGTATTCTTGTTG | |
| | GCGAACCGATCCCTTGGACCGGCCTACTTTCAGTGTCCTTAGATTGCAA | |
| | CTTGAGAAATTGCTCGAGTCTTTGCCGGATGTGCGAAACCAGGCAGACG | |
| | TGATCTATGTCAATACCCAACTTTTGGAAAGTTCTGAGGGCCTCGCACA | |
| | GGGTTCTACCCTTGCCCCGTTGGATCTTAACATAGACCCAGACAGCATA | |
| | ATTGCTTCTTGTACACCTCGCGCTGCCATATCAGTTGTAACAGCGGAGG | |
| | TCCATGATAGTAAACCTCACGAGGGTCGCTATATCCTGAACGGCGGGTC | |
| | AGAAGAATGGGAAGACCTGACATCAGCGCCGAGCGCCGCCGTTACTGCT | |
| | GAGAAAAACTCTGTCCTGCCCGGAGAGCGCTTGGTTCGGAACGGGGTAA | |
| | GTTGGAGCCATAGCTCAATGCTCCCCCTGGGTTCAAGTCTCCCGGACGA | |
| | GCTTCTTTTTGCGGACGACTCATCTGAGGGGTCCGAAGTTCTGATG | |
| C3-sc19- | SEQ ID NO: 82: | SEQ ID NO: 100: |
| MEG CPR | ATGGCCTTACCAGTGACCGCCTTGCTCCTGCCGCTGGCCTTGCTGCTCC | MALPVTALLLPLALLLHAARPGSEVKLQESGPGLVAPSQSLSVT |
| | ACGCCGCCAGGCCGGGATCCGAGGTGAAACTGCAGGAGTCAGGACCTGG | CTVSGVSLPDYGVSWIRQPPRKGLEWLGVIWGSETTYYNSALKS |
| | CCTGGTGGCGCCCTCACAGAGCCTGTCCGTCACATGCACTGTCTCAGGG | RLTIIKDNSKSQVFLKMNSLOTDDTAIYYCAKHYYYGGSYAMDY |
| | GTCTCATTACCCGACTATGGTGTAAGCTGGATTCGCCAGCCTCCACGAA | WGQGTSVTVSSGGGGSGGGGSGGGGSDIQMTQTTSSLSASLGDR |
| | AGGGTCTGGAGTGGCTGGGAGTAATATGGGGTAGTGAAACCACATACTA | VTISCRASQDISKYLNWYQQKPDGTVKLLIYHTSRLHSGVPSRF |
| | TAATTCAGCTCTCAAATCCAGACTGACCATCATCAAGGACAACTCCAAG | SGSGSGTDYSLTISNLEQEDIATYFCQQGNTLPYTFGGGTKLEI |
| | AGCCAAGTTTTCTTAAAAATGAACAGTCTGCAAACTGATGACACAGCCA | TRADAAPTVSIFPPSSNGSFVPVFLPAKPTTTPAPRPPTPAPTI |
| | TTTACTACTGTGCCAAACATTATTACTACGGTGGTAGCTATGCTATGGA | ASQPLSLRPEACRPAAGGAVHTRGLDFACDAIAGIIILVLVVLF |
| | CTACTGGGGTCAAGGAACCTCAGTCACCGTCTCCTCAGGCGGCGGTGGC | LLALFIIYRHKQKGKESSMPAVTYTPAMRVVNADYTISGTLPHS |
| | TCTGGCGGTGGCGGTTCCGGCGGTGGTGGTAGCGACATCCAGATGACAC | NGGNANSHYFTNPSYHTLTQCATSPHVNNRDRMTVTKSKNNQLF |
| | AGACTACATCCTCCCTGTCTGCCTCTCTGGGAGACAGAGTCACCATCAG | VNLKNVNPGKRGPVGDCTGTLPADWKHGGYLNELGAFGLDRSYM |
| | TTGCAGGGCAAGTCAGGACATTAGTAAATATTTAAATTGGTATCAGCAG | GKSLKDLGKNSEYNSSNCSLSSSENPYATIKDPPVLIPKSSECG |
| | AAACCAGATGGAACTGTTAAACTCCTGATCTACCATACATCAAGATTAC | YVEMKSPARRDSPYAEINNSTSANRNVYEVEPTVSVVQGVFSNN |
| | ACTCAGGAGTCCCATCAAGGTTCAGTGGCAGTGGGTCTGGAACAGATTA | GRLSQDPYDLPKNSHIPCHYDLLPVRDSSSSPKQEDSGGSSSNS |
| | TTCTCTCACCATTAGCAACCTGGAGCAAGAAGATATTGCCACTTACTTT | SSSSE |
| | TGCCAACAGGGTAATACGCTTCCGTACACGTTCGGAGGGGGGACTAAGT | |
| | TGGAAATAACACGGGCTGATGCTGCACCAACTGTATCCATCTTCCCACC | |
| | ATCCAGTAATGGATCCTTCGTGCCGGTCTTCCTGCCAGCGAAGCCCACC | |
| | ACGACGCCAGCGCCGCGACCACCAACACCGGCGCCCACCATCGCGTCGC | |
| | AGCCCCTGTCCCTGCGCCCAGAGGCGTGCCGGCCAGCGGCGGGGGGCGC | |
| | AGTGCACACGAGGGGGCTGGACTTCGCCTGTGATGCTATCGCGGGGATC | |
| | ATTATATTGGTCTTGGTGGTGCTGTTTCTGCTCGCGCTTTTCATTATAT | |
| | ACCGCCATAAGCAGAAGGGCAAAGAGTCCTCCATGCCAGCCGTGACCTA | |
| | TACGCCTGCGATGCGCGTTGTCAACGCCGATTACACCATCAGTGGTACC | |
| | CTCCCGCACAGTAACGGCGGAAATGCAAACTCTCATTACTTTACAAATC | |
| | CTAGTTACCATACACTCACTCAGTGTGCTACCTCTCCCCATGTGAACAA | |
| | TCGGGACAGGATGACCGTTACGAAAAGCAAAAATAACCAGTTGTTTGTG | |
| | AACCTTAAGAATGTGAATCCCGGCAAGAGGGGTCCGGTGGGTGACTGCA | |
| | CCGGAACTCTCCCCGCTGACTGGAAGCATGGCGGGTACCTGAACGAACT | |
| | CGGCGCGTTTGGGCTCGACCGAAGCTACATGGGTAAAAGTCTTAAGGAC | |
| | CTCGGTAAGAATAGCGAGTATAATAGCTCTAACTGTTCCCTTTCCAGCT | |
| | CCGAGAATCCGTACGCTACTATAAAAGACCCCCCGGTGCTCATTCCCAA | |
| | ATCCAGTGAGTGCGGGTACGTGGAGATGAAAAGTCCCGCTCGAAGAGAC | |
| | AGTCCATACGCGGAAATCAATAACTCCACCAGTGCGAACCGCAATGTGT | |
| | ACGAAGTGGAGCCCACCGTTTCCGTTGTACAAGGTGTATTTTCAAACAA | |
| | TGGGAGGCTTAGCCAGGACCCCTATGATCTTCCAAAGAACAGCCACATC | |
| | CCGTGTCATTATGATCTGTTGCCGGTGAGGGATTCTAGCTCTTCTCCTA | |
| | AACAAGAGGACTCAGGTGGCTCTAGTTCCAACTCCTCCAGTTCTTCAGA | |
| | G | |
| C5-sc19-Dec | SEQ ID NO: 83: | SEQ ID NO: 101: |
| CPR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGFVPVFLPAKPTTTPAPRPPTPAP |
| | AAAGGGCAGTTGTGCTGCCAGTCCACCGTGGCGGTTGATTGCCGTCATC | TIASQPLSLRPEACRPAAGGAVHTRGLDFACDGSEVKLQESGPG |
| | CTCGGCATTCTGTGCTTGGTTATTCTCGTTATAGCGGTGGTGCTTGGGT | LVAPSQSLSVTCTVSGVSLPDYGVSWIRQPPRKGLEWLGVIWGS |
| | TCGTGCCGGTCTTCCTGCCAGCGAAGCCCACCACGACGCCAGCGCCGCG | ETTYYNSALKSRLTIIKDNSKSQVFLKMNSLOTDDTAIYYCAKH |
| | ACCACCAACACCGGCGCCCACCATCGCGTCGCAGCCCCTGTCCCTGCGC | YYYGGSYAMDYWGQGTSVTVSSGGGGSGGGGSGGGGSDIQMTQT |
| | CCAGAGGCGTGCCGGCCAGCGGCGGGGGGCGCAGTGCACACGAGGGGGC | TSSLSASLGDRVTISCRASQDISKYLNWYQQKPDGTVKLLIYHT |
| | TGGACTTCGCCTGTGATGGATCCGAGGTGAAACTGCAGGAGTCAGGACC | SRLHSGVPSRFSGSGSGTDYSLTISNLEQEDIATYFCQQGNTLP |
| | TGGCCTGGTGGCGCCCTCACAGAGCCTGTCCGTCACATGCACTGTCTCA | YTFGGGTKLEITRADAAPTVSIFPPSSN |
| | GGGGTCTCATTACCCGACTATGGTGTAAGCTGGATTCGCCAGCCTCCAC | |
| | GAAAGGGTCTGGAGTGGCTGGGAGTAATATGGGGTAGTGAAACCACATA | |
| | CTATAATTCAGCTCTCAAATCCAGACTGACCATCATCAAGGACAACTCC | |
| | AAGAGCCAAGTTTTCTTAAAAATGAACAGTCTGCAAACTGATGACACAG | |
| | CCATTTACTACTGTGCCAAACATTATTACTACGGTGGTAGCTATGCTAT | |
| | GGACTACTGGGGTCAAGGAACCTCAGTCACCGTCTCCTCAGGCGGCGGT | |
| | GGCTCTGGCGGTGGCGGTTCCGGCGGTGGTGGTAGCGACATCCAGATGA | |
| | CACAGACTACATCCTCCCTGTCTGCCTCTCTGGGAGACAGAGTCACCAT | |
| | CAGTTGCAGGGCAAGTCAGGACATTAGTAAATATTTAAATTGGTATCAG | |
| | CAGAAACCAGATGGAACTGTTAAACTCCTGATCTACCATACATCAAGAT | |
| | TACACTCAGGAGTCCCATCAAGGTTCAGTGGCAGTGGGTCTGGAACAGA | |
| | TTATTCTCTCACCATTAGCAACCTGGAGCAAGAAGATATTGCCACTTAC | |
| | TTTTGCCAACAGGGTAATACGCTTCCGTACACGTTCGGAGGGGGGACTA | |
| | AGTTGGAAATAACACGGGCTGATGCTGCACCAACTGTATCCATCTTCCC | |
| | ACCATCCAGTAAT | |
| C7-sc19- | SEQ ID NO: 84: | SEQ ID NO: 102: |
| DecFull CPR | ATGGAGTATCACCCCGACCTGGAGAATTTGGACGAGGATGGCTACACTC | MEYHPDLENLDEDGYTQLHFDSQSNTRIAVVSEKGSCAASPPWR |
| | AACTGCATTTCGATAGTCAGTCCAATACAAGGATCGCTGTTGTGTCTGA | LIAVILGILCLVILVIAVVLGTMAIWRSNSGSNTLENGYFLSRN |
| | AAAGGGCAGTTGTGCTGCCAGTCCACCGTGGCGGTTGATTGCCGTCATC | KENHSQPTQSSLEDSVTPTKAVKTTGVLSSPCPPNWIIYEKSCY |
| | CTCGGCATTCTGTGCTTGGTTATTCTCGTTATAGCGGTGGTGCTTGGGA | LFSMSLNSWDGSKRQCWQLGSNLLKIDSSNELGFIVKQVSSQPD |
| | CCATGGCGATCTGGCGCTCCAACTCTGGAAGTAACACCCTTGAAAATGG | NSFWIGLSRPQTEVPWLWEDGSTESSNLFQIRTTATQENPSPNC |
| | TTACTTCCTCAGTAGGAACAAAGAGAACCATTCCCAGCCGACACAGTCA | VWIHVSVIYDQLCSVPSYSICEKKESMGSEVKLQESGPGLVAPS |
| | AGCCTTGAAGATTCAGTCACCCCTACAAAGGCCGTAAAAACGACAGGTG | QSLSVTCTVSGVSLPDYGVSWIRQPPRKGLEWLGVIWGSETTYY |
| | TCCTGTCCTCTCCGTGTCCGCCTAACTGGATCATCTACGAGAAAAGTTG | NSALKSRLTIIKDNSKSQVFLKMNSLQTDDTAIYYCAKHYYYGG |
| | TTATCTGTTTAGCATGAGCCTTAACAGTTGGGATGGCTCAAAAAGGCAG | SYAMDYWGQGTSVTVSSGGGGSGGGGSGGGGSDIQMTQTTSSLS |
| | TGCTGGCAACTGGGGAGCAACCTTTTGAAGATAGACAGTTCCAACGAAC | ASLGDRVTISCRASQDISKYLNWYQQKPDGTVKLLIYHTSRLHS |
| | TGGGCTTCATAGTCAAACAGGTGTCCTCTCAACCTGATAACTCATTCTG | GVPSRFSGSGSGTDYSLTISNLEQEDIATYFCQQGNTLPYTFGG |
| | GATCGGGCTCAGTCGACCCCAAACTGAGGTTCCATGGCTTTGGGAAGAC | GTKLEITRADAAPTVSIFPPSSN |
| | GGCAGCACTTTCTCTTCAAATTTGTTTCAAATAAGAACCACCGCTACGC | |
| | AGGAGAATCCGAGTCCGAACTGTGTTTGGATTCACGTCTCAGTCATTTA | |
| | CGACCAACTTTGTAGTGTCCCTAGCTATTCCATCTGCGAGAAAAAGTTC | |
| | AGTATGGGATCCGAGGTGAAACTGCAGGAGTCAGGACCTGGCCTGGTGG | |
| | CGCCCTCACAGAGCCTGTCCGTCACATGCACTGTCTCAGGGGTCTCATT | |
| | ACCCGACTATGGTGTAAGCTGGATTCGCCAGCCTCCACGAAAGGGTCTG | |
| | GAGTGGCTGGGAGTAATATGGGGTAGTGAAACCACATACTATAATTCAG | |
| | CTCTCAAATCCAGACTGACCATCATCAAGGACAACTCCAAGAGCCAAGT | |
| | TTTCTTAAAAATGAACAGTCTGCAAACTGATGACACAGCCATTTACTAC | |
| | TGTGCCAAACATTATTACTACGGTGGTAGCTATGCTATGGACTACTGGG | |
| | GTCAAGGAACCTCAGTCACCGTCTCCTCAGGCGGCGGTGGCTCTGGCGG | |
| | TGGCGGTTCCGGCGGTGGTGGTAGCGACATCCAGATGACACAGACTACA | |
| | TCCTCCCTGTCTGCCTCTCTGGGAGACAGAGTCACCATCAGTTGCAGGG | |
| | CAAGTCAGGACATTAGTAAATATTTAAATTGGTATCAGCAGAAACCAGA | |
| | TGGAACTGTTAAACTCCTGATCTACCATACATCAAGATTACACTCAGGA | |
| | GTCCCATCAAGGTTCAGTGGCAGTGGGTCTGGAACAGATTATTCTCTCA | |
| | CCATTAGCAACCTGGAGCAAGAAGATATTGCCACTTACTTTTGCCAACA | |
| | GGGTAATACGCTTCCGTACACGTTCGGAGGGGGGACTAAGTTGGAAATA | |
| | ACACGGGCTGATGCTGCACCAACTGTATCCATCTTCCCACCATCCAGTA | |
| | AT | |
| C9-sc19-163 | SEQ ID NO: 85: | SEQ ID NO: 103: |
| CPR | ATGGCCTTACCAGTGACCGCCTTGCTCCTGCCGCTGGCCTTGCTGCTCC | MALPVTALLLPLALLLHAARPGSEVKLQESGPGLVAPSQSLSVT |
| | ACGCCGCCAGGCCGGGATCCGAGGTGAAACTGCAGGAGTCAGGACCTGG | CTVSGVSLPDYGVSWIRQPPRKGLEWLGVIWGSETTYYNSALKS |
| | CCTGGTGGCGCCCTCACAGAGCCTGTCCGTCACATGCACTGTCTCAGGG | RLTIIKDNSKSQVFLKMNSLQTDDTAIYYCAKHYYYGGSYAMDY |
| | GTCTCATTACCCGACTATGGTGTAAGCTGGATTCGCCAGCCTCCACGAA | WGQGTSVTVSSGGGGSGGGGSGGGGSDIQMTQTTSSLSASLGDR |
| | AGGGTCTGGAGTGGCTGGGAGTAATATGGGGTAGTGAAACCACATACTA | VTISCRASQDISKYLNWYQQKPDGTVKLLIYHTSRLHSGVPSRF |
| | TAATTCAGCTCTCAAATCCAGACTGACCATCATCAAGGACAACTCCAAG | SGSGSGTDYSLTISNLEQEDIATYFCQQGNTLPYTFGGGTKLEI |
| | AGCCAAGTTTTCTTAAAAATGAACAGTCTGCAAACTGATGACACAGCCA | TRADAAPTVSIFPPSSNGSFVPVFLPAKPTTTPAPRPPTPAPTI |
| | TTTACTACTGTGCCAAACATTATTACTACGGTGGTAGCTATGCTATGGA | ASQPLSLRPEACRPAAGGAVHTRGLDFACDSSLGGTDKELRLVD |
| | CTACTGGGGTCAAGGAACCTCAGTCACCGTCTCCTCAGGCGGCGGTGGC | GENKCSGRVEVKVQEEWGTVCNNGWSMEAVSVICNQLGCPTAIK |
| | TCTGGCGGTGGCGGTTCCGGCGGTGGTGGTAGCGACATCCAGATGACAC | APGWANSSAGSGRIWMDHVSCRGNESALWDCKHDGWGKHSNCTH |
| | AGACTACATCCTCCCTGTCTGCCTCTCTGGGAGACAGAGTCACCATCAG | QQDAGVTCSDGSNLEMRLTRGGNMCSGRIEIKFQGRWGTVCDDN |
| | TTGCAGGGCAAGTCAGGACATTAGTAAATATTTAAATTGGTATCAGCAG | FNIDHASVICRQLECGSAVSFSGSSNFGEGSGPIWFDDLICNGN |
| | AAACCAGATGGAACTGTTAAACTCCTGATCTACCATACATCAAGATTAC | ESALWNCKHQGWGKHNCDHAEDAGVICSKGADLSLRLVDGVTEC |
| | ACTCAGGAGTCCCATCAAGGTTCAGTGGCAGTGGGTCTGGAACAGATTA | SGRLEVRFQGEWGTICDDGWDSYDAAVACKQLGCPTAVTAIGRV |
| | TTCTCTCACCATTAGCAACCTGGAGCAAGAAGATATTGCCACTTACTTT | NASKGFGHIWLDSVSCQGHEPAIWOCKHHEWGKHYCNHNEDAGV |
| | TGCCAACAGGGTAATACGCTTCCGTACACGTTCGGAGGGGGGACTAAGT | TCSDGSDLELRLRGGGSRCAGTVEVEIQRLLGKVCDRGWGLKEA |
| | TGGAAATAACACGGGCTGATGCTGCACCAACTGTATCCATCTTCCCACC | DVVCRQLGCGSALKTSYQVYSKIQATNTWLFLSSCNGNETSLWD |
| | ATCCAGTAATGGATCCTTCGTGCCGGTCTTCCTGCCAGCGAAGCCCACC | CKNWQWGGLTCDHYEEAKITCSAHREPRLVGGDIPCSGRVEVKH |
| | ACGACGCCAGCGCCGCGACCACCAACACCGGCGCCCACCATCGCGTCGC | GDTWGSICDSDFSLEAASVLCRELQCGTVVSILGGAHFGEGNGQ |
| | AGCCCCTGTCCCTGCGCCCAGAGGCGTGCCGGCCAGCGGCGGGGGGCGC | IWAEEFQCEGHESHLSLCPVAPRPEGTCSHSRDVGVVCSRYTEI |
| | AGTGCACACGAGGGGGCTGGACTTCGCCTGTGATTCATCCCTGGGTGGA | RLVNGKTPCEGRVELKTLGAWGSLCNSHWDIEDAHVLCQQLKCG |
| | ACAGATAAGGAGTTGAGGTTGGTGGATGGAGAGAATAAGTGCAGTGGAC | VALSTPGGARFGKGNGQIWRHMFHCTGTEQHMGDCPVTALGASL |
| | GGGTTGAGGTCAAGGTGCAGGAAGAGTGGGGTACTGTTTGTAACAACGG | CPSEQVASVICSGNOSQTLSSCNSSSLGPTRPTIPEESAVACIE |
| | CTGGAGTATGGAAGCAGTCTCCGTGATATGCAATCAACTTGGTTGCCCT | SGQLRLVNGGGRCAGRVEIYHEGSWGTICDDSWDLSDAHVVCRQ |
| | ACAGCCATAAAAGCTCCTGGATGGGCTAACTCATCTGCCGGAAGCGGTC | LGCGEAINATGSAHFGEGTGPIWLDEMKCNGKESRIWQCHSHGW |
| | GAATATGGATGGACCATGTATCCTGTAGGGGCAATGAGTCAGCACTGTG | GQQNCRHKEDAGVICSEFMSLRLTSEASREACAGRLEVFYNGAW |
| | GGACTGCAAGCATGACGGGTGGGGAAAACACTCTAATTGTACCCACCAG | GTVGKSSMSETTVGVVCRQLGCADKGKINPASLDKAMSIPMWVD |
| | CAAGATGCCGGTGTGACGTGTAGCGACGGCAGCAACTTGGAGATGCGGT | NVQCPKGPDTLWQCPSSPWEKRLASPSEETWITCDNKIRLQEGP |
| | TGACGCGCGGCGGGAACATGTGCAGCGGTCGCATTGAGATAAAGTTCCA | TSCSGRVEIWHGGSWGTVCDDSWDLDDAQVVCQQLGCGPALKAF |
| | AGGGCGCTGGGGGACCGTTTGTGACGACAACTTCAATATAGATCATGCT | KEAEFGQGTGPIWLNEVKCKGNESSLWDCPARRWGHSECGHKED |
| | AGTGTGATATGCAGGCAGTTGGAGTGCGGTAGCGCCGTCTCATTTAGTG | AAVNCTDISVQKTPQKATTGRSSRQSSFIAVGILGVVLLAIFVA |
| | GTTCTAGCAATTTCGGTGAGGGATCCGGGCCTATATGGTTCGACGACCT | LFFLTKKRRQRQRLAVSSRGENLVHQIQYREMNSCLNADDLDLM |
| | TATCTGCAATGGAAATGAGAGCGCCCTCTGGAACTGCAAGCACCAAGGT | NSSENSHESADFSAAELISVSKFLPISGMEKEAILSHTEKENGN |
| | TGGGGTAAGCATAACTGCGATCATGCTGAGGATGCGGGTGTGATTTGTA | L |
| | GCAAGGGAGCCGACTTGTCACTTCGACTCGTAGATGGCGTGACGGAATG | |
| | TAGCGGTCGCCTTGAGGTACGCTTCCAAGGTGAATGGGGCACAATCTGT | |
| | GATGACGGTTGGGACTCATATGACGCTGCGGTTGCTTGCAAACAGCTGG | |
| | GCTGTCCGACAGCGGTAACGGCAATTGGGCGGGTAAATGCCAGTAAGGG | |
| | GTTTGGACATATATGGTTGGACAGTGTTTCTTGTCAGGGACACGAACCT | |
| | GCAATATGGCAATGTAAACATCACGAGTGGGGGAAGCATTACTGCAATC | |
| | ATAATGAAGACGCAGGCGTCACATGCTCTGACGGATCCGACCTGGAATT | |
| | GCGGCTGAGAGGGGGGGGTTCACGGTGCGCAGGAACCGTGGAAGTTGAG | |
| | ATTCAACGCCTGCTCGGGAAAGTTTGTGACAGGGGGTGGGGCCTTAAGG | |
| | AAGCAGACGTAGTCTGCCGACAACTGGGTTGCGGGAGCGCCCTCAAGAC | |
| | GTCCTATCAAGTTTACAGCAAAATTCAAGCAACTAATACCTGGCTGTTT | |
| | CTTTCCTCCTGCAATGGTAACGAAACGAGCCTCTGGGATTGTAAAAATT | |
| | GGCAATGGGGAGGCCTTACATGTGATCACTATGAAGAGGCCAAAATCAC | |
| | CTGCAGCGCGCACCGAGAGCCTAGGCTGGTTGGAGGGGATATTCCCTGT | |
| | TCAGGTAGAGTTGAAGTCAAGCATGGTGATACCTGGGGGTCCATATGCG | |
| | ACTCCGATTTCAGTTTGGAAGCAGCGAGCGTGCTGTGCCGAGAGCTTCA | |
| | ATGTGGGACAGTAGTTTCCATTCTTGGTGGCGCCCACTTCGGGGAAGGT | |
| | AACGGACAGATTTGGGCGGAGGAATTCCAATGTGAGGGCCATGAAAGTC | |
| | ACCTGAGTCTTTGTCCTGTAGCGCCTCGGCCGGAAGGTACTTGTTCTCA | |
| | CTCCAGAGACGTTGGCGTGGTTTGTTCTAGGTACACTGAGATAAGGCTG | |
| | GTGAATGGCAAAACGCCTTGTGAAGGAAGGGTGGAGCTCAAAACGCTTG | |
| | GCGCCTGGGGGTCTCTGTGCAACTCCCACTGGGACATAGAGGACGCACA | |
| | TGTCTTGTGCCAGCAACTCAAATGCGGTGTCGCGCTTTCAACCCCTGGG | |
| | GGCGCTAGATTCGGGAAAGGTAACGGCCAGATATGGCGCCATATGTTCC | |
| | ATTGCACCGGAACTGAACAGCATATGGGAGATTGTCCTGTGACTGCCTT | |
| | GGGGGCAAGTCTGTGTCCCTCAGAACAAGTGGCTTCAGTTATTTGCAGC | |
| | GGTAACCAGAGTCAGACGCTCAGCTCCTGCAACAGCAGCAGTCTGGGTC | |
| | CAACAAGACCCACAATACCCGAGGAGTCAGCGGTCGCGTGCATCGAATC | |
| | CGGGCAATTGAGACTCGTTAATGGCGGGGGTCGGTGCGCAGGGCGCGTG | |
| | GAGATCTACCACGAGGGTAGTTGGGGCACAATTTGTGACGACAGCTGGG | |
| | ACCTTTCCGACGCTCATGTCGTATGCCGACAACTGGGGTGCGGTGAGGC | |
| | CATCAATGCCACCGGAAGCGCGCATTTCGGTGAAGGCACGGGCCCAATT | |
| | TGGCTTGATGAGATGAAATGTAATGGAAAGGAGTCCCGCATTTGGCAAT | |
| | GCCATAGCCATGGCTGGGGTCAACAAAATTGTCGACACAAAGAAGATGC | |
| | CGGGGTGATCTGCTCAGAATTCATGTCCTTGCGGCTTACTAGCGAAGCG | |
| | TCCCGCGAGGCCTGTGCTGGGAGACTGGAAGTTTTTTATAACGGGGCTT | |
| | GGGGAACGGTTGGTAAGTCATCAATGAGTGAAACCACAGTTGGTGTTGT | |
| | GTGTAGACAACTCGGTTGCGCCGACAAGGGAAAGATCAACCCGGCGAGC | |
| | CTTGATAAGGCCATGAGCATCCCCATGTGGGTGGATAACGTTCAGTGTC | |
| | CGAAAGGTCCCGATACCTTGTGGCAATGCCCAAGTTCTCCTTGGGAGAA | |
| | GAGACTCGCCTCACCAAGTGAAGAGACGTGGATTACGTGTGATAACAAA | |
| | ATTAGGCTCCAAGAAGGACCGACCAGTTGCAGCGGAAGAGTTGAGATAT | |
| | GGCATGGAGGAAGCTGGGGAACCGTGTGCGATGACAGCTGGGACCTGGA | |
| | CGACGCCCAGGTGGTGTGCCAACAGCTGGGTTGCGGGCCTGCCCTCAAA | |
| | GCATTTAAGGAAGCCGAATTCGGTCAGGGTACTGGGCCAATCTGGCTGA | |
| | ACGAGGTAAAGTGCAAAGGTAACGAAAGTAGCCTGTGGGACTGTCCGGC | |
| | ACGAAGGTGGGGCCACAGCGAGTGTGGCCATAAGGAAGACGCGGCCGTG | |
| | AACTGTACAGACATATCCGTACAAAAAACGCCCCAAAAGGCGACGACCG | |
| | GGCGATCATCAAGACAATCTAGCTTTATTGCCGTGGGAATTCTCGGTGT | |
| | AGTGCTTCTTGCTATATTTGTCGCTTTGTTCTTTCTGACTAAAAAGCGC | |
| | AGGCAAAGGCAGCGGCTTGCTGTGAGCTCTCGGGGAGAAAACCTCGTTC | |
| | ACCAAATCCAATACCGAGAAATGAACTCCTGTCTCAACGCCGACGATCT | |
| | TGACCTGATGAACTCATCTGAGAACTCACACGAGTCCGCCGATTTCAGC | |
| | GCGGCGGAATTGATCTCTGTCAGCAAATTTCTGCCTATAAGTGGCATGG | |
| | AAAAAGAAGCCATACTCTCTCACACGGAAAAGGAAAATGGCAACCTT | |
| ACE2- | SEQ ID NO: 106: | SEQ ID NO: 107: |
| sCD163 | ATGGGTTGGAGCTGCATTATCTTGTTTCTTGTCGCCACGGCTACGGGCG | MGWSCIILFLVATATGVHSHHHHHHSTIEEQAKTFLDKFNHEAE |
| BME | TTCATTCACACCATCACCACCATCATAGCACCATCGAGGAGCAGGCAAA | DLFYQSSLASWNYNTNITEENVQNMNNAGDKWSAFLKEQSTLAQ |
| | AACTTTTCTTGACAAGTTCAACCATGAGGCCGAAGACTTGTTCTATCAA | MYPLQEIQNLTVKLQLQALQQNGSSVLSEDKSKRLNTILNTMST |
| | AGCTCATTGGCGAGCTGGAATTATAATACAAACATCACGGAGGAAAATG | IYSTGKVCNPDNPQECLLLEPGLNEIMANSLDYNERLWAWESWR |
| | TACAGAACATGAACAATGCAGGGGATAAATGGTCCGCTTTTCTGAAAGA | SEVGKQLRPLYEEYVVLKNEMARANHYEDYGDYWRGDYEVNGVD |
| | GCAATCCACTCTCGCACAAATGTATCCCTTGCAAGAGATACAAAACTTG | GYDYSRGQLIEDVEHTFEEIKPLYEHLHAYVRAKLMNAYPSYIS |
| | ACAGTGAAGCTTCAGCTCCAGGCCCTGCAGCAGAATGGGTCCAGCGTCT | PIGCLPAHLLGDMWGRFWTNLYSLTVPFGQKPNIDVTDAMVDQA |
| | TGAGCGAGGATAAATCCAAGCGCCTTAATACGATTCTTAACACGATGAG | WDAQRIFKEAEKFFVSVGLPNMTQGFWENSMLTDPGNVQKAVCH |
| | CACTATATACAGTACGGGCAAGGTGTGCAACCCCGACAATCCTCAAGAG | PTAWDLGKGDFRILMCTKVTMDDFLTAHHEMGHIQYDMAYAAQP |
| | TGCTTGCTTCTCGAGCCAGGCCTTAACGAAATCATGGCAAACTCATTGG | FLLRNGANEGFHEAVGEIMSLSAATPKHLKSIGLLSPDFQEDNE |
| | ACTATAATGAGCGCCTCTGGGCGTGGGAATCTTGGAGATCTGAGGTTGG | TEINFLLKQALTIVGTLPFTYMLEKWRWMVFKGEIPKDQWMKKW |
| | TAAGCAGCTTCGACCTTTGTATGAAGAATACGTGGTATTGAAAAACGAA | WEMKREIVGVVEPVPHDETYCDPASLFHVSNDYSFIRYYTRTLY |
| | ATGGCGCGAGCTAATCATTACGAAGACTACGGTGACTACTGGCGAGGAG | QFQFQEALCQAAKHEGPLHKCDISNSTEAGQKLFNMLRLGKSEP |
| | ATTATGAAGTGAATGGGGTAGACGGCTACGACTACTCTCGAGGGCAACT | WTLALENVVGAKNMNVRPLLNYFEPLFTWLKDONKNSFVGWSTD |
| | CATCGAAGATGTTGAGCACACATTCGAAGAAATCAAACCACTTTATGAG | WSPYADQSIKVRISLKSALGDKAYEWNDNEMYLFRSSVAYAMRQ |
| | CATCTCCATGCGTACGTACGAGCGAAACTCATGAACGCGTACCCCAGTT | YFLKVKNQMILFGEEDVRVANLKPRISENFFVTAPKNVSDIIPR |
| | ATATAAGTCCCATCGGTTGCCTCCCCGCGCATCTTCTTGGAGACATGTG | TEVEKAIRMSRSRINDAFRLNDNSLEFLGIQPTLGPPNQPPVSS |
| | GGGGAGATTCTGGACCAACCTCTATAGTCTTACTGTACCCTTCGGGCAA | SLGGTDKELRLVDGENKCSGRVEVKVQEEWGTVCNNGWSMEAVS |
| | AAGCCGAATATAGATGTGACTGATGCTATGGTGGACCAGGCCTGGGACG | VICNQLGCPTAIKAPGWANSSAGSGRIWMDHVSCRGNESALWDC |
| | CACAAAGGATTTTTAAGGAAGCAGAAAAGTTCTTTGTATCTGTGGGGCT | KHDGWGKHSNCTHQQDAGVTCSDGSNLEMRLTRGGNMCSGRIEI |
| | CCCCAATATGACTCAAGGGTTCTGGGAAAACTCCATGCTGACAGATCCT | KFQGRWGTVCDDNFNIDHASVICRQLECGSAVSFSGSSNFGEGS |
| | GGGAACGTGCAAAAGGCCGTGTGTCACCCTACAGCGTGGGACCTTGGGA | GPIWFDDLICNGNESALWNCKHQGWGKHNCDHAEDAGVICSKGA |
| | AAGGTGACTTTAGAATTCTGATGTGTACCAAGGTGACTATGGACGATTT | DLSLRLVDGVTECSGRLEVRFQGEWGTICDDGWDSYDAAVACKQ |
| | TTTGACCGCTCATCATGAGATGGGACATATCCAGTACGATATGGCTTAC | LGCPTAVTAIGRVNASKGFGHIWLDSVSCQGHEPAIWQCKHHEW |
| | GCAGCTCAGCCTTTCCTCCTGAGGAATGGCGCCAATGAGGGATTTCATG | GKHYCNHNEDAGVTCSDGSDLELRLRGGGSRCAGTVEVEIQRLL |
| | AAGCCGTGGGCGAAATAATGTCTCTGAGCGCTGCTACTCCTAAGCATTT | GKVCDRGWGLKEADVVCRQLGCGSALKTSYQVYSKIQATNTWLF |
| | GAAAAGCATAGGCCTCCTCTCTCCCGACTTCCAAGAGGACAACGAGACA | LSSCNGNETSLWDCKNWOWGGLTCDHYEEAKITCSAHREPRLVG |
| | GAAATTAATTTCCTCCTTAAACAGGCGCTCACCATAGTAGGGACATTGC | GDIPCSGRVEVKHGDTWGSICDSDFSLEAASVLCRELQCGTVVS |
| | CTTTCACATACATGCTTGAGAAATGGAGATGGATGGTTTTCAAAGGGGA | ILGGAHFGEGNGQIWAEEFQCEGHESHLSLCPVAPRPEGTCSHS |
| | GATCCCCAAAGATCAGTGGATGAAGAAATGGTGGGAGATGAAGCGGGAA | RDVGVVCSRYTEIRLVNGKTPCEGRVELKTLGAWGSLCNSHWDI |
| | ATAGTTGGTGTGGTGGAGCCGGTCCCGCATGACGAGACCTATTGCGATC | EDAHVLCQQLKCGVALSTPGGARFGKGNGQIWRHMFHCTGTEQH |
| | CAGCATCACTCTTTCACGTCAGCAATGACTACTCTTTCATTAGATATTA | MGDCPVTALGASLCPSEQVASVICSGNOSQTLSSCNSSSLGPTR |
| | TACCCGCACTCTGTATCAATTTCAGTTCCAAGAGGCGTTGTGCCAAGCG | PTIPEESAVACIESGQLRLVNGGGRCAGRVEIYHEGSWGTICDD |
| | GCAAAACATGAGGGCCCCCTTCACAAATGTGACATATCCAACTCCACTG | SWDLSDAHVVCRQLGCGEAINATGSAHFGEGTGPIWLDEMKCNG |
| | AAGCAGGCCAGAAATTGTTTAATATGCTGAGACTGGGTAAGAGTGAACC | KESRIWOCHSHGWGQQNCRHKEDAGVICSEFMSLRLTSEASREA |
| | ATGGACTCTTGCCCTCGAAAACGTAGTCGGCGCCAAAAATATGAACGTT | CAGRLEVFYNGAWGTVGKSSMSETTVGVVCRQLGCADKGKINPA |
| | CGCCCCCTGCTGAATTACTTTCAACCCCTCTTTACGTGGCTCAAAGATC | SLDKAMSIPMWVDNVQCPKGPDTLWQCPSSPWEKRLASPSEETW |
| | AGAACAAAAATTCCTTCGTGGGGTGGTCCACAGACTGGTCACCTTACGC | ITCDNKIRLQEGPTSCSGRVEIWHGGSWGTVCDDSWDLDDAQVV |
| | CGACCAGAGTATAAAAGTAAGGATETCCCTTAAGAGTGCCCTGGGTGAC | COQLGCGPALKAFKEAEFGQGTGPIWLNEVKCKGNESSLWDCPA |
| | AAAGCGTACGAGTGGAACGACAATGAGATGTACCTTTTTCGCTCTAGCG | RRWGHSECGHKEDAAVNCTDISVQKTPQKATTGRSSRQSS |
| | TTGCATACGCTATGCGGCAGTACTTCCTGAAGGTTAAGAATCAAATGAT | |
| | TCTTTTCGGTGAAGAAGATGTGCGAGTAGCAAATTTGAAGCCGCGGATT | |
| | AGCTTCAACTTTTTCGTAACCECACCCAAAAACGTCTCAGATATTATCC | |
| | CTAGGACGGAAGTCGAGAAAGCGATCCGGATGAGTAGGAGTAGAATTAA | |
| | TGATGCCTTTCGGCTGAACGACAACTCCCTTGAATTTCTTGGCATCCAG | |
| | CCGACCCTTGGCCCGCCCAATCAGCCTCCAGTGAGCAGCTCTCTGGGTG | |
| | GGACGGATAAAGAATTGAGACTCGETGACGGCGAGAATAAGTGCTCCGG | |
| | ACGCGTCGAAGTGAAGGTTCAAGAGGAGTGGGGGACCGTGTGCAATAAT | |
| | GGCTGGAGCATGGAAGCCGTCAGCGTTATTTGTAATCAACTCGGATGCC | |
| | CGACTGCTATCAAAGCACCAGGATGGGCCAATTCTTCTGCTGGGAGCGG | |
| | ACGCATTTGGATGGATCATGTTAGETGTCGGGGTAATGAGAGTGCGTTG | |
| | TGGGACTGCAAACATGATGGGTGGGGTAAACACTCTAATTGTACACATC | |
| | AACAAGATGCGGGAGTGACGTGTAGTGACGGCTCCAATCTCGAAATGCG | |
| | CCTTACAAGAGGAGGAAATATGTGCTCTGGGAGGATTGAAATCAAATTC | |
| | CAAGGCCGGTGGGGCACAGTGTGCGATGATAATTTTAACATAGACCATG | |
| | CCAGTGTGATCTGCCGGCAGCTTGAATGTGGTTCTGCAGTCAGTTTCAG | |
| | CGGCTCATCCAACTTCGGGGAGGGCTCAGGGCCTATATGGTTTGATGAC | |
| | TTGATTTGCAACGGGAATGAGTCAGCACTGTGGAATTGTAAACACCAGG | |
| | GATGGGGCAAGCATAACTGTGACCATGCCGAAGATGCCGGCGTAATATG | |
| | CTCCAAAGGTGCGGACCTCTCTCTCCGGCTTGTGGACGGTGTCACGGAG | |
| | TGCTCTGGACGGTTGGAGGTCCGCTTTCAGGGGGAGTGGGGTACTATTT | |
| | GCGACGATGGTTGGGACTCCTACGATGCGGCTGTTGCATGCAAACAATT | |
| | GGGATGTCCTACTGCTGTTACGGCAATCGGTCGGGTAAACGCATCAAAG | |
| | GGGTTTGGGCATATATGGCTTGACAGCGTATCATGTCAAGGTCATGAAC | |
| | CAGCTATCTGGCAGTGTAAACATCATGAGTGGGGAAAACACTACTGCAA | |
| | TCACAACGAGGATGCCGGGGTCACGTGCTCTGATGGTAGTGATCTCGAG | |
| | TTGAGGCTTCGGGGTGGCGGTTCAAGATGCGCAGGCACTGTCGAAGTCG | |
| | AAATTCAGCGACTCCTGGGGAAAGTATGCGATAGGGGCTGGGGTCTCAA | |
| | AGAGGCCGATGTCGTTTGTAGACAGTTGGGTTGCGGCTCCGCTCTTAAA | |
| | ACATCCTACCAAGTTTATTCTAAAATCCAAGCGACTAATACTTGGCTCT | |
| | TCTTGTCTTCCTGTAATGGTAATGAGACGTCACTCTGGGATTGCAAAAA | |
| | TTGGCAATGGGGAGGTCTGACCTGTGACCACTACGAAGAAGCTAAGATT | |
| | ACGTGTAGTGCGCATCGAGAGCCTCGCTTGGTAGGAGGAGACATTCCTT | |
| | GCTCAGGCCGCGTAGAAGTCAAACACGGGGATACTTGGGGTTCTATCTG | |
| | TGATTCAGATTTTTCACTTGAAGCTGCGTCTGTGCTGTGTAGGGAACTT | |
| | CAATGTGGTACAGTCGTTAGTATTCTCGGGGGCGCCCATTTTGGTGAGG | |
| | GAAATGGGCAAATTTGGGCAGAAGAATTCCAATGCGAGGGACACGAGAG | |
| | TCATCTTAGCTTGTGCCCCGTGGCGCCAAGGCCGGAAGGGACATGCTCT | |
| | CACTCAAGAGATGTGGGAGTGGTGTGCTCAAGATATACAGAGATCAGGT | |
| | TGGTGAACGGGAAAACTCCTTGTGAGGGTCGAGTCGAACTTAAGACGTT | |
| | GGGTGCCTGGGGATCACTTTGCAATAGCCACTGGGACATTGAAGATGCC | |
| | CATGTGCTCTGCCAACAACTCAAGTGTGGAGTCGCTTTGTCCACCCCAG | |
| | GCGGCGCTCGATTCGGTAAGGGAAACGGTCAAATCTGGCGGCACATGTT | |
| | CCACTGCACTGGGACGGAGCAGCATATGGGTGACTGTCCGGTGACGGCT | |
| | TTGGGCGCCAGCTTGTGTCCAAGCGAACAGGTTGCCTCCGTGATCTGCA | |
| | GTGGCAATCAGTCTCAAACACTGAGCAGCTGCAACAGTTCAAGCTTGGG | |
| | GCCGACTCGGCCGACCATACCTGAGGAAAGTGCAGTCGCCTGCATCGAA | |
| | AGTGGGCAATTGCGCTTGGTTAATGGCGGCGGGCGGTGCGCTGGCCGAG | |
| | TAGAGATTTATCATGAAGGTTCCTGGGGGACCATCTGTGATGACTCATG | |
| | GGATCTTAGCGACGCCCACGTGGTATGTCGCCAGCTGGGTTGTGGCGAA | |
| | GCAATTAATGCGACAGGTTCTGCGCACTTCGGTGAAGGAACGGGGCCGA | |
| | TATGGCTTGACGAGATGAAATGCAACGGTAAAGAATCAAGGATTTGGCA | |
| | ATGTCACAGCCACGGTTGGGGGCAACAGAACTGTAGACACAAGGAAGAC | |
| | GCCGGCGTCATATGTTCAGAGTTTATGTCCTTGAGATTGACGAGCGAGG | |
| | CCAGTCGAGAAGCTTGCGCCGGGCGGCTTGAAGTTTTCTACAATGGAGC | |
| | CTGGGGGACCGTGGGTAAAAGTAGTATGAGCGAAACCACAGTAGGAGTA | |
| | GTTTGTCGCCAACTTGGGTGTGCCGATAAGGGCAAGATTAATCCCGCTT | |
| | CCCTTGATAAGGCGATGTCCATACCGATGTGGGTCGACAACGTGCAATG | |
| | CCCAAAAGGACCTGATACACTTTGGCAGTGCCCTAGTAGTCCTTGGGAG | |
| | AAGAGATTGGCCAGTCCGTCTGAAGAAACTTGGATAACATGTGACAACA | |
| | AGATACGACTTCAAGAGGGACCTACGTCATGTTCAGGTCGAGTGGAAAT | |
| | CTGGCACGGAGGATCATGGGGGACGGTTTGTGACGATAGCTGGGATCTG | |
| | GATGATGCCCAGGTAGTCTGCCAACAGCTCGGATGCGGTCCGGCGTTGA | |
| | AGGCGTTCAAGGAAGCCGAGTTTGGCCAAGGCACAGGACCAATTTGGCT | |
| | TAATGAAGTGAAATGCAAGGGTAACGAAAGCTCTCTTTGGGACTGTCCG | |
| | GCACGGCGGTGGGGGCACAGTGAGTGTGGCCATAAGGAAGACGCAGCAG | |
| | TGAACTGCACGGATATTAGTGTTCAGAAGACCCCGCAAAAAGCGACGAC | |
| | CGGGCGGAGCTCCCGCCAGTCCAGT | |

* Underlined text indicates optional signal peptide sequence and/or tag peptide sequence.

In some embodiments, the chimeric polypeptide of the present disclosure may further comprise one or more linkers between the various portions of the chimeric polypeptide. For example, a CBR or a CPR of the present disclosure may include one or more linkers between the extracellular portion and the transmembrane portion and/or between the transmembrane portion and the intracellular portion. A BME may include one or more linkers between the binding region and the endocytic receptor ligand. An AME may include one or more linkers between the antibody and the endocytic receptor ligand. Those skilled in the art will appreciate that any linker can be used which maintains the function of the chimeric polypeptide. There is no particular limitation with respect to the linkers that can be used in the chimeric polypeptides described herein. In some embodiments, the linker includes a peptide linker/spacer sequence. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In some embodiments, the linker includes a peptide linker sequence. In principle, there are no particular limitations to the length and/or amino acid composition of the linker peptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues. In some embodiments, the linker peptide sequence may include up to 300 amino acids, preferably 10 to 100 amino acids and most preferably 25 to 50 amino acids. In some embodiments, a short oligo- or polypeptide linker, preferably between 2 and 10 amino acids in length may form the linkage between the extracellular domain and the transmembrane domain of the chimeric polypeptide of the disclosure.

In some embodiments, the length and amino acid composition of the linker peptide sequence can be optimized to vary the orientation and/or proximity of the polypeptide domains to one another to achieve a desired activity of the chimeric polypeptide. In some embodiments, the orientation and/or proximity of the polypeptide domains to one another can be optimized to create a partial to full phagocytic versions of the chimeric polypeptide. In certain embodiments, the linker contains only glycine and/or serine residues (e.g., glycine-serine linker). Examples of such peptide linkers include: Gly(x) Ser, where x is 0 to 6; or Ser Gly(x), where x is 0 to 6; (Gly Gly Gly Gly Ser)n, wherein n is an integer of one or more; and (Ser Gly Gly Gly Gly)n, wherein n is an integer of one or more. In some embodiments, the linker peptides are modified such that the amino acid sequence GSG (that occurs at the junction of traditional Gly/Ser linker peptide repeats) is not present. For example, in some embodiments, the peptide linker includes an amino acid sequence selected from the group consisting of: (GGGXX)nGGGGS and GGGGS(XGGGS)n, where X is any amino acid that can be inserted into the sequence and not result in a polypeptide including the sequence GSG, and n is 0 to 4. In some embodiments, the sequence of a linker peptide is (GGGX1X2)nGGGGS and X1 is P and X2 is S and n is 0 to 4. In some other embodiments, the sequence of a linker peptide is (GGGX1X2)nGGGGS and X1 is G and X2 is Q and n is 0 to 4. In some other embodiments, the sequence of a linker peptide is (GGGX1X2)nGGGGS and X1 is G and X2 is A and n is 0 to 4. In yet other embodiments, the sequence of a linker peptide is GGGGS(XGGGS)n, and X is P and n is 0 to 4. In some embodiments, a linker peptide of the disclosure comprises or consists of the amino acid sequence (GGGGA)₂GGGGS. In some embodiments, a linker peptide comprises or consists of the amino acid sequence (GGGGQ)₂GGGGS. In another embodiment, a linker peptide comprises or consists of the amino acid sequence (GGGPS)₂GGGGS. In another embodiment, a linker peptide comprises or consists of the amino acid sequence GGGGS(PGGGS)₂. In yet a further embodiment, a linker peptide comprises or consists of the amino acid sequence GSGGS or SGGSGS. In some embodiments, a linker peptide comprising or consists of the amino acid sequence GGGGSGGGGSGGGSGGGGS.

In some embodiments, the linker is a hinge region of a protein, such as a CD8 hinge region.

In some embodiments, the chimeric polypeptide further includes a signal peptide operably linked upstream (e.g., N-terminally) to the extracellular domain. Any signal peptide that targets a protein to the cell membrane of a phagocytic cell can be used. In some embodiments, the signal peptide is derived from a phagocytic receptor. In some embodiments, the signal peptide is derived from a T cell receptor or co-receptor. In certain embodiments, the signal peptide is selected from a CD8 signal peptide and a mannose receptor signal peptide. In some embodiments, the chimeric polypeptide does not comprise its corresponding signal peptide.

In some embodiments, the chimeric polypeptide further includes a tag. Suitable tags for use in protein detection and/or purification are known in the art, any of which may be included in the chimeric polypeptides described herein. Exemplary tags include, but are not limited to, poly-His tags, maltose-binding protein tags, glutathione-S-transferase tags, and calmodulin binding protein tags. In some embodiments, the chimeric polypeptide does not comprise a tag.

### Viral-Binding Polypeptides

In some embodiments, the chimeric polypeptides described herein include an extracellular portion containing a binding region that a virus specifically binds to (e.g., a viral-binding polypeptide.

In some embodiments, the viral-binding polypeptide is a portion of angiotensin converting enzyme 2 (ACE2), the receptor SARS-CoV-2 virus binds to in order to infect cells. Accordingly, in some embodiments, the viral binding polypeptide is a portion of ACE2 sufficient for binding to SARS-CoV-2 spike protein. In some embodiments, the ACE2 is human ACE2 (SEQ ID NO: 2). In some embodiments, the viral-binding polypeptide comprises amino acids 19-358, 19-605, or 19-740 of SEQ ID NO:2, provided herein as SEQ ID NOS: 4, 6, and 8, respectively. Thus, in some embodiments, the viral-binding polypeptide comprises, or alternatively consists of, an amino acid sequence selected from the group consisting of SEQ ID NO: 2, SEQ ID NO: 4, SEQ ID NO: 6, and SEQ ID NO: 8.

In some embodiments, the viral-binding polypeptide comprises an amino acid sequence that has at least 80% sequence identity to an amino acid sequence selected from the group consisting of SEQ ID NO: 2, SEQ ID NO: 4, SEQ ID NO: 6, and SEQ ID NO: 8. In some embodiments, the viral-binding polypeptide comprises an amino acid sequence that has at least 85%, at least 90%, at least 95%, at least 96%, at least 97%, at least 98%, at least 99%, or 100% sequence identity to an amino acid sequence selected from SEQ ID NO: 2, SEQ ID NO: 4, SEQ ID NO: 6, and SEQ ID NO: 8.

In some embodiments, the viral-binding polypeptide is a portion of a receptor that HIV virus bind to in order to infect cells, including, for example, CD4, CCR5, and CXCR4. In some embodiments, the viral-binding polypeptide is a portion of CD4 sufficient for binding to HIV, such as, for example, a CD4 extracellular domain or fragment thereof. In some embodiments, the viral-binding polypeptide is a portion of CCR5 sufficient for binding to HIV, such as, for example, a CCR5 extracellular domain or fragment thereof. In some embodiments, the viral-binding polypeptide is a portion of CXCR4 sufficient for binding to HIV, such as, for example, a CXCR4 extracellular domain or fragment thereof.

In some embodiments, the viral-binding polypeptide is a portion of a receptor that a filovirus (such as Ebola virus or Marburg virus) binds to in order to infect cells, including, for example, T-cell Ig and mucin domain 1 (TIM1). In some embodiments, the viral-binding polypeptide is a portion of TIM-1 sufficient for binding to a filovirus, such as, for example, a TIM-1 extracellular domain or fragment thereof.

In some embodiments, the viral-binding polypeptide is a portion of a receptor that a Measles virus binds to in order to infect cells, including, for example, CD46 and SLAMF1 (CD150) In some embodiments, the viral binding protein is a portion of CD46 sufficient for binding to Measles virus, such as, for example, CD46 extracellular domain or fragment thereof. In some embodiments, the viral-binding protein is a portion of SLAMF1 sufficient for binding to Measles virus, such as, for example, SLAMI1 extracellular domain or fragment thereof.

### Antigen-Binding Polypeptides

In some embodiments, the chimeric polypeptides described herein include an antigen-binding portion that binds to one or more target antigens of interest. In some embodiments, the antigen-binding portion binds to one or more target antigens expressed on the surface of a target cell (e.g., cell surface markers). Examples of cell surface markers that can act as an antigen that binds to the antigen binding portion of the chimeric polypeptide include those associated with viral, bacterial and parasitic infections, autoimmune disease, and cancer cells. In some embodiments, the antigen-binding portion binds to a cancer-associated antigen, e.g., tumor antigen, such as an antigen that is specific for a tumor or cancer of interest. Accordingly, in some embodiments, the extracellular domain of the chimeric polypeptide includes an antigen-binding portion that binds to one or more specific for one or more cancer-associated antigen. Generally, the cancer-associated antigen can be any cancer-associated antigen. Suitable cancer-associated antigens include, but are not limited to, CD19, CD22, HER2 (ERBB2/neu), Mesothelin, PSCA, CD123, CD30, CD171, CD138, CS-1, CLECL1, CD33, CD79b, EGFRvIII, GD2, GD3, BCMA, PSMA, RORI, FLT3, TAG72, CD38, CD44v6, CEA, EPCAM, B7H3 (CD276), KIT (CD 117), CD213A2, IL-1 IRa, PRSS21, VEGFR2, CD24, MUC-16, PDGFR-beta, SSEA-4, CD20, MUC1, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, and IGLLE. In some embodiments, the one or more cancer-associated antigens is selected from the group consisting of FLT3, CD19 and CD20.

A binding domain includes any naturally occurring, synthetic, semi-synthetic, or recombinantly produced binding partner for a biological molecule or other target of interest. In some embodiments, the binding region is an antigen-binding region, such as an antibody or functional binding domain or antigen-binding portion thereof. The antigen-binding region can include any domain that binds to the antigen and may include, but is not limited to, a monoclonal antibody, a polyclonal antibody, a synthetic antibody, a human antibody, a humanized antibody, a non-human antibody, and any fragment thereof. Thus, in some embodiments, the antigen binding domain portion includes a mammalian antibody or a fragment thereof. Non-limiting examples of antigen-binding regions suitable for the chimeric polypeptides of this disclosure include an antigen-binding fragment (Fab), a single chain variable fragment (scFv), a nanobody, a VH domain, a VL domain, a single domain antibody (sdAb), a VNAR domain, and a VHH domain, a bispecific antibody, a diabody, or a functional fragment of any thereof.

In some embodiments, “antigen-binding fragment” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody including one or more CDRs. Blocking antibodies and non-blocking antibodies are both suitable. As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-CD 19 antibody can prevent, inhibit, block, or reduce the binding interaction between CD 19 and its natural ligand (e.g., CD77), thus preventing, blocking, inhibiting, or reducing the immunosuppressive functions associated with the CD19/CD77 interaction. The term “non-blocking” antibody refers to an antibody that does not interfere, inhibits, blocks, or reduces biological or functional activity of the antigen to which it binds.

Accordingly, in some embodiments, the antigen-binding portion of the chimeric polypeptides described herein includes an amino acid sequence for an antibody selected from the group consisting of antigen-binding fragments (Fab), single-chain variable fragments (scFv), nanobodies, VH domains, VL domains, single domain antibodies (dAb), VNAR domains, and VHH domains, bispecific antibodies, diabodies, or a functional fragment of any one of theforegoing. In some embodiments, the antigen-binding portion includes a heavy chain variable region and a light chain variable region.

In some embodiments, the heavy chain variable region and the light chain variable region of the antigen-binding region are operably linked to each other via one or more intervening amino acid residues that are positioned between the heavy chain variable region and the light chain variable region. In some embodiments, the one or more intervening amino acid residues include a linker peptide sequence.

In some embodiments, the antigen-binding region is derived from the same cell type or the same species in which the chimeric polypeptide will ultimately be used. For example, for use in humans, the antigen-binding region of the chimeric polypeptide includes a human antibody, a humanized antibody, or a fragment thereof.

### Endocytic Receptors

In some embodiments, the chimeric polypeptides described herein include a portion of an endocytic receptor. In some embodiments, the chimeric polypeptides described herein include a portion of an endocytic receptor including one or more of an extracellular domain or fragment thereof, a transmembrane domain or fragment thereof, and an intracellular domain or fragment thereof. In some embodiments, the chimeric polypeptides described herein include an intracellular signaling region of an endocytic receptor. In some embodiments, the chimeric polypeptides described herein include a transmembrane domain and an intracellular domain of an endocytic receptor.

An intracellular signaling region of an endocytic receptor refers to an intracellular effector domain, which, upon binding of the target molecule (e.g., a viral antigen or a tumor-associated antigen) targeted by the extracellular domain of the chimeric polypeptide expressed by a host cell, activates one or more signaling pathways in the host cell resulting in endocytosis, including, in certain embodiments, cytoskeletal rearrangement of the host cell and internalization of the target cell, microbe, or particle associated with the antigen. In some embodiments, an intracellular signaling domain activates one or more signaling pathways resulting in phagocytosis of the target cell, microbe, or particle.

In some embodiments, the intracellular signaling domain from the endocytic receptor is capable of mediating an endogenous phagocytic signaling pathway. In some embodiments, the intracellular domain of the chimeric polypeptide includes a domain responsible for signal activation and/or transduction. Non-limiting examples of an intracellular domain suitable for the chimeric polypeptides disclosed herein include the cytoplasmic portion of a surface receptor capable of initiating signal transduction in a phagocytic cell (e.g., monocyte, macrophage or dendritic cell), as well as any derivative or variant of these elements and any synthetic sequence that has the same functional capability. In some embodiments, the chimeric polypeptide of the disclosure includes at least one intracellular domain derived from endocytic receptors such as, e.g., mannose receptor, MER proto-oncogene tyrosine kinase (MERTK), dectin-1, and a scavenger receptor.

In some embodiments, the scavenger receptor is a member of class A, B, C, D, E, F, G, H, I K, J, K or L scavenger receptors. Examples of scavenger receptors suitable for use in chimeric polypeptides of the present disclosure include, but are not limited to, scavenger receptor class A type I/II (SR AI/II), macrophage receptor with collagenous structure (MARCO), SCARA5 receptor, scavenger receptor with C-type lectin (SRCL), CD36, scavenger receptor class B type I (SR-BI), CD68, lectin-like oxLDL receptor 1 (LOX-1), scavenger receptor expressed by endothelial cell (SREC), multiple EGF like portions 10 (MEGF10), scavenger receptor for phosphatidylserine and oxidized lipoprotein (SR-PSOX), link domain-containing scavenger receptor-1 (FEEL-1), CD163, receptor for advanced glycation end products (RAGE), CD44, and scavenger receptor class L type I (SR-L1).

In some embodiments, chimeric polypeptides of the present disclosure comprise a portion of an endocytic receptor containing an amino acid sequence that has at least 80% sequence identity to an amino acid sequence selected from SEQ ID NO: 10 (mannose receptor 82 aa C-terminal fragment); SEQ ID NO: 12 (mannose receptor 96 aa C-terminal fragment); SEQ ID NO: 14 (MERTK C-terminal fragment); SEQ ID NO: 16 (dectin-1 N-terminal fragment); SEQ ID NO: 18 (dectin-1 full-length protein); SEQ ID NO: 20 (FcγR intracellular fragment); SEQ ID NO: 22 (SR-AI/II fragment); SEQ ID NO: 24 (MARCO fragment); SEQ ID NO: 26 (SCARA5 receptor fragment); SEQ ID NO: 28 (SRCL fragment); SEQ ID NO: 30 (CD36 fragment); SEQ ID NO: 32 (SR-BI fragment); SEQ ID NO: 34 (CD68 fragment); SEQ ID NO: 36 (LOX-1 fragment); SEQ ID NO: 38 (SREC fragment); SEQ ID NO: 40 (MEGF10 fragment); SEQ ID NO: 42 (SR-PSOX fragment); SEQ ID NO: 44 (FEEL-1 fragment); SEQ ID NO: 46 (CD163 fragment); SEQ ID NO: 48 (RAGE fragment); SEQ ID NO: 50 (CD44 fragment); and SEQ ID NO: 52 (SR-L1 fragment).

In some embodiments, chimeric polypeptides of the present disclosure comprise a portion of an endocytic receptor containing an amino acid sequence that has at least 85%, at least 90%, at least 95%, at least 96%, at least 97%, at least 98%, at least 99%, or 100% sequence identity to an amino acid sequence selected from SEQ ID NO: 10 (mannose receptor 82 aa C-terminal fragment); SEQ ID NO: 12 (mannose receptor 96 aa C-terminal fragment); SEQ ID NO: 14 (MERTK C-terminal fragment); SEQ ID NO: 16 (dectin-1 N-terminal fragment); SEQ ID NO: 18 (dectin-1 full-length protein); SEQ ID NO: 20 (FcγR intracellular fragment); SEQ ID NO: 22 (SR-AI/II fragment); SEQ ID NO: 24 (MARCO fragment); SEQ ID NO: 26 (SCARA5 receptor fragment); SEQ ID NO: 28 (SRCL fragment); SEQ ID NO: 30 (CD36 fragment); SEQ ID NO: 32 (SR-BI fragment); SEQ ID NO: 34 (CD68 fragment); SEQ ID NO: 36 (LOX-1 fragment); SEQ ID NO: 38 (SREC fragment); SEQ ID NO: 40 (MEGF10 fragment); SEQ ID NO: 42 (SR-PSOX fragment); SEQ ID NO: 44 (FEEL-1 fragment); SEQ ID NO: 46 (CD163 fragment); SEQ ID NO: 48 (RAGE fragment); SEQ ID NO: 50 (CD44 fragment); and SEQ ID NO: 52 (SR-L1 fragment).

In some embodiments, the chimeric polypeptides of the present disclosure do not comprise a recruitment portion. In the context of the present disclosure, a recruitment portion does not encompass an endocytic receptor intracellular region. Instead, a recruitment portion binds to a cytosolic protein of a phagocytic signaling pathway and typically is used to activate and/or enhance activity of the endogenous phagocytic signaling pathway. Examples of recruitment portions include, but are not limited to, a p85-recruitment portion that binds a p85 regulatory subunit of phosphoinositide 3-kinase (PI3K); an SH3 portion derived from Crk, Cdc25, Phospholipase, Ras, Vav, GRB2, FAK, Pyk2, TRIP10 or Gads; and a proline-rich peptide sequence from C3G, p41, PEP, p4′7, HPK1, SLP-1, CD3.epsilon., PAK, AIP4, or Sos, wherein the proline-rich peptide sequence binds to an SH3 portion-containing protein. In some embodiments, the recruitment portion is the p85-recruitment portion derived from CD19, Gab2, IREM-1, PDGF receptor, CSFR-1, c-Kit, ErbB3, or CD7.

### Endocytic Receptor Ligands

In some embodiments, the chimeric polypeptides described herein include a ligand for an endocytic receptor. The endocytic receptor can be any endocytic receptor described herein. In some embodiments, the ligand is one or more of the following: soluble CD163 (sCD163), mannose, growth arrest specific factor 6 (Gas6), Protein S (Pros1), Low Density Cholesterol (LDL), acetylated LDL (AcLDL), oxidised LDL (OxLDL) polyanions, ferritin, ferritin light chain, beta-glucans, N-acetylgalactosamine, GAL-type ligands (beta-D-galactopyranose), L-fucose, D-fucose, diacylated lipopeptides, High Density Cholesterol (HDL), lectins, selectins, Clq, hemoglobin, haptoglobin, amyloid-beta peptide, hyaluronic acid (HA aka hyaluronan), microtubule-associated protein Tau (MAPT), or a fragment of any ligand described herein. Examples of ligands for endocytic receptors include, but are not limited to, a ligand for a mannose receptor, such as mannose or a fragment thereof; a ligand for a MERTK, such as growth arrest specific factor 6 (Gas6) or Protein S (Pros1), or fragments thereof; a ligand for a dectin-1, such as beta-glucans; a ligand for a MEGF10, such as C1q or a fragment thereof; and a ligand for a CD163, such as soluble CD163 of a fragment thereof. In some embodiments, the ligand is soluble CD163 (sCD163). In some embodiments, the ligand comprises an amino acid sequence at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 105.

### Vectors

In one aspect, provided herein are recombinant vectors comprising nucleic acid molecules capable of expressing one or more chimeric polypeptides of the present disclosure. Some embodiments disclosed herein relate to isolated, synthetic, or recombinant nucleic acid molecules encoding the chimeric polypeptides of the disclosure, expression cassettes, and expression vectors containing these nucleic acid molecules. In some embodiments, an isolated, synthetic, or recombinant nucleic acid molecule of the disclosure is operably linked to regulatory sequences which allow expression of the chimeric polypeptides in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

In some embodiments, nucleic acid molecules are provided that include a nucleotide sequence encoding a polypeptide that includes an amino acid sequence having at least 80% sequence identity to the amino acid sequence of a chimeric polypeptide as disclosed herein or a functional fragment thereof. In some embodiments, the nucleic acid molecules include a nucleotide sequence encoding a polypeptide that includes an amino acid sequence having at least 85%, at least 90%, at least 95%, at least 96%, at least 97%, at least 98%, at least 99%, or 100% sequence identity to the amino acid sequence of a chimeric polypeptide as disclosed herein or a functional fragment thereof.

In some embodiments, the nucleic acid molecules include a nucleotide sequence encoding a polypeptide that includes an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 61-67 or a functional fragment thereof. In some embodiments, the nucleic acid molecules include a nucleotide sequence encoding a polypeptide that includes an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 86-103 or a functional fragment thereof. In some embodiments, the nucleic acid molecules include a nucleotide sequence encoding a polypeptide that includes an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 107, or a functional fragment thereof.

In some embodiments, the nucleic acid molecules include a nucleotide sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NOs: 54-60 or a functional fragment thereof. In some embodiments, the nucleic acid molecules include a nucleotide sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NOs: 68-85, or a functional fragment thereof. In some embodiments, the nucleic acid molecules include a nucleotide sequence that has at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence of SEQ ID NO: 106, or a functional fragment thereof.

In some embodiments, the nucleic acid molecule as disclosed herein is operably linked to a heterologous nucleic acid sequence. Some embodiments disclosed herein relate to vectors or expression cassettes including an isolated, synthetic, or recombinant nucleic acid molecule as disclosed herein. The expression cassette generally contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. As such, the term expression cassette may be used interchangeably with the term “expression construct”. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In some embodiments, the vector comprises one or more additional elements. Additional elements include, but are not limited to, promoters, enhancers, polyadenylation (polyA) sequences, and selection genes.

In some embodiments, the vector comprises a polynucleotide sequence that encodes for a selectable marker that confers a specific trait on cells in which the selectable marker is expressed enabling artificial selection of those cells. Exemplary selectable markers include, but are not limited to, antibiotic resistance genes, e.g., resistance to kanamycin, ampicillin, or triclosan.

In some embodiments, the vector comprises a transcriptional regulatory element. Exemplary transcriptional regulatory elements include, but are not limited to promoters and enhancers.

A DNA vector can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N. Y.) and other standard molecular biology laboratory manuals.

The nucleic acid sequences encoding the chimeric polypeptides of the present disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric polypeptides as disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Non-limiting examples of vectors suitable for use include T7-based vectors for use in bacteria, the pMSXND expression vector for use in mammalian cells, and baculovirus-derived vectors for use in insect cells. In some embodiments nucleic acid inserts, which encode the subject chimeric polypeptide in such vectors, can be operably linked to a promoter, which is selected based on, for example, the cell type in which expression is sought. A non-limiting exemplification of suitable promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, MoMuLV promoter, an avian leukemia virus promoter, an Epstein-Barr virus immediate early promoter, mouse mammary tumor virus (MMTV), human immunodeficiency virus (HIV) long terminal repeat (LTR) promoter, a Rous sarcoma virus promoter, the elongation factor-1a promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans will readily appreciate numerous promoters and other regulatory elements which can be used to direct expression of nucleic acids.

In selecting an expression control sequence, a variety of factors should also be considered. These include, for example, the relative strength of the sequence, its controllability, and its compatibility with the actual DNA sequence encoding the subject chimeric polypeptide, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma vims, adenovirus and cytomegalovirus. Non-limiting examples of useful expression vectors for bacterial hosts include known bacterial plasmids, such as plasmids from E. coli, including col EI, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2m plasmid and derivatives thereof. Non-limiting examples of useful vectors for insect cells include pVL 941 and pFastBac™ 1.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Recombinant prokaryotic or eukaryotic cells that contain a chimeric polypeptide as disclosed herein, and/or contain and express a nucleic acid molecule that encodes any one of the chimeric polypeptides disclosed herein are also features of the disclosure. In some embodiments, a recombinant cell of the disclosure is a transfected cell, e.g., a cell into which a nucleic acid molecule, for example a nucleic acid molecule encoding a chimeric polypeptide disclosed herein, has been introduced by means of recombinant methodologies and techniques. The progeny of such a cell are also considered within the scope of the disclosure. Cell cultures containing at least one recombinant cell as disclosed herein are also within the scope of the present disclosure.

The precise components of the expression system are not critical. For example, a chimeric polypeptide as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). In some embodiments, the recombinant cell is a phagocytic cell, e.g., macrophage. Both professional phagocytes and non-professional phagocytes are suitable. In some embodiments, the phagocytic cell is a professional phagocyte. In some embodiments, the phagocytic cell is a non-professional phagocyte. In some embodiments, the phagocytic cell is selected from the group consisting of macrophages, dendritic cells, mast cells, monocytes, neutrophils, microglia, and astrocytes. In some embodiments, the phagocytic cell is a BMDM or a BMDC. In some embodiments, the phagocytic cell is a Thp-1 monocyte. In some embodiments, the phagocytic cell is a J774A.1 macrophage. These cells are available from many sources, including the American Type Culture Collection (Manassas, Va.). In selecting an expression system, it matters only that the components are compatible with one another. Artisans of ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, chimeric polypeptides obtained will be glycosylated or unglycosylated depending on the host organism used to produce the chimeric polypeptides. If bacteria are chosen as the host, then the chimeric polypeptide produced will be unglycosylated. Eukaryotic cells, on the other hand, will typically glycosylate the chimeric polypeptides, although perhaps not in the same way as native polypeptides is glycosylated. The chimeric polypeptides produced by the transformed host cell can be purified according to any suitable methods known in the art. Produced chimeric polypeptides can be isolated from inclusion bodies generated in bacteria such as E. coli, or from conditioned medium from either mammalian or yeast cultures producing a given chimeric polypeptide using cation exchange, gel filtration, and or reverse phase liquid chromatography.

Accordingly, another exemplary method of constructing a DNA sequence encoding the chimeric polypeptides of the disclosure is by chemical synthesis. This includes direct synthesis of a peptide by chemical means of the protein sequence encoding for a chimeric polypeptide exhibiting the properties described. This method can incorporate both natural and unnatural amino acids at positions that affect the binding affinity of the chimeric polypeptide with the target antigen and/or target protein. Alternatively, a gene which encodes the desired chimeric polypeptide can be synthesized by chemical means using an oligonucleotide synthesizer. Such oligonucleotides are designed based on the amino acid sequence of the desired chimeric polypeptide, and preferably selecting those codons that are favored in the host cell in which the recombinant chimeric polypeptide will be produced. In this regard, it is well recognized in the art that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular chimeric polypeptide, there will be many DNA degenerate sequences that will code for that chimeric polypeptide. For example, it will be appreciated that in addition to the DNA sequences for chimeric polypeptides provided in the Sequence Listing, there will be many degenerate DNA sequences that code for the chimeric polypeptides disclosed herein. These degenerate DNA sequences are considered within the scope of this disclosure. Therefore, “degenerate variants thereof” in the context of this disclosure means all DNA sequences that code for and thereby enable expression of a particular chimeric polypeptide.

The DNA sequence encoding the subject chimeric polypeptide, whether prepared by site directed mutagenesis, chemical synthesis or other methods, can also include DNA sequences that encode a signal sequence. Such signal sequence, if present, should be one recognized by the cell chosen for expression of the chimeric polypeptide. It can be prokaryotic, eukaryotic or a combination of the two. In general, the inclusion of a signal sequence depends on whether it is desired to secrete the chimeric polypeptide as disclosed herein from the recombinant cells in which it is made. If the chosen cells are prokaryotic, it generally is preferred that the DNA sequence not encode a signal sequence. If the chosen cells are eukaryotic, it generally is preferred that a signal sequence be included.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides; some or all of the non-coding sequences that lie upstream or downstream from a coding sequence (e.g., the coding sequence of a chimeric polypeptide disclosed herein) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a chimeric polypeptide disclosed herein) is incorporated into a vector (e.g., a plasmid or viral vector) or into the genome of a heterologous cell (or the genome of a homologous cell, at a position other than the natural chromosomal location).

### Methods of Use

In another aspect, the instant disclosure provides a method of treating a subject using the chimeric bait receptors (CBRs), chimeric phagocytic receptors (CPRs), bait macrophage engagers (BMEs), antigen macrophage engagers (AMEs), recombinant vectors, engineered cells (e.g., a cell comprising a heterologous and/or recombinant nucleic acid), or pharmaceutical compositions disclosed herein. Any disease or disorder in a subject that would benefit from treatment with a recombinant cell of the present disclosure, or a polypeptide or polynucleotide or vector of the present disclosure can be treated using the methods disclosed herein. The chimeric polypeptides, nucleic acid molecules, and/or pharmaceutical compositions of the disclosure can be used to treat individuals who have, who are suspected of having, or who may be at high risk for developing one or more health conditions or disorders. Exemplary health conditions and disorders of interest can include, without limitation, those associated with acute and chronic infections, inflammatory diseases, immune diseases, and various cancers. In some embodiments, the methods disclosed herein are useful in treating the one or more health conditions or disorders by enhancing the removal of infected, transformed, malignant, apoptotic, damaged or necrotic cells or particles from the individual's body.

In certain embodiments, the method comprises administering to the subject an effective amount of a recombinant cell or population thereof as disclosed herein.

Cells administered to the subject can be autologous or allogeneic.

The number of cells that are employed will depend upon a number of circumstances including, the lifetime of the cells, the protocol to be used (e.g., the number of administrations), the ability of the cells to multiply, the stability of the recombinant construct, and the like. In certain embodiments, the cells are applied as a dispersion, generally being injected at or near the site of interest. The cells may be administered in any physiologically acceptable medium.

In certain embodiments, the viral infection is caused by an enveloped RNA virus. Examples of enveloped RNA viruses include, but are not limited to, Togaviridae (e.g., Chikungunya virus (CHIKV)), Coronaviridae (e.g., SARS-CoV-2), Flaviviridae (e.g., dengue, zika), Orthomyxoviridae (e.g., influenza), Filoviridae (e.g., Ebola), Paramyxoviridae (e.g., measles, respiratory syncytial virus), Retroviridae (e.g., HIV), and Bunyaviridae (e.g., hantavirus).

In some embodiments, the viral infection is caused by a coronavirus. As used herein, the term “coronavirus” refers to the group of related RNA viruses that constitute the subfamily Orthocoronavirinae, belonging to the family Coronaviridae, order Nidovirales. Coronaviruses are further divided into the four genera: alphacoronavirus, betacoronavirus, gammacoronavirus, and deltacoronavirus. Accordingly, in some embodiments, the viral infection is caused by an alphacoronavirus, e.g., human coronavirus 229E (HCoV-229E), porcine epidemic diarrhea virus (PEDV), human coronavirus NL63 (HCoV-NL63), and alphacoronavirus 1. In some embodiments, the viral infection is caused by a betacoronavirus, e.g., betacoronavirus 1, human coronavirus OC43 (HCoV-OC43), severe acute respiratory syndrome coronavirus (SARS-CoV), human coronavirus HKU1 (HCoV-HKU1), Middle East respiratory syndrome-related coronavirus (MERS-CoV), and severe acute respiratory syndrome coronavirus 2 (SARS-CoV-2). In some embodiments, the viral infection is caused by a gammacoronavirus. In some embodiments, the viral infection is caused by a deltacoronavirus.

In a preferred embodiment, the viral infection is caused by betacoronavirus. In some embodiments, the viral infection is caused by human coronavirus OC43 (HCoV-OC43), severe acute respiratory syndrome coronavirus (SARS-CoV), human coronavirus HKU1, Middle East respiratory syndrome-related coronavirus (MERS-CoV), or severe acute respiratory syndrome coronavirus 2 (SARS-CoV-2). In some embodiments, the viral infection is caused by severe acute respiratory syndrome coronavirus 2 (SARS-CoV-2).

In some embodiments, the viral infection is caused by a virus that uses lysosomes for egress.

In humans, coronaviruses cause respiratory tract infections that can be mild, such as some cases of the common cold and others that can be lethal, such as SARS-CoV, MERS-CoV, and SARS-CoV-2. Symptoms associated with coronavirus infection in general include fever, cough, shortness of breath, pain or pressure in the chest, confusion, bluish lips or face, pneumonia, bronchitis, runny nose, sneezing, chills, exacerbated asthma, acute respiratory distress syndrome (ARDS), RNAaemia, acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, malnutrition, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, abdominal pain, secondary infection, and multi-organ failure.

Common symptoms associated with SARS-CoV-2, in particular, include fever, cough, shortness of breath, difficult breathing, fatigue, loss of appetite, muscle or body aches, and production of mucus or phlegm. Less common symptoms include sore throat, headache, chills, loss of taste or smell, congestion or runny nose, nausea, vomiting, diarrhea, pain or pressure in the chest, confusion, inability to wake or stay awake, and bluish lips or face. While the majority of cases result in mild symptoms, some progress to severe pneumonia, acute respiratory distress symptom (ARDS), cardiac injury, and multi-organ failure.

In some embodiments, the viral infection is caused by a virus from the family Togaviridae, such as an Alphavirus. In some embodiments, the Alphavirus is selected from the group consisting of Aura virus, Barmah Forest virus, Bebaru virus, Caaingua virus, Cabassou virus, Chikungunya virus, Eastern equine encephalitis virus, Eilat virus, Everglades virus, Fort Morgan virus, Getah virus, Highlands Jvirus, Madariaga virus, Mayaro virus, Middelburg virus, Mosso das Pedras virus, Mucambo virus, Ndumu virus, O'nyong'nyong virus, Pixuna virus, Rio Negro virus, Ross River virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, and Whataroa virus. In some embodiments, the Alphavirus is Chikungunya virus (CHIKV).

Common symptoms associated with CHIKV, in particular, include fever, joint pain and rash. Less common symptoms include headache, fatigue, digestive pathology, and conjunctivitis. Chronic symptoms associated with CHIKV include arthritis, long term musculoskeletal pain and asthenia.

In some embodiments, the viral infection is caused by a virus from the family Flaviviridae. In some embodiments, the virus from the family Flaviviridae is selected from the group consisting of Apoi virus, Aroa virus, Bagaza virus, Banzi virus, Bouboui virus, Bukalasa bat virus, Cacipacore virus, Carey Island virus, Cowbone Ridge virus, Dakar bat virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy virus, Uganda S virus, Usutu virus, Wesselsbron virus, West Nile virus, Yaounde virus, Yellow fever virus, Yokose virus, and Zika virus.

In some embodiments, the viral infection is caused by a virus from the family Orthomyxoviridae such as an Alphainfluenzavirus, Betainfluenzavirus, Deltainfluenzavirus, Gammainfluenzavirus, Isavirus, Thogotovirus, or a Quaranjavirus. In some embodiments, the virus from the family Orthomyxoviridae is selected from the group consisting of Influenza A virus, Influenza B virus, Influenza C virus, and Influenza D virus. Examples of Influenza A virus include, but are not limited to, H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

In some embodiments, the viral infection is caused by a virus from the family Filoviridae such as a Cuevavirus, Dianlovirus, Ebolavirus, Marburgvirus, Striavirus, or a Thamnovirus. In some embodiments, the virus from the family Filoviridae is selected from the group consisting of Lloviu cuevavirus, Mengla dianlovirus, Bombali ebolavirus, Bundibugyo ebolavirus, Reston ebolavirus, Sudan ebolavirus, Tai Forest ebolavirus, Zaire ebolavirus, Marburg marburgvirus, Xilang striavirus, and Huangjiao thamnovirus.

In some embodiments, the viral infection is caused by a virus from the family Paramyxoviridae such as a Metaavulavirus, Orthoavulavirus, Paraavulavirus, Metaparamyxyovirus, Aquaparamyxovirus, Ferlavirus, Henipavirus, Jeilongvirus, Morbillivirus, Narmovirus, Respirovirus, Salemvirus, Orthorubulavirus, Pararubulavirus, Pneumovirus, Cynoglossusvirus, Hoplichthysvirus, or a Scoliodonvirus. In some embodiments, the virus from the family Paramyxoviridae is selected from the group consisting of Canine distemper virus (CDV), Cetacean morbillivirus (CeMV), Feline morbillivirus (FeMV), Measles virus (MeV), Peste-des-petits-ruminants virus (PPRV), Phocine distemper virus (PDV), Newcastle disease virus, Rinderpest virus (RPV), Mumps virus, Hendra virus (HeV), Nipah virus (NiV), Human Parainfluenza Virus (HPIV-1, HPIV-2, HPIV-3, HPIV-4), avian metapneumovirus (AMPV), human metapneumovirus (HMPV), bovine respiratory syncytial virus (BRSV), human respiratory syncytial virus (HRSV), and murine pneumonia virus (MPV).

In some embodiments, the viral infection is caused by a virus from the family Retroviridae. In some embodiments, the virus from the family Retroviridae is selected from the group consisting of Human immunodeficiency virus 1 (HIV-1), Human immunodeficiency virus 2 (HIV-2), Human T-lymphotropic virus (HTLV), Murine leukemia virus (MLV), Avian leukosis virus, Rous sarcoma virus, Mouse mammary tumor virus (MMTV), Feline leukemia virus, Bovine leukemia virus, Simian immunodeficiency virus (SIV), and Feline immunodeficiency virus (FIV).

In some embodiments, the viral infection is caused by a virus from the family Bunyaviridae such as Peribunyaviridae, Phenuiviridae, Arenaviridae, Nairoviridae, and Hantaviridae. In some embodiments, the virus from the family Bunyaviridae is selected from the group consisting of Calfornia encephalitis virus, La Crosse encephalitis virus, Jamestown Canyon virus, Akabane virus, Oropouche virus, Group C virus, Guama virus, Tahyna virus, Snowshoe hare virus, Hantaan virus, Crimean-Congo hemorrhagic fever virus, Rift Valley, fever virus, Sandfly fever virus, Hazara virus, Dobrava virus, Seoul virus, Puumala virus, Sin Nombre virus, and severe fever with thrombocytopenia syndrome virus (SFTSV).

In certain embodiments, the cancer is cancer of the lung, bile duct cancer (e.g., cholangiocarcinoma), pancreatic cancer, colorectal cancer, ovarian, or gynecologic cancer. In certain embodiments, the cancer is leukemia (e.g., mixed lineage leukemia, acute lymphocytic leukemia, acute myeloid leukemia, chronic lymphocytic leukemia, or chronic myeloid leukemia), alveolar rhabdomyosarcoma, bone cancer, brain cancer (e.g., glioma, e.g., glioblastoma), breast cancer, cancer of the anus, anal canal, or anorectum, cancer of the eye, cancer of the intrahepatic bile duct (e.g., intrahepatic cholangiocellular cancer), cancer of the joints, cancer of the neck, gallbladder, or pleura, cancer of the nose, nasal cavity, or middle ear, cancer of the oral cavity, cancer of the vulva, myeloma (e.g., chronic myeloid cancer), colon cancer, esophageal cancer, cervical cancer, gastrointestinal cancer, gastrointestinal carcinoid tumor. Hodgkin's lymphoma, hypopharynx cancer, kidney cancer, larynx cancer, liver cancer (e.g., hepatocellular carcinoma), lung cancer (e.g., non-small cell lung cancer), malignant mesothelioma, melanoma, multiple myeloma, nasopharynx cancer, non-Hodgkin's lymphoma, ovarian cancer, pancreatic cancer, peritoneum, omentum, and mesentery cancer, pharynx cancer, prostate cancer, rectal cancer, renal cancer (e.g., renal cell carcinoma (RCC)), gastric cancer, small intestine cancer, soft tissue cancer, stomach cancer, carcinoma, sarcoma (e.g., synovial sarcoma, rhabdomyosarcoma), skin cancer, testicular cancer, thyroid cancer, head and neck cancer, ureter cancer, and urinary bladder cancer. In certain embodiments, the cancer is melanoma, breast cancer, lung cancer, prostate cancer, thyroid cancer, ovarian cancer, or synovial sarcoma. In one embodiment, the cancer is synovial sarcoma or liposarcoma (e.g., myxoid/round cell liposarcoma). In certain embodiments, the cancer is lung, cholangiocarcinoma, pancreatic, colorectal, gynecological or ovarian cancer.

A polypeptide, polynucleotide, recombinant vector, engineered cell, or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intrathecal, and subcutaneous routes. Pulmonary administration can also be employed, e.g., by use of an inhaler or nebulizer, and formulation with an aerosolizing agent for use as a spray. In certain embodiments, the polypeptide, polynucleotide, recombinant vector, engineered cell, or pharmaceutical composition described herein is delivered intravenously. In certain embodiments, the polypeptide, polynucleotide, vector, engineered cell, or pharmaceutical composition described herein is delivered subcutaneously. In certain embodiments, the polypeptide, polynucleotide, recombinant vector, engineered cell, or pharmaceutical composition described herein is delivered intranasally. In certain embodiments, the polypeptide, polynucleotide, recombinant vector, engineered cell, or pharmaceutical composition described herein is delivered intramuscally. In certain embodiments, the polypeptide, polynucleotide, recombinant vector, engineered cell, or pharmaceutical composition described herein is delivered intratumorally. In certain embodiments, the polypeptide, polynucleotide, recombinant vector, engineered cell, or pharmaceutical composition described herein is delivered into a tumor draining lymph node.

The amount of the polypeptide, polynucleotide, recombinant vector, engineered cell, or pharmaceutical composition which will be effective in the treatment and/or prevention of a condition will depend on the nature of the disease, and can be determined by standard clinical techniques.

The precise dose to be employed in a composition will also depend on the route of administration, and the seriousness of the infection or disease caused by it and should be decided according to the judgment of the practitioner and each subject's circumstances. For example, effective doses may also vary depending upon means of administration, target site, physiological state of the patient (including age, body weight, and health), whether the patient is a human or an animal, other medications administered, or whether treatment is prophylactic or therapeutic. Usually, the patient is a human, but non-human mammals including transgenic mammals can also be treated. Treatment dosages are optimally titrated to optimize safety and efficacy.

### Methods of Manufacture

Engineered cells described herein can be manufactured by any method known in the art. Some embodiments of the disclosure relate to a method for modifying a cell including introducing into the cell (a) a chimeric polypeptide as described herein; and/or (b) an isolated, synthetic, or recombinant nucleic acid molecule as described herein, to produce a recombinant (e.g., engineered or transgenic). For example, a chimeric polypeptide or nucleic molecule as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). In some embodiments, the recombinant cell is a phagocytic cell, e.g., phagocyte. Both professional phagocytes and non-professional phagocytes are suitable. In some embodiments, the phagocytic cell is a professional phagocyte. In some embodiments, the phagocytic cell is a non-professional phagocyte. In some embodiments, the phagocytic cell is selected from the group consisting of macrophages, dendritic cells, mast cells, monocytes, neutrophils, microglia, and astrocytes. In some embodiments, the phagocytic cell is a BMDM or a BMDC. In some embodiments, the phagocytic cell is a Thp1 monocyte. These cells are available from many sources, including the American Type Culture Collection (Manassas, Va.). In some embodiments, the phagocytic cell is a macrophage derived from pluripotent stem cells (iPSC-macrophages). Such iPSC-macrophages can be generated by knocking out B2M to eliminate all MHC I and subsequently knocking in HLA E. The modified iPSC can then be differentiated and polarized in culture into mature M1 macrophages using protocols known in the art (e.g., Cao et al., Stem Cell Reports, 2019). The source of iPSC or phagocytic cell may be an allogenic or an autologous donor.

In some embodiments, the recombinant cell expresses the chimeric polypeptide and possesses targeted effector activity. In some embodiments, introducing the chimeric polypeptide into the cell includes introducing a nucleic acid sequence encoding the chimeric polypeptide. In some embodiments, introducing the nucleic acid sequence includes electroporating an mRNA encoding the chimeric polypeptide.

Methods of introducing and expressing genes, such as the nucleic acid molecules and the chimeric polypeptides encoded thereby, into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells including vectors and/or exogenous nucleic acids are well-known in the art. For example, nucleic acids can be introduced into target cells using commercially available methods which include electroporation. Nucleic acids can also be introduced into cells using cationic liposome mediated transfection using lipofection, using polymer encapsulation, using peptide mediated transfection, or using biolistic particle delivery systems such as “gene guns”.

Biological methods for introducing a polynucleotide of interest into a host cell include the use of DNA and RNA vectors. RNA vectors include vectors having a RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes

The use of lipid formulations is contemplated for the introduction of the nucleic acids into a host cell (in vitro, ex vivo or in vivo). In some embodiments, the nucleic acid molecule or chimeric polypeptide may be associated with a lipid. The nucleic acid molecule or chimeric polypeptide associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Pharmaceutical Compositions

Provided herein are pharmaceutical compositions comprising a population of engineered immune effector cells disclosed herein having the desired degree of purity in a physiologically acceptable carrier, excipient or stabilizer (see, e.g., Remington's Pharmaceutical Sciences (1990) Mack Publishing Co., Easton, PA). In some embodiments, the cells are phagocytic cells. In some embodiments, the pharmaceutical composition is a protein therapeutic administered in a cell free manner. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

Pharmaceutical compositions described herein can be useful in inducing an immune response in a subject and treating a condition, such as cancer. In one embodiment, the present disclosure provides a pharmaceutical composition comprising a population of engineered immune effector cells described herein for use as a medicament. In another embodiment, the disclosure provides a pharmaceutical composition for use in a method for the treatment of cancer. In some embodiments, pharmaceutical compositions comprise a population of engineered immune effector cells disclosed herein, and optionally one or more additional prophylactic or therapeutic agents, in a pharmaceutically acceptable carrier.

A pharmaceutical composition may be formulated for any route of administration to a subject. Specific examples of routes of administration include parenteral administration (e.g., intravenous, subcutaneous, intramuscular). In some embodiments, the pharmaceutical composition is formulated for intravenous administration. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In some embodiments, the pharmaceutical composition is formulated for intravenous administration. Suitable carriers for intravenous administration include physiological saline or phosphate buffered saline (PBS), and solutions containing thickening and solubilizing agents, such as glucose, polyethylene glycol, and polypropylene glycol and mixtures thereof.

The compositions to be used for in vivo administration can be sterile. This is readily accomplished by filtration through, e.g., sterile filtration membranes.

Pharmaceutically acceptable carriers used in parenteral preparations include for example, aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN© 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether treatment is prophylactic or therapeutic. Treatment dosages are optimally titrated to optimize safety and efficacy.

### Kits

In one aspect, provided herein are kits comprising one or more pharmaceutical composition, population of engineered effector cells (e.g., recombinant phagocytic cells), protein, polynucleotide, or vector described herein and instructions for use. Such kits may include, e.g., a carrier, package, or container that is compartmentalized to receive one or more containers such as vials, tubes, and the like. Suitable containers include, for example, bottles, vials, syringes, and test tubes. In one embodiment, the containers are formed from a variety of materials such as glass or plastic.

In a specific embodiment, provided herein is a pharmaceutical kit comprising one or more containers filled with one or more of the ingredients of the pharmaceutical compositions described herein, population of engineered immune effector cells, polynucleotides, or vectors provided herein. In one embodiment, the kit comprises a pharmaceutical composition comprising a population of engineered immune effector cells described herein. In one embodiment, the kit comprises a pharmaceutical composition comprising a population of immune effector cells engineered according to a method described herein. In some embodiments, the kit contains a pharmaceutical composition described herein and a prophylactic or therapeutic agent. Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

The examples of the present disclosure are offered by way of illustration and explanation, and are not intended to limit the scope of the present disclosure.

### Example 1: Initial Design of Chimeric Bait Receptor and Chimeric Phagocytic Receptor Constructs

Initially, chimeric bait receptor (CBR) and chimeric phagocytic receptor (CPR) constructs were designed based on traditional chimeric antigen receptors (“CAR”). The CAR-based construct chosen for modification contained the following components from N terminus to C terminus:
CD8 signal peptide+anti-FLT3 scFv+CD8 hinge+CD8 transmembrane (TM) domain+CD3ζ intracellular domain+T2A+CopGFP (in frame with T2A).

The nucleotide sequence of the CAR-based construct is provided as SEQ ID NO: 53:

ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTGGATCCTTCGTGCCGGTCTTCCTGCCAGCGA
AGCCCACCACGACGCCAGCGCCGCGACCACCAACACCGGCGCCCACCATCGCGTCGCAGCCCCTGTCCCTGCGCCCA
GAGGCGTGCCGGCCAGCGGCGGGGGGCGCAGTGCACACGAGGGGGCTGGACTTCGCCTGTGATATCTACATCTGGGC
GCCCTTGGCCGGGACTTGTGGGGTCCTTCTCCTGTCACTGGTTATCACCCTTTACTGCAACCACAGGAACCGACTGA
AGATCCAAGTGCGAAAGGCAGCTATAACCAGCTATGAGAAATCAGATGGTGTTTACACGGGCCTGAGCACCAGGAAC
CAGGAGACTTACGAGACTCTGAAGCATGAGAAACCACCACAGTAG

Various constructs described below were developed starting from this base construct to test the CBR/CPR concept.

First, the CD8 TM domain and CD3ζ intracellular domain was replaced with the last 67 amino acids at the C terminus of mannose receptor (MR), which includes the TM and intracellular domains of MR. The resulting construct contained the following components from N terminus to C terminus:
CD8 signal peptide+anti-FLT3 scFv+CD8 hinge+MR™ domain+MR intracellular domain+T2A+CopGFP (in frame with T2A).

The nucleotide sequence of this construct containing the last 67 amino acids of Mannose Receptor is provided as SEQ ID NO: 68:

ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTGGATCCTTCGTGCCGGTCTTCCTGCCAGCGA
AGCCCACCACGACGCCAGCGCCGCGACCACCAACACCGGCGCCCACCATCGCGTCGCAGCCCCTGTCCCTGCGCCCA
GAGGCGTGCCGGCCAGCGGCGGGGGGCGCAGTGCACACGAGGGGGCTGGACTTCGCCTGTGATGGAGTAGTCATCAT
TGTGATCCTCCTGATTTTAACGGGTGCTGGCCTTGCCGCCTATTTCTTTTATAAGAAAAGACGTGTGCACCTACCTC
AAGAGGGCGCCTTTGAAAACACTCTGTATTTTAACAGTCAGTCAAGCCCAGGAACTAGTGATATGAAAGATCTCGTG
GGCAATATTGAACAGAATGAACACTCGGTCATCG

Second, to test the approach of using bait instead of scFv, various regions of ACE2 were tested that exhibit binding to Spike protein of SARS-CoV-2. Three separate constructs were designed containing three different ACE2 fragments. The components of the three constructs (N terminus to C terminus) are:
1. CD8 signal peptide+ACE2 (19-358)+CD8 hinge+CD8 TM domain+CD3ζ intracellular domain+T2A+CopGFP (in frame with T2A).
2. CD8 signal peptide+ACE2 (19-605)+CD8 hinge+CD8 TM domain+CD3ζ intracellular domain+T2A+CopGFP (in frame with T2A).
3. CD8 signal peptide+ACE2 (19-740)+CD8 TM domain+CD3ζ intracellular domain+T2A+CopGFP (in frame with T2A).

The nucleotide sequences of the ACE2 sequences included in constructs 1-3 is provided below.

ACE2 (19-358) (SEQ ID NO: 3):
CTGGCCTTACCAGTGACCGCCTTGCTCCTGCCGCTGGCCTTGCTGCTCCACGCCGCCAGGCCGGGATCCTCCACCAT
TGAGGAACAGGCCAAGACATTTTTGGACAAGTTTAACCACGAAGCCGAAGACCTGTTCTATCAAAGTTCACTTGCTT
CTTGGAATTATAACACCAATATTACTGAAGAGAATGTCCAAAACATGAATAATGCTGGGGACAAATGGTCTGCCTTT
TTAAAGGAACAGTCCACACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACACAATTCTAAATACAATGA
GCACCATCTACAGTACTGGAAAAGTTTGTAACCCAGATAATCCACAAGAATGCTTATTACTTGAACCAGGTTTGAAT
GAAATAATGGCAAACAGTTTAGACTACAATGAGAGGCTCTGGGCTTGGGAAAGCTGGAGATCTGAGGTCGGCAAGCA
GCTGAGGCCATTATATGAAGAGTATGTGGTCTTGAAAAATGAGATGGCAAGAGCAAATCATTATGAGGACTATGGGG
ATTATTGGAGAGGAGACTATGAAGTAAATGGGGTAGATGGCTATGACTACAGCCGCGGCCAGTTGATTGAAGATGTG
GAACATACCTTTGAAGAGATTAAACCATTATATGAACATCTTCATGCCTATGTGAGGGCAAAGTTGATGAATGCCTA
TCCTTCCTATATCAGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGACCAGGCCTGGGAT
GCACAGAGAATATTCAAGGAGGCCGAGAAGTTCTTTGTATCTGTTGGTCTTCCTAATATGACTCAAGGATTCTGGGA
AAATTCCATGCTAACGGACCCAGGAAATGTTCAGAAAGCAGTCTGCCATCCCACAGCTTGGGACCTGGGGAAGGGCG
ACTTCAGGATCGGATCCTTCGTGCCGGTCTTCCTGCCAGCGAAGCCCACCACGACGCCAGCGCCGCGACCACCAACA
CCGGCGCCCACCATCGCGTCGCAGCCCCTGTCCCTGCGCCCAGAGGCGTGCCGGCCAGCGGCGGGGGGCGCAGTGCA
CACGAGGGGGCTGGACTTCGCCTGTGATATCTACATCTGGGCGCCCTTGGCCGGGACTTGTGGGGTCCTTCTCCTGT
CACTGGTTATCACCCTTTACTGCAACCACAGGAACAGAGTGAAGTTCAGCAGGAGCGCAGACGCCCCCGCGTACCAG
CAGGGCCAGAACCAGCTCTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGCCCCCTCGC
ACE2 (19-605) (SEQ ID NO: 5):
ATGGCCTTACCAGTGACCGCCTTGCTCCTGCCGCTGGCCTTGCTGCTCCACGCCGCCAGGCCGGGATCCTCCACCAT
TGAGGAACAGGCCAAGACATTTTTGGACAAGTTTAACCACGAAGCCGAAGACCTGTTCTATCAAAGTTCACTTGCTT
CTTGGAATTATAACACCAATATTACTGAAGAGAATGTCCAAAACATGAATAATGCTGGGGACAAATGGTCTGCCTTT
TTAAAGGAACAGTCCACACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACACAATTCTAAATACAATGA
GCACCATCTACAGTACTGGAAAAGTTTGTAACCCAGATAATCCACAAGAATGCTTATTACTTGAACCAGGTTTGAAT
GAAATAATGGCAAACAGTTTAGACTACAATGAGAGGCTCTGGGCTTGGGAAAGCTGGAGATCTGAGGTCGGCAAGCA
GCTGAGGCCATTATATGAAGAGTATGTGGTCTTGAAAAATGAGATGGCAAGAGCAAATCATTATGAGGACTATGGGG
ATTATTGGAGAGGAGACTATGAAGTAAATGGGGTAGATGGCTATGACTACAGCCGCGGCCAGTTGATTGAAGATGTG
GAACATACCTTTGAAGAGATTAAACCATTATATGAACATCTTCATGCCTATGTGAGGGCAAAGTTGATGAATGCCTA
TCCTTCCTATATCAGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGACCAGGCCTGGGAT
GCACAGAGAATATTCAAGGAGGCCGAGAAGTTCTTTGTATCTGTTGGTCTTCCTAATATGACTCAAGGATTCTGGGA
AAATTCCATGCTAACGGACCCAGGAAATGTTCAGAAAGCAGTCTGCCATCCCACAGCTTGGGACCTGGGGAAGGGCG
ACTTCAGGATCCTTATGTGCACAAAGGTGACAATGGACGACTTCCTGACAGCTCATCATGAGATGGGGCATATCCAG
TATGATATGGCATATGCTGCACAACCTTTTCTGCTAAGAAATGGAGCTAATGAAGGATTCCATGAAGCTGTTGGGGA
AATCATGTCACTTTCTGCAGCCACACCTAAGCATTTAAAATCCATTGGTCTTCTGTCACCCGATTTTCAAGAAGACA
ATGAAACAGAAATAAACTTCCTGCTCAAACAAGCACTCACGATTGTTGGGACTCTGCCATTTACTTACATGTTAGAG
AAGTGGAGGTGGATGGTCTTTAAAGGGGAAATTCCCAAAGACCAGTGGATGAAAAAGTGGTGGGAGATGAAGCGAGA
GATAGTTGGGGTGGTGGAACCTGTGCCCCATGATGAAACATACTGTGACCCCGCATCTCTGTTCCATGTTTCTAATG
ATTACTCATTCATTCGATATTACACAAGGACCCTTTACCAATTCCAGTTTCAAGAAGCACTTTGTCAAGCAGCTAAA
CATGAAGGCCCTCTGCACAAATGTGACATCTCAAACTCTACAGAAGCTGGACAGAAACTGTTCAATATGCTGAGGCT
TGGAAAATCAGAACCCTGGACCCTAGCATTGGAAAATGTTGTAGGAGCAAAGAACATGAATGTAAGGCCACTGCTCA
ACTACTTTGAGCCCTTATTTACCTGGCTGAAAGACCAGAACAAGAATTCTTTTGTGGGAGGATCCTTCGTGCCGGTC
TTCCTGCCAGCGAAGCCCACCACGACGCCAGCGCCGCGACCACCAACACCGGCGCCCACCATCGCGTCGCAGCCCCT
GTCCCTGCGCCCAGAGGCGTGCCGGCCAGCGGCGGGGGGCGCAGTGCACACGAGGGGGCTGGACTTCGCCTGTGATA
TCTACATCTGGGCGCCCTTGGCCGGGACTTGTGGGGTCCTTCTCCTGTCACTGGTTATCACCCTTTACTGCAACCAC
AGGAACAGAGTGAAGTTCAGCAGGAGCGCAGACGCCCCCGCGTACCAGCAGGGCCAGAACCAGCTCTATAACGAGCT
CAATCTAGGACGAAGAGAGGAGTACGATGTTTTGGACAAGAGACGTGGCCGGGACCCTGAGATGGGGGGAAAGCCGA
GAAGGAAGAACCCTCAGGAAGGCCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCAGTACAGCCACCAAGGACACCTA
CGACGCCCTTCACATGCAGGCCCTGCCCCCTCGC
ACE2 (19-740) (SEQ ID NO: 7):
ATGGCCTTACCAGTGACCGCCTTGCTCCTGCCGCTGGCCTTGCTGCTCCACGCCGCCAGGCCGGGATCCTCCACCAT
TGAGGAACAGGCCAAGACATTTTTGGACAAGTTTAACCACGAAGCCGAAGACCTGTTCTATCAAAGTTCACTTGCTT
CTTGGAATTATAACACCAATATTACTGAAGAGAATGTCCAAAACATGAATAATGCTGGGGACAAATGGTCTGCCTTT
TTAAAGGAACAGTCCACACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACACAATTCTAAATACAATGA
GCACCATCTACAGTACTGGAAAAGTTTGTAACCCAGATAATCCACAAGAATGCTTATTACTTGAACCAGGTTTGAAT
GAAATAATGGCAAACAGTTTAGACTACAATGAGAGGCTCTGGGCTTGGGAAAGCTGGAGATCTGAGGTCGGCAAGCA
GCTGAGGCCATTATATGAAGAGTATGTGGTCTTGAAAAATGAGATGGCAAGAGCAAATCATTATGAGGACTATGGGG
ATTATTGGAGAGGAGACTATGAAGTAAATGGGGTAGATGGCTATGACTACAGCCGCGGCCAGTTGATTGAAGATGTG
GAACATACCTTTGAAGAGATTAAACCATTATATGAACATCTTCATGCCTATGTGAGGGCAAAGTTGATGAATGCCTA
TCCTTCCTATATCAGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGACCAGGCCTGGGAT
GCACAGAGAATATTCAAGGAGGCCGAGAAGTTCTTTGTATCTGTTGGTCTTCCTAATATGACTCAAGGATTCTGGGA
AAATTCCATGCTAACGGACCCAGGAAATGTTCAGAAAGCAGTCTGCCATCCCACAGCTTGGGACCTGGGGAAGGGCG
ACTTCAGGATCCTTATGTGCACAAAGGTGACAATGGACGACTTCCTGACAGCTCATCATGAGATGGGGCATATCCAG
TATGATATGGCATATGCTGCACAACCTTTTCTGCTAAGAAATGGAGCTAATGAAGGATTCCATGAAGCTGTTGGGGA
AATCATGTCACTTTCTGCAGCCACACCTAAGCATTTAAAATCCATTGGTCTTCTGTCACCCGATTTTCAAGAAGACA
ATGAAACAGAAATAAACTTCCTGCTCAAACAAGCACTCACGATTGTTGGGACTCTGCCATTTACTTACATGTTAGAG
AAGTGGAGGTGGATGGTCTTTAAAGGGGAAATTCCCAAAGACCAGTGGATGAAAAAGTGGTGGGAGATGAAGCGAGA
GATAGTTGGGGTGGTGGAACCTGTGCCCCATGATGAAACATACTGTGACCCCGCATCTCTGTTCCATGTTTCTAATG
ATTACTCATTCATTCGATATTACACAAGGACCCTTTACCAATTCCAGTTTCAAGAAGCACTTTGTCAAGCAGCTAAA
CATGAAGGCCCTCTGCACAAATGTGACATCTCAAACTCTACAGAAGCTGGACAGAAACTGTTCAATATGCTGAGGCT
TGGAAAATCAGAACCCTGGACCCTAGCATTGGAAAATGTTGTAGGAGCAAAGAACATGAATGTAAGGCCACTGCTCA
ACTACTTTGAGCCCTTATTTACCTGGCTGAAAGACCAGAACAAGAATTCTTTTGTGGGATGGAGTACCGACTGGAGT
CCATATGCAGACCAAAGCATCAAAGTGAGGATAAGCCTAAAATCAGCTCTTGGAGATAAAGCATATGAATGGAACGA
CAATGAAATGTACCTGTTCCGATCATCTGTTGCATATGCTATGAGGCAGTACTTTTTAAAAGTAAAAAATCAGATGA
TTCTTTTTGGGGAGGAGGATGTGCGAGTGGCTAATTTGAAACCAAGAATCTCCTTTAATTTCTTTGTCACTGCACCT
AAAAATGTGTCTGATATCATTCCTAGAACTGAAGTTGAAAAGGCCATCAGGATGTCCCGGAGCCGTATCAATGATGC
TTTCCGTCTGAATGACAACAGCCTAGAGTTTCTGGGGATACAGCCAACACTTGGACCTCCTAACCAGCCCCCTGTTT
CCATCTACATCTGGGCGCCCTTGGCCGGGACTTGTGGGGTCCTTCTCCTGTCACTGGTTATCACCCTTTACTGCAAC
CACAGGAACAGAGTGAAGTTCAGCAGGAGCGCAGACGCCCCCGCGTACCAGCAGGGCCAGAACCAGCTCTATAACGA
GCTCAATCTAGGACGAAGAGAGGAGTACGATGTTTTGGACAAGAGACGTGGCCGGGACCCTGAGATGGGGGGAAAGC
CGAGAAGGAAGAACCCTCAGGAAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCTCAGTACAGCCACCAAGGACAC
CTACGACGCCCTTCACATGCAGGCCCTGCCCCCTCGC

Only construct #3, containing ACE2 19-740 AA showed binding to spike and was chosen as a bait to be placed on the extracellular domain of the CBR construct.

To improve the mannose receptor-based CBR, various constructs (F1-F5) were tested containing different regions of mannose receptor (MR), hinge, and signal peptides. The components of the constructs are as follows (N terminus to C terminus):
F1: CD8 signal peptide+anti-FLT3 scFv+CD8 hinge+last 96 AA of MR+T2A+GFP (in frame with T2A).
F2: CD8 signal peptide+anti-FLT3 scFv+last 96 AA of MR+T2A+GFP (in frame with T2A).
F3: CD8 signal peptide+anti-FLT3 scFv+CD8 hinge+last 83AA of MR+T2A+GFP (in frame with T2A).
F4: CD8 signal peptide+anti-FLT3 scFv or ACE2(19-740)+last 83AA of MR+T2A+GFP (in frame with T2A).
F5: MR signal peptide+anti-FLT3 scFv or ACE2(19-740)+CD8 hinge+last 96 AA+T2A+GFP (in frame with T2A).

The nucleotide sequences for each of these constructs (excluding T2A-GFP) is provided below.

F1 (SEQ ID NO: 69):
ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTGGATCCTTCGTGCCGGTCTTCCTGCCAGCGA
AGCCCACCACGACGCCAGCGCCGCGACCACCAACACCGGCGCCCACCATCGCGTCGCAGCCCCTGTCCCTGCGCCCA
GAGGCGTGCCGGCCAGCGGCGGGGGGCGCAGTGCACACGAGGGGGCTGGACTTCGCCTGTGATATTGATGCTAAACC
TACTCATGAATTACTTACAACAAAAGCTGACACAAGGAAGATGGACCCTTCTAAACCGTCTTCCAACGTGGCCGGAG
TAGTCATCATTGTGATCCTCCTGATTTTAACGGGTGCTGGCCTTGCCGCCTATTTCTTTTATAAGAAAAGACGTGTG
CACCTACCTCAAGAGGGCGCCTTTGAAAACACTCTGTATTTTAACAGTCAGTCAAGCCCAGGAACTAGTGATATGAA
AGATCTCGTGGGCAATATTGAACAGAATGAACACTCGGTCATC
F2 (SEQ ID NO: 70):
ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTATTGATGCTAAACCTACTCATGAATTACTTA
CAACAAAAGCTGACACAAGGAAGATGGACCCTTCTAAACCGTCTTCCAACGTGGCCGGAGTAGTCATCATTGTGATC
CTCCTGATTTTAACGGGTGCTGGCCTTGCCGCCTATTTCTTTTATAAGAAAAGACGTGTGCACCTACCTCAAGAGGG
CGCCTTTGAAAACACTCTGTATTTTAACAGTCAGTCAAGCCCAGGAACTAGTGATATGAAAGATCTCGTGGGCAATA
TTGAACAGAATGAACACTCGGTCATC
F3 (SEQ ID NO: 71):
ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTGGATCCTTCGTGCCGGTCTTCCTGCCAGCGA
AGCCCACCACGACGCCAGCGCCGCGACCACCAACACCGGCGCCCACCATCGCGTCGCAGCCCCTGTCCCTGCGCCCA
GAGGCGTGCCGGCCAGCGGGGGGGGCGCAGTGCACACGAGGGGGCTGGACTTCGCCTGTGATGACACAAGGAAGAT
GGACCCTTCTAAACCGTCTTCCAACGTGGCCGGAGTAGTCATCATTGTGATCCTCCTGATTTTAACGGGTGCTGGCC
TTGCCGCCTATTTCTTTTATAAGAAAAGACGTGTGCACCTACCTCAAGAGGGCGCCTTTGAAAACACTCTGTATTTT
AACAGTCAGTCAAGCCCAGGAACTAGTGATATGAAAGATCTCGTGGGCAATATTGAACAGAATGAACACTCGGTCAT
C
F4-FLT3-scFv (SEQ ID NO: 72):
ATGGCCTTACCAGTGACCGCCTTGCTCCTGCCGCTGGCCTTGCTGCTCCACGCCGCCAGGCCGGGATCCGACATCCA
GATGACCCAGAGCCCCTCCTCTTTATCCGCCTCTGTGGGCGACAGAGTGACCATCACTTGTCGTGCCAGCCAAGAAA
TCTCCGGCTATCTGTCTTGGCTGCAGCAGAAGCCCGGTAAGGCTATCAAGAGACTCATCTACGCCGCCAGCACTTTA
CAGAGCGGAGTGCCTAGCAGATTTAGCGGCTCTCGTAGCGGCAGCGATTATACTTTAACCATCTCCTCTTTACAGCC
CGAAGACTTTGCTACCTACTACTGTTTACAGTACGCCAGCTACCCCTTCACCTTCGGTCAAGGTACCAAGCTGGAGA
TCAAAGGTGGCGGCGGCTCTGGCGGCGGTGGCTCTGGCGGTGGCTCCGGCGGTGGTGGTAGCCAAGTTACCCTCAAG
GAGAGCGGCCCCACTTTAGTGAAGCCTACCCAGACTTTAACTTTAACTTGTACCTTCAGCGGCTTCTCTTTAAGCAC
CTCCACAATGGGCGTGGGCTGGATCAGACAGCCTCCCGGCAAGGCTCTGGAGTGGCTGGCCCACATCCTCTGGAACG
ACAGCAAGAGGTACAACCCCTCTTTAAAGTCTCGTCTGACCATCACCAAGGACACCTCCAAGAAGCAAGTTGTGCTG
ACCATGACCAATATGGACCCCGTGGACACCGCCACCTATTACTGCGCTCGTATCGTGTACTACTCCACCTACGTGGG
CTACTTCGACGTCTGGGGACAAGGTACCACCGTGACCGTGAGCTCTGACACAAGGAAGATGGACCCTTCTAAACCGT
CTTCCAACGTGGCCGGAGTAGTCATCATTGTGATCCTCCTGATTTTAACGGGTGCTGGCCTTGCCGCCTATTTCTTT
TATAAGAAAAGACGTGTGCACCTACCTCAAGAGGGCGCCTTTGAAAACACTCTGTATTTTAACAGTCAGTCAAGCCC
AGGAACTAGTGATATGAAAGATCTCGTGGGCAATATTGAACAGAATGAACACTCGGTCATC
F4-ACE2(19-740) (SEQ ID NO: 54):
ATGGCCTTACCAGTGACCGCCTTGCTCCTGCCGCTGGCCTTGCTGCTCCACGCCGCCAGGCCGGGATCCTCCACCAT
TGAGGAACAGGCCAAGACATTTTTGGACAAGTTTAACCACGAAGCCGAAGACCTGTTCTATCAAAGTTCACTTGCTT
CTTGGAATTATAACACCAATATTACTGAAGAGAATGTCCAAAACATGAATAATGCTGGGGACAAATGGTCTGCCTTT
TTAAAGGAACAGTCCACACTTGCCCAAATGTATCCACTACAAGAAATTCAGAATCTCACAGTCAAGCTTCAGCTGCA
GGCTCTTCAGCAAAATGGGTCTTCAGTGCTCTCAGAAGACAAGAGCAAACGGTTGAACACAATTCTAAATACAATGA
GCACCATCTACAGTACTGGAAAAGTTTGTAACCCAGATAATCCACAAGAATGCTTATTACTTGAACCAGGTTTGAAT
GAAATAATGGCAAACAGTTTAGACTACAATGAGAGGCTCTGGGCTTGGGAAAGCTGGAGATCTGAGGTCGGCAAGCA
GCTGAGGCCATTATATGAAGAGTATGTGGTCTTGAAAAATGAGATGGCAAGAGCAAATCATTATGAGGACTATGGGG
ATTATTGGAGAGGAGACTATGAAGTAAATGGGGTAGATGGCTATGACTACAGCCGCGGCCAGTTGATTGAAGATGTG
GAACATACCTTTGAAGAGATTAAACCATTATATGAACATCTTCATGCCTATGTGAGGGCAAAGTTGATGAATGCCTA
TCCTTCCTATATCAGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGACCAGGCCTGGGAT
GCACAGAGAATATTCAAGGAGGCCGAGAAGTTCTTTGTATCTGTTGGTCTTCCTAATATGACTCAAGGATTCTGGGA
AAATTCCATGCTAACGGACCCAGGAAATGTTCAGAAAGCAGTCTGCCATCCCACAGCTTGGGACCTGGGGAAGGGCG
ACTTCAGGATCCTTATGTGCACAAAGGTGACAATGGACGACTTCCTGACAGCTCATCATGAGATGGGGCATATCCAG
TATGATATGGCATATGCTGCACAACCTTTTCTGCTAAGAAATGGAGCTAATGAAGGATTCCATGAAGCTGTTGGGGA
AATCATGTCACTTTCTGCAGCCACACCTAAGCATTTAAAATCCATTGGTCTTCTGTCACCCGATTTTCAAGAAGACA
ATGAAACAGAAATAAACTTCCTGCTCAAACAAGCACTCACGATTGTTGGGACTCTGCCATTTACTTACATGTTAGAG
AAGTGGAGGTGGATGGTCTTTAAAGGGGAAATTCCCAAAGACCAGTGGATGAAAAAGTGGTGGGAGATGAAGCGAGA
GATAGTTGGGGTGGTGGAACCTGTGCCCCATGATGAAACATACTGTGACCCCGCATCTCTGTTCCATGTTTCTAATG
ATTACTCATTCATTCGATATTACACAAGGACCCTTTACCAATTCCAGTTTCAAGAAGCACTTTGTCAAGCAGCTAAA
CATGAAGGCCCTCTGCACAAATGTGACATCTCAAACTCTACAGAAGCTGGACAGAAACTGTTCAATATGCTGAGGCT
TGGAAAATCAGAACCCTGGACCCTAGCATTGGAAAATGTTGTAGGAGCAAAGAACATGAATGTAAGGCCACTGCTCA
ACTACTTTGAGCCCTTATTTACCTGGCTGAAAGACCAGAACAAGAATTCTTTTGTGGGATGGAGTACCGACTGGAGT
CCATATGCAGACCAAAGCATCAAAGTGAGGATAAGCCTAAAATCAGCTCTTGGAGATAAAGCATATGAATGGAACGA
CAATGAAATGTACCTGTTCCGATCATCTGTTGCATATGCTATGAGGCAGTACTTTTTAAAAGTAAAAAATCAGATGA
TTCTTTTTGGGGAGGAGGATGTGCGAGTGGCTAATTTGAAACCAAGAATCTCCTTTAATTTCTTTGTCACTGCACCT
AAAAATGTGTCTGATATCATTCCTAGAACTGAAGTTGAAAAGGCCATCAGGATGTCCCGGAGCCGTATCAATGATGC
TTTCCGTCTGAATGACAACAGCCTAGAGTTTCTGGGGATACAGCCAACACTTGGACCTCCTAACCAGCCCCCTGTTT
CCGACACAAGGAAGATGGACCCTTCTAAACCGTCTTCCAACGTGGCCGGAGTAGTCATCATTGTGATCCTCCTGATT
TTAACGGGTGCTGGCCTTGCCGCCTATTTCTTTTATAAGAAAAGACGTGTGCACCTACCTCAAGAGGGCGCCTTTGA
AAACACTCTGTATTTTAACAGTCAGTCAAGCCCAGGAACTAGTGATATGAAAGATCTCGTGGGCAATATTGAACAGA
ATGAACACTCGGTCATC
F5-FLT3-scFv (SEQ ID NO: 73):
ATGAGGCTACCCCTGCTCCTGGTTTTTGCCTCTGTCATTCCGGGTGCTGTTCTCGACATCCAGATGACCCAGAGCCC
CTCCTCTTTATCCGCCTCTGTGGGCGACAGAGTGACCATCACTTGTCGTGCCAGCCAAGAAATCTCCGGCTATCTGT
CTTGGCTGCAGCAGAAGCCCGGTAAGGCTATCAAGAGACTCATCTACGCCGCCAGCACTTTACAGAGCGGAGTGCCT
AGCAGATTTAGCGGCTCTCGTAGCGGCAGCGATTATACTTTAACCATCTCCTCTTTACAGCCCGAAGACTTTGCTAC
CTACTACTGTTTACAGTACGCCAGCTACCCCTTCACCTTCGGTCAAGGTACCAAGCTGGAGATCAAAGGTGGCGGCG
GCTCTGGCGGCGGTGGCTCTGGCGGTGGCTCCGGCGGTGGTGGTAGCCAAGTTACCCTCAAGGAGAGCGGCCCCACT
TTAGTGAAGCCTACCCAGACTTTAACTTTAACTTGTACCTTCAGCGGCTTCTCTTTAAGCACCTCCACAATGGGCGT
GGGCTGGATCAGACAGCCTCCCGGCAAGGCTCTGGAGTGGCTGGCCCACATCCTCTGGAACGACAGCAAGAGGTACA
ACCCCTCTTTAAAGTCTCGTCTGACCATCACCAAGGACACCTCCAAGAAGCAAGTTGTGCTGACCATGACCAATATG
GACCCCGTGGACACCGCCACCTATTACTGCGCTCGTATCGTGTACTACTCCACCTACGTGGGCTACTTCGACGTCTG
GGGACAAGGTACCACCGTGACCGTGAGCTCTGGATCCTTCGTGCCGGTCTTCCTGCCAGCGAAGCCCACCACGACGC
CAGCGCCGCGACCACCAACACCGGCGCCCACCATCGCGTCGCAGCCCCTGTCCCTGCGCCCAGAGGCGTGCCGGCCA
GCGGCGGGGGGCGCAGTGCACACGAGGGGGCTGGACTTCGCCTGTGATATTGATGCTAAACCTACTCATGAATTACT
TACAACAAAAGCTGACACAAGGAAGATGGACCCTTCTAAACCGTCTTCCAACGTGGCCGGAGTAGTCATCATTGTGA
TCCTCCTGATTTTAACGGGTGCTGGCCTTGCCGCCTATTTCTTTTATAAGAAAAGACGTGTGCACCTACCTCAAGAG
GGCGCCTTTGAAAACACTCTGTATTTTAACAGTCAGTCAAGCCCAGGAACTAGTGATATGAAAGATCTCGTGGGCAA
TATTGAACAGAATGAACACTCGGTCATC
F5-ACE2(19-740) (SEQ ID NO: 55):
ATGAGGCTACCCCTGCTCCTGGTTTTTGCCTCTGTCATTCCGGGTGCTGTTCTCTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCCGGATCCTTCGTGC
CGGTCTTCCTGCCAGCGAAGCCCACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCACACGAGGGGGCTGGACTTCGCCTG
TGATATTGATGCTAAACCTACTCATGAATTACTTACAACAAAAGCTGACACAAGGAAGATGGACCCTTCTAAACCGT
CTTCCAACGTGGCCGGAGTAGTCATCATTGTGATCCTCCTGATTTTAACGGGTGCTGGCCTTGCCGCCTATTTCTTT
TATAAGAAAAGACGTGTGCACCTACCTCAAGAGGGCGCCTTTGAAAACACTCTGTATTTTAACAGTCAGTCAAGCCC
AGGAACTAGTGATATGAAAGATCTCGTGGGCAATATTGAACAGAATGAACACTCGGTCATC

Mannose-based CBR development was continued using F4 construct, which contains CD8 signal peptide, scFv or ACE2 (19-740) bait, and the last 83 amino acids of mannose receptor (transmembrane and intracellular domains). Kruskal et al., 1992. J. Exp. Med.; Harris et al., 1993. Biochem. Biophys. Res. Commun.

### Example 2: Analysis of CBR-Induced Phagocytosis

Thp1 cells were transduced with lentivirus carrying F4-AC construct under EFla promoter, the cells were stained with biotinylated SARS-CoV-2 Spike protein (MyBioSource, San Diego, CA) followed by PE-streptavidin (BioLegend, San Diego, CA), and positive cells were sorted using Melody sorter (BD, Franklin Lakes, NJ). Streptavidin-coated 5.06 μm beads (Spherotech, Lake Forest, IL) were sterilized using 70% isopropanol and labeled with 10 uM pHrodo red dye (Thermo Fisher, Waltham, MA) according to manufacturer instructions. Washed beads were then incubated with biotinylated Spike protein at a ratio of 0.025 nmole biotinylated Spike protein (MyBioSource, San Diego, CA) per 0.5 mg beads. Beads were incubated overnight with cells at a ratio of 1:5 (cells: beads).

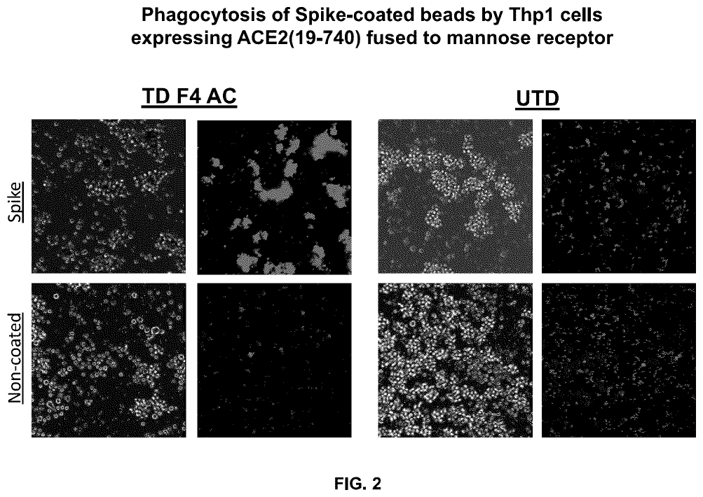

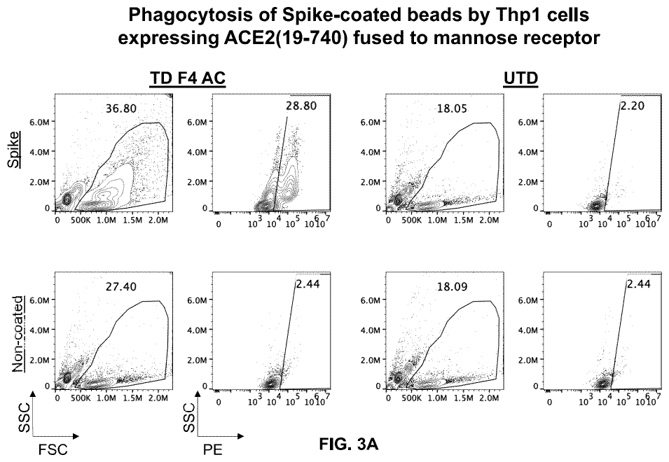

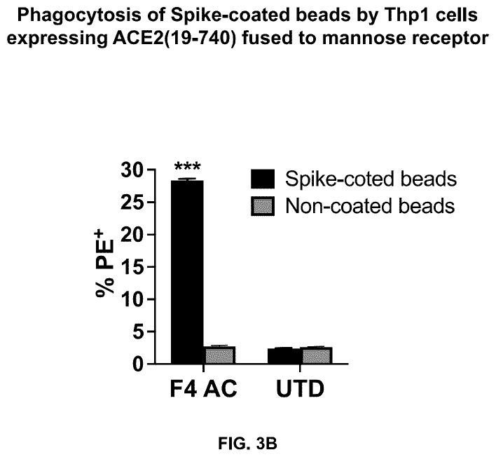

As shown in FIG. 2 and FIG. 3A-3B, only Spike-coated beads were phagocytosed and only by Thp1 that were transduced with mannose receptor-based CBR construct containing ACE2 bait on the surface. Non-coated beads were not phagocytosed and untransduced (UTD) cells did not phagocytose any beads.

The results of this test demonstrate that CBRs can bind to viral proteins and induce phagocytosis in a binding-specific manner.

### Example 3: Analysis of CBR-Induced Phagocytosis of SARS-CoV-2 Variants

The experiment described in Example 2 was repeated to test the ability of CBR-transduced cells to recognize and phagocytose spike proteins from two different SARS-CoV-2 variants (UK alpha variant B.1.1.7 & Delta variant B.1.617.2). Specifically, Thp1 cells were transduced with lentivirus carrying F4-AC construct under EF1a promoter, the cells were stained with biotinylated SARS-CoV-2 Spike protein from either the UK alpha variant, B.1.1.7 (ACROBiosystems, Delaware Technology Park, DE) or the Delta variant, B.1.617.2 (SinoBiological, Wayne, PA), followed by PE-streptavidin (BioLegend, San Diego, CA), and positive cells were sorted using Melody sorter (BD, Franklin Lakes, NJ). Streptavidin-coated 5.06 μm beads (Spherotech, Lake Forest, IL) were sterilized using 70% isopropanol and labeled with 10 uM pHrodo red dye (Thermo Fisher, Waltham, MA) according to manufacturer instructions. Washed beads were then incubated with biotinylated Spike protein at a ratio of 0.025 nmole biotinylated Spike protein per 0.5 mg beads. Beads were incubated overnight with cells at a ratio of 1:5 (cells:beads).

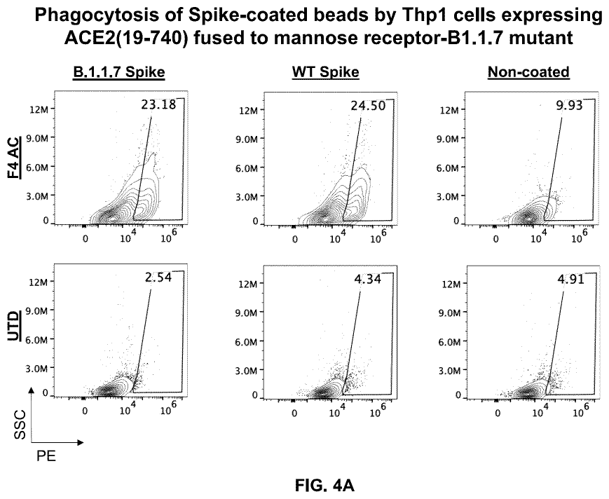

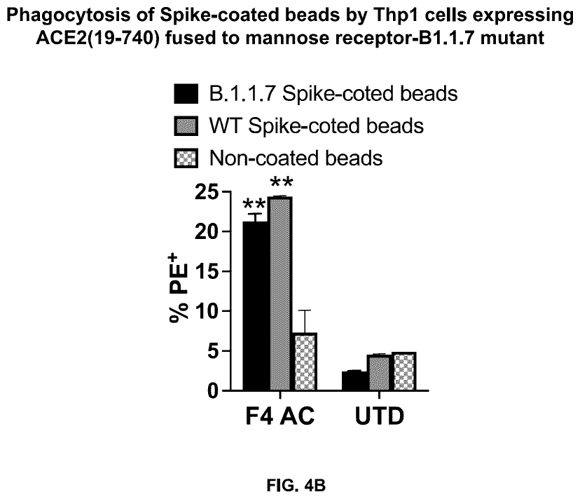

As shown in FIG. 4A-4B, both wild-type Spike-coated beads and B1.1.7 Spike-coated beads were phagocytosed and only by Thp 1 that were transduced with the mannose receptor-based CBR construct containing ACE2 bait on the surface. Non-coated beads were not phagocytosed and untransduced (UTD) cells did not phagocytose any beads.

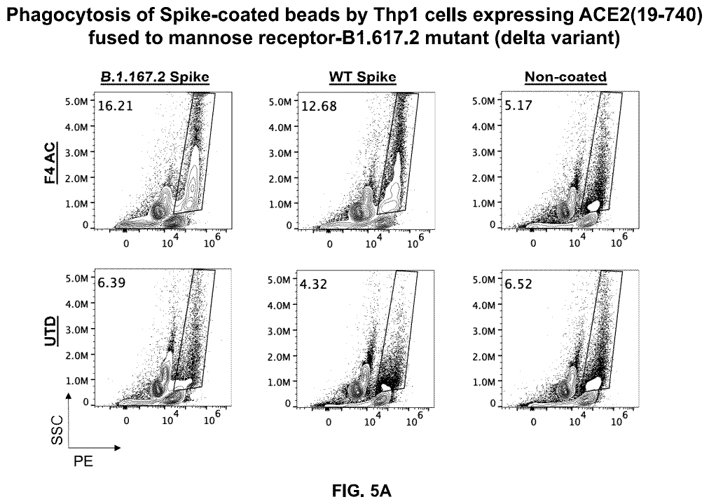

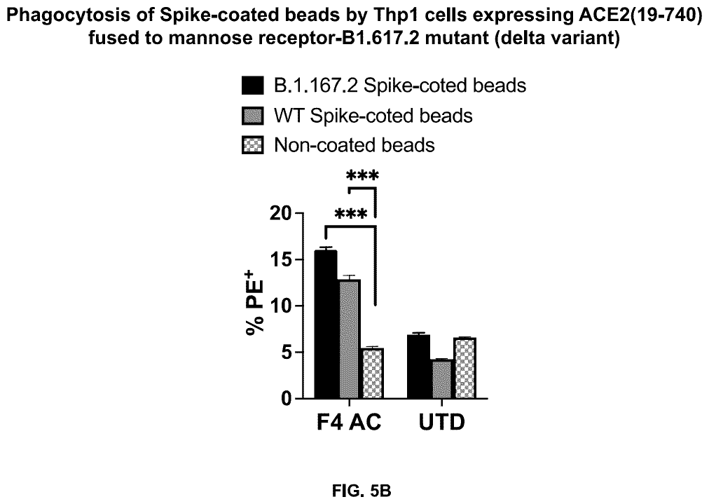

Similarly, both wild-type Spike-coated beads and B.1.617.2 Spike-coated beads were phagocytosed and only by Thp1 that were transduced with the mannose receptor-based CBR construct containing ACE2 bait on the surface (FIG. 5A-5B). Non-coated beads were not phagocytosed and untransduced (UTD) cells did not phagocytose any beads.

The results of this test demonstrate that CBRs can bind to viral proteins from multiple variant strains and induce phagocytosis in a binding-specific manner.

### Example 4: Analysis of CBR-Induced SARS-CoV-2 Neutralization

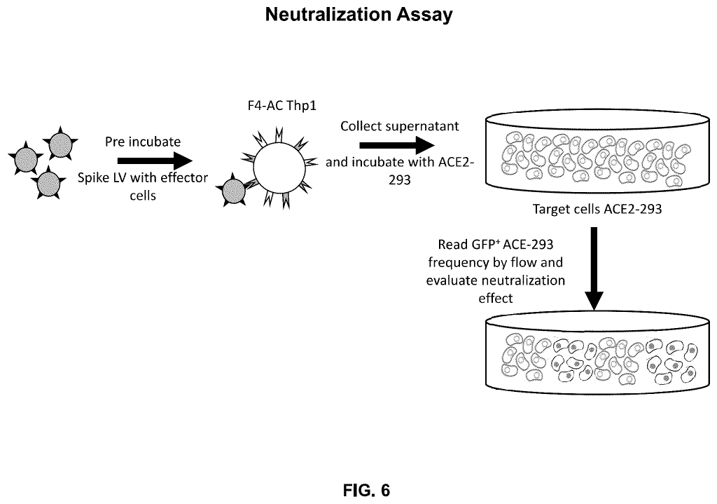

A neutralization assay (illustrated in FIG. 6) was used to test the ability of Thp1 cells transduced with mannose receptor-based CBR construct containing ACE2 bait on the surface (F4 AC) to neutralize SARS-CoV-2 pseduotyped lentivirus. Specifically, lentivirus carrying GFP transfer plasmid was pseudotyped with Spike envelope protein (BEI Resources, Manassas, VA), to generate Spike-LV. Spike-LV particles were pre-incubated with either Thp1 effector cells expressing the F4 AC CBR construct or with control cells, ACE2-293 or untransfected (“UTD”) Thp1 or no cells on pre-incubation. After 2 hr pre-incubation, cells were spun down at 300 g for 5 minutes, and supernatant was collected and was incubated with HEK 293T cells expressing hACE2 receptor on the cell surface (ACE2-293). After 2 days, GFP⁺ frequency was determined by flow cytometer to test neutralization effect. MOI 0.1 or 0.85 were determined based on the target cells. The ratio of effector: target cells was 100:1.

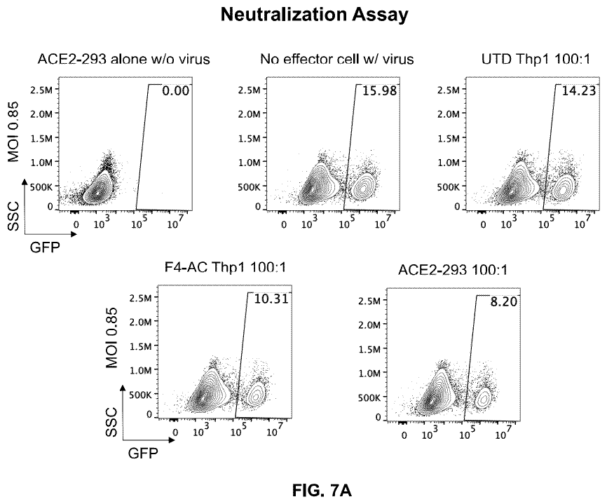

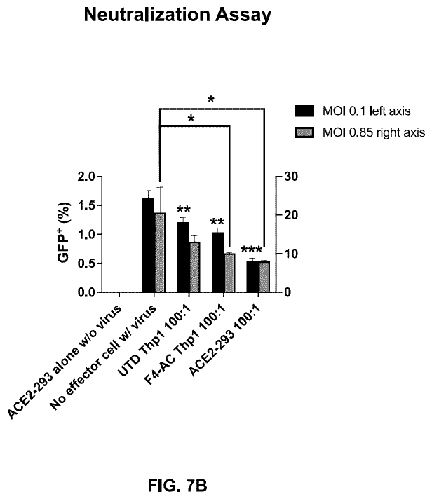

As shown in FIG. 7A-7B, pre incubation with either F4-AC Thp1 or ACE2-293 reduced the viral load in the supernatant as observed by lower transduction efficiency of target cells after a subsequent incubation.

### Example 5: CBR/CPR Constructs Based on Additional Phagocytic Receptors

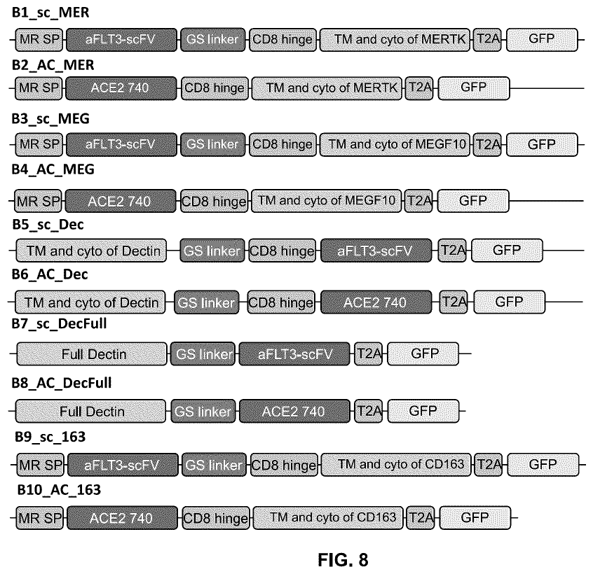

Additional CBR and CPR constructs were designed containing anti-FLT3 scFv or ACE2 (19-740) bait on top of the following phagocytic receptors: MERTK, MEGF10, Dectin-1, and CD163. Schematics for each of the constructs is shown in FIG. 8.

The nucleotide sequences for each of these constructs (excluding T2A-GFP) are provided below.

B1_sc_MER (SEQ ID NO: 74):
ATGAGGCTACCCCTGCTCCTGGTTTTTGCCTCTGTCATTCCGGGTGCTGTTCTCGACATCCAGATGACCCAGAGCCC
CTCCTCTTTATCCGCCTCTGTGGGCGACAGAGTGACCATCACTTGTCGTGCCAGCCAAGAAATCTCCGGCTATCTGT
CTTGGCTGCAGCAGAAGCCCGGTAAGGCTATCAAGAGACTCATCTACGCCGCCAGCACTTTACAGAGCGGAGTGCCT
AGCAGATTTAGCGGCTCTCGTAGCGGCAGCGATTATACTTTAACCATCTCCTCTTTACAGCCCGAAGACTTTGCTAC
CTACTACTGTTTACAGTACGCCAGCTACCCCTTCACCTTCGGTCAAGGTACCAAGCTGGAGATCAAAGGTGGCGGCG
GCTCTGGCGGCGGTGGCTCTGGCGGTGGCTCCGGCGGTGGTGGTAGCCAAGTTACCCTCAAGGAGAGCGGCCCCACT
TTAGTGAAGCCTACCCAGACTTTAACTTTAACTTGTACCTTCAGCGGCTTCTCTTTAAGCACCTCCACAATGGGCGT
GGGCTGGATCAGACAGCCTCCCGGCAAGGCTCTGGAGTGGCTGGCCCACATCCTCTGGAACGACAGCAAGAGGTACA
ACCCCTCTTTAAAGTCTCGTCTGACCATCACCAAGGACACCTCCAAGAAGCAAGTTGTGCTGACCATGACCAATATG
GACCCCGTGGACACCGCCACCTATTACTGCGCTCGTATCGTGTACTACTCCACCTACGTGGGCTACTTCGACGTCTG
GGGACAAGGTACCACCGTGACCGTGAGCTCTGGATCCTTCGTGCCGGTCTTCCTGCCAGCGAAGCCCACCACGACGC
CAGCGCCGCGACCACCAACACCGGCGCCCACCATCGCGTCGCAGCCCCTGTCCCTGCGCCCAGAGGCGTGCCGGCCA
GCGGCGGGGGGCGCAGTGCACACGAGGGGGCTGGACTTCGCCTGTGATTTTGGATGTTTTTGTGGTTTCATCCTCAT
CGGTTTGATATTGTACATAAGTCTGGCGATAAGGAAGAGAGTTCAAGAGACAAAGTTCGGAAATGCCTTTACAGAGG
AAGACAGTGAGCTCGTTGTAAACTACATCGCAAAAAAAAGCTTCTGTAGAAGAGCAATAGAGCTCACGTTGCACTCA
CTCGGTGTGTCCGAAGAACTCCAGAATAAACTGGAAGACGTCGTTATCGATCGGAACCTCCTCATACTTGGAAAAAT
ACTGGGAGAAGGAGAGTTCGGCAGTGTCATGGAAGGTAACTTGAAACAAGAGGATGGTACCTCACTCAAGGTAGCTG
TCAAGACGATGAAACTTGATAACAGTTCACAAAGGGAGATCGAAGAATTTCTGTCTGAGGCCGCCTGTATGAAAGAC
TTCTCACATCCTAATGTCATCAGACTTCTTGGCGTTTGTATCGAGATGTCTAGCCAAGGAATCCCAAAACCTATGGT
CATATTGCCTTTCATGAAATATGGCGATCTGCATACATATTTGCTCTACTCTAGACTTGAGACAGGGCCCAAACATA
TTCCTCTCCAGACATTGCTCAAGTTTATGGTCGATATTGCCCTGGGTATGGAGTACTTGAGCAACCGAAATTTTCTG
CATCGGGATCTTGCCGCACGCAACTGCATGCTGCGCGATGACATGACCGTCTGCGTGGCTGATTTTGGGCTGTCAAA
AAAAATATATTCTGGAGACTACTACCGACAAGGGCGGATTGCAAAGATGCCCGTCAAATGGATTGCGATTGAAAGTT
TGGCGGACAGGGTATATACTTCCAAATCAGATGTTTGGGCTTTTGGAGTCACTATGTGGGAAATAGCTACACGCGGT
ATGACCCCGTACCCCGGAGTACAAAATCATGAAATGTATGACTATCTCCTTCATGGACACAGGCTGAAGCAGCCCGA
GGACTGCCTGGACGAACTGTATGAAATAATGTATTCTTGTTGGCGAACCGATCCCTTGGACCGGCCTACTTTCAGTG
TCCTTAGATTGCAACTTGAGAAATTGCTCGAGTCTTTGCCGGATGTGCGAAACCAGGCAGACGTGATCTATGTCAAT
ACCCAACTTTTGGAAAGTTCTGAGGGCCTCGCACAGGGTTCTACCCTTGCCCCGTTGGATCTTAACATAGACCCAGA
CAGCATAATTGCTTCTTGTACACCTCGCGCTGCCATATCAGTTGTAACAGCGGAGGTCCATGATAGTAAACCTCACG
AGGGTCGCTATATCCTGAACGGCGGGTCAGAAGAATGGGAAGACCTGACATCAGCGCCGAGCGCCGCCGTTACTGCT
GAGAAAAACTCTGTCCTGCCCGGAGAGCGCTTGGTTCGGAACGGGGTAAGTTGGAGCCATAGCTCAATGCTCCCCCT
GGGTTCAAGTCTCCCGGACGAGCTTCTTTTTGCGGACGACTCATCTGAGGGGTCCGAAGTTCTGATG
B2_AC_MER (SEQ ID NO: 56):
ATGAGGCTACCCCTGCTCCTGGTTTTTGCCTCTGTCATTCCGGGTGCTGTTCTCTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCCGGATCCTTCGTGC
CGGTCTTCCTGCCAGCGAAGCCCACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCACACGAGGGGGCTGGACTTCGCCTG
TGATTTTGGATGTTTTTGTGGTTTCATCCTCATCGGTTTGATATTGTACATAAGTCTGGCGATAAGGAAGAGAGTTC
AAGAGACAAAGTTCGGAAATGCCTTTACAGAGGAAGACAGTGAGCTCGTTGTAAACTACATCGCAAAAAAAAGCTTC
TGTAGAAGAGCAATAGAGCTCACGTTGCACTCACTCGGTGTGTCCGAAGAACTCCAGAATAAACTGGAAGACGTCGT
TATCGATCGGAACCTCCTCATACTTGGAAAAATACTGGGAGAAGGAGAGTTCGGCAGTGTCATGGAAGGTAACTTGA
AACAAGAGGATGGTACCTCACTCAAGGTAGCTGTCAAGACGATGAAACTTGATAACAGTTCACAAAGGGAGATCGAA
GAATTTCTGTCTGAGGCCGCCTGTATGAAAGACTTCTCACATCCTAATGTCATCAGACTTCTTGGCGTTTGTATCGA
GATGTCTAGCCAAGGAATCCCAAAACCTATGGTCATATTGCCTTTCATGAAATATGGCGATCTGCATACATATTTGC
TCTACTCTAGACTTGAGACAGGGCCCAAACATATTCCTCTCCAGACATTGCTCAAGTTTATGGTCGATATTGCCCTG
GGTATGGAGTACTTGAGCAACCGAAATTTTCTGCATCGGGATCTTGCCGCACGCAACTGCATGCTGCGCGATGACAT
GACCGTCTGCGTGGCTGATTTTGGGCTGTCAAAAAAAATATATTCTGGAGACTACTACCGACAAGGGCGGATTGCAA
AGATGCCCGTCAAATGGATTGCGATTGAAAGTTTGGCGGACAGGGTATATACTTCCAAATCAGATGTTTGGGCTTTT
GGAGTCACTATGTGGGAAATAGCTACACGCGGTATGACCCCGTACCCCGGAGTACAAAATCATGAAATGTATGACTA
TCTCCTTCATGGACACAGGCTGAAGCAGCCCGAGGACTGCCTGGACGAACTGTATGAAATAATGTATTCTTGTTGGC
GAACCGATCCCTTGGACCGGCCTACTTTCAGTGTCCTTAGATTGCAACTTGAGAAATTGCTCGAGTCTTTGCCGGAT
GTGCGAAACCAGGCAGACGTGATCTATGTCAATACCCAACTTTTGGAAAGTTCTGAGGGCCTCGCACAGGGTTCTAC
CCTTGCCCCGTTGGATCTTAACATAGACCCAGACAGCATAATTGCTTCTTGTACACCTCGCGCTGCCATATCAGTTG
TAACAGCGGAGGTCCATGATAGTAAACCTCACGAGGGTCGCTATATCCTGAACGGCGGGTCAGAAGAATGGGAAGAC
CTGACATCAGCGCCGAGCGCCGCCGTTACTGCTGAGAAAAACTCTGTCCTGCCCGGAGAGCGCTTGGTTCGGAACGG
GGTAAGTTGGAGCCATAGCTCAATGCTCCCCCTGGGTTCAAGTCTCCCGGACGAGCTTCTTTTTGCGGACGACTCAT
CTGAGGGGTCCGAAGTTCTGATG
B3_sc_MEG (SEQ ID NO: 75):
ATGAGGCTACCCCTGCTCCTGGTTTTTGCCTCTGTCATTCCGGGTGCTGTTCTCGACATCCAGATGACCCAGAGCCC
CTCCTCTTTATCCGCCTCTGTGGGCGACAGAGTGACCATCACTTGTCGTGCCAGCCAAGAAATCTCCGGCTATCTGT
CTTGGCTGCAGCAGAAGCCCGGTAAGGCTATCAAGAGACTCATCTACGCCGCCAGCACTTTACAGAGCGGAGTGCCT
AGCAGATTTAGCGGCTCTCGTAGCGGCAGCGATTATACTTTAACCATCTCCTCTTTACAGCCCGAAGACTTTGCTAC
CTACTACTGTTTACAGTACGCCAGCTACCCCTTCACCTTCGGTCAAGGTACCAAGCTGGAGATCAAAGGTGGCGGCG
GCTCTGGCGGCGGTGGCTCTGGCGGTGGCTCCGGCGGTGGTGGTAGCCAAGTTACCCTCAAGGAGAGCGGCCCCACT
TTAGTGAAGCCTACCCAGACTTTAACTTTAACTTGTACCTTCAGCGGCTTCTCTTTAAGCACCTCCACAATGGGCGT
GGGCTGGATCAGACAGCCTCCCGGCAAGGCTCTGGAGTGGCTGGCCCACATCCTCTGGAACGACAGCAAGAGGTACA
ACCCCTCTTTAAAGTCTCGTCTGACCATCACCAAGGACACCTCCAAGAAGCAAGTTGTGCTGACCATGACCAATATG
GACCCCGTGGACACCGCCACCTATTACTGCGCTCGTATCGTGTACTACTCCACCTACGTGGGCTACTTCGACGTCTG
GGGACAAGGTACCACCGTGACCGTGAGCTCTGGATCCTTCGTGCCGGTCTTCCTGCCAGCGAAGCCCACCACGACGC
CAGCGCCGCGACCACCAACACCGGCGCCCACCATCGCGTCGCAGCCCCTGTCCCTGCGCCCAGAGGCGTGCCGGCCA
GCGGCGGGGGGCGCAGTGCACACGAGGGGGCTGGACTTCGCCTGTGATGCTATCGCGGGGATCATTATATTGGTCTT
GGTGGTGCTGTTTCTGCTCGCGCTTTTCATTATATACCGCCATAAGCAGAAGGGCAAAGAGTCCTCCATGCCAGCCG
TGACCTATACGCCTGCGATGCGCGTTGTCAACGCCGATTACACCATCAGTGGTACCCTCCCGCACAGTAACGGCGGA
AATGCAAACTCTCATTACTTTACAAATCCTAGTTACCATACACTCACTCAGTGTGCTACCTCTCCCCATGTGAACAA
TCGGGACAGGATGACCGTTACGAAAAGCAAAAATAACCAGTTGTTTGTGAACCTTAAGAATGTGAATCCCGGCAAGA
GGGGTCCGGTGGGTGACTGCACCGGAACTCTCCCCGCTGACTGGAAGCATGGCGGGTACCTGAACGAACTCGGCGCG
TTTGGGCTCGACCGAAGCTACATGGGTAAAAGTCTTAAGGACCTCGGTAAGAATAGCGAGTATAATAGCTCTAACTG
TTCCCTTTCCAGCTCCGAGAATCCGTACGCTACTATAAAAGACCCCCCGGTGCTCATTCCCAAATCCAGTGAGTGCG
GGTACGTGGAGATGAAAAGTCCCGCTCGAAGAGACAGTCCATACGCGGAAATCAATAACTCCACCAGTGCGAACCGC
AATGTGTACGAAGTGGAGCCCACCGTTTCCGTTGTACAAGGTGTATTTTCAAACAATGGGAGGCTTAGCCAGGACCC
CTATGATCTTCCAAAGAACAGCCACATCCCGTGTCATTATGATCTGTTGCCGGTGAGGGATTCTAGCTCTTCTCCTA
AACAAGAGGACTCAGGTGGCTCTAGTTCCAACTCCTCCAGTTCTTCAGAG
B4_AC_MEG (SEQ ID NO: 57):
ATGAGGCTACCCCTGCTCCTGGTTTTTGCCTCTGTCATTCCGGGTGCTGTTCTCTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCCGGATCCTTCGTGC
CGGTCTTCCTGCCAGCGAAGCCCACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCACACGAGGGGGCTGGACTTCGCCTG
TGATGCTATCGCGGGGATCATTATATTGGTCTTGGTGGTGCTGTTTCTGCTCGCGCTTTTCATTATATACCGCCATA
AGCAGAAGGGCAAAGAGTCCTCCATGCCAGCCGTGACCTATACGCCTGCGATGCGCGTTGTCAACGCCGATTACACC
ATCAGTGGTACCCTCCCGCACAGTAACGGCGGAAATGCAAACTCTCATTACTTTACAAATCCTAGTTACCATACACT
CACTCAGTGTGCTACCTCTCCCCATGTGAACAATCGGGACAGGATGACCGTTACGAAAAGCAAAAATAACCAGTTGT
TTGTGAACCTTAAGAATGTGAATCCCGGCAAGAGGGGTCCGGTGGGTGACTGCACCGGAACTCTCCCCGCTGACTGG
AAGCATGGCGGGTACCTGAACGAACTCGGCGCGTTTGGGCTCGACCGAAGCTACATGGGTAAAAGTCTTAAGGACCT
CGGTAAGAATAGCGAGTATAATAGCTCTAACTGTTCCCTTTCCAGCTCCGAGAATCCGTACGCTACTATAAAAGACC
CCCCGGTGCTCATTCCCAAATCCAGTGAGTGCGGGTACGTGGAGATGAAAAGTCCCGCTCGAAGAGACAGTCCATAC
GCGGAAATCAATAACTCCACCAGTGCGAACCGCAATGTGTACGAAGTGGAGCCCACCGTTTCCGTTGTACAAGGTGT
ATTTTCAAACAATGGGAGGCTTAGCCAGGACCCCTATGATCTTCCAAAGAACAGCCACATCCCGTGTCATTATGATC
TGTTGCCGGTGAGGGATTCTAGCTCTTCTCCTAAACAAGAGGACTCAGGTGGCTCTAGTTCCAACTCCTCCAGTTCT
TCAGAG
B5_sc_Dec (SEQ ID NO: 76):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGTTCGTGCCGGTCTTCCTGCCAGCGAAGCCCACCACG
ACGCCAGCGCCGCGACCACCAACACCGGCGCCCACCATCGCGTCGCAGCCCCTGTCCCTGCGCCCAGAGGCGTGCCG
GCCAGCGGCGGGGGGCGCAGTGCACACGAGGGGGCTGGACTTCGCCTGTGATGGATCCGACATCCAGATGACCCAGA
GCCCCTCCTCTTTATCCGCCTCTGTGGGCGACAGAGTGACCATCACTTGTCGTGCCAGCCAAGAAATCTCCGGCTAT
CTGTCTTGGCTGCAGCAGAAGCCCGGTAAGGCTATCAAGAGACTCATCTACGCCGCCAGCACTTTACAGAGCGGAGT
GCCTAGCAGATTTAGCGGCTCTCGTAGCGGCAGCGATTATACTTTAACCATCTCCTCTTTACAGCCCGAAGACTTTG
CTACCTACTACTGTTTACAGTACGCCAGCTACCCCTTCACCTTCGGTCAAGGTACCAAGCTGGAGATCAAAGGTGGC
GGCGGCTCTGGCGGCGGTGGCTCTGGCGGTGGCTCCGGCGGTGGTGGTAGCCAAGTTACCCTCAAGGAGAGCGGCCC
CACTTTAGTGAAGCCTACCCAGACTTTAACTTTAACTTGTACCTTCAGCGGCTTCTCTTTAAGCACCTCCACAATGG
GCGTGGGCTGGATCAGACAGCCTCCCGGCAAGGCTCTGGAGTGGCTGGCCCACATCCTCTGGAACGACAGCAAGAGG
TACAACCCCTCTTTAAAGTCTCGTCTGACCATCACCAAGGACACCTCCAAGAAGCAAGTTGTGCTGACCATGACCAA
TATGGACCCCGTGGACACCGCCACCTATTACTGCGCTCGTATCGTGTACTACTCCACCTACGTGGGCTACTTCGACG
TCTGGGGACAAGGTACCACCGTGACCGTGAGCTCT
B6_AC_Dec (SEQ ID NO: 58):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGTTCGTGCCGGTCTTCCTGCCAGCGAAGCCCACCACG
ACGCCAGCGCCGCGACCACCAACACCGGCGCCCACCATCGCGTCGCAGCCCCTGTCCCTGCGCCCAGAGGCGTGCCG
GCCAGCGGCGGGGGGCGCAGTGCACACGAGGGGGCTGGACTTCGCCTGTGATGGATCCTCCACCATTGAGGAACAGG
CCAAGACATTTTTGGACAAGTTTAACCACGAAGCCGAAGACCTGTTCTATCAAAGTTCACTTGCTTCTTGGAATTAT
AACACCAATATTACTGAAGAGAATGTCCAAAACATGAATAATGCTGGGGACAAATGGTCTGCCTTTTTAAAGGAACA
GTCCACACTTGCCCAAATGTATCCACTACAAGAAATTCAGAATCTCACAGTCAAGCTTCAGCTGCAGGCTCTTCAGC
AAAATGGGTCTTCAGTGCTCTCAGAAGACAAGAGCAAACGGTTGAACACAATTCTAAATACAATGAGCACCATCTAC
AGTACTGGAAAAGTTTGTAACCCAGATAATCCACAAGAATGCTTATTACTTGAACCAGGTTTGAATGAAATAATGGC
AAACAGTTTAGACTACAATGAGAGGCTCTGGGCTTGGGAAAGCTGGAGATCTGAGGTCGGCAAGCAGCTGAGGCCAT
TATATGAAGAGTATGTGGTCTTGAAAAATGAGATGGCAAGAGCAAATCATTATGAGGACTATGGGGATTATTGGAGA
GGAGACTATGAAGTAAATGGGGTAGATGGCTATGACTACAGCCGCGGCCAGTTGATTGAAGATGTGGAACATACCTT
TGAAGAGATTAAACCATTATATGAACATCTTCATGCCTATGTGAGGGCAAAGTTGATGAATGCCTATCCTTCCTATA
TCAGTCCAATTGGATGCCTCCCTGCTCATTTGCTTGGTGATATGTGGGGTAGATTTTGGACAAATCTGTACTCTTTG
ACAGTTCCCTTTGGACAGAAACCAAACATAGATGTTACTGATGCAATGGTGGACCAGGCCTGGGATGCACAGAGAAT
ATTCAAGGAGGCCGAGAAGTTCTTTGTATCTGTTGGTCTTCCTAATATGACTCAAGGATTCTGGGAAAATTCCATGC
TAACGGACCCAGGAAATGTTCAGAAAGCAGTCTGCCATCCCACAGCTTGGGACCTGGGGAAGGGCGACTTCAGGATC
CTTATGTGCACAAAGGTGACAATGGACGACTTCCTGACAGCTCATCATGAGATGGGGCATATCCAGTATGATATGGC
ATATGCTGCACAACCTTTTCTGCTAAGAAATGGAGCTAATGAAGGATTCCATGAAGCTGTTGGGGAAATCATGTCAC
TTTCTGCAGCCACACCTAAGCATTTAAAATCCATTGGTCTTCTGTCACCCGATTTTCAAGAAGACAATGAAACAGAA
ATAAACTTCCTGCTCAAACAAGCACTCACGATTGTTGGGACTCTGCCATTTACTTACATGTTAGAGAAGTGGAGGTG
GATGGTCTTTAAAGGGGAAATTCCCAAAGACCAGTGGATGAAAAAGTGGTGGGAGATGAAGCGAGAGATAGTTGGGG
TGGTGGAACCTGTGCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTAAACATGAAGGCCC
TCTGCACAAATGTGACATCTCAAACTCTACAGAAGCTGGACAGAAACTGTTCAATATGCTGAGGCTTGGAAAATCAG
AACCCTGGACCCTAGCATTGGAAAATGTTGTAGGAGCAAAGAACATGAATGTAAGGCCACTGCTCAACTACTTTGAG
CCCTTATTTACCTGGCTGAAAGACCAGAACAAGAATTCTTTTGTGGGATGGAGTACCGACTGGAGTCCATATGCAGA
CCAAAGCATCAAAGTGAGGATAAGCCTAAAATCAGCTCTTGGAGATAAAGCATATGAATGGAACGACAATGAAATGT
ACCTGTTCCGATCATCTGTTGCATATGCTATGAGGCAGTACTTTTTAAAAGTAAAAAATCAGATGATTCTTTTTGGG
GAGGAGGATGTGCGAGTGGCTAATTTGAAACCAAGAATCTCCTTTAATTTCTTTGTCACTGCACCTAAAAATGTGTC
TGATATCATTCCTAGAACTGAAGTTGAAAAGGCCATCAGGATGTCCCGGAGCCGTATCAATGATGCTTTCCGTCTGA
ATGACAACAGCCTAGAGTTTCTGGGGATACAGCCAACACTTGGACCTCCTAACCAGCCCCCTGTTTCC
B7_sc_DecFull (SEQ ID NO: 77):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGACCATGGCGATCTGGCGCTCCAACTCTGGAAGTAAC
ACCCTTGAAAATGGTTACTTCCTCAGTAGGAACAAAGAGAACCATTCCCAGCCGACACAGTCAAGCCTTGAAGATTC
AGTCACCCCTACAAAGGCCGTAAAAACGACAGGTGTCCTGTCCTCTCCGTGTCCGCCTAACTGGATCATCTACGAGA
AAAGTTGTTATCTGTTTAGCATGAGCCTTAACAGTTGGGATGGCTCAAAAAGGCAGTGCTGGCAACTGGGGAGCAAC
CTTTTGAAGATAGACAGTTCCAACGAACTGGGCTTCATAGTCAAACAGGTGTCCTCTCAACCTGATAACTCATTCTG
GATCGGGCTCAGTCGACCCCAAACTGAGGTTCCATGGCTTTGGGAAGACGGCAGCACTTTCTCTTCAAATTTGTTTC
AAATAAGAACCACCGCTACGCAGGAGAATCCGAGTCCGAACTGTGTTTGGATTCACGTCTCAGTCATTTACGACCAA
CTTTGTAGTGTCCCTAGCTATTCCATCTGCGAGAAAAAGTTCAGTATGGGATCCGACATCCAGATGACCCAGAGCCC
CTCCTCTTTATCCGCCTCTGTGGGCGACAGAGTGACCATCACTTGTCGTGCCAGCCAAGAAATCTCCGGCTATCTGT
CTTGGCTGCAGCAGAAGCCCGGTAAGGCTATCAAGAGACTCATCTACGCCGCCAGCACTTTACAGAGCGGAGTGCCT
AGCAGATTTAGCGGCTCTCGTAGCGGCAGCGATTATACTTTAACCATCTCCTCTTTACAGCCCGAAGACTTTGCTAC
CTACTACTGTTTACAGTACGCCAGCTACCCCTTCACCTTCGGTCAAGGTACCAAGCTGGAGATCAAAGGTGGCGGCG
GCTCTGGCGGCGGTGGCTCTGGCGGTGGCTCCGGCGGTGGTGGTAGCCAAGTTACCCTCAAGGAGAGCGGCCCCACT
TTAGTGAAGCCTACCCAGACTTTAACTTTAACTTGTACCTTCAGCGGCTTCTCTTTAAGCACCTCCACAATGGGCGT
GGGCTGGATCAGACAGCCTCCCGGCAAGGCTCTGGAGTGGCTGGCCCACATCCTCTGGAACGACAGCAAGAGGTACA
ACCCCTCTTTAAAGTCTCGTCTGACCATCACCAAGGACACCTCCAAGAAGCAAGTTGTGCTGACCATGACCAATATG
GACCCCGTGGACACCGCCACCTATTACTGCGCTCGTATCGTGTACTACTCCACCTACGTGGGCTACTTCGACGTCTG
GGGACAAGGTACCACCGTGACCGTGAGCTCT
B8_AC_DecFull (SEQ ID NO: 59):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGACCATGGCGATCTGGCGCTCCAACTCTGGAAGTAAC
ACCCTTGAAAATGGTTACTTCCTCAGTAGGAACAAAGAGAACCATTCCCAGCCGACACAGTCAAGCCTTGAAGATTC
AGTCACCCCTACAAAGGCCGTAAAAACGACAGGTGTCCTGTCCTCTCCGTGTCCGCCTAACTGGATCATCTACGAGA
AAAGTTGTTATCTGTTTAGCATGAGCCTTAACAGTTGGGATGGCTCAAAAAGGCAGTGCTGGCAACTGGGGAGCAAC
CTTTTGAAGATAGACAGTTCCAACGAACTGGGCTTCATAGTCAAACAGGTGTCCTCTCAACCTGATAACTCATTCTG
GATCGGGCTCAGTCGACCCCAAACTGAGGTTCCATGGCTTTGGGAAGACGGCAGCACTTTCTCTTCAAATTTGTTTC
AAATAAGAACCACCGCTACGCAGGAGAATCCGAGTCCGAACTGTGTTTGGATTCACGTCTCAGTCATTTACGACCAA
CTTTGTAGTGTCCCTAGCTATTCCATCTGCGAGAAAAAGTTCAGTATGGGATCCTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCC
B9_sc_163 (SEQ ID NO: 78):
ATGAGGCTACCCCTGCTCCTGGTTTTTGCCTCTGTCATTCCGGGTGCTGTTCTCGACATCCAGATGACCCAGAGCCC
CTCCTCTTTATCCGCCTCTGTGGGCGACAGAGTGACCATCACTTGTCGTGCCAGCCAAGAAATCTCCGGCTATCTGT
CTTGGCTGCAGCAGAAGCCCGGTAAGGCTATCAAGAGACTCATCTACGCCGCCAGCACTTTACAGAGCGGAGTGCCT
AGCAGATTTAGCGGCTCTCGTAGCGGCAGCGATTATACTTTAACCATCTCCTCTTTACAGCCCGAAGACTTTGCTAC
CTACTACTGTTTACAGTACGCCAGCTACCCCTTCACCTTCGGTCAAGGTACCAAGCTGGAGATCAAAGGTGGCGGCG
GCTCTGGCGGCGGTGGCTCTGGCGGTGGCTCCGGCGGTGGTGGTAGCCAAGTTACCCTCAAGGAGAGCGGCCCCACT
TTAGTGAAGCCTACCCAGACTTTAACTTTAACTTGTACCTTCAGCGGCTTCTCTTTAAGCACCTCCACAATGGGCGT
GGGCTGGATCAGACAGCCTCCCGGCAAGGCTCTGGAGTGGCTGGCCCACATCCTCTGGAACGACAGCAAGAGGTACA
ACCCCTCTTTAAAGTCTCGTCTGACCATCACCAAGGACACCTCCAAGAAGCAAGTTGTGCTGACCATGACCAATATG
GACCCCGTGGACACCGCCACCTATTACTGCGCTCGTATCGTGTACTACTCCACCTACGTGGGCTACTTCGACGTCTG
GGGACAAGGTACCACCGTGACCGTGAGCTCTGGATCCTTCGTGCCGGTCTTCCTGCCAGCGAAGCCCACCACGACGC
CAGCGCCGCGACCACCAACACCGGCGCCCACCATCGCGTCGCAGCCCCTGTCCCTGCGCCCAGAGGCGTGCCGGCCA
GCGGCGGGGGGCGCAGTGCACACGAGGGGGCTGGACTTCGCCTGTGATTCATCCCTGGGTGGAACAGATAAGGAGTT
GAGGTTGGTGGATGGAGAGAATAAGTGCAGTGGACGGGTTGAGGTCAAGGTGCAGGAAGAGTGGGGTACTGTTTGTA
ACAACGGCTGGAGTATGGAAGCAGTCTCCGTGATATGCAATCAACTTGGTTGCCCTACAGCCATAAAAGCTCCTGGA
TGGGCTAACTCATCTGCCGGAAGCGGTCGAATATGGATGGACCATGTATCCTGTAGGGGCAATGAGTCAGCACTGTG
GGACTGCAAGCATGACGGGTGGGGAAAACACTCTAATTGTACCCACCAGCAAGATGCCGGTGTGACGTGTAGCGACG
GCAGCAACTTGGAGATGCGGTTGACGCGCGGCGGGAACATGTGCAGCGGTCGCATTGAGATAAAGTTCCAAGGGCGC
TGGGGGACCGTTTGTGACGACAACTTCAATATAGATCATGCTAGTGTGATATGCAGGCAGTTGGAGTGCGGTAGCGC
CGTCTCATTTAGTGGTTCTAGCAATTTCGGTGAGGGATCCGGGCCTATATGGTTCGACGACCTTATCTGCAATGGAA
ATGAGAGCGCCCTCTGGAACTGCAAGCACCAAGGTTGGGGTAAGCATAACTGCGATCATGCTGAGGATGCGGGTGTG
ATTTGTAGCAAGGGAGCCGACTTGTCACTTCGACTCGTAGATGGCGTGACGGAATGTAGCGGTCGCCTTGAGGTACG
CTTCCAAGGTGAATGGGGCACAATCTGTGATGACGGTTGGGACTCATATGACGCTGCGGTTGCTTGCAAACAGCTGG
GCTGTCCGACAGCGGTAACGGCAATTGGGCGGGTAAATGCCAGTAAGGGGTTTGGACATATATGGTTGGACAGTGTT
TCTTGTCAGGGACACGAACCTGCAATATGGCAATGTAAACATCACGAGTGGGGGAAGCATTACTGCAATCATAATGA
AGACGCAGGCGTCACATGCTCTGACGGATCCGACCTGGAATTGCGGCTGAGAGGGGGGGGTTCACGGTGCGCAGGAA
CCGTGGAAGTTGAGATTCAACGCCTGCTCGGGAAAGTTTGTGACAGGGGGTGGGGCCTTAAGGAAGCAGACGTAGTC
TGCCGACAACTGGGTTGCGGGAGCGCCCTCAAGACGTCCTATCAAGTTTACAGCAAAATTCAAGCAACTAATACCTG
GCTGTTTCTTTCCTCCTGCAATGGTAACGAAACGAGCCTCTGGGATTGTAAAAATTGGCAATGGGGAGGCCTTACAT
GTGATCACTATGAAGAGGCCAAAATCACCTGCAGCGCGCACCGAGAGCCTAGGCTGGTTGGAGGGGATATTCCCTGT
TCAGGTAGAGTTGAAGTCAAGCATGGTGATACCTGGGGGTCCATATGCGACTCCGATTTCAGTTTGGAAGCAGCGAG
CGTGCTGTGCCGAGAGCTTCAATGTGGGACAGTAGTTTCCATTCTTGGTGGCGCCCACTTCGGGGAAGGTAACGGAC
AGATTTGGGCGGAGGAATTCCAATGTGAGGGCCATGAAAGTCACCTGAGTCTTTGTCCTGTAGCGCCTCGGCCGGAA
GGTACTTGTTCTCACTCCAGAGACGTTGGCGTGGTTTGTTCTAGGTACACTGAGATAAGGCTGGTGAATGGCAAAAC
GCCTTGTGAAGGAAGGGTGGAGCTCAAAACGCTTGGCGCCTGGGGGTCTCTGTGCAACTCCCACTGGGACATAGAGG
ACGCACATGTCTTGTGCCAGCAACTCAAATGCGGTGTCGCGCTTTCAACCCCTGGGGGCGCTAGATTCGGGAAAGGT
AACGGCCAGATATGGCGCCATATGTTCCATTGCACCGGAACTGAACAGCATATGGGAGATTGTCCTGTGACTGCCTT
GGGGGCAAGTCTGTGTCCCTCAGAACAAGTGGCTTCAGTTATTTGCAGCGGTAACCAGAGTCAGACGCTCAGCTCCT
GCAACAGCAGCAGTCTGGGTCCAACAAGACCCACAATACCCGAGGAGTCAGCGGTCGCGTGCATCGAATCCGGGCAA
TTGAGACTCGTTAATGGCGGGGGTCGGTGCGCAGGGCGCGTGGAGATCTACCACGAGGGTAGTTGGGGCACAATTTG
TGACGACAGCTGGGACCTTTCCGACGCTCATGTCGTATGCCGACAACTGGGGTGCGGTGAGGCCATCAATGCCACCG
GAAGCGCGCATTTCGGTGAAGGCACGGGCCCAATTTGGCTTGATGAGATGAAATGTAATGGAAAGGAGTCCCGCATT
TGGCAATGCCATAGCCATGGCTGGGGTCAACAAAATTGTCGACACAAAGAAGATGCCGGGGTGATCTGCTCAGAATT
CATGTCCTTGCGGCTTACTAGCGAAGCGTCCCGCGAGGCCTGTGCTGGGAGACTGGAAGTTTTTTATAACGGGGCTT
GGGGAACGGTTGGTAAGTCATCAATGAGTGAAACCACAGTTGGTGTTGTGTGTAGACAACTCGGTTGCGCCGACAAG
GGAAAGATCAACCCGGCGAGCCTTGATAAGGCCATGAGCATCCCCATGTGGGTGGATAACGTTCAGTGTCCGAAAGG
TCCCGATACCTTGTGGCAATGCCCAAGTTCTCCTTGGGAGAAGAGACTCGCCTCACCAAGTGAAGAGACGTGGATTA
CGTGTGATAACAAAATTAGGCTCCAAGAAGGACCGACCAGTTGCAGCGGAAGAGTTGAGATATGGCATGGAGGAAGC
TGGGGAACCGTGTGCGATGACAGCTGGGACCTGGACGACGCCCAGGTGGTGTGCCAACAGCTGGGTTGCGGGCCTGC
CCTCAAAGCATTTAAGGAAGCCGAATTCGGTCAGGGTACTGGGCCAATCTGGCTGAACGAGGTAAAGTGCAAAGGTA
ACGAAAGTAGCCTGTGGGACTGTCCGGCACGAAGGTGGGGCCACAGCGAGTGTGGCCATAAGGAAGACGCGGCCGTG
AACTGTACAGACATATCCGTACAAAAAACGCCCCAAAAGGCGACGACCGGGCGATCATCAAGACAATCTAGCTTTAT
TGCCGTGGGAATTCTCGGTGTAGTGCTTCTTGCTATATTTGTCGCTTTGTTCTTTCTGACTAAAAAGCGCAGGCAAA
GGCAGCGGCTTGCTGTGAGCTCTCGGGGAGAAAACCTCGTTCACCAAATCCAATACCGAGAAATGAACTCCTGTCTC
AACGCCGACGATCTTGACCTGATGAACTCATCTGAGAACTCACACGAGTCCGCCGATTTCAGCGCGGCGGAATTGAT
CTCTGTCAGCAAATTTCTGCCTATAAGTGGCATGGAAAAAGAAGCCATACTCTCTCACACGGAAAAGGAAAATGGCA
ACCTT
B10_AC_163 (SEQ ID NO: 60):
ATGAGGCTACCCCTGCTCCTGGTTTTTGCCTCTGTCATTCCGGGTGCTGTTCTCTCCACCATTGAGGAACAGGCCAA
GACATTTTTGGACAAGTTTAACCACGAAGCCGAAGACCTGTTCTATCAAAGTTCACTTGCTTCTTGGAATTATAACA
CCAATATTACTGAAGAGAATGTCCAAAACATGAATAATGCTGGGGACAAATGGTCTGCCTTTTTAAAGGAACAGTCC
ACACTTGCCCAAATGTATCCACTACAAGAAATTCAGAATCTCACAGTCAAGCTTCAGCTGCAGGCTCTTCAGCAAAA
TGGGTCTTCAGTGCTCTCAGAAGACAAGAGCAAACGGTTGAACACAATTCTAAATACAATGAGCACCATCTACAGTA
CTGGAAAAGTTTGTAACCCAGATAATCCACAAGAATGCTTATTACTTGAACCAGGTTTGAATGAAATAATGGCAAAC
AGTTTAGACTACAATGAGAGGCTCTGGGCTTGGGAAAGCTGGAGATCTGAGGTCGGCAAGCAGCTGAGGCCATTATA
TGAAGAGTATGTGGTCTTGAAAAATGAGATGGCAAGAGCAAATCATTATGAGGACTATGGGGATTATTGGAGAGGAG
ACTATGAAGTAAATGGGGTAGATGGCTATGACTACAGCCGCGGCCAGTTGATTGAAGATGTGGAACATACCTTTGAA
GAGATTAAACCATTATATGAACATCTTCATGCCTATGTGAGGGCAAAGTTGATGAATGCCTATCCTTCCTATATCAG
TCCAATTGGATGCCTCCCTGCTCATTTGCTTGGTGATATGTGGGGTAGATTTTGGACAAATCTGTACTCTTTGACAG
TTCCCTTTGGACAGAAACCAAACATAGATGTTACTGATGCAATGGTGGACCAGGCCTGGGATGCACAGAGAATATTC
AAGGAGGCCGAGAAGTTCTTTGTATCTGTTGGTCTTCCTAATATGACTCAAGGATTCTGGGAAAATTCCATGCTAAC
GGACCCAGGAAATGTTCAGAAAGCAGTCTGCCATCCCACAGCTTGGGACCTGGGGAAGGGCGACTTCAGGATCCTTA
TGTGCACAAAGGTGACAATGGACGACTTCCTGACAGCTCATCATGAGATGGGGCATATCCAGTATGATATGGCATAT
GCTGCACAACCTTTTCTGCTAAGAAATGGAGCTAATGAAGGATTCCATGAAGCTGTTGGGGAAATCATGTCACTTTC
TGCAGCCACACCTAAGCATTTAAAATCCATTGGTCTTCTGTCACCCGATTTTCAAGAAGACAATGAAACAGAAATAA
ACTTCCTGCTCAAACAAGCACTCACGATTGTTGGGACTCTGCCATTTACTTACATGTTAGAGAAGTGGAGGTGGATG
GTCTTTAAAGGGGAAATTCCCAAAGACCAGTGGATGAAAAAGTGGTGGGAGATGAAGCGAGAGATAGTTGGGGTGGT
GGAACCTGTGCCCCATGATGAAACATACTGTGACCCCGCATCTCTGTTCCATGTTTCTAATGATTACTCATTCATTC
GATATTACACAAGGACCCTTTACCAATTCCAGTTTCAAGAAGCACTTTGTCAAGCAGCTAAACATGAAGGCCCTCTG
CACAAATGTGACATCTCAAACTCTACAGAAGCTGGACAGAAACTGTTCAATATGCTGAGGCTTGGAAAATCAGAACC
CTGGACCCTAGCATTGGAAAATGTTGTAGGAGCAAAGAACATGAATGTAAGGCCACTGCTCAACTACTTTGAGCCCT
TATTTACCTGGCTGAAAGACCAGAACAAGAATTCTTTTGTGGGATGGAGTACCGACTGGAGTCCATATGCAGACCAA
AGCATCAAAGTGAGGATAAGCCTAAAATCAGCTCTTGGAGATAAAGCATATGAATGGAACGACAATGAAATGTACCT
GTTCCGATCATCTGTTGCATATGCTATGAGGCAGTACTTTTTAAAAGTAAAAAATCAGATGATTCTTTTTGGGGAGG
AGGATGTGCGAGTGGCTAATTTGAAACCAAGAATCTCCTTTAATTTCTTTGTCACTGCACCTAAAAATGTGTCTGAT
ATCATTCCTAGAACTGAAGTTGAAAAGGCCATCAGGATGTCCCGGAGCCGTATCAATGATGCTTTCCGTCTGAATGA
CAACAGCCTAGAGTTTCTGGGGATACAGCCAACACTTGGACCTCCTAACCAGCCCCCTGTTTCCGGATCCTTCGTGC
CGGTCTTCCTGCCAGCGAAGCCCACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCACACGAGGGGGCTGGACTTCGCCTG
TGATTCATCCCTGGGTGGAACAGATAAGGAGTTGAGGTTGGTGGATGGAGAGAATAAGTGCAGTGGACGGGTTGAGG
TCAAGGTGCAGGAAGAGTGGGGTACTGTTTGTAACAACGGCTGGAGTATGGAAGCAGTCTCCGTGATATGCAATCAA
CTTGGTTGCCCTACAGCCATAAAAGCTCCTGGATGGGCTAACTCATCTGCCGGAAGCGGTCGAATATGGATGGACCA
TGTATCCTGTAGGGGCAATGAGTCAGCACTGTGGGACTGCAAGCATGACGGGTGGGGAAAACACTCTAATTGTACCC
ACCAGCAAGATGCCGGTGTGACGTGTAGCGACGGCAGCAACTTGGAGATGCGGTTGACGCGCGGCGGGAACATGTGC
AGCGGTCGCATTGAGATAAAGTTCCAAGGGCGCTGGGGGACCGTTTGTGACGACAACTTCAATATAGATCATGCTAG
TGTGATATGCAGGCAGTTGGAGTGCGGTAGCGCCGTCTCATTTAGTGGTTCTAGCAATTTCGGTGAGGGATCCGGGC
CTATATGGTTCGACGACCTTATCTGCAATGGAAATGAGAGCGCCCTCTGGAACTGCAAGCACCAAGGTTGGGGTAAG
CATAACTGCGATCATGCTGAGGATGCGGGTGTGATTTGTAGCAAGGGAGCCGACTTGTCACTTCGACTCGTAGATGG
CGTGACGGAATGTAGCGGTCGCCTTGAGGTACGCTTCCAAGGTGAATGGGGCACAATCTGTGATGACGGTTGGGACT
CATATGACGCTGCGGTTGCTTGCAAACAGCTGGGCTGTCCGACAGCGGTAACGGCAATTGGGCGGGTAAATGCCAGT
AAGGGGTTTGGACATATATGGTTGGACAGTGTTTCTTGTCAGGGACACGAACCTGCAATATGGCAATGTAAACATCA
CGAGTGGGGGAAGCATTACTGCAATCATAATGAAGACGCAGGCGTCACATGCTCTGACGGATCCGACCTGGAATTGC
GGCTGAGAGGGGGGGGTTCACGGTGCGCAGGAACCGTGGAAGTTGAGATTCAACGCCTGCTCGGGAAAGTTTGTGAC
AGGGGGTGGGGCCTTAAGGAAGCAGACGTAGTCTGCCGACAACTGGGTTGCGGGAGCGCCCTCAAGACGTCCTATCA
AGTTTACAGCAAAATTCAAGCAACTAATACCTGGCTGTTTCTTTCCTCCTGCAATGGTAACGAAACGAGCCTCTGGG
ATTGTAAAAATTGGCAATGGGGAGGCCTTACATGTGATCACTATGAAGAGGCCAAAATCACCTGCAGCGCGCACCGA
GAGCCTAGGCTGGTTGGAGGGGATATTCCCTGTTCAGGTAGAGTTGAAGTCAAGCATGGTGATACCTGGGGGTCCAT
ATGCGACTCCGATTTCAGTTTGGAAGCAGCGAGCGTGCTGTGCCGAGAGCTTCAATGTGGGACAGTAGTTTCCATTC
TTGGTGGCGCCCACTTCGGGGAAGGTAACGGACAGATTTGGGCGGAGGAATTCCAATGTGAGGGCCATGAAAGTCAC
CTGAGTCTTTGTCCTGTAGCGCCTCGGCCGGAAGGTACTTGTTCTCACTCCAGAGACGTTGGCGTGGTTTGTTCTAG
GTACACTGAGATAAGGCTGGTGAATGGCAAAACGCCTTGTGAAGGAAGGGTGGAGCTCAAAACGCTTGGCGCCTGGG
GGTCTCTGTGCAACTCCCACTGGGACATAGAGGACGCACATGTCTTGTGCCAGCAACTCAAATGCGGTGTCGCGCTT
TCAACCCCTGGGGGCGCTAGATTCGGGAAAGGTAACGGCCAGATATGGCGCCATATGTTCCATTGCACCGGAACTGA
ACAGCATATGGGAGATTGTCCTGTGACTGCCTTGGGGGCAAGTCTGTGTCCCTCAGAACAAGTGGCTTCAGTTATTT
GCAGCGGTAACCAGAGTCAGACGCTCAGCTCCTGCAACAGCAGCAGTCTGGGTCCAACAAGACCCACAATACCCGAG
GAGTCAGCGGTCGCGTGCATCGAATCCGGGCAATTGAGACTCGTTAATGGCGGGGGTCGGTGCGCAGGGCGCGTGGA
GATCTACCACGAGGGTAGTTGGGGCACAATTTGTGACGACAGCTGGGACCTTTCCGACGCTCATGTCGTATGCCGAC
AACTGGGGTGCGGTGAGGCCATCAATGCCACCGGAAGCGCGCATTTCGGTGAAGGCACGGGCCCAATTTGGCTTGAT
GAGATGAAATGTAATGGAAAGGAGTCCCGCATTTGGCAATGCCATAGCCATGGCTGGGGTCAACAAAATTGTCGACA
CAAAGAAGATGCCGGGGTGATCTGCTCAGAATTCATGTCCTTGCGGCTTACTAGCGAAGCGTCCCGCGAGGCCTGTG
CTGGGAGACTGGAAGTTTTTTATAACGGGGCTTGGGGAACGGTTGGTAAGTCATCAATGAGTGAAACCACAGTTGGT
GTTGTGTGTAGACAACTCGGTTGCGCCGACAAGGGAAAGATCAACCCGGCGAGCCTTGATAAGGCCATGAGCATCCC
CATGTGGGTGGATAACGTTCAGTGTCCGAAAGGTCCCGATACCTTGTGGCAATGCCCAAGTTCTCCTTGGGAGAAGA
GACTCGCCTCACCAAGTGAAGAGACGTGGATTACGTGTGATAACAAAATTAGGCTCCAAGAAGGACCGACCAGTTGC
AGCGGAAGAGTTGAGATATGGCATGGAGGAAGCTGGGGAACCGTGTGCGATGACAGCTGGGACCTGGACGACGCCCA
GGTGGTGTGCCAACAGCTGGGTTGCGGGCCTGCCCTCAAAGCATTTAAGGAAGCCGAATTCGGTCAGGGTACTGGGC
CAATCTGGCTGAACGAGGTAAAGTGCAAAGGTAACGAAAGTAGCCTGTGGGACTGTCCGGCACGAAGGTGGGGCCAC
AGCGAGTGTGGCCATAAGGAAGACGCGGCCGTGAACTGTACAGACATATCCGTACAAAAAACGCCCCAAAAGGCGAC
GACCGGGCGATCATCAAGACAATCTAGCTTTATTGCCGTGGGAATTCTCGGTGTAGTGCTTCTTGCTATATTTGTCG
CTTTGTTCTTTCTGACTAAAAAGCGCAGGCAAAGGCAGCGGCTTGCTGTGAGCTCTCGGGGAGAAAACCTCGTTCAC
CAAATCCAATACCGAGAAATGAACTCCTGTCTCAACGCCGACGATCTTGACCTGATGAACTCATCTGAGAACTCACA
CGAGTCCGCCGATTTCAGCGCGGCGGAATTGATCTCTGTCAGCAAATTTCTGCCTATAAGTGGCATGGAAAAAGAAG
CCATACTCTCTCACACGGAAAAGGAAAATGGCAACCTT

### Example 6: Analysis of CBR Construct Based on MEGF10

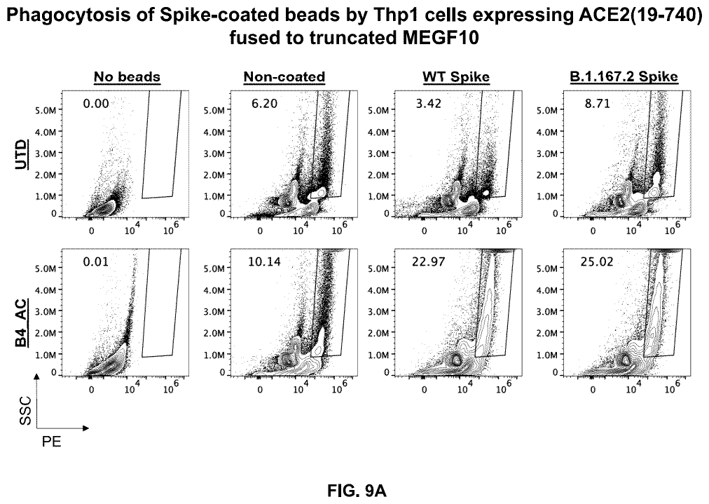

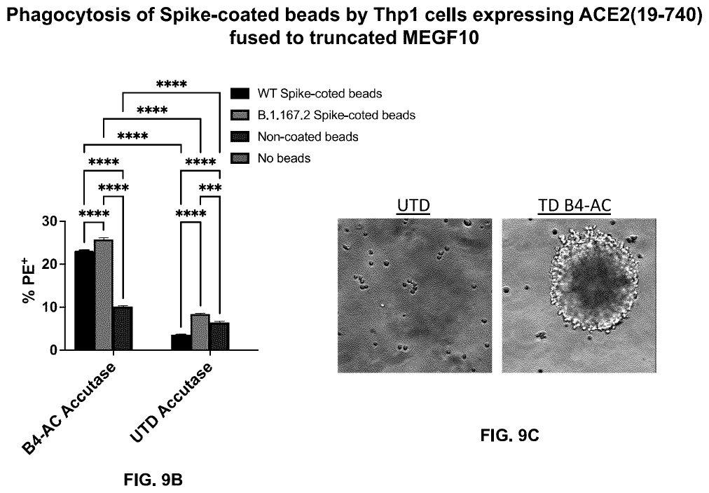

Thp1 cells were transduced with lentivirus carrying ACE2 (19-740 aa) on top of MEGF10 construct (B4-AC) under EF1a promoter, the cells were stained with biotinylated SARS-CoV-2 Spike protein (MyBioSource, San Diego, CA) followed by PE-streptavidin (BioLegend, San Diego, CA), and positive cells were sorted using Melody sorter (BD, Franklin Lakes, NJ). Streptavidin-coated 5.06 um beads (Spherotech, Lake Forest, IL) were sterilized using 70% isopropanol and labeled with 10 uM pHrodo red dye (Thermo Fisher, Waltham, MA) according to manufacturer instructions. Washed beads were then incubated with biotinylated WT SARS-CoV-2 Spike protein (MyBioSource, San Diego, CA) or with Delta variant, B.1.617.2 SARS-CoV-2 Spike protein (SinoBiological, Wayne, PA) at a ratio of 0.025 nmole biotinylated spike protein per 0.5 mg beads. Beads were incubated overnight with cells at a ratio of 1:5 (cells:beads). Since MEGF10 is involved in cell adhesion, and because all the cells were clustered together and did not break apart by pipetting (FIG. 9C), the cells were treated with Accutase (Sigma-Aldrich, St. Louis, M0) for 15 minutes prior to flow cytometry analysis.

As shown in FIG. 9A-9B, only Spike-coated beads (WT and B.1.167.2) were phagocytosed and only by Thp1 that were transduced with MEGF10-based B4-AC CBR construct containing ACE2 (19-740 AA) bait on the surface. Non-coated beads were not phagocytosed and untransduced (UTD) cells did not phagocytose any beads.

A neutralization assay was used to test the ability of Thp1 cells transduced with MEGF10-based CBR construct containing ACE2 bait on the surface (B4-AC) to neutralize SARS-CoV-2 pseudotyped lentivirus. Similar to Example 4, Spike-LV-GFP was pre-incubated with either B4-AC Thp 1 or with UTD Thp 1 or with ACE2-293 effector cells for 2 hr before incubation with ACE2-293 target cells. E:T ratio was 100:1 and during the pre-incubation period, the cells were mixed every 15 minutes. Based on target cells amount, the tested MOI was 2.3. UTD and B4-AC Thp1 were treated with Accutase (Sigma-Aldrich, St. Louis, MO) for 15 minutes before counting for pre-incubation.

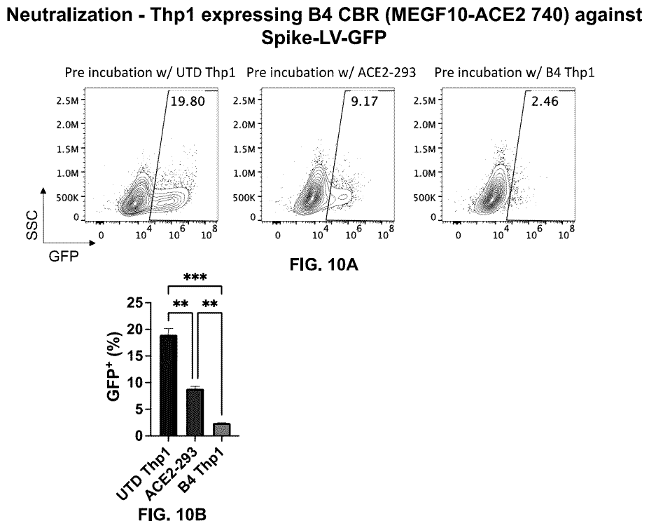

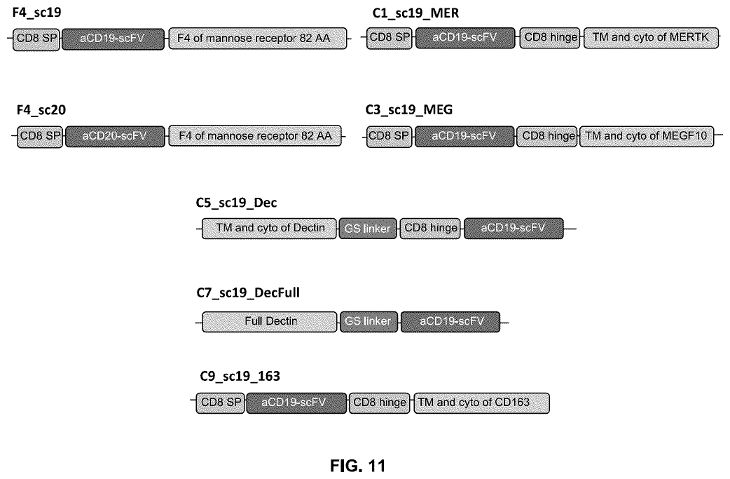

As shown in FIG. 10A-10B, pre-incubation with either B4-AC Thp1 cells or ACE2-293 cells reduced the viral load in the supernatant as observed by lower transduction efficiency of target cells after a subsequent incubation. Pre-incubation with B4-AC Thp 1 reduced the viral load almost 10-fold.

### Example 7: CPR Constructs Based on Additional scFvs

Additional CPR constructs were designed containing anti-CD19 scFv or anti-CD20 scFv on top of the following phagocytic receptors: mannose receptor (F4), MERTK, MEGF10, Dectin-1, and CD163. Schematics for each of the constructs is shown in FIG. 11.

The nucleotide sequences for each of these constructs are provided below.

F4_sc19 (SEQ ID NO: 79):
ATGGCCTTACCAGTGACCGCCTTGCTCCTGCCGCTGGCCTTGCTGCTCCACGCCGCCAGGCCGGGATCCGAGGTGAA
ACTGCAGGAGTCAGGACCTGGCCTGGTGGCGCCCTCACAGAGCCTGTCCGTCACATGCACTGTCTCAGGGGTCTCAT
TACCCGACTATGGTGTAAGCTGGATTCGCCAGCCTCCACGAAAGGGTCTGGAGTGGCTGGGAGTAATATGGGGTAGT
GAAACCACATACTATAATTCAGCTCTCAAATCCAGACTGACCATCATCAAGGACAACTCCAAGAGCCAAGTTTTCTT
AAAAATGAACAGTCTGCAAACTGATGACACAGCCATTTACTACTGTGCCAAACATTATTACTACGGTGGTAGCTATG
CTATGGACTACTGGGGTCAAGGAACCTCAGTCACCGTCTCCTCAGGCGGCGGTGGCTCTGGCGGTGGCGGTTCCGGC
GGTGGTGGTAGCGACATCCAGATGACACAGACTACATCCTCCCTGTCTGCCTCTCTGGGAGACAGAGTCACCATCAG
TTGCAGGGCAAGTCAGGACATTAGTAAATATTTAAATTGGTATCAGCAGAAACCAGATGGAACTGTTAAACTCCTGA
TCTACCATACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGTACACGTTCGG
AGGGGGGACTAAGTTGGAAATAACACGGGCTGATGCTGCACCAACTGTATCCATCTTCCCACCATCCAGTAATGACA
CAAGGAAGATGGACCCTTCTAAACCGTCTTCCAACGTGGCCGGAGTAGTCATCATTGTGATCCTCCTGATTTTAACG
GGTGCTGGCCTTGCCGCCTATTTCTTTTATAAGAAAAGACGTGTGCACCTACCTCAAGAGGGCGCCTTTGAAAACAC
TCTGTATTTTAACAGTCAGTCAAGCCCAGGAACTAGTGATATGAAAGATCTCGTGGGCAATATTGAACAGAATGAAC
ACTCGGTCATC
F4_sc20 (SEQ ID NO: 80):
ATGGCCTTACCAGTGACCGCCTTGCTCCTGCCGCTGGCCTTGCTGCTCCACGCCGCCAGGCCGGGATCCCAAATTGT
TCTCTCCCAGTCTCCAGCAATCCTTTCTGCATCTCCAGGGGAGAAGGTCACAATGACTTGCAGGGCCAGCTCAAGTT
TAAGTTTCATGCACTGGTACCAGCAGAAGCCAGGATCCTCCCCCAAACCCTGGATTTATGCCACATCCAACCTGGCT
TCTGGAGTCCCTGCTCGCTTCAGTGGCAGTGGGTCTGGGACCTCTTACTCTCTCACAATCAGCAGAGTGGAGGCTGA
AGATGCTGCCACTTATTTCTGCCATCAGTGGAGTAGTAACCCGCTCACGTTCGGTGCTGGGACCAAGCTGGAGCTCA
AACGGGGGGCGGCGGCTCTGGCGGCGGTGGCTCTGGCGGTGGCGGTTCCGGCGGTGGTGGTAGCCAGGTGCAACTG
CGGCAGCCTGGGGCTGAGCTGGTGAAGCCTGGGGCCTCAGTGAAGATGTCCTGCAAGGCTTCTGGCTACACATTTAC
CAGTTACAATATGCACTGGGTAAAGCAGACACCTGGACAGGGCCTGGAATGGATTGGAGCTATTTATCCAGGAAATG
GTGATACTTCCTACAATCAGAAGTTCAAAGGCAAGGCCACATTGACTGCAGACAAATCCTCCAGCACAGCCTACATG
CAGCTCAGCAGTCTGACATCTGAGGACTCTGCGGTCTATTACTGTGCAAGATCGCACTACGGTAGTAACTACGTAGA
CTACTTTGACTACTGGGGCCAAGGCACCACTCTCACAGTCTCCTCTGACACAAGGAAGATGGACCCTTCTAAACCGT
CTTCCAACGTGGCCGGAGTAGTCATCATTGTGATCCTCCTGATTTTAACGGGTGCTGGCCTTGCCGCCTATTTCTTT
TATAAGAAAAGACGTGTGCACCTACCTCAAGAGGGCGCCTTTGAAAACACTCTGTATTTTAACAGTCAGTCAAGCCC
AGGAACTAGTGATATGAAAGATCTCGTGGGCAATATTGAACAGAATGAACACTCGGTCATC
C1_sc19_MER (SEQ ID NO: 81):
ATGGCCTTACCAGTGACCGCCTTGCTCCTGCCGCTGGCCTTGCTGCTCCACGCCGCCAGGCCGGGATCCGAGGTGAA
ACTGCAGGAGTCAGGACCTGGCCTGGTGGCGCCCTCACAGAGCCTGTCCGTCACATGCACTGTCTCAGGGGTCTCAT
TACCCGACTATGGTGTAAGCTGGATTCGCCAGCCTCCACGAAAGGGTCTGGAGTGGCTGGGAGTAATATGGGGTAGT
GAAACCACATACTATAATTCAGCTCTCAAATCCAGACTGACCATCATCAAGGACAACTCCAAGAGCCAAGTTTTCTT
AAAAATGAACAGTCTGCAAACTGATGACACAGCCATTTACTACTGTGCCAAACATTATTACTACGGTGGTAGCTATG
CTATGGACTACTGGGGTCAAGGAACCTCAGTCACCGTCTCCTCAGGCGGCGGTGGCTCTGGCGGTGGCGGTTCCGGC
GGTGGTGGTAGCGACATCCAGATGACACAGACTACATCCTCCCTGTCTGCCTCTCTGGGAGACAGAGTCACCATCAG
TTGCAGGGCAAGTCAGGACATTAGTAAATATTTAAATTGGTATCAGCAGAAACCAGATGGAACTGTTAAACTCCTGA
TCTACCATACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGTACACGTTCGG
AGGGGGGACTAAGTTGGAAATAACACGGGCTGATGCTGCACCAACTGTATCCATCTTCCCACCATCCAGTAATGGAT
CCTTCGTGCCGGTCTTCCTGCCAGCGAAGCCCACCACGACGCCAGCGCCGCGACCACCAACACCGGCGCCCACCATC
GCGTCGCAGCCCCTGTCCCTGCGCCCAGAGGCGTGCCGGCCAGCGGCGGGGGGCGCAGTGCACACGAGGGGGCTGGA
CTTCGCCTGTGATTTTGGATGTTTTTGTGGTTTCATCCTCATCGGTTTGATATTGTACATAAGTCTGGCGATAAGGA
AGAGAGTTCAAGAGACAAAGTTCGGAAATGCCTTTACAGAGGAAGACAGTGAGCTCGTTGTAAACTACATCGCAAAA
AAAAGCTTCTGTAGAAGAGCAATAGAGCTCACGTTGCACTCACTCGGTGTGTCCGAAGAACTCCAGAATAAACTGGA
AGACGTCGTTATCGATCGGAACCTCCTCATACTTGGAAAAATACTGGGAGAAGGAGAGTTCGGCAGTGTCATGGAAG
GTAACTTGAAACAAGAGGATGGTACCTCACTCAAGGTAGCTGTCAAGACGATGAAACTTGATAACAGTTCACAAAGG
GAGATCGAAGAATTTCTGTCTGAGGCCGCCTGTATGAAAGACTTCTCACATCCTAATGTCATCAGACTTCTTGGCGT
TTGTATCGAGATGTCTAGCCAAGGAATCCCAAAACCTATGGTCATATTGCCTTTCATGAAATATGGCGATCTGCATA
CATATTTGCTCTACTCTAGACTTGAGACAGGGCCCAAACATATTCCTCTCCAGACATTGCTCAAGTTTATGGTCGAT
ATTGCCCTGGGTATGGAGTACTTGAGCAACCGAAATTTTCTGCATCGGGATCTTGCCGCACGCAACTGCATGCTGCG
CGATGACATGACCGTCTGCGTGGCTGATTTTGGGCTGTCAAAAAAAATATATTCTGGAGACTACTACCGACAAGGGC
GGATTGCAAAGATGCCCGTCAAATGGATTGCGATTGAAAGTTTGGCGGACAGGGTATATACTTCCAAATCAGATGTT
TGGGCTTTTGGAGTCACTATGTGGGAAATAGCTACACGCGGTATGACCCCGTACCCCGGAGTACAAAATCATGAAAT
GTATGACTATCTCCTTCATGGACACAGGCTGAAGCAGCCCGAGGACTGCCTGGACGAACTGTATGAAATAATGTATT
CTTGTTGGCGAACCGATCCCTTGGACCGGCCTACTTTCAGTGTCCTTAGATTGCAACTTGAGAAATTGCTCGAGTCT
TTGCCGGATGTGCGAAACCAGGCAGACGTGATCTATGTCAATACCCAACTTTTGGAAAGTTCTGAGGGCCTCGCACA
GGGTTCTACCCTTGCCCCGTTGGATCTTAACATAGACCCAGACAGCATAATTGCTTCTTGTACACCTCGCGCTGCCA
TATCAGTTGTAACAGCGGAGGTCCATGATAGTAAACCTCACGAGGGTCGCTATATCCTGAACGGCGGGTCAGAAGAA
TGGGAAGACCTGACATCAGCGCCGAGCGCCGCCGTTACTGCTGAGAAAAACTCTGTCCTGCCCGGAGAGCGCTTGGT
TCGGAACGGGGTAAGTTGGAGCCATAGCTCAATGCTCCCCCTGGGTTCAAGTCTCCCGGACGAGCTTCTTTTTGCGG
ACGACTCATCTGAGGGGTCCGAAGTTCTGATG
C3_sc19_MEG (SEQ ID NO: 82):
ATGGCCTTACCAGTGACCGCCTTGCTCCTGCCGCTGGCCTTGCTGCTCCACGCCGCCAGGCCGGGATCCGAGGTGAA
ACTGCAGGAGTCAGGACCTGGCCTGGTGGCGCCCTCACAGAGCCTGTCCGTCACATGCACTGTCTCAGGGGTCTCAT
TACCCGACTATGGTGTAAGCTGGATTCGCCAGCCTCCACGAAAGGGTCTGGAGTGGCTGGGAGTAATATGGGGTAGT
GAAACCACATACTATAATTCAGCTCTCAAATCCAGACTGACCATCATCAAGGACAACTCCAAGAGCCAAGTTTTCTT
AAAAATGAACAGTCTGCAAACTGATGACACAGCCATTTACTACTGTGCCAAACATTATTACTACGGTGGTAGCTATG
CTATGGACTACTGGGGTCAAGGAACCTCAGTCACCGTCTCCTCAGGCGGCGGTGGCTCTGGCGGTGGCGGTTCCGGC
GGTGGTGGTAGCGACATCCAGATGACACAGACTACATCCTCCCTGTCTGCCTCTCTGGGAGACAGAGTCACCATCAG
TTGCAGGGCAAGTCAGGACATTAGTAAATATTTAAATTGGTATCAGCAGAAACCAGATGGAACTGTTAAACTCCTGA
TCTACCATACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGTACACGTTCGG
AGGGGGGACTAAGTTGGAAATAACACGGGCTGATGCTGCACCAACTGTATCCATCTTCCCACCATCCAGTAATGGAT
CCTTCGTGCCGGTCTTCCTGCCAGCGAAGCCCACCACGACGCCAGCGCCGCGACCACCAACACCGGCGCCCACCATC
GCGTCGCAGCCCCTGTCCCTGCGCCCAGAGGCGTGCCGGCCAGCGGCGGGGGGCGCAGTGCACACGAGGGGGCTGGA
CTTCGCCTGTGATGCTATCGCGGGGATCATTATATTGGTCTTGGTGGTGCTGTTTCTGCTCGCGCTTTTCATTATAT
ACCGCCATAAGCAGAAGGGCAAAGAGTCCTCCATGCCAGCCGTGACCTATACGCCTGCGATGCGCGTTGTCAACGCC
GATTACACCATCAGTGGTACCCTCCCGCACAGTAACGGCGGAAATGCAAACTCTCATTACTTTACAAATCCTAGTTA
CCATACACTCACTCAGTGTGCTACCTCTCCCCATGTGAACAATCGGGACAGGATGACCGTTACGAAAAGCAAAAATA
ACCAGTTGTTTGTGAACCTTAAGAATGTGAATCCCGGCAAGAGGGGTCCGGTGGGTGACTGCACCGGAACTCTCCCC
GCTGACTGGAAGCATGGCGGGTACCTGAACGAACTCGGCGCGTTTGGGCTCGACCGAAGCTACATGGGTAAAAGTCT
TAAGGACCTCGGTAAGAATAGCGAGTATAATAGCTCTAACTGTTCCCTTTCCAGCTCCGAGAATCCGTACGCTACTA
TAAAAGACCCCCCGGTGCTCATTCCCAAATCCAGTGAGTGCGGGTACGTGGAGATGAAAAGTCCCGCTCGAAGAGAC
AGTCCATACGCGGAAATCAATAACTCCACCAGTGCGAACCGCAATGTGTACGAAGTGGAGCCCACCGTTTCCGTTGT
ACAAGGTGTATTTTCAAACAATGGGAGGCTTAGCCAGGACCCCTATGATCTTCCAAAGAACAGCCACATCCCGTGTC
ATTATGATCTGTTGCCGGTGAGGGATTCTAGCTCTTCTCCTAAACAAGAGGACTCAGGTGGCTCTAGTTCCAACTCC
TCCAGTTCTTCAGAG
C5_sc19_Dec (SEQ ID NO: 83):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGTTCGTGCCGGTCTTCCTGCCAGCGAAGCCCACCACG
ACGCCAGCGCCGCGACCACCAACACCGGCGCCCACCATCGCGTCGCAGCCCCTGTCCCTGCGCCCAGAGGCGTGCCG
GCCAGCGGCGGGGGGCGCAGTGCACACGAGGGGGCTGGACTTCGCCTGTGATGGATCCGAGGTGAAACTGCAGGAGT
CAGGACCTGGCCTGGTGGCGCCCTCACAGAGCCTGTCCGTCACATGCACTGTCTCAGGGGTCTCATTACCCGACTAT
GGTGTAAGCTGGATTCGCCAGCCTCCACGAAAGGGTCTGGAGTGGCTGGGAGTAATATGGGGTAGTGAAACCACATA
CTATAATTCAGCTCTCAAATCCAGACTGACCATCATCAAGGACAACTCCAAGAGCCAAGTTTTCTTAAAAATGAACA
GTCTGCAAACTGATGACACAGCCATTTACTACTGTGCCAAACATTATTACTACGGTGGTAGCTATGCTATGGACTAC
TGGGGTCAAGGAACCTCAGTCACCGTCTCCTCAGGCGGCGGTGGCTCTGGCGGTGGCGGTTCCGGCGGTGGTGGTAG
CGACATCCAGATGACACAGACTACATCCTCCCTGTCTGCCTCTCTGGGAGACAGAGTCACCATCAGTTGCAGGGCAA
GTCAGGACATTAGTAAATATTTAAATTGGTATCAGCAGAAACCAGATGGAACTGTTAAACTCCTGATCTACCATACA
TCAAGATTACACTCAGGAGTCCCATCAAGGTTCAGTGGCAGTGGGTCTGGAACAGATTATTCTCTCACCATTAGCAA
CCTGGAGCAAGAAGATATTGCCACTTACTTTTGCCAACAGGGTAATACGCTTCCGTACACGTTCGGAGGGGGGACTA
AGTTGGAAATAACACGGGCTGATGCTGCACCAACTGTATCCATCTTCCCACCATCCAGTAAT
C7_sc19_DecFull (SEQ ID NO: 84):
ATGGAGTATCACCCCGACCTGGAGAATTTGGACGAGGATGGCTACACTCAACTGCATTTCGATAGTCAGTCCAATAC
AAGGATCGCTGTTGTGTCTGAAAAGGGCAGTTGTGCTGCCAGTCCACCGTGGCGGTTGATTGCCGTCATCCTCGGCA
TTCTGTGCTTGGTTATTCTCGTTATAGCGGTGGTGCTTGGGACCATGGCGATCTGGCGCTCCAACTCTGGAAGTAAC
ACCCTTGAAAATGGTTACTTCCTCAGTAGGAACAAAGAGAACCATTCCCAGCCGACACAGTCAAGCCTTGAAGATTC
AGTCACCCCTACAAAGGCCGTAAAAACGACAGGTGTCCTGTCCTCTCCGTGTCCGCCTAACTGGATCATCTACGAGA
AAAGTTGTTATCTGTTTAGCATGAGCCTTAACAGTTGGGATGGCTCAAAAAGGCAGTGCTGGCAACTGGGGAGCAAC
CTTTTGAAGATAGACAGTTCCAACGAACTGGGCTTCATAGTCAAACAGGTGTCCTCTCAACCTGATAACTCATTCTG
GATCGGGCTCAGTCGACCCCAAACTGAGGTTCCATGGCTTTGGGAAGACGGCAGCACTTTCTCTTCAAATTTGTTTC
AAATAAGAACCACCGCTACGCAGGAGAATCCGAGTCCGAACTGTGTTTGGATTCACGTCTCAGTCATTTACGACCAA
CTTTGTAGTGTCCCTAGCTATTCCATCTGCGAGAAAAAGTTCAGTATGGGATCCGAGGTGAAACTGCAGGAGTCAGG
ACCTGGCCTGGTGGCGCCCTCACAGAGCCTGTCCGTCACATGCACTGTCTCAGGGGTCTCATTACCCGACTATGGTG
TAAGCTGGATTCGCCAGCCTCCACGAAAGGGTCTGGAGTGGCTGGGAGTAATATGGGGTAGTGAAACCACATACTAT
AATTCAGCTCTCAAATCCAGACTGACCATCATCAAGGACAACTCCAAGAGCCAAGTTTTCTTAAAAATGAACAGTCT
GCAAACTGATGACACAGCCATTTACTACTGTGCCAAACATTATTACTACGGTGGTAGCTATGCTATGGACTACTGGG
GTCAAGGAACCTCAGTCACCGTCTCCTCAGGCGGCGGTGGCTCTGGCGGTGGCGGTTCCGGCGGTGGTGGTAGCGAC
ATCCAGATGACACAGACTACATCCTCCCTGTCTGCCTCTCTGGGAGACAGAGTCACCATCAGTTGCAGGGCAAGTCA
GGACATTAGTAAATATTTAAATTGGTATCAGCAGAAACCAGATGGAACTGTTAAACTCCTGATCTACCATACATCAA
GATTACACTCAGGAGTCCCATCAAGGTTCAGTGGCAGTGGGTCTGGAACAGATTATTCTCTCACCATTAGCAACCTG
GAGCAAGAAGATATTGCCACTTACTTTTGCCAACAGGGTAATACGCTTCCGTACACGTTCGGAGGGGGGACTAAGTT
GGAAATAACACGGGCTGATGCTGCACCAACTGTATCCATCTTCCCACCATCCAGTAAT
C9_sc19_163 (SEQ ID NO: 85):
ATGGCCTTACCAGTGACCGCCTTGCTCCTGCCGCTGGCCTTGCTGCTCCACGCCGCCAGGCCGGGATCCGAGGTGAA
ACTGCAGGAGTCAGGACCTGGCCTGGTGGCGCCCTCACAGAGCCTGTCCGTCACATGCACTGTCTCAGGGGTCTCAT
TACCCGACTATGGTGTAAGCTGGATTCGCCAGCCTCCACGAAAGGGTCTGGAGTGGCTGGGAGTAATATGGGGTAGT
GAAACCACATACTATAATTCAGCTCTCAAATCCAGACTGACCATCATCAAGGACAACTCCAAGAGCCAAGTTTTCTT
AAAAATGAACAGTCTGCAAACTGATGACACAGCCATTTACTACTGTGCCAAACATTATTACTACGGTGGTAGCTATG
CTATGGACTACTGGGGTCAAGGAACCTCAGTCACCGTCTCCTCAGGCGGCGGTGGCTCTGGCGGTGGCGGTTCCGGC
GGTGGTGGTAGCGACATCCAGATGACACAGACTACATCCTCCCTGTCTGCCTCTCTGGGAGACAGAGTCACCATCAG
TTGCAGGGCAAGTCAGGACATTAGTAAATATTTAAATTGGTATCAGCAGAAACCAGATGGAACTGTTAAACTCCTGA
TCTACCATACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGTACACGTTCGG
AGGGGGGACTAAGTTGGAAATAACACGGGCTGATGCTGCACCAACTGTATCCATCTTCCCACCATCCAGTAATGGAT
CCTTCGTGCCGGTCTTCCTGCCAGCGAAGCCCACCACGACGCCAGCGCCGCGACCACCAACACCGGCGCCCACCATC
GCGTCGCAGCCCCTGTCCCTGCGCCCAGAGGCGTGCCGGCCAGCGGCGGGGGGCGCAGTGCACACGAGGGGGCTGGA
CTTCGCCTGTGATTCATCCCTGGGTGGAACAGATAAGGAGTTGAGGTTGGTGGATGGAGAGAATAAGTGCAGTGGAC
GGGTTGAGGTCAAGGTGCAGGAAGAGTGGGGTACTGTTTGTAACAACGGCTGGAGTATGGAAGCAGTCTCCGTGATA
TGCAATCAACTTGGTTGCCCTACAGCCATAAAAGCTCCTGGATGGGCTAACTCATCTGCCGGAAGCGGTCGAATATG
GATGGACCATGTATCCTGTAGGGGCAATGAGTCAGCACTGTGGGACTGCAAGCATGACGGGTGGGGAAAACACTCTA
ATTGTACCCACCAGCAAGATGCCGGTGTGACGTGTAGCGACGGCAGCAACTTGGAGATGCGGTTGACGCGCGGCGGG
AACATGTGCAGCGGTCGCATTGAGATAAAGTTCCAAGGGCGCTGGGGGACCGTTTGTGACGACAACTTCAATATAGA
TCATGCTAGTGTGATATGCAGGCAGTTGGAGTGCGGTAGCGCCGTCTCATTTAGTGGTTCTAGCAATTTCGGTGAGG
GATCCGGGCCTATATGGTTCGACGACCTTATCTGCAATGGAAATGAGAGCGCCCTCTGGAACTGCAAGCACCAAGGT
TGGGGTAAGCATAACTGCGATCATGCTGAGGATGCGGGTGTGATTTGTAGCAAGGGAGCCGACTTGTCACTTCGACT
CGTAGATGGCGTGACGGAATGTAGCGGTCGCCTTGAGGTACGCTTCCAAGGTGAATGGGGCACAATCTGTGATGACG
GTTGGGACTCATATGACGCTGCGGTTGCTTGCAAACAGCTGGGCTGTCCGACAGCGGTAACGGCAATTGGGCGGGTA
AATGCCAGTAAGGGGTTTGGACATATATGGTTGGACAGTGTTTCTTGTCAGGGACACGAACCTGCAATATGGCAATG
TAAACATCACGAGTGGGGGAAGCATTACTGCAATCATAATGAAGACGCAGGCGTCACATGCTCTGACGGATCCGACC
TGGAATTGCGGCTGAGAGGGGGGGGTTCACGGTGCGCAGGAACCGTGGAAGTTGAGATTCAACGCCTGCTCGGGAAA
GTTTGTGACAGGGGGTGGGGCCTTAAGGAAGCAGACGTAGTCTGCCGACAACTGGGTTGCGGGAGCGCCCTCAAGAC
GTCCTATCAAGTTTACAGCAAAATTCAAGCAACTAATACCTGGCTGTTTCTTTCCTCCTGCAATGGTAACGAAACGA
GCCTCTGGGATTGTAAAAATTGGCAATGGGGAGGCCTTACATGTGATCACTATGAAGAGGCCAAAATCACCTGCAGC
GCGCACCGAGAGCCTAGGCTGGTTGGAGGGGATATTCCCTGTTCAGGTAGAGTTGAAGTCAAGCATGGTGATACCTG
GGGGTCCATATGCGACTCCGATTTCAGTTTGGAAGCAGCGAGCGTGCTGTGCCGAGAGCTTCAATGTGGGACAGTAG
TTTCCATTCTTGGTGGCGCCCACTTCGGGGAAGGTAACGGACAGATTTGGGCGGAGGAATTCCAATGTGAGGGCCAT
GAAAGTCACCTGAGTCTTTGTCCTGTAGCGCCTCGGCCGGAAGGTACTTGTTCTCACTCCAGAGACGTTGGCGTGGT
TTGTTCTAGGTACACTGAGATAAGGCTGGTGAATGGCAAAACGCCTTGTGAAGGAAGGGTGGAGCTCAAAACGCTTG
GCGCCTGGGGGTCTCTGTGCAACTCCCACTGGGACATAGAGGACGCACATGTCTTGTGCCAGCAACTCAAATGCGGT
GTCGCGCTTTCAACCCCTGGGGGCGCTAGATTCGGGAAAGGTAACGGCCAGATATGGCGCCATATGTTCCATTGCAC
CGGAACTGAACAGCATATGGGAGATTGTCCTGTGACTGCCTTGGGGGCAAGTCTGTGTCCCTCAGAACAAGTGGCTT
CAGTTATTTGCAGCGGTAACCAGAGTCAGACGCTCAGCTCCTGCAACAGCAGCAGTCTGGGTCCAACAAGACCCACA
ATACCCGAGGAGTCAGCGGTCGCGTGCATCGAATCCGGGCAATTGAGACTCGTTAATGGCGGGGGTCGGTGCGCAGG
GCGCGTGGAGATCTACCACGAGGGTAGTTGGGGCACAATTTGTGACGACAGCTGGGACCTTTCCGACGCTCATGTCG
TATGCCGACAACTGGGGTGCGGTGAGGCCATCAATGCCACCGGAAGCGCGCATTTCGGTGAAGGCACGGGCCCAATT
TGGCTTGATGAGATGAAATGTAATGGAAAGGAGTCCCGCATTTGGCAATGCCATAGCCATGGCTGGGGTCAACAAAA
TTGTCGACACAAAGAAGATGCCGGGGTGATCTGCTCAGAATTCATGTCCTTGCGGCTTACTAGCGAAGCGTCCCGCG
AGGCCTGTGCTGGGAGACTGGAAGTTTTTTATAACGGGGCTTGGGGAACGGTTGGTAAGTCATCAATGAGTGAAACC
ACAGTTGGTGTTGTGTGTAGACAACTCGGTTGCGCCGACAAGGGAAAGATCAACCCGGCGAGCCTTGATAAGGCCAT
GAGCATCCCCATGTGGGTGGATAACGTTCAGTGTCCGAAAGGTCCCGATACCTTGTGGCAATGCCCAAGTTCTCCTT
GGGAGAAGAGACTCGCCTCACCAAGTGAAGAGACGTGGATTACGTGTGATAACAAAATTAGGCTCCAAGAAGGACCG
ACCAGTTGCAGCGGAAGAGTTGAGATATGGCATGGAGGAAGCTGGGGAACCGTGTGCGATGACAGCTGGGACCTGGA
CGACGCCCAGGTGGTGTGCCAACAGCTGGGTTGCGGGCCTGCCCTCAAAGCATTTAAGGAAGCCGAATTCGGTCAGG
GTACTGGGCCAATCTGGCTGAACGAGGTAAAGTGCAAAGGTAACGAAAGTAGCCTGTGGGACTGTCCGGCACGAAGG
TGGGGCCACAGCGAGTGTGGCCATAAGGAAGACGCGGCCGTGAACTGTACAGACATATCCGTACAAAAAACGCCCCA
AAAGGCGACGACCGGGCGATCATCAAGACAATCTAGCTTTATTGCCGTGGGAATTCTCGGTGTAGTGCTTCTTGCTA
TATTTGTCGCTTTGTTCTTTCTGACTAAAAAGCGCAGGCAAAGGCAGCGGCTTGCTGTGAGCTCTCGGGGAGAAAAC
CTCGTTCACCAAATCCAATACCGAGAAATGAACTCCTGTCTCAACGCCGACGATCTTGACCTGATGAACTCATCTGA
GAACTCACACGAGTCCGCCGATTTCAGCGCGGCGGAATTGATCTCTGTCAGCAAATTTCTGCCTATAAGTGGCATGG
AAAAAGAAGCCATACTCTCTCACACGGAAAAGGAAAATGGCAACCTT

### Example 8: Design of BME Construct

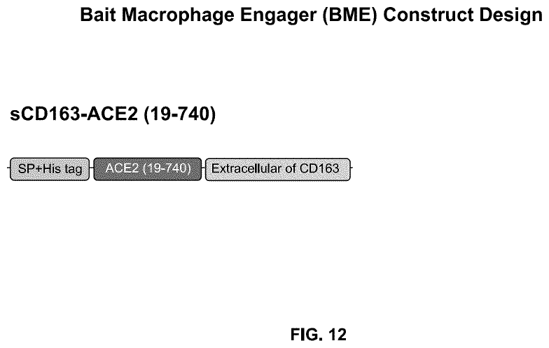

A bait macrophage engager (BME) construct was designed containing soluble CD163 (sCD163) fused to ACE2 (19-740). This sCD163-ACE2 (19-740) BME construct can be used as a substitution for neutralizing antibodies for SARS-CoV-2, instead of recruiting immune cells via Fc, it will recruit macrophages via CD163. A schematic for the BME construct is shown in FIG. 12.

The nucleotide sequence for sCD163-ACE2 (19-740) BME construct is provided as SEQ ID NO: 106:

ATGGGTTGGAGCTGCATTATCTTGTTTCTTGTCGCCACGGCTACGGGCGTTCATTCACACCATCACCACCATCATAG
CACCATCGAGGAGCAGGCAAAAACTTTTCTTGACAAGTTCAACCATGAGGCCGAAGACTTGTTCTATCAAAGCTCAT
TGGCGAGCTGGAATTATAATACAAACATCACGGAGGAAAATGTACAGAACATGAACAATGCAGGGGATAAATGGTCC
GCTTTTCTGAAAGAGCAATCCACTCTCGCACAAATGTATCCCTTGCAAGAGATACAAAACTTGACAGTGAAGCTTCA
GCTCCAGGCCCTGCAGCAGAATGGGTCCAGCGTCTTGAGCGAGGATAAATCCAAGCGCCTTAATACGATTCTTAACA
CGATGAGCACTATATACAGTACGGGCAAGGTGTGCAACCCCGACAATCCTCAAGAGTGCTTGCTTCTCGAGCCAGGC
CTTAACGAAATCATGGCAAACTCATTGGACTATAATGAGCGCCTCTGGGCGTGGGAATCTTGGAGATCTGAGGTTGG
TAAGCAGCTTCGACCTTTGTATGAAGAATACGTGGTATTGAAAAACGAAATGGCGCGAGCTAATCATTACGAAGACT
ACGGTGACTACTGGCGAGGAGATTATGAAGTGAATGGGGTAGACGGCTACGACTACTCTCGAGGGCAACTCATCGAA
GATGTTGAGCACACATTCGAAGAAATCAAACCACTTTATGAGCATCTCCATGCGTACGTACGAGCGAAACTCATGAA
CGCGTACCCCAGTTATATAAGTCCCATCGGTTGCCTCCCCGCGCATCTTCTTGGAGACATGTGGGGGAGATTCTGGA
CCAACCTCTATAGTCTTACTGTACCCTTCGGGCAAAAGCCGAATATAGATGTGACTGATGCTATGGTGGACCAGGCC
TGGGACGCACAAAGGATTTTTAAGGAAGCAGAAAAGTTCTTTGTATCTGTGGGGCTCCCCAATATGACTCAAGGGTT
CTGGGAAAACTCCATGCTGACAGATCCTGGGAACGTGCAAAAGGCCGTGTGTCACCCTACAGCGTGGGACCTTGGGA
AAGGTGACTTTAGAATTCTGATGTGTACCAAGGTGACTATGGACGATTTTTTGACCGCTCATCATGAGATGGGACAT
ATCCAGTACGATATGGCTTACGCAGCTCAGCCTTTCCTCCTGAGGAATGGCGCCAATGAGGGATTTCATGAAGCCGT
GGGCGAAATAATGTCTCTGAGCGCTGCTACTCCTAAGCATTTGAAAAGCATAGGCCTCCTCTCTCCCGACTTCCAAG
AGGACAACGAGACAGAAATTAATTTCCTCCTTAAACAGGCGCTCACCATAGTAGGGACATTGCCTTTCACATACATG
CTTGAGAAATGGAGATGGATGGTTTTCAAAGGGGAGATCCCCAAAGATCAGTGGATGAAGAAATGGTGGGAGATGAA
GCGGGAAATAGTTGGTGTGGTGGAGCCGGTCCCGCATGACGAGACCTATTGCGATCCAGCATCACTCTTTCACGTCA
GCAATGACTACTCTTTCATTAGATATTATACCCGCACTCTGTATCAATTTCAGTTCCAAGAGGCGTTGTGCCAAGCG
GCAAAACATGAGGGCCCCCTTCACAAATGTGACATATCCAACTCCACTGAAGCAGGCCAGAAATTGTTTAATATGCT
GAGACTGGGTAAGAGTGAACCATGGACTCTTGCCCTCGAAAACGTAGTCGGCGCCAAAAATATGAACGTTCGCCCCC
TGCTGAATTACTTTGAACCCCTCTTTACGTGGCTCAAAGATCAGAACAAAAATTCCTTCGTGGGGTGGTCCACAGAC
TGGTCACCTTACGCCGACCAGAGTATAAAAGTAAGGATTTCCCTTAAGAGTGCCCTGGGTGACAAAGCGTACGAGTG
GAACGACAATGAGATGTACCTTTTTCGCTCTAGCGTTGCATACGCTATGCGGCAGTACTTCCTGAAGGTTAAGAATC
AAATGATTCTTTTCGGTGAAGAAGATGTGCGAGTAGCAAATTTGAAGCCGCGGATTAGCTTCAACTTTTTCGTAACC
GCACCCAAAAACGTCTCAGATATTATCCCTAGGACGGAAGTCGAGAAAGCGATCCGGATGAGTAGGAGTAGAATTAA
TGATGCCTTTCGGCTGAACGACAACTCCCTTGAATTTCTTGGCATCCAGCCGACCCTTGGCCCGCCCAATCAGCCTC
CAGTGAGCAGCTCTCTGGGTGGGACGGATAAAGAATTGAGACTCGTTGACGGCGAGAATAAGTGCTCCGGACGCGTC
GAAGTGAAGGTTCAAGAGGAGTGGGGGACCGTGTGCAATAATGGCTGGAGCATGGAAGCCGTCAGCGTTATTTGTAA
TCAACTCGGATGCCCGACTGCTATCAAAGCACCAGGATGGGCCAATTCTTCTGCTGGGAGCGGACGCATTTGGATGG
ATCATGTTAGTTGTCGGGGTAATGAGAGTGCGTTGTGGGACTGCAAACATGATGGGTGGGGTAAACACTCTAATTGT
ACACATCAACAAGATGCGGGAGTGACGTGTAGTGACGGCTCCAATCTCGAAATGCGCCTTACAAGAGGAGGAAATAT
GTGCTCTGGGAGGATTGAAATCAAATTCCAAGGCCGGTGGGGCACAGTGTGCGATGATAATTTTAACATAGACCATG
CCAGTGTGATCTGCCGGCAGCTTGAATGTGGTTCTGCAGTCAGTTTCAGCGGCTCATCCAACTTCGGGGAGGGCTCA
GGGCCTATATGGTTTGATGACTTGATTTGCAACGGGAATGAGTCAGCACTGTGGAATTGTAAACACCAGGGATGGGG
CAAGCATAACTGTGACCATGCCGAAGATGCCGGCGTAATATGCTCCAAAGGTGCGGACCTCTCTCTCCGGCTTGTGG
ACGGTGTCACGGAGTGCTCTGGACGGTTGGAGGTCCGCTTTCAGGGGGAGTGGGGTACTATTTGCGACGATGGTTGG
GACTCCTACGATGCGGCTGTTGCATGCAAACAATTGGGATGTCCTACTGCTGTTACGGCAATCGGTCGGGTAAACGC
ATCAAAGGGGTTTGGGCATATATGGCTTGACAGCGTATCATGTCAAGGTCATGAACCAGCTATCTGGCAGTGTAAAC
ATCATGAGTGGGGAAAACACTACTGCAATCACAACGAGGATGCCGGGGTCACGTGCTCTGATGGTAGTGATCTCGAG
TTGAGGCTTCGGGGTGGCGGTTCAAGATGCGCAGGCACTGTCGAAGTCGAAATTCAGCGACTCCTGGGGAAAGTATG
CGATAGGGGCTGGGGTCTCAAAGAGGCCGATGTCGTTTGTAGACAGTTGGGTTGCGGCTCCGCTCTTAAAACATCCT
ACCAAGTTTATTCTAAAATCCAAGCGACTAATACTTGGCTCTTCTTGTCTTCCTGTAATGGTAATGAGACGTCACTC
TGGGATTGCAAAAATTGGCAATGGGGAGGTCTGACCTGTGACCACTACGAAGAAGCTAAGATTACGTGTAGTGCGCA
TCGAGAGCCTCGCTTGGTAGGAGGAGACATTCCTTGCTCAGGCCGCGTAGAAGTCAAACACGGGGATACTTGGGGTT
CTATCTGTGATTCAGATTTTTCACTTGAAGCTGCGTCTGTGCTGTGTAGGGAACTTCAATGTGGTACAGTCGTTAGT
ATTCTCGGGGGCGCCCATTTTGGTGAGGGAAATGGGCAAATTTGGGCAGAAGAATTCCAATGCGAGGGACACGAGAG
TCATCTTAGCTTGTGCCCCGTGGCGCCAAGGCCGGAAGGGACATGCTCTCACTCAAGAGATGTGGGAGTGGTGTGCT
CAAGATATACAGAGATCAGGTTGGTGAACGGGAAAACTCCTTGTGAGGGTCGAGTCGAACTTAAGACGTTGGGTGCC
TGGGGATCACTTTGCAATAGCCACTGGGACATTGAAGATGCCCATGTGCTCTGCCAACAACTCAAGTGTGGAGTCGC
TTTGTCCACCCCAGGCGGCGCTCGATTCGGTAAGGGAAACGGTCAAATCTGGCGGCACATGTTCCACTGCACTGGGA
CGGAGCAGCATATGGGTGACTGTCCGGTGACGGCTTTGGGCGCCAGCTTGTGTCCAAGCGAACAGGTTGCCTCCGTG
ATCTGCAGTGGCAATCAGTCTCAAACACTGAGCAGCTGCAACAGTTCAAGCTTGGGGCCGACTCGGCCGACCATACC
TGAGGAAAGTGCAGTCGCCTGCATCGAAAGTGGGCAATTGCGCTTGGTTAATGGCGGCGGGCGGTGCGCTGGCCGAG
TAGAGATTTATCATGAAGGTTCCTGGGGGACCATCTGTGATGACTCATGGGATCTTAGCGACGCCCACGTGGTATGT
CGCCAGCTGGGTTGTGGCGAAGCAATTAATGCGACAGGTTCTGCGCACTTCGGTGAAGGAACGGGGCCGATATGGCT
TGACGAGATGAAATGCAACGGTAAAGAATCAAGGATTTGGCAATGTCACAGCCACGGTTGGGGGCAACAGAACTGTA
GACACAAGGAAGACGCCGGCGTCATATGTTCAGAGTTTATGTCCTTGAGATTGACGAGCGAGGCCAGTCGAGAAGCT
TGCGCCGGGCGGCTTGAAGTTTTCTACAATGGAGCCTGGGGGACCGTGGGTAAAAGTAGTATGAGCGAAACCACAGT
AGGAGTAGTTTGTCGCCAACTTGGGTGTGCCGATAAGGGCAAGATTAATCCCGCTTCCCTTGATAAGGCGATGTCCA
TACCGATGTGGGTCGACAACGTGCAATGCCCAAAAGGACCTGATACACTTTGGCAGTGCCCTAGTAGTCCTTGGGAG
AAGAGATTGGCCAGTCCGTCTGAAGAAACTTGGATAACATGTGACAACAAGATACGACTTCAAGAGGGACCTACGTC
ATGTTCAGGTCGAGTGGAAATCTGGCACGGAGGATCATGGGGGACGGTTTGTGACGATAGCTGGGATCTGGATGATG
CCCAGGTAGTCTGCCAACAGCTCGGATGCGGTCCGGCGTTGAAGGCGTTCAAGGAAGCCGAGTTTGGCCAAGGCACA
GGACCAATTTGGCTTAATGAAGTGAAATGCAAGGGTAACGAAAGCTCTCTTTGGGACTGTCCGGCACGGCGGTGGGG
GCACAGTGAGTGTGGCCATAAGGAAGACGCAGCAGTGAACTGCACGGATATTAGTGTTCAGAAGACCCCGCAAAAAG
CGACGACCGGGCGGAGCTCCCGCCAGTCCAGT

### Example 9: CBR-Macrophage Cell Manufacturing

To avoid cell manufacturing for each individual, off-the-shelf CBR programed macrophage (CBRM) will be used. CBRM will be manufactured in advance using universal induced pluripotent stem cell-derived macrophages (iPSC-macrophages) that will be generated by knocking out B2M to eliminate all MHC I and subsequently knocking in HLA E (Hoerster et al., Frontiers in Immunology, 2021).

iPSC were generated from a healthy donor using Sendai virus kit (Thermo Fisher, Waltham, MA). iPSC were differentiated and polarized in culture into mature M1 macrophages using the protocol from Cao et al., Stem Cell Reports, 2019.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

1. A chimeric bait receptor (CBR) comprising:
a) an extracellular portion comprising a binding region that a virus specifically binds to, wherein the binding region is not an antibody;
b) a transmembrane portion; and
c) an intracellular portion comprising an intracellular signaling region of an endocytic receptor.

2-64. (canceled)

65. A nucleic acid encoding the CBR of claim 1.

66-67. (canceled)

68. A recombinant vector comprising the nucleic acid of claim 65.

69. A cell comprising the nucleic acid of claim 65.

70-72. (canceled)

73. A pharmaceutical composition comprising a therapeutically effective amount of the CBR of claim 1 and a pharmaceutically acceptable carrier.

74. A method of treating or preventing a viral infection in a subject in need thereof comprising administering to the subject a therapeutically effective amount of the CBR of claim 1.

75-78. (canceled)

79. A chimeric phagocytic receptor (CPR) comprising:
a) an extracellular portion comprising an antigen binding portion,
b) a transmembrane portion; and
c) an intracellular portion comprising an intracellular signaling region of an endocytic receptor, wherein the CPR does not comprise a recruitment portion, wherein the recruitment portion is not the endocytic receptor intracellular region, and wherein the recruitment portion binds to a cytosolic protein of a phagocytic signaling pathway.

80-141. (canceled)

142. A nucleic acid encoding the CPR of claim 79.

143-144. (canceled)

145. A recombinant vector comprising the nucleic acid of claim 142.

146. A cell comprising the vector of claim 145.

147-149. (canceled)

150. A pharmaceutical composition comprising a therapeutically effective amount of the CPR of claim 79 and a pharmaceutically acceptable carrier.

151. A method of treating cancer in a subject in need thereof comprising administering to the subject a therapeutically effective amount of the CPR of claim 79.

152. (canceled)

153. A bait macrophage engager (BME) comprising:
a) a binding region that a virus specifically binds to, wherein the binding region is not an antibody; and
b) a ligand for an endocytic receptor.

154-170. (canceled)

171. A nucleic acid encoding the BME of claim 153.

172-173. (canceled)

174. A recombinant vector comprising the nucleic acid of claim 171.

175. A cell comprising the vector of claim 174.

176. (canceled)

177. A pharmaceutical composition comprising a therapeutically effective amount of the BME of claim 153 and a pharmaceutically acceptable carrier.

178. A method of treating or preventing a viral infection in a subject in need thereof comprising administering to the subject a therapeutically effective amount of the BME of claim 153.

179-182. (canceled)

183. An antigen macrophage engager (AME) comprising:
a) an antibody that binds to an antigen expressed on a surface of a cancer cell; and
b) a ligand for an endocytic receptor.

184-194. (canceled)

195. A nucleic acid encoding the AME of claim 183.

196. A recombinant vector comprising the nucleic acid of claim 195.

197-198. (canceled)

199. A pharmaceutical composition comprising a therapeutically effective amount of the AME of claim 183 and a pharmaceutically acceptable carrier.

200. A method of treating cancer in a subject in need thereof comprising administering to the subject a therapeutically effective amount of the AME of claim 183.

201. (canceled)
